(19) United States
(12) Patent Application Publication
(10) Pub. No.: US 2005/0249697 A1
(43) Pub. Date: Nov. 10, 2005

(54) Compositions and methods for the inhibition of bone growth and resorption

(76) Inventors: Kathryn E. Uhrich, Plainfield, NJ (US); Robert D. Harten, Kennett Square, PA (US)

Correspondence Address: MAYER, BROWN, ROWE & MAW LLP, P.O. BOX 2828, CHICAGO, IL, 60690-2828, US

(21) Appl. No.: 10/949,955

(22) Filed: Sep. 24, 2004

Related U.S. Application Data
(60) Provisional application No. 60/505,402, filed on Sep. 24, 2003.

(51) Int. Cl. A61K 31/765

(52) U.S. Cl. 424/78.37

(57) ABSTRACT

A composition, or article for inhibition of bone growth and resorption comprises an anti-inflammatory agent(s), optionally other agents and carriers, monomer(s), oligomer(s), polymer(s), salt(s), mixtures(s), dispersion(s) and/or blend(s) thereof, which composition, device, or implant upon polymer erosion releases a bone growth and/or bone resorption retarding, reducing or inhibiting amount of the agent(s). The monomers, oligomers and polymers, releasing active or activatable agent(s), have pre-selected properties such as molecular weight, flexibility, hardness, adhesiveness, and other valuable properties. The monomers, oligomers and polymers may be prepared by a process involving various alternative and sequential steps that allow the design a priori of products with specific characteristics. The composition, device, implant or dressing of this patent are suitable for retarding, reducing or inhibiting bone growth or bone resorption, comprising administering or applying to a subject's pre-selected site a bone growth or resorption reducing amount of the agent(s).

### RELATED APPLICATIONS

This application claims priority to U.S. Provisional Application 60/505,402 filed Sep. 24, 2003; Ser. No. 10/823,435 filed Apr. 12, 2004 entitled “Therapeutic Polymers and Uses thereof”; U.S. Ser. No. 10/861,881 and PCT US 04/17916 filed Jun. 4, 2004 entitled “High Molecular Weight Polymers and Methods for Making Same”; USSN PCT US 03/34183 filed Oct. 28, 2003 entitled “Therapeutic Compositions”; U.S. Ser. No. 10/716,577 and PCT US 03/36925 filed Nov. 18, 2003 entitled “Medical Devices Employing Novel Polymers”, the contents of all of which are incorporated herein by reference to the extent permitted by law.

### BACKGROUND OF THE INVENTION

1. Field of the Invention

This invention relates to the local administration of anti-inflammatory agents, and other optional agents, in the form of a composition, and articles such as a device, implant or dressing employing a monomer(s) that comprises at least one anti-inflammatory agent(s) and other optional agents and linkers, or oligomer(s), polymer(s), salt(s), mixtures(s) or blend(s) thereof. The composition and articles, upon erosion under appropriate conditions, release a bone growth or bone resorption reducing amount of the agent(s).

2. Description of the Background

The safe and effective delivery of an agent(s) to a specific location associated in general with lesser side effects than more widespread delivery. Site-specific delivery is particularly desirable for the treatment of localized conditions such as cancer, orthopedic and dental conditions, wounds, and arthritic conditions, to name a few. The use of polymers for drug delivery began with the development of controlled-release oral formulations coated with a non-therapeutic polymer. Many such formulations, however, induce inflammation or host responses at the delivery site, or have low and/or unpredictable potency, breakdown products, non-zero-order release rates, burst effects (drug delivery spikes), or other untoward effects. Devices such as grafts, implants, and surgical and bone healing devices frequently induce, or are associated with, undesirable side effects that include pain, inflammation, swelling, infection, adjacent tissue hyperproliferation, capsule, and foreign body response, such as granuloma or fibroma formation surrounding their insertion. Although more biocompatible polymer coatings and other surface technologies were developed in order to reduce these effects, the polymers employed are in some instances either not biodegradable, or are inherently highly inflammatory and unpredictable in nature. Non-biodegradable coatings, in addition, may sometimes suffer from fatigue over time and/or delaminate in situ.

Polymers containing therapeutic and other agents incorporated into a polymer backbone in formulations and devices have been described for use in medical and other applications. Many polymers, however, have limitations associated with, for example, adhesion characteristics, and temperature dependency that detract from widespread use. Certain applications require the use of resilient materials and tenacious films that are composed of polymers of substantial molecular weight, many times in excess of 100,000 Dalton. As is known in the art, the physical characteristics of a polymer depend on its molecular structure. Discreet monomer units of regular structure, for instance, tend to form crystalline or semi-crystalline materials, whereas polymers of irregular structure such as random copolymers tend to be amorphous. For some applications, a polymer needs to be solvent-cast into a tough film or coating, or molded under pressure into a shaped article, and then subjected to sterilization by ionizing radiation or electron beam bombardment, which seriously affect the polymer's molecular weight. High molecular weight polymers of desirable qualities such as a pre-determined polydispersity index (MW/Mn) are produced by controlled branching and the formation of large ring macrocyclic oligomers exhibiting flexibility (or rigidity), adhesiveness, hardness, biocompatibility, processability temperature range, loading capacity, duration of delivery, and others, while at the same time limiting or avoiding the above described disadvantages.

Anti-inflammatory agents have been reported suitable for delivery by themselves, or in various compositions that include sustained release compositions and device. Polymers such as anhydride, ester, and amide polymers carrying anti-inflammatory agents in their backbones or appended to polymer side chains are known. These polymers degrade to release the agents under physiologic conditions. Other types of sustained release anti-inflammatory polymers and devices are also known. The local administration of anti-inflammatory agents to the palatal bone has been disclosed as promoters of bone growth. In many instances it is desirable to inhibit bone growth. For example, osteophytes (bone spurs), commonly occurring around joints and in the spine, are a very common condition characterized by a bone outgrowth. Osteophytes are associated often with osteoarthritis, and are known increase in frequency with age. Another condition involving unwanted bone growth is heterotopic ossification (HO) that may be characterized by inappropriate differentiation of cells into bone-forming cells. This condition leads to bone formation, usually near joints, where the bone formation often limits the mobility of the joint. HO may follow neurological injury and direct injury to soft tissue such as muscles or connective tissue around the joint in which HO later develops. In the case of an elbow fracture or dislocation, the subsequent incidence of HO at the elbow is said to approach 90%. It may be desirable as well to inhibit bone growth following a bone fracture because new bone growth prior to setting may impair proper healing of the fracture site afterwards. Surgical procedures, for instance following a spinal laminectomy, new bone growth can impinge on the spinal cord and cause complications such as pain, numbness, paralysis, and may lead to undesirable bone growth. In addition, there is also a need to inhibit bone loss that is generally associated with inflammation and bone injuries. The drug celecoxib® reduces inflammation by inhibition of the COX-2 enzyme, and its systemic administration reduced bone loss produced by titanium particle placement on calvaria bones in mice. Osteomyelitis, an acute or chronic bone infection caused by bacteria or fungi, is another condition that will benefit from bone loss inhibition. Often bone infections are associated with the implantation of an orthopedic device, and treating the infection often requires the removal of the device. Bone infections typically cause at least some bone loss through degradation or resorption of bone, and surgery is required often to remove dead bone or to fill the open space left behind by bone resorption with a bone graft or packing material. In some instances the bone loss may be severe enough to require amputation. In such cases, a reduction of bone resorption would decrease the need for surgery and amputations while promoting recovery from bone infection by maintaining healthy bone tissue at the infection site. Thus, there is a need for novel articles and methods to slow, lessen, prevent and treat bone degradation or resorption and bone growth at the site of a wound, injury, and articular joint, as well as at a site of surgery.

### SUMMARY OF THE INVENTION

This invention relates to a composition and articles such as a device, implant and dressing, among others employing a monomer comprising at least one unit of an anti-inflammatory agent(s), and optionally additional agents and linkers. The composition and article are suitable for delivering the agent(s) at a site of injury, surgery, bone replacement or bone implantation, among others, at a high concentration and/or for a prolonged period of time, to reduce, preclude or inhibit tissue and/or bone growth at the site. A more complete appreciation of the invention and other intended advantages will be seen by reference to the following drawings accompanying this patent.

### BRIEF DESCRIPTION OF THE DRAWINGS

FIG. 1 shows the extent of bone resorption and bone formation at a bone defect site in rats treated with: a homopolymer containing salicylic acid (filled column), a copolymer containing salicylic acid (open column), and a collagen control (stripped column).

FIG. 2 shows a graph that includes some of the possible bonds and their distribution that may be incorporated into the compounds suitable for inhibiting bone growth and resorption in accordance with this invention. A more complete appreciation of the invention and other intended advantages may be readily obtained by reference to the following detailed description of embodiments of the invention.

### DETAILED DESCRIPTION OF THE PREFERRED EMBODIMENTS

This invention arose from a desire by the inventors to provide a significant improvement on the management of bone injury and of surgery by delivering an agent(s) to a bone and its tissue environment for preventative, therapeutic and other purposes in a manner such that avoids or minimizes bone growth and resorption. Prior reports in the literature claimed to have fostered bone growth upon local administration of anti-an inflammatory agent(s) to a palatal bone. In contrast to the prior art disclosure, the present inventors found that the site specific administration of an anti-inflammatory agent(s), such as salicylic acid, inhibits bone growth and resorption and, thus, may be employed to preserve the status of, for example, a fractured bone until it is set. Following removal of an implanted device, e.g. an artificial joint, anti-inflammatory administration is effective for bone preservation and maintenance until a new device may be implanted. The anti-inflammatory agent(s) may be administered by itself or in the form of a monomer, oligomer, polymer, polymer blend, mixture, dispersion, etc., article and the like, optionally with an additional agent(s). Briefly, it is believed that prostaglandins have a biphasic effect on bone growth; that is, low concentrations of prostaglandins stimulate the activity of osteoblasts, the cells responsible for new bone synthesis and, thereby stimulate bone formation. Higher concentrations of prostaglandins, however, decrease osteoblast activity. Based on their results, the inventors believe that the presence of extremely low prostaglandin concentrations or in their absence decreased osteoblast activity results in inhibition of bone formation. Similarly, in the presence of low prostaglandin concentrations or absence thereof, the activity of osteoclasts, the cells responsible for bone resorption, decreases and results in inhibition of bone resorption. Thus, the localized delivery of anti-inflammatory agents prevents bone formation that leads to bone fusion and, therefore, prevents ectopic bone formation.

This invention therefore relates to the delivery of anti-inflammatory agents, their monomers, oligomers, polymers, formulations, and medical articles, all of which release, under appropriate conditions, one or more agents that are active upon delivery, or are activated in situ by hydrolysis or other processes. The agent(s) may be administered by itself(themselves), as a composition comprising its(their) monomer(s), oligomer(s), polymer(s), salt(s), blend(s), mixture(s), dispersion(s), and the like, or as an article, e.g. a device, implant or dressing, among others, all of which may contain high loads of the active or activatable agent(s), e.g. about 70 wt % to about 90 wt %, and in many instances even higher, and/or release the agent(s) over a prolonged period of time. These are highly potent products that provide an excellent means for controlled or sustained delivery of the anti-inflammatory agent(s). These polymers and compositions may be used to form, or to coat, medical and veterinary articles, or they may be provided as a delivery formulation comprising nano- or micro-particles in the form of spheres or other desired shapes. The compositions and articles may be used as carriers for other agents to be released as the polymer degrades as well. In addition, the anti-inflammatory agent(s) itself(themselves) may be carried by any biocompatible polymer, either dispersed, appended, blended, or otherwise associated with the polymer and/or other agents carried therewithin. For historical reasons many of the polymers, their chemical structures, physical characteristics, and synthetic routes will be described in this patent with reference to specific anti-inflammatory drugs, e.g. salicylic acid and diflunisal. Some of the characteristics of these compounds are shown in Table 1a below. The overall concepts and description, however, are intended broadly to encompass all types of agents, compositions, formulations, and devices, and their applications.

As indicated above, the inventors surprisingly observed that the local delivery of salicylic acid to a site of a mammal's bone defect or injury prevents new bone growth, degradation and resorption. This observation was made subsequent to the placing of an implant made of polymer microspheres containing ≧0.5 g salicylic acid/cm³ at a site of injury in a femur containing both osteoclasts, i.e. cells responsible for bone resorption, and osteoblasts, i.e. cells responsible for new bone synthesis. It became evident, thus, to the inventors that the site directed or localized administration of an anti-inflammatory agent could be used to delay or prevent growth of unwanted bone at, or about, an injury site as well as at a lesion site until such time that the injury or lesion may be properly treated and/or fully healed. The inventors additionally envisioned the application or delivery of the agents of the invention at the time of new artificial joint implantation and following the removal of an artificial joint. In the latter case, when an artificial joint replacement becomes infected it must be removed, and the surrounding area becomes intensely inflamed, and in the absence of treatment results in bone loss. The localized administration of an anti-inflammatory agent taught by this patent will prevent the invasion of new bone or other tissue, and prevent bone loss while the infection may be being treated, sometimes up to several weeks or months.

TABLE 1
Anti-Inflammatory Properties of Salicylic Acid and Diflunisal

| | Salicylic Acid | Diflunisal |
|---|---|---|
| Property | 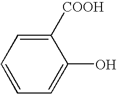 | 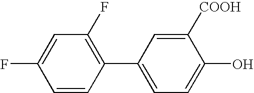 |
| Molecular Weight | 138 | 250 |
| Water Solubility | High | Very Low |
| Plasma half-life (hours) | 2.5 | 8 to 12 |
| Clinical Use | | |
| Single Oral Dose (mg) | 650 | 500 |
| Repeated Dosing | 650 mg (4 × Day) | 250 to 500 mg (2 × Day) |
| Plasma Levels* (μg/ml) | 150 to 300 | 50 to 190 |
| LD₅₀ (μg/kg) | 1,300 | 439 |
| Metabolism | | |
| No. Metabolites | ≧10 | 2 |
| Where Metabolized | Liver, Intestine and | Liver, Intestine |
| | Other Tissues | |

*Anti-Inflammatory Effectiveness

I. Glossary

The following definitions are used throughout this patent, unless otherwise indicated. The article “a” and “an” as used herein refers to one or to more than one, i.e. at least one, of the grammatical object of the article. By way of example, “an agent or agent(s)” means either one or more than one agent. As used herein, an “agent” may be a chemical or biological compound that is suitable for direct administration or for incorporation into the polymer, formulation, or device of this patent, and includes “inactive”, “active” and “activatable” forms of the agent(s); an “active agent” refers to a substance that has a physiological effect when present in a living system; an “activatable agent” refers to an agent or agent precursor that may be activated either upon, or subsequent to, its release by any mechanism. More generally an agent may be a compound that has utility. For example, an agent may be a marker or tracer, a compound that has an effect for a certain application, be it for use to ascertain, diagnose, foster or impede biological life, or otherwise. An “agent” may be a drug or therapeutic compound, or compound precursor, etc. used to treat a specific disease or medical condition. “Biologically active” refers to an agent that may be active or activated and/or exhibits some effect when applied to a living system. A “therapeutically active” agent refers to an agent having therapeutic properties in a living system, e.g. aiding in the prevention or treatment of an undesired occurrence or condition such as inflammation or bone growth or resorption. A “physiologic effect” refers, for example, to an effect on the functioning of an organism, such as altering normal or abnormal function, and/or obliteration or restoration of function. A physiologic effect may include, but is not limited to, binding to a biomolecule, i.e. DNA, protein, carbohydrate, or lipid, inhibiting of enzyme activity, and sequestering of small molecule cofactors, e.g. metal ions or amino acids, and the like. The term ester linkage refers to —OC(═O)— or —C(═O)O—; the term thioester linkage refers to —SC(═O)—, —OC(═S)—, or —C(═O)S—; the term amide linkage refers to —N(R⁷)C(═O)— or —C(═O)N(R⁷)—, the term urethane or carbamate linkage refers to —OC(═O)N(R⁷)— or —N(R⁷)C(═O)O—, wherein each R⁷ may be a suitable organic radical, such as, for example, hydrogen, (C₁-C₄₀)alkyl, (C₃-C₄₀)cycloalkyl, (C₃-C₄₀)cycloalkyl (C₁-C₄₀)alkyl, aryl, heteroaryl, aryl (C₁-C₄₀)alkyl, or heteroaryl (C₁-C₄₀)alkyl; and the term carbonate linkage refers to —OC(═O)O—. “Aryl” denotes any aromatic residue, including phenyl and ortho-fused bi- or tri-cyclic carbo- or hetero-cyclic residue having about 4 to 40 ring atoms in which at least one ring may be aromatic. “Heteroaryl” refers to a radical attached via a ring carbon or heteroatom, or via an appended chain of an aromatic ring containing 3 to 40 ring atoms consisting of carbon and heteroatoms comprising e.g. O, S, P, or N, which may be substituted by R, wherein R may be absent or H, O, halogen, (C₁-C₄₀)alkyl, (C₃-C₄₀)cycloalkyl, (C₃-C₄₀)aryl, including phenyl, benzyl, and bicyclic structures, all of which may be further substituted by a heteroatom, e.g. a (C₃-C₄₀)heterocyclic group, particularly a benzyl derivative or one derived by fusing a propylene, trimethylene, or tetramethylene diradical thereto. As used herein, “administering an agent near the site” means applying the agent at, or proximal to, a given site to produce a desired or stated therapeutic effect in a localized manner, e.g. to reduce bone resorption, stop bleeding, or foster bone growth at the site. “Alkyl”, “alkoxyl”, etc. may denote both straight and branched groups; a reference to an individual radical such as “propyl” may denote a straight chain radical; a branched chain isomer such as “isopropyl” being specifically referred to. As used herein, an agent may be “appended” to a polymer when the agent may be bonded or complexed to the polymer as a side chain or side group, but is not part of the polymer backbone. As used herein, an agent or functional group may be “associated” with the polymer by one of many forms, including by direct, linear integration (i.e. chemical bonding) into the polymer backbone, as a side chain or side residue chemically bonded to the polymer backbone not part of the backbone, electrostatic bonding to the polymer backbone, linkage to the polymer backbone through a linking group, pendent (i.e. an off-shoot of the backbone) neither oligomeric nor polymeric, attachment to the polymer backbone, or bonding to one or more endings of the backbone. The association used will depend on the functional characteristics (e.g. number and type of reactive groups) of the functional group. The agent may be bonded to the polymer preferably through a breakable linkage that will release it when applied or administered according to the methods of the invention. For example, an agent or compound may be linked to a polymer through a hydrolyzable linkage such as an anhydride or ester linkage. Others, however, are also suitable.

A substance is said to be “bioabsorbable”, but not necessarily biocompatible or biodegradable, when it may be absorbed by, whether integrated or not into, a living system. A substance is said to be generally “compatible”, e.g. “biocompatible”, when it has properties that do not conflict with a system, e.g. a living system, i.e. it is not detrimental to the general existence and functioning of the system, e.g. neither toxic to, nor causes e.g. a detrimental immunological reaction in a living system, so that it would make it undesirable to continue its use. A substance is said to be “degradable”, e.g. “biodegradable”, when it is broken down into components smaller than its original size when in a target, e.g. living, system. A “diagnostic agent or compound” refers to a substance that may be employed to assess a certain status or presence by a known means. A “tracer” or “marker” refers to an agent or compound that, although it may or may not have its own activity, may be located when placed in a pre-determined position, or it may be followed to ascertain where it lodges, therefore providing information on the path it followed and its current location. “Therapeutically active compounds or agents”, or “detectable, diagnostically, veterinarily or therapeutically active compounds or agents” include diagnostic and therapeutic agents or compounds that provide a diagnostic, preventative or therapeutic effect when administered to a subject, e.g. an animal such as a mammal including a human whether they are active upon, or are activated after, delivery. A “functional group” refers to a chemical residue or moiety that may be incorporated into a polymer, e.g., into an ester, thioester, or amide linkage of a polymer as discussed in detail below, such that it releases the agent or its precursor upon erosion or breakage of the polymer, e.g. hydrolysis, enzymatic breakage for example by esterases. These groups may independently be a hydroxy group (—OH), a mercapto group (—SH), an amine group (—NHR), a carboxylic acid (—COOH), a halo that comprises fluoro, chloro, bromo, or iodo, and others known in the art. The term “amino acid” refers to residues of the natural amino acids, e.g. the D or L forms of alanine (Ala), arginine (Arg), asparagine (Asn), aspartic acid (Asp), cysteine (Cys), glutamic acid (Glu), glutamine (Gln), glycine (Gly), histamine (His), isoleucine (Ile), leucine (Leu), lysine (Lys), methionine (Met), phenylalanine (Phe), proline (Pro), serine (Ser), threonine (Thr), tryptophan (Trp), tyrosine (Tyr), and valine (Val), and non-natural amino acids, e.g. phosphoserine, phosphothreonine, phosphotyrosine, hydroxyproline, gamma-carboxyglutamate; hippuric acid, octahydroindole-2-carboxylic acid, statine, 1,2,3,4-tetrahydroisoquinoline-3-carboxylic acid, penicillamine, ornithine, citruline, c-methyl-alanine, para-benzoylphenylalanine, phenylglycine, propargylglycine, sarcosine, and tert-butylglycine, among many others. The term “amino acid” also comprises natural and non-natural amino acids bearing a conventional amino protecting group, e.g. acetyl or benzyloxycarbonyl, as well as natural and non-natural amino acids protected at the carboxy terminus, e.g. as a (C₁-C₆) alkyl, phenyl or benzyl ester or amide; or as an α-methylbenzyl amide. Other suitable amino and carboxy protecting groups are known to those skilled in the art, and are included within the context of this invention. See, for example, Greene, T. W. and Wutz, P. G. M. “Protecting Groups in Organic Synthesis”, Second Edition, New York, John Wiley & Sons, Inc., 1991, and references cited therein. Similarly, the term “carbohydrate”, “oligosaccharide” or “polysaccharide” refers to known natural and synthetic forms of these compounds, and “nucleic acid”, “DNA”, “RNA”, “iRNA”, and others refer to their single and double stranded forms and have the accepted meaning in the art. The term “peptide”, “oligosaccharide” and oligonucleotide” refer to sequences of about 2, 3, or 5 to about 15, 20, or 35 and more amino acids, carbohydrates and nucleic acids or residues thereof as defined above. The peptides or peptidyl residues may be linear or cyclic, such as those that may be prepared or result from the formation of disulfide bridges between two cysteine residues. Peptide derivatives may be prepared as disclosed in U.S. Pat. Nos. 4,612,302; 4,853,371; 4,684,620, or by employing other methods known in the art. As used herein, “physiological conditions” refers to conditions in a physiological system or environment, such as a mammal, e.g. a human, and may be “normal physiological conditions” such as those encountered in a normal, healthy subject or patient, or “abnormal physiological conditions” such as those in an unhealthy, sick, or injured subject or patient. Physiological conditions may be found, for example, inside a mammal, or on the surface of a mammal, such as in skin or hair. An agent may be considered to be “physiologically irrelevant” when it does not perform a physiological or biological function, such as for example when incorporated into a polymer backbone. When an agent may be chemically coupled within the polymeric structure it is generally unavailable to interact with a target or perform a physiologic function, and may be considered “physiologically inactive”, even if freely available in the biological milieu. An agent may be considered “physiologically relevant” when in a chemical form in which it may perform its desired biological function, e.g. interacting with a biological molecule, or sequestering of a relevant substance. Even though it may be present in a physiologically relevant form, an agent may not be “active” in a physiological environment, when it is for example dispersed in, or sequestered inside empty spaces of the polymer and, therefore unavailable to the surrounding milieu. As a result, even though it may be present in a biologically active form, its biological activity may be nil until released. A physiologically relevant agent is said to be “physiologically active” when it is available to the surrounding biological milieu and actively involved in its biological role. An agent is said to be “activatable” when it becomes active or adopts its active form upon release, hydrolysis or other action effect on its “precursor”. As used herein, the term “healing” means the repair of a defect or non-normal condition or state, and it may be applied to a living or non-living entity. When applied to a living entity healing refers to the restoration of health or the process of a return to health. When applied to a non-living entity, “healing” refers to the return to a normal or acceptable state, or to the fixing of a condition so that the entity may be operational.

The term “injury” includes physical trauma, as well as a localized infection or a localized disease process, such as the spontaneous development of a bone spur or heterotopic ossification at a site. The term “injury” includes a surgical procedure, such as implanting or removing an orthopedic device, or a deep bone infection as well. “Inhibiting”, “retarding”, “reducing” and “impeding” bone growth and/or resorption are intended for use as either equivalent terms or terms designating different degrees of prevention of bone growth and resorption. Thus, “inhibiting bone growth or resorption” refers to the administration of an agent under conditions, e.g. concentration, rate and/or release of the agent and/or its administration length and/or conditions, such that the amount of bone growth may be less than the amount that is observed when the agent is not administered. For example, in certain embodiments of the invention, bone growth can be inhibited by at least about 99%, 95%, 90%, 75%, 50%, or 25% in the presence of an agent or composition of the invention when compare to growth of bone in the absence of an agent or composition of the invention. In other embodiments of the invention, bone growth can be completely eliminated, or eliminated over a selected time period. The administration of an agent “at a site of injury” means locally administering the agent so that it may be in direct contact with the injured bone; or locally administering the agent to a location proximal to the injured bone, so that the agent can produce the desired or stated therapeutic effect, e.g. reduce bone resorption or bone re-growth at the site. An agent “formulated for controlled release” means that it may be formulated so that it will be released over an extended period of time when administered according to this invention. The agent may conveniently be formulated, for example, so that it will be released over a period of at least about 2, about 5, about 12 about 24, or about 48 hours, or over at least about 2, about 5, about 10, about 20, or about 40 days. Preferably, the agent may be formulated so that it may be released over at least about 5 or about 10 days. The agent can also preferably be formulated so that it may be released over a period of about 30 to about 90 days. An agent is said to be “appended” to a polymer when the agent may be bonded to the polymer as a side chain or side group, but is not part of the polymer backbone. The agent may be bonded preferably to the polymer through a linkage that is suitable to release the agent when the polymer is administered according to the methods of the invention. The agent may conveniently be linked to a polymer through a hydrolyzable linkage such as an anhydride or ester linkage. An agent is said to be “entrapped or dispersed in a polymeric matrix” when it is located within the matrix of a polymer such that it can be released in a controlled fashion when placed within the body. Preferably, the polymer matrix comprises a bio-degradable polymer. The term “setting or fixing the fracture” means to hold the fractured bone pieces together, or to stabilize the fracture. “An article of manufacture” is meant to include any product for medical or veterinary use, including “devices”, “implants”, “dressings”, and many others known in the art. A “device” includes all articles that may have medical or veterinary applications, whether for external or internal application. A “device” may be an “implantable device” when intended for internal application, whether permanent or removable. An “implant” may include a device such as an orthopedic device, e.g. an artificial joint, an artificial tooth, a film, paste, gel, fiber, chip, microspheres, nanoparticles or coating on a device, intended for insertion into a wound, or completely or partially inserted into a mammalian body. The term “dressing” refers to an object that may be placed in contact with a wound or injured or exposed tissue to cover the injured or exposed tissue, or deliver an agent to the tissue. The “release” of an agent refers to the delivery of an agent in a form that may be bioavailable and/or free, and includes the degradation of a polymer where the agent may be incorporated into, or appended to, the backbone. The term “release” also includes the degradation of a polymer that entraps agent molecules within its matrix, thereby allowing the free agent to make direct contact with the surrounding tissue or bone. The term “release” also encompasses administration of an agent in a form that may be immediately bioavailable, i.e. not a sustained release formulation. The agents of the invention may be administered by themselves in amounts effective to prevent, diminish or inhibit bone growth or resorption. The polymers of the invention form biodegradable bonds within the backbone of the polymer that may be broken by regular hydrolysis, proteolysis, or other biological or biochemical processes when placed in contact with an aqueous environment, microorganisms, body tissues, fluids, and the like. A substance is said to be “resorbable”, e.g. “bioresorbable”, when its material may be a naturally occurring material, e.g. in a living system, and may be capable of being absorbed by, and integrated into, a system, e.g. the living system, when placed into it or when created and subsequently placed in the system. As used herein, the term “dispersed through the polymer matrix” means that an agent or compound may be located within a matrix, for example a polymer by mixing, spreading, sprinkling, thoroughly mixing, physically admixing, or dispersing in the polymer matrix, among others, so that it may be released in a controlled manner over a period of time when placed in a system, e.g. within a living host. As used herein, the term “dissociate” indicates that an agent, compound, or substance may be separated or broken into smaller parts that may be chemically similar to the undissociated whole or they may be chemically dissimilar to the undissociated whole. Chemically dissimilar dissociation products may be heterogeneous or homogeneous with respect to either chemical properties or size, or both. Dissociation products may also be able to recombine to recreate the original undissociated whole, or they may remain permanently dissociated. Dissociation may occur spontaneously, as an inherent property of the undissociated whole, or as a result of a physical or chemical process, such as hydrolysis of the undissociated whole. The term “formed into” includes a polymer, compound, composition, or formulation of the invention that may be physically placed into various shapes, geometries, structures and configurations including, but not limited to a film(s), coating(s), fiber, rod, coil, suture, closure, sealer, sphere, pin, corkscrew, hook, cone, pellet, tablet, tube (smooth or fluted), disc, membrane, formulations comprising microparticles, nanoparticles, and/or “biobullets” (i.e., bullet shaped), seed (i.e., bullet shaped or targeted seeds), sleeve, cuff, free standing film, sheath, wrap, tube, cuff, stitches, formed gel, etc. A “sleeve” may be a physical conformation of a compound, agent or article that may be placed adjacent to and fits around or covers a second compound, agent or article, for example a medical or therapeutic device. A plastic coating surrounding a metal rod may be considered to be a sleeve for the rod. A sleeve may also be placed adjacent to a separate compound, agent or article without completely enclosing the latter. A sleeve may describe a compound, agent or article that may be formed into, for example, a coating, a film, a sheath, a wrap, a tube, a cuff, or a formed gel partially or wholly surrounding a second compound, agent or article, such as a medical device or implant.

As used herein, a substance is said to be “solid” when it has three dimensions and has the properties of a solid; namely it is not in liquid or gaseous form For example, a piece of paper, a metal rod, and a steel needle are all considered to be solids in the context of this patent. A substance is said to be “semi-solid” when it has some properties of a solid, and some of a liquid; for example it may be easily deformable by physical or chemical action. For example, gel and clay are “semi-solids” in accordance with this definition. As used herein formulated for “controlled release” refers to an agent formulated to be released over an extended period of time when administered according to this invention. For example, the agent may be formulated for release over a period of at least about 1, 2, 5, or 10 hours, about 1, 2, 5, 10, 20, 40, or 90 days, about 1, 2, 4, 6, 9, or 12 months, or 1 or more years. The agent may be formulated for release over about 1 to about 10 or more days, or for a longer period that may extend over months, and sometimes years if needed by regulation of the polymer characteristics. For the treatment of hard tissue, the agent may be formulated for release over about 1, 4, 8, 15, 30 days, months or years to about 45, 60, 90 days, months, or years, and for the treatment of soft tissue over about 1, 2, or 3 to about 5, 10, or 30 days or longer. As used herein, the term “hard tissue” includes tissue that has become mineralized, such as, for example, bone, cartilage, or both. The term “host” includes animals, such as a mammal, e.g. a human. For purposes of the present invention a “low molecular weight agent” includes any compound with, but not limited to at least two functional groups that may be employed for its polymerization; that may be for example one carboxylic acid group and at least one amine, thiol, carboxyl, amide, alcohol, azo or phenol, the agent having a specific activity, e.g. pharmaceutical activity, and up to about 1000 molecular weight. By “article” or “article of manufacture” it is meant a device, implant, or other product intended for medical or veterinary use. By “device” it is meant a structure that may be formed of, or covered by, a polymer of the invention. Devices may be used for different applications on living systems. Devices of different shapes may be designed for implantation close to the bone to be “fixed”, as an aid to affix the bone parts during fusion, or as a full or partial replacement of that bone, among others. A “medical device” or “medical implant” refers to a therapeutic device or a therapeutic implant, respectively, that may be used specifically for a medically-related purpose. For example, a bone, bone “screw”, “cuff”, “tube”, “wafer”, “tablet”, or “pin” are both medical devices and medical implants. Other forms of the device are also suitable. An article of manufacture or device, whether therapeutic or otherwise, may comprise more than one component. A therapeutic device that may be either temporarily or permanently placed either partially or wholly inside a living system may also be referred to as a “therapeutic implant”, and the agent may become active upon implantation, or activated thereafter. The administration or application of an agent “to” or “near a tissue” refers to the delivery of agent to a location proximal to, or in direct contact with, the tissue to produce the desired localized therapeutic effect. A “veterinary device” refers to a device that may be adapted specifically for use in an animal, whether wild, domesticated, marine, or zoological animal, among many others. As used herein, the term “injury” includes physical trauma, as well as a localized infection or a localized disease process, such as the spontaneous development of a bone spur or heterotopic ossification at a site. The term “injury” also includes a surgical procedure, such as implanting or removing an orthopedic device, or a deep bone infection.

The term “inhibiting bone growth” refers to administering an agent under conditions, e.g. concentration of the agent and duration of administration, such that the amount of bone growth may be less than the amount that is observed when the agent is not administered. Under certain circumstances and embodiments the agent(s) will produce a bone growth inhibition of at least about 99%, about 95%, about 90%, about 75%, about 50%, or about 25 when compared to a control in the absence of an agent(s) or composition of the invention. In some instances bone growth inhibition may be substantially or completely eliminated, or substantially or eliminated over a period of time. The administration of an agent “at a site of injury” includes the delivery, administration or application to a specific site or promotion of local contact between the agent and the inflamed or injured site, e.g. an injured bone; it also includes the local administration of an agent in accordance with this invention to a location proximal to the injured bone or site so that the agent may produce the desired or stated therapeutic effect, e.g. reduce bone resorption and/or bone regrowth at the site. “Formulated for controlled release” means that the agent, composition or device may be formulated to release the agent over an extended period of time when administered according to the invention. The agent, for example, may be formulated for release over a period of at least about 2, about 5, about 12 about 24, or about 48 hours, or over at least about 2, about 5, about 10, about 20, or about 40 days, months or years depending on the need of a specific application. In one preferred embodiment the agent may be formulated for release over at least about 5 days to about 10 days. In another preferred embodiment the agent may be formulated for release over a period of about 1 day, several days, about 1 week to about 6 months, about 1 year, about 2 years, and even longer. In still another embodiment the agent may be formulated for released over a period of about 30, about 45, about 60 days to about 90, about 105, about 120 days, and even longer. An agent is said to be “appended” to a polymer when the agent is bound to the polymer as a side chain, side group, or terminal cap but may be not part of the polymeric backbone. In one preferred embodiment the agent(s) of the invention may be bound to the polymer by a labile linkage; that may be a linkage that will release the agent when the polymer is administered or applied in accordance with the methods of the invention. An agent may be linked to a polymer, for example, through a hydrolyzable linkage such as an anhydride, ester or other linkages, some of which are described in this patent by means of example. The term “entrapped in the polymer matrix” means that an agent may be located within the matrix of a polymer so that it may be released in a controlled manner when placed within the body of an animal or human. The agents may also be “mixed”, “blended” and/or “dispersed” within any polymer as these terms are understood in the art. In one preferred embodiment the polymer matrix comprises a biodegradable polymer. As used herein, the term “setting or fixing a fracture” includes holding fractured bone pieces together and/or stabilizing the fracture. The term “implant” includes a device, e.g. an orthopedic device such as an artificial joint, bone, bone part, a film, paste, gel, fiber, chip, microsphere, nanoparticle or a coating on a composition or device intended for insertion into a wound, bone, or completely or partially inserted into a mammalian body. The term “dressing” refers to an object that may be placed in contact with a wound, injury or injured or exposed tissue to cover the injured or exposed tissue, or deliver an agent to the tissue. The “release” of an agent refers to a delivery of an agent by a composition, matrix(ces), monomer(s), oligomer(s), polymer(s), etc., in a form that may be or becomes bioavailable in situ. The term “release” includes the release of an appended agent(s) by degradation of a polymer. The term “release” includes also the release of agent(s) by degradation of a polymer where the agent(s) is(are) entrapped, dispersed, mixed, blended, or otherwise associated with any polymeric matrix(ces). Either of these places the agent(s) in direct contact with the surrounding tissue or bone. The term “release” also encompasses administration of an agent(s) in free form or as a mixture(s) or salt(s) thereof, or in a form that may be immediately bioavailable, i.e. not a sustained release formulation.

II. Embodiments of the Invention

The invention provides a composition, an article of manufacture and a method of inhibiting bone growth by administering or applying at, or in proximity to, a pre-selected site of injury an amount of an anti-inflammatory agent(s) for a time effective to retard, reduce or inhibit bone growth and/or bone resorption. In one particular application the agent(s) generally prevents bone growth and/or resorption. In one application, the injury involves or may be associated with a hard tissue injury, such as a bone injury, and sometimes more specifically a bone fracture. In a particular important application the injury may be associated with, involves or comprises, a disease process such as heterotopic ossification or an osteophyte, among many others. In yet another, the agent(s), compositions and/or articles of the invention are applied to inhibit bone growth occurring through or associated with intramembranous ossification. In still another application the present agents, compositions and/or articles are applied to retard, impede or inhibit bone growth occurring through or associated with endochondral bone formation, among others. In a particularly important application the agent(s) of this invention are applied to an injury that involves or may be associated with the implantation of a medical device or article and/or the removal of an already implanted device or article. Examples of the latter are orthopedic device, e.g. an artificial joint, and bone or bone fragment replacements, among others. The invention also provides compositions, articles, and a method of inhibiting bone resorption or breakdown at, or in the surroundings of, a site of injury by administering at least one anti-inflammatory agent(s) at, or in the vicinity of, a site of injury in an amount and for a period of time effective to inhibit bone resorption or breakdown. Examples of injury occurrences where the present method finds utility include deep bone infections, surgical procedures, bone fractures, joint injuries, implantation of devices, removal of devices, and the like. In one preferred embodiment the anti-inflammatory agent(s) comprise(s) a non-steroidal anti-inflammatory agent(s) (NSAID(s)), or a mixture(s) or inorganic or organic salt(s) thereof. In another embodiment the anti-inflammatory agent(s) comprise(s) a steroidal anti-inflammatory agent (SAID(s)) or a mixture(s) or salt(s) thereof with an NSAID(s). In still another embodiment the anti-inflammatory agent(s) comprise(s) a prostaglandin synthesis inhibitor(s), a mixture(s) or salt(s) thereof. In another embodiment the anti-inflammatory agent(s) comprise(s) salicylic acid, a salicylate salt(s) or other derivative(s) thereof that are known in the art. In another embodiment the anti-inflammatory agent(s) comprise(s) a cyclooxygenase inhibitor(s), e.g. a cyclooxygenase-2 inhibitor(s), a mixture(s) or salt(s) thereof. In yet another embodiment the anti-inflammatory agent(s) may be formulated for controlled release for administration or application at a pre-selected site, such as a site of injury. The anti-inflammatory agent(s), for example, may be entrapped in a matrix formed by a biodegradable monomer(s), oligomer(s), polymer(s), or salt(s) or mixture(s) or blend(s) thereof, appended to or incorporated into a biodegradable polymer backbone of any chemical composition. In a further embodiment the monomer(s), oligomer(s), polymer(s), blend(s), mixture(s) or salt(s) thereof may be incorporated into a film, paste, gel, fiber, chip, powder, tablets, capsules, microparticles, or nanoparticles, among many others. In a specific embodiment they may be incorporated into a microparticle(s). Any oligomer(s) and polymer(s) that is(are) compatible with ani8mal tissue is(are) suitable for use in this invention. Examples are oligomers and polymers having amide, ester, ether, carbonate, azo, thioether, thioamide, thiocarbonate, and many other labile bonds known in the art. Examples of polymers that may be employed for this purpose are shown throughout this patent. One specific polymer employed for this purpose comprises a poly[(1,8-bis(o-dicarboxyphenyl)octanoate-(1,6-bis(p-carboxyphenoxy)hexane] co-polymer, or its salts, blends, and other compositions, formulations, mixtures with other agents, monomers, oligomers and polymers of the invention. In another particularly important embodiment the anti-inflammatory agent(s) in any of its forms may be administered in an implant, device or dressing. The concentration of the anti-inflammatory agent(s) present or loaded into an implant, device or dressing will depend on the application and on the period of time required for release. One example includes, for instance, at least about 0.02, about 0.05, about 0.1, about 0.4, about 0.1 to about 2 g/cm³, or more of the drug. However, other amounts are also contemplated within the confines of this invention as a practitioner will know how to vary the load for different applications. In particular embodiments of the invention at least about 1 μg, about 5 μg, about 10 μg, about 100 μg, about 1 mg, about 5 mg and up to about 2 g and higher anti-inflammatory agent(s) per day is(are) desirably released at the site, i.e. released from a sustained release device or directly administered as free agent, or released from a formulation or an article of manufacture. In a particularly useful embodiment at least about 0.1 μg, about 0.5 μg, about 1 μg, about 10 μg, about 0.1 mg, about 0.5 mg and up to about 5 mg, and higher per hour anti-inflammatory agent(s) may be released at the site, i.e. released from a sustained release formulation, device, implant or dressing, or directly administered as free anti-inflammatory agent(s). In another important embodiment the agent(s) may be released in a bone growth or bone resorption retarding, reducing or inhibiting effective amount at the site for a desired period of time, or the concentration of the agent(s) in tissue, or extracellular fluid at or near the site may be desirably at least about 0.05, about 0.1, about 0.3, about 0.5, about 1.0, about 2.0 mg/cm³ to up to about 5 g/cm³ and sometimes even higher. In other useful embodiments these concentrations are sustained for at least about 6, about 12, about 24 hours, about 5 days, about 10 days up to about 24 hours, about 5 days, about 10 days, and even longer periods of time.

The compositions, articles and method of the invention may be employed in in- and out-patient medical office or hospital procedures as well as in emergency or field situations to maintain or preserve injured bone until a patient reaches a hospital or other health care facility. The present compositions, articles and methods may be employed by paramedics and ambulance personnel in the treatment of persons afflicted with wounds and physical trauma, particularly those with fractured bones. The products and methods of the invention will allow for the injured site to remain in a status quo for a period of time until the wounds are treated or the fractured bones set. The present compositions, articles and methods may be also applied to subjects suffering from a back or spinal injury. One extremely valuable application for the methods of the invention is to prevent bone growth that causes disc fusion, among other s such as treating spinal spurs.

III. Specific Compositions of the Invention

a. Controlled Release

Numerous controlled release mechanisms are known in the art. See, for example, Langer, R., Science 249: 1527-1533 (1990); WO 02/09768; WO 02/09767; WO 01/41753; WO 99/12990. Any and all controlled release mechanisms and formulations may be employed in practicing the invention, provided it allows for the controlled release of the anti-inflammatory agent(s) at, or near, the site of interest. One preferred form of controlled release of an anti-inflammatory agent(s) and other optional agent(s) may be by incorporation into a monomer(s), oligomer(s) or polymer(s), preferably bio-degradable. The anti-inflammatory agent(s) may be dispersed through the polymer matrix, appended to the polymeric backbone, attached as an end-cap to the matrix(ces), or incorporated directly into a biodegradable polymer backbone. Typically, any anti-inflammatory agent(s) may be dispersed through, or mixed into a polymer matrix(ces) to provide a suitable controlled release formulation. The ability of any particular anti-inflammatory agent(s) to be appended to or incorporated into a monomer(s), oligomer(s), or polymer(s) may depend on the functional groups present in the agent(s), which anti-inflammatory agents are suitable for appending to or incorporation into any polymer to provide a suitable controlled release formulation are described in greater detail below and would otherwise become apparent to an artisan upon inspection of their chemical formulas.

b. Anti-Inflammatory Agents

Anti-inflammatory agents are a generally well-known class of pharmaceutical agents that reduce inflammation by acting on body responses without directly antagonizing the causative mechanism. See, Stedman's Medical Dictionary, 26th Ed., Williams and Wilkins (1996); Physicians Desk Reference, 51st Ed., Medical Economics (1997). One group of anti-inflammatory agents suitable for incorporation into the compositions and use in the methods of the invention include non-steroidal anti-inflammatory agents (NSAIDs), which agents are said to typically inhibit the body's ability to synthesize prostaglandins. Prostaglandins are a family of hormone-like chemicals, some of which are naturally produced by the body in response to cell injury. Specific NSAIDs that are already approved for administration to humans include naproxen sodium, diclofenac, sulindac, oxaprozin, diflunisal, aspirin, piroxicam, indomethocin, etodolac, ibuprofen, fenoprofen, ketoprofen, mefenamic acid, nabumetone, tolmetin sodium, and ketorolac tromethamine. Examples of NSAIDs useful for incorporation into the present polymers, compositions and for use in the methods of the invention include salicylates, such as salicylic acid, choline salicylate, magnesium salicylate, sodium salicylate, olsalazine, and salsalate, among others. Other anti-inflammatory agents suitable for incorporation into the invention's polymers, compositions and methods include inhibitors of cyclooxygenase (COX), also known as prostaglandin H synthase, or PGH synthase. The COX enzyme catalyzes the conversion of arachidonic acid to prostaglandin H2 (PGH2), and a COX inhibitor inhibits the progress of this reaction. Two COX genes named COX-1 and COX-2 have been isolated so far in several species. The expression of the COX-2 gene may be tightly regulated in most tissues, and usually only induced under abnormal body conditions, such as may be the case in inflammation, rheumatic and osteo-arthritis, kidney disease, and osteoporosis, among others. The COX-1 gene, on the other hand, is believed to be constitutively expressed so as to maintain platelet and kidney function and integral homeostasis. In one embodiment the anti-inflammatory agent useful in the invention includes COX-2 inhibitors. Typical COX inhibitors useful in the methods of the invention include etodolac, Celebrex, meloxicam, piroxicam, nimesulide, nabumetone, and rofecoxib, among others. Other examples of anti-inflammatory agents include Isonixin, Amtolmetin Guacil, Proglumetacin, Piketoprofen, Difenamizole, Epirizole, Apazone, Feprazone, Morazone, Phenylbutazone, Pipebuzone, Propyphenazone, Ramifenazone, Thiazolinobutazone, Aspirin, Benorylate, Calcium Acetylsalicylate, Etersalate, Imidazole Salicylate, Lysine Acetylsalicylate, Morpholine Salicylate, I-Naphthyl Salicylate, Phenyl Acetylsalicylate, Ampiroxicam, Droxicam, S-Adenosylmethionine, Amixetrine, Benzydamine, Bucolome, Difenpiramide, Emorfazone, Guaiazulene, Nabumetone, Nimesulide, Proquazone, and Superoxide Dismutase, among many others that are known to an artisan. In another preferred embodiment, at least one anti-inflammatory agent(s) may be appended to a polymer of this invention for use in compositions and methods of administration of the invention include Etofenamate, Talniflumate, Terofenamate, Acemetacin, Alclofenac, Bufexamac, Cinmetacin, Clopirac, Felbinac, Fenclozic Acid, Fentiazac, Ibufenac, Indomethacin, Isofezolac, Isoxepac, Lonazolac, Metiazinic Acid, Mofezolac, Oxametacine, Pirazolac, Sulindac, Tiaramide, Tolmetin, Tropesin, Zomepirac, Bumadizon, Butibufen, Fenbufen, Xenbucin, Clidanac, Ketorolac, Tinoridine, Benoxaprofen, Bermoprofen, Bucloxic Acid, Fenoprofen, Flunoxaprofen, Flurbiprofen, Ibuprofen, Ibuproxam, Indoprofen, Ketoprofen, Loxoprofen, Naproxen, Oxaprozin, Pirprofen, Pranoprofen, Protizinic Acid, Suprofen, Tiaprofenic Acid, Zaltoprofen, Benzpiperylon, Mofebutazone, Oxyphenbutazone, Suxibuzone, Acetaminosalol, Parsalmide, Phenyl Salicylate, Salacetamide, Salicylsulfuric Acid, Isoxicam, Lomoxicam, Piroxicam, Tenoxicam, ε-Acetamidocaproic Acid, Bendazac, α-Bisabolol, Paranyline, Perisoxal, and Tenidap, among others known in the art. In another preferred embodiment, anti-inflammatory agents that may be incorporated into the backbone of the polymers of the invention for use in compositions, formulations and devices as well as for administration in the methods of the invention include Enfenamic Acid, Aceclofenac, Glucametacin, Alminoprofen, Carprofen, Ximoprofen, Salsalate, 3-Amino-4-hydroxybutyric Acid, Ditazol, Fepradinol, Oxaceprol, and Zileuton, among many others. Other Anti-inflammatory agents that posses suitable functionalities for incorporation into the backbone of a polymer as described herein include Flufenamic Acid, Meclofenamic Acid, Mefenamic Acid, Niflumic Acid, Tolfenamic Acid, Amfenac, Bromfenac, Diclofenac Sodium, Etodolac, Bromosaligenin, Diflunisal, Fendosal, Gentisic Acid, Glycol Salicylate, Salicylic Acid, Mesalamine, Olsalazine, Salicylamide O-Acetic Acid, Sulfasalazine, 5-Chlorosalicylic acid, and 5-Trifluoromethylsalicylic acid, among others. It is understood that all anti-inflammatory and other agents referred to in this patent by a trade name include not only the trade named product, but also the agent or ingredient present in the product that possesses anti-inflammatory activity, and other products comprising it including generics and other formulations. Most anti-inflammatory agents identified herein as suitable for incorporation into a polymer backbone are also suitable for appending to the polymer or for incorporation into the polymer matrix, e.g. by dispersion, mixing, blending, and the like, and for use in other compositions of the invention.

c. Administration of Anti-Inflammatory Agents

This patent provides for the in situ administration or implantation of an anti-inflammatory agent(s) in the form of a pharmaceutical composition and/or various articles. The composition may further comprise a carrier or diluent, such as are known in the art. In addition, the composition may further comprise other therapeutic and traceable agents, such as are described below. The dosages of all agents employed in this invention are readily ascertained by an artisan from the information currently in the public domain, and need not be described in detail in this patent.

IV. Structure of Monomers, Oligomers and Polymers

a. Introduction

The monomers, oligomers and polymers of the invention are suitable for delivering an anti-inflammatory agent(s) to a pre-selected site. The monomer, oligomer and polymer releases at least one agent(s) upon degradation and/or hydrolysis of the polymer under appropriate conditions, e.g. physiological conditions, and optionally at least one additional agent(s), for use in monitoring, diagnostic, prophylactic and therapeutic applications. Monomers, oligomers and polymers include any backbones that are suitable as delivery systems. They may incorporate the anti-inflammatory agent(s) and other agents into the backbone, e.g. in the form of units linked by labile bonds such as esters, thioesters, amides, thioamides, urethanes, carbamates, carbonates, ethers, azo and carbonate linkages, among others. Or they may have the agent(s) appended, dispersed, occluded, or blended into them as is known in the art.

When delivered into a host a suitable substance in the form of a monomer, oligomer or polymer will degrade over a period of time to produce relatively high, localized levels of the agent(s). In one embodiment the substance is biocompatible. In another embodiment, the substance may be biodegradable and demonstrates favorable solubility and processability, as well as degradation properties suitable for the desired use. In yet another embodiment, the agent(s) is(are) released over a period of time as the substance hydrolyzes under physiological conditions, providing for an extended-release formulation giving a consistent and continuous release of the agent(s). Suitable substances include mono-, oligo- and poly-esters, such as mono-, oligo- and poly(ester-esters), poly(ester-carbonates), polyamides, polycarbonates, and polyanhydrides such as poly(anhydride-esters) and poly(azo-anhydrides), among others. Examples may found in U.S. Pat. Nos. 6,328,988; 6,365,146; 6,468,519; 6,486,214; 6,497,895; 6,602,915; 6,613,807; 4,916,204; and 4,868,265; U.S. Published Patent Applications 2002/0071822 A1; 2002/0106345 A1; 2003/0035787 A1; 2003/0059469 A1; 2003/0104614A1; 2003/0170202A1; U.S. Ser. Nos. 09/508,217; 10/368,288; 10/622,072; 10/646,336; 10/647,701; WO 99/12990; WO 01/28492; WO 01/41753; WO 01/58502; WO 02/09767; WO 02/09768; WO 02/09769; WO 03/005959; WO 03/046034; WO 03/065928; and WO 03/072020; and Erdmann, L., Uhrich, K. E., Biomaterials, 21: 1941-1946 (2000), the relevant portions of all of which are incorporated herein by reference. The polymer of the invention may be a monomer, oligomer and/or a polyanhydride, preferably having a backbone comprising one or more groups that will release a compound upon hydrolysis or enzymatic degradation of the polymer. Exemplary polymers provided throughout this patent are listed in Table 2 below.

TABLE 2
Exemplary Polymers* throughout the Patent

| Compound | |
|---|---|
| No. | Compound Description |
| 125PL | poly (ester-anhydride) made from monomer of (salicylic |
| | acid-C₁₂-salicylic acid)ₓ by a melt polymerization |
| | process. |
| 261PL | poly (ester-anhydride) made from monomer of (salicylic |
| | acid-C₈-salicylic acid)ₓ by a melt polymerization |
| | process. |
| 510PL | poly (ester-anhydride) made from monomer of (salicylic |
| | acid-C₆-salicylic acid)ₓ by a melt polymerization |
| | process. |
| 657PL | poly (ester-anhydride) made from monomer of (diflunisal- |
| | C₁₄-diflunisal)ₓ by a melt polymerization process. |
| 749PL | poly (ester-anhydride) made from monomer of (salicylic |
| | acid-C₁₀-salicylic acid)ₓ by a melt polymerization |
| | process. |

*Polymerix Corp Polymers.
ₓis a positive integer showing the degree of polymerization

b. General Polymer Formulas

The present invention provides for the delivery or in situ administration of a monomer comprising at least one an anti-inflammatory agent(s) by itself or operatively attached to an additional agent(s) and/or linker(s), or admixed with them. One example of a polymer suitable for use in the compositions and for practicing the methods of the invention comprises a polymer having the chemical formula —C(═O)—R¹-L¹-R¹—C(═O)—O— (formula I of WO 99/12990), wherein each R¹, independently from one another, comprises a substituted or unsubstituted aromatic residue, and each L¹, independently from one another, comprises a difunctional organic residue substituted on each R1 ortho to the anhydride. R¹ and L¹ are preferably selected so that the hydrolysis products released by the polyanhydrides have a chemical structure resembling pharmaceutical agents, e.g. salicylates, and the like. In particular, R¹ preferably comprises a phenyl group and L¹ preferably comprises -A-L¹-A-, wherein each L¹, independently from one another, comprises a difunctional residue, and each A, independently from one another, comprises a labile bond such as ester, thioester, amide, thioamide, anhydride, carbonate, urethane or sulfide, among others. Each L¹, independently from one another preferably comprises straight, branched or cyclic (C₁-C₅₀) alkyl, alkenyl, or alkynyl, or C₂-C₅₀ (—CH₂—CH₂—O—)ₘ, (CH₂—CH₂—CH₂—O—)ₘ or (—CH₂—CHCH₃—O—)ₘ, or each L¹, independently from one another, comprises -L²-A²-L³-, wherein each L² and L³, independently from one another, comprises linear, branched or cyclic (C₁-C₅₀) alkyl, alkenyl or alkynyl, or C₂-C₅₀ (—CH₂—CH₂—O—)ₘ, (—CH₂—CH₂—CH₂—O—)ₘ and/or (—CH₂—CHCH₃—O—)ₘ, and each A², independently from one another, comprises a difunctional residue as described above with respect to A. In one preferred embodiment all linking residues, e.g. L¹, L² and L³, may be further substituted by O, N, S, P, and/or halo, among other atoms. In another preferred embodiment each carbonyl group may be directly substituted on the corresponding aromatic residue. Particularly preferred polymers include those having at least one, and may carry repeating units of a, residue of the chemical formula shown above, wherein R¹ may comprise phenyl, and each L¹, independently from one another, may comprise -A-(—CH₂—)ₙ-A-, -A(-CH₂—CH₂—O—)ₘ-A-, -A(-CH₂—CH₂—CH₂—O—)ₘ-A- and/or -A(-CH₂—CHCH₃—O—)ₘ-A-, wherein each A, independently from one another, comprises ester, thioester, amide or thioamide, each m may be selected so that L¹ comprises about 2 to about 50 inclusive, and preferably about 6, and n comprises about 1 to about 50 inclusive, and preferably about 6. Another polymer suitable for use in the composition and methods of the invention includes a polymer as described in WO 02/009768, the polymer comprising at least one residue of the chemical formula —R¹-A-L-A-, wherein each R¹, independently from one another, comprises a residue that may release at least one anti-inflammatory agent(s) upon hydrolysis of the polymer; each A, independently from one another, comprises amide, thioester, or ester; and L comprises a linking group. Another polymer suitable for use in the compositions and methods of the invention may be one described in WO 02/009768), which polymer comprises the chemical formula —R²-A-L-A-R²-A-L-A-, wherein each R² and R³, independently from one another, comprise a residue that will release at least one anti-inflammatory agent(s) upon polymer erosion; each A, independently from one another, comprises amide, thioester, or ester; and each L, independently from one another comprises a linking group. Still another polymer suitable for use in the compositions and methods of the invention comprises at least one unit(s) of the chemical formula described in WO 02/009767 —C(═O)R¹-A-R²-A-R¹—C(═O)—O—, wherein each R¹, independently from one another, comprises a residue that releases at least one anti-inflammatory agent(s) upon polymer hydrolysis; each A, independently from one another, may comprise amide, thioester, or ester; and R² comprises a linking group.

The polyanhydrides may be prepared by the method described by Conix in Macromol. Synth. 2: 95-99 (1966), in which dicarboxylic acids are acetylated in an excess of acetic anhydride followed by melt condensation of the resulting carboxylic acid anhydride at 180° C. for 2-3 hours. Other suitable methods for preparing these polymers are described below or in WO02/09768, WO02/09767, WO01/41753, WO99/12990, or are otherwise in the public domain. In one embodiment the monomer may comprise a compound of the formula R¹-A-P, or wherein each R¹ comprises at least one anti-inflammatory agent(s), A comprises a linker or may be absent, and P comprises at least one additional agent(s) or may be absent. In another embodiment the invention provides a compound of formula H—Y—C(═Y)—R¹-A-R¹—C(═Y)—Y—H (“Formula Ia”), wherein each R¹ comprises, independently from one another, a residue(s) of a diagnostically, traceably, biologically or therapeutically active or activatable agent(s) or compound(s) that is(are) released upon monomer, oligomer or polymer degradation; each Y comprises independently O, S, NR⁷, where R⁷ comprises H, alkyl, alkenyl, alkynyl, all of which may be substituted with O, N, S, P or halogen; each A, independently from one another, comprises ester, amide, thioester, azo, or thioamide, or their combination. In another embodiment, the compound, and the monomer, oligomer or polymer comprising unit(s) of this compound, comprise the chemical formula H—Y—C(═Y)—R¹-A-L-A-R¹—C(═Y)—Y—H (“Formula Ib”), wherein all variables are defined as above; and L comprises a linking group. In one embodiment, A comprises an amide, an ester, or both, and in another embodiment, A comprises a thioamide, a thioester, or combinations thereof. Typically, the R¹ may comprise monomers, dimers, trimers, tetramers, and higher meric or repeating units of the agent's residue. These individual residues may be bound directly to one another, or through a linking group(s). Suitable linking groups are those described in this patent and include all other suitable functional groups and residues known in the art. The monomer, oligomer or polymers of the present invention comprise an agent(s) or compound(s), and an optional linker group(s) bonded through a labile linkage such as an ester, thioester, amide, thioamide, azo, anhydride, carbonate, ether, thioether, or a combination thereof. Due to the presence of the ester, thioester, amide, and/or thioamide linkages, the monomer, oligomer or polymers may be hydrolyzed, enzymatically or otherwise degraded, under physiological conditions to provide biologically active compounds. Thus, the monomer, oligomer or polymers of the present invention are particularly useful as for controlled release of agents, whether for biological or other types of applications, and as a means for localized delivery of agents to a selected site or target. The monomer, oligomer or polymers of the invention may be used, for example, for the localized delivery of an agent to a targeted site within the human body, e.g. within or near a tumor, where the polymer provides a localized, controlled release of the agent. The polymers prepared using the processes of the invention may have an average molecular weight (MW_{AVE}) of about 1,500; 3,000; 10,000; 30,000; 50,000; 100,000; 250,000; 500,000; or 1,000,000 Dalton to about 20,000; 50,000; 100,000; 200,000; 350,000; 500,000; 750,000; 1,000,000; 1,200,000; 1,350,000; or 1,500,000 Dalton, and even higher, as determined by Gel Permeation Chromatography (GPC) relative to narrow molecular weight polystyrene standards as is known in the art. The present monomer, oligomer or polymers thus may exhibit a backbone that links one or more agents or compounds into a delivery system. The monomer, oligomer or polymers are typically biocompatible and biodegradable, and preferably demonstrate excellent solubility and processability, as well as suitable degradation properties, such as erodability, due to the presence of bonds such as anhydride, ester, amide, urethane, carbamate, azo, and carbonate, among many others, that are breakable under specified conditions. Suitable monomer, oligomer or polymer bonds for use in the present invention include, for example, ester, polyamide and anhydride polymers of the type described in WO 99/12990; U.S. patent applications Ser. Nos. 09/917,231; 09/917,194; 09/508,217; 09/422,294; 09/732,516; 60/220,707; 60/261,337; 60/058,328; and 60/220,998.

This patent also provides a compound of the chemical formula -(M)ₓ- (IIa) and/or B-[(M)ₓ]_{y} (IIb), wherein M comprises a residue suitable for polymerization, B comprises a residue with multiple functional groups. x represents the number of repeating units, e.g. m may be about 2, about 5, about 10, about 15, about 20, about 30, about 50 to about 100 or any higher number as needed to reach a desired average molecular weight of about 1,500; 3,000; 5,000; 7,500; 10,000; 20,000; 50,000; or 100,000 Dalton to about 50,000, about 75,000; about 100,000; about 250,000; about 500,000; about 1,000,000, and higher Dalton; and y may be a positive integer between about 2 and about 8. B may be a residue with multiple functional groups suitable to start polymerization, such as COOH, NH₂, SH, and many others. Examples of compounds for use as B are 1,3,5-benzene tricarboxylic acid, 1,2,3,4-butane tetracarboxylic acid, cis-aconitic acid, and trans-aconitic acid, among others known in the art. In one embodiment M comprises one or more units of the chemical formula —R¹-A-R¹-(IIIa) and/or —R¹-A-L-A-R¹- (IIIb), wherein each R¹, independently from one another, comprises one or more residues comprising an anti-inflammatory agent(s) that may be released upon degradation; each A, independently from one another, comprises a labile group such as amide, thioamide, ester, thioester, carbonate, azo, or thiocarbonate, among others; and L, which may or may not be present in the backbone, independently from one another, comprises one or more units of a linking residue(s). Such a monomer, oligomer or polymer may be particularly useful for the administration of a combination of more than one agent(s). In one embodiment, R¹ comprises a monomer, dimer, trimer, tetramer, pentamer, and higher mers or repeating units such as a decamer, dodecamer, hexadecamer, etc., of the same or different agent(s). Oligo- and polyanhydrides made of formulas (IIIa) and/or (IIIb) or combinations thereof serve as the backbone of a delivery system that provides a controlled delivery of an agent(s) or compound(s) to any targeted site, e.g. of a host such as a human, or animal. In one embodiment, the oligomer or polymer of formula (III) comprises a low molecular weight agent(s) with functional groups such as carboxylic acid, thioacid, amine, amide, thiol, thioamide, carbonate, azo, alcohol or phenol, among many that form labile bonds, including those comprising heteroatoms such as P, S, N, and the like. In another embodiment, the oligomer or polymer comprises a unit(s) comprising formula (IIIa) and/or (IIIb), wherein each R¹, independently from one another, comprises and may be capable of releasing an aromatic agent(s), such as a non-steroidal anti-inflammatory drug (NSAID), or any other agent(s) to be delivered. Examples of suitable NSAIDs include, but are not limited to, salycilates, diflunisal, diflucan, thymotic acid, 4,4-sulfinyldinailine, 4-sulfanilamidosalicylic acid, sulfanilic acid, sulfanilylbenzylamine, sulfaloxic acid, succisulfone, salicylsulfuric acid, salsallate, salicylic alcohol, salicylic acid, orthocaine, mesalamine, gentisic acid, enfenamic acid, cresotic acid, aminosalicylic acid, aminophenylacetic acid, acetylsalicylic acid, and the like. The identification of suitable R¹ and A to release an aromatic agent(s), e.g. a salicylate, may be readily determined by those of ordinary skill in the art without undue experimentation. In one embodiment, the agent may be salicylic acid or one of its derivatives that are well known in the art. In another embodiment suitable azo monomers are polymerized to provide polyazo compounds and then polyazo anhydrides. In a preferred embodiment the monomer, oligomer and polymer may be a mono-, oligo- or polyester or polyamide, and it comprises units containing at least two free hydroxyl, phenols, amines, or combinations thereof available for co-polymerization with carboxylic acids or bis(acyl) chlorides.

Another preferred monomer, oligomer or polymer may comprise one or more units of the chemical formula —R¹-A-L-A- (IV), wherein all variables are as defined above. Another exemplary product or substance of the invention may be a co-oligo or co-polymer that comprises one or more units of the chemical formula —R¹-A-L-A-R¹-A- (V), wherein all variables are as defined above. In one embodiment, the monomer, oligomer or polymer comprises one or more units of the chemical formula —R²-A-L-A-R³-A-L-A- (VI), wherein R² and R³, independently from one another, comprise a residue that will yield a compound(s) upon hydrolysis or enzymatic degradation; and other variables are as defined above. Monomers, oligomers and polymers where R² and R³ comprise residues that will yield different compounds upon degradation are particularly useful for the administration of combination therapy. Another preferred embodiment comprises a co-oligomer or co-polymer of one or more units of the chemical formula —R¹-A-L²-A-R¹-A-L³-A- (VII), wherein each L² and L³ comprises a linking group; each A, independently from one another, comprises amide, thioamide, carbonate, azo, ether, thioester, or ester, and/or other labile bonds; and each R¹, independently from one another, comprises a group that will yield an active or activatable compound upon polymer erosion, hydrolysis or enzymatic degradation. In this embodiment L² and L³ are linking groups that impart different physical properties to the monomer, oligomer or polymer that makes them particularly useful for customizing the physical characteristics of the monomer, oligomer or polymer for a specific application. In one embodiment, the agent may be salicylic acid, and the oligomer or polymer comprises an oligo or poly(ester-ester). In one embodiment, the monomer, oligomer or polymer comprises one or more units of formula -A-R¹—N═N—R¹-(A-L)ₙ- (VIIIa) and/or -A-R¹—N═N—R¹-(A-L)ₙ-(VIIIb), wherein each R¹—N, independently from one another, comprises a group that will provide a biologically active compound upon degradation; each A, independently from one another, comprises anhydride, amide, thioamide, thioester, carbonate, ether, or ester; L comprises a linking group as already described; n may be 0 to 10. Suitable monomers are polymerized to provide the mono-, oligo-, and polyazo compounds. In one embodiment, the polyazo compound comprises at least one free amine group to form the azo group and at least one free carboxylic acid, alcohol or amine available for self-polymerization or co-polymerization with other carboxylic acids or bis(acyl) chlorides. In one embodiment, the monomer, oligomer or polymer comprises more than one agent(s) incorporated into a poly(azo-anhydride) that serves as a polymeric drug delivery system for oral delivery of a cancer drug. The monomer, oligomer or polymer may have two, three, or more different R groups, each of which will provide a different agent(s) upon degradation, and each R group may have one or more repeats of the same or different agent(s), e.g. monomer, dimer, etc. In one preferred embodiment, the monomer, oligomer or polymer comprises a non-steroidal anti-inflammatory agent (NSAID), such as, e.g., salicylic acid and/or diflunisal. Such oligomers and polymers may comprise repeating units of chemical formula II, III, VII and/or X.

In another embodiment the monomer, oligomer or polymer comprises mono-. Oligo- and/or poly(ester-anhydride) bonds. One preferred oligomer and polymer comprises units of the chemical formula 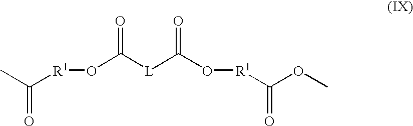
wherein all variables are as defined above. In another embodiment the monomer, oligomer or polymer comprises poly(ester-ester) bonds. One preferred oligomer and polymer comprises units of the chemical formula 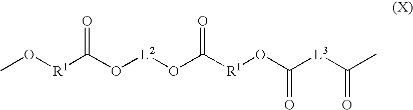
wherein all variables are as defined above. In another embodiment the monomer, oligomer and polymer comprises mono-, oligo- and poly(ester-carbonate) bonds, respectively. One preferred oligomer and polymer comprises units of the chemical formula 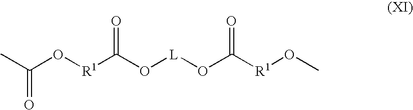
wherein all variables are as defined above. The monomer, oligomer or polymer may have two, three, or more different R¹ groups, each of which may provide a different agent(s) upon monomer, oligomer or polymer degradation. Such mono-, oligo and polymers are particularly useful for the application or administration of a combination of two or more agents to a host, such as an animal or plant, or an article of manufacture. In another embodiment the monomer, oligomer or polymer comprises a homomonomer, oligomer or polymer, and in another it comprises a monomer having more than one agent(s), and/or a co-oligo or co-polymer.

The monomer, oligomer or polymer(s) described herein will release their agent(s) when placed at a pH of about 3, about 4, about 5, about 6, about 7 to about 8, about 9, about 10, about 11, about 12, about 13, and higher over a period of time of about 1, about 2, about 3, about 5, about 10, about 20, about 50, about 75 days to about 2, about 3, about 5, about 7, about 9, about 12, about 24 months, or longer. When the monomer, oligomer or polymer is placed at a pH below its pKa it will degrade slowly, for example over a period of 6 months or longer. When R¹ comprises a drug residue(s), the monomer, oligomer or polymer may function as a drug(s) delivery system that provides a controlled effective amount of the agent(s) as a function of monomer, oligomer or polymer degradation at any pre-determined site to which it may be applied, or delivered. Polyanhydride materials have been extensively described. See, for example, U.S. Pat. Nos. 4,757,128; 4,997,904; 4,888,176; 4,857,311; 5,264,540; and WO 99/12990; WO 02/09769; WO 02/09767. In general, anhydride monomers, oligomers or polymers of higher average molecular weights possess unexpected and advantageous properties, such as greater mechanical strength and higher stability that those of lower average molecular weights do not. Because of this higher molecular weight, these polyanhydrides may be laid as harder and thicker coatings. In one embodiment, the polymer of the invention may have an average molecular weight (MW_{AVE}) of at least about 200,000, and preferably above about 250,000 Dalton, and up to 1,000,000 Dalton and higher. The polymer of the invention typically have a glass transition temperature (T_{g}) about −10, about −5, about 0, about 10, about 30, about 50 to about 60, about 70, about 80, about 100, about 130, about 160, about 200° C., with a most preferred T_{g} may be in the vicinity of or below about 50° C.

The monomer, oligomer or polymer may comprise any number of agents, whether biologically, diagnostically, prophylactically, therapeutically or otherwise active or inactive, or whether the agents have other activities that make them suitable for applications other than to microorganisms, plants, animals, humans, or articles of manufacture. In fact any type of agent that may be polymerized or appended, or mixed, blended, dispersed or otherwise incorporated into a formulation, and released from its structure is suitable for use in this application. Such agent(s) may be loaded in amounts of about 0, about 5, about 10, about 15, about 20% w/w to about 25, about 30, about 35, about 40, about 45, about 50% w/w, although other amounts are also contemplated including up to 70 wt %, and 90 wt %, and even higher. In one embodiment, the monomer, oligomer or polymer comprises a non-steroidal anti-inflammatory agent (NSAID) such as salicylic acid and/or diflunisal, and units of chemical formula I, among others, or their combinations, where each R¹ may be a monomer, dimer, trimer, tetramer, or higher mer of an agent(s). In another embodiment the monomer(s), oligomer(s) and/or polymer(s) is(are) combined with one or more agents in any suitable manner, such as by physically admixing, blending, embedding, appending, or dispersing the additional agent(s) in the matrix. The agent(s) may be also incorporated into the backbone, chemically linked in the backbone directly or through a linker or spacer, directly or indirectly chemically linked to a chemical group attached to the backbone, or electrostatically or in any other manner attached to the monomer, oligomer or polymer or its backbone. In one embodiment the agent(s) may be attached to a unit(s) of the monomer, oligomer or polymers of the present invention by covalent bonds linked to an aromatic (Ar) ring or a linear, branched, or cyclic aliphatic (R) organic residue, providing for sustained release of the agent(s). In another embodiment the agent(s) may merely residue in the unoccupied spaces present. In another embodiment the agent(s) may form(s) a salt(s) with the monomer, oligomer or polymer or its backbone. In still another embodiment the agent(s) may be located in the unoccupied spaces of a monomer, oligomer or polymer, and may be present as a homogeneous functional group, or incorporated into a salt(s), micelle(s), liposome(s), or heterogeneous aggregate(s). The polymer may comprise various segments comprising one or more similar or different residues of an agent(s) that will be released either directly or indirectly by degradation. The monomer, oligomer or polymer may also comprise a second or additional agent(s) that may be physically admixed, embedded or dispersed in, or combined with the monomer, oligomer or polymer as is known in the art.

In another embodiment, the compound(s), and monomer, oligomer and polymer comprise a unit(s) of the agent(s) or of the compound(s) of chemical formula (Ia) or (Ib) shown above comprises a diagnostically, traceably, biologically or pharmaceutically active or activatable agent(s) or compound(s) of the chemical formula 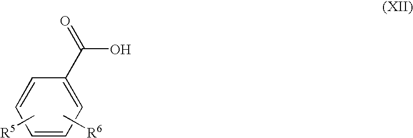
wherein each R⁵, independently from one another, may comprise hydroxy, amine, thiol, or an aliphatic or aromatic organic residue that may further comprise hydroxy, amine, or thiol; and each R⁶, independently from one another, may comprise H, halo, NHR⁷, a cycloaliphatic residue, or aryl, and may be further substituted with HO, halo or halo (C₁-C₄)alkyl; wherein each R, independently from one another, may comprise H, (C₁-C₆)alkyl, (C₃-C₆)cycloalkyl, (C₃-C₆)cycloalkyl (C₁-C₆)alkyl, aryl, heteroaryl, aryl (C₁-C₆)alkyl, or heteroaryl (C₁-C₆)alkyl or (C₁-C₄)alkyl carbonyl. Preferred R⁵ may comprise H, (C₁-C₆)alkyl, (C₃-C₆)cycloalkyl, (C₃-C₆)cycloalkyl (C₁-C₆)alkyl, aryl, heteroaryl, aryl (C₁-C₆)alkyl, or heteroaryl (C₁-C₆)alkyl or (C₁-C₄)alkyl carbonyl. Preferred R⁵ may comprise, but are not limited to, —NH₂, —NHAc, Cl, 2,4-diflurophenyl, chloromethyl, difluoromethyl, —CF₃ and the like. The diacids of chemical formulas (Ia) and (Ib), including those comprising monomers, dimers, trimers, tetramers, and higher numbers of units of the agent(s) or compound(s), may be incorporated into the backbone of this invention, and may be employed also by appending, dispersing, blending, or admixing them, in the monomer, oligomer or polymer. Biocompatible, hydrophobic polyanhydride matrices of this invention are suitable for use in many applications, including surgical, wound healing, hemostatic, orthopedic and dental applications, such as prosthesis and implants. The biodegradable polymer networks of the invention for use in these and other applications may be formed by polymerizing anhydride pre-polymers and employing the solution method(s) described here. Controlled or sustained release polyanhydrides prepared as described in this patent release biologically or pharmaceutically active or activatable agents, e.g. salicylate or difluorophenyl derivatives, or their precursors, e.g. pharmacophores, by in vivo biodegradation, as well as other agents that are incorporated either into the oligomer or polymer backbone, or appended thereto, or added into a formulation of the monomer, oligomer or polymer. Some other suitable polymers are shown below. 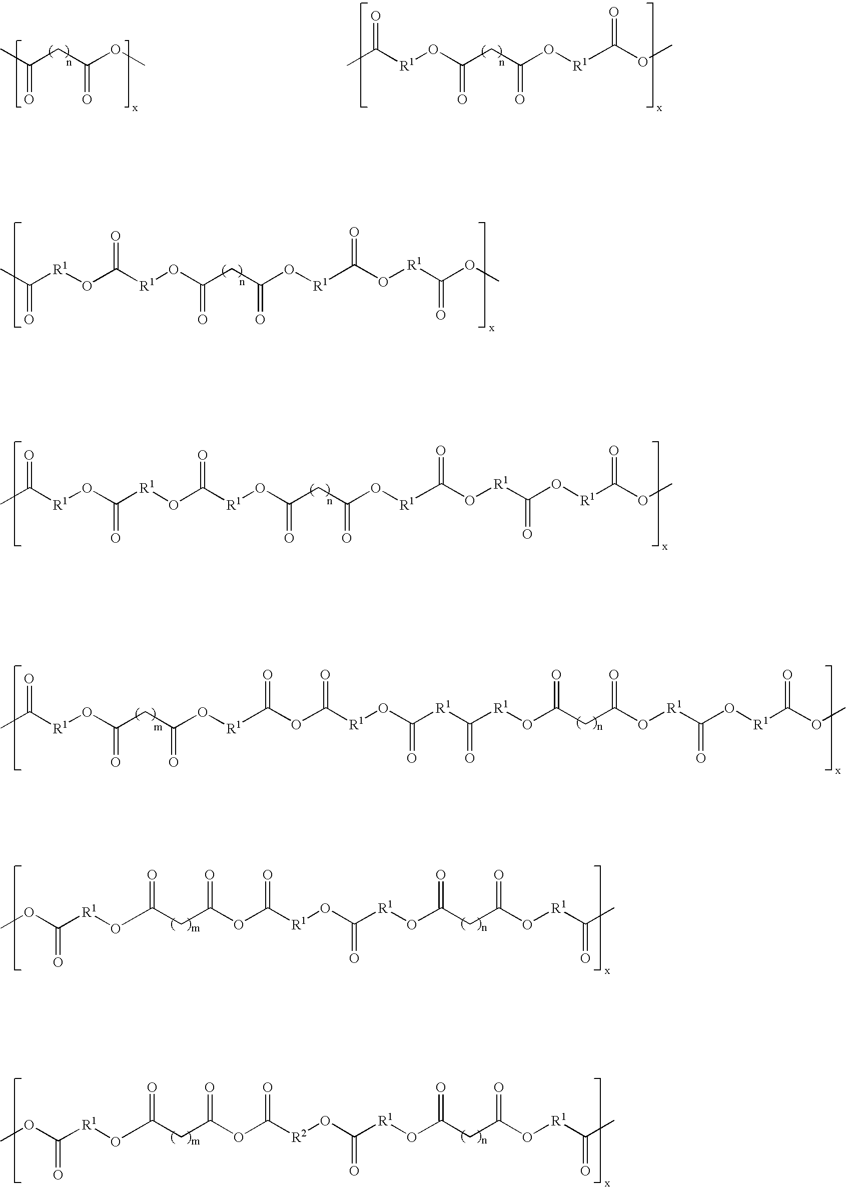 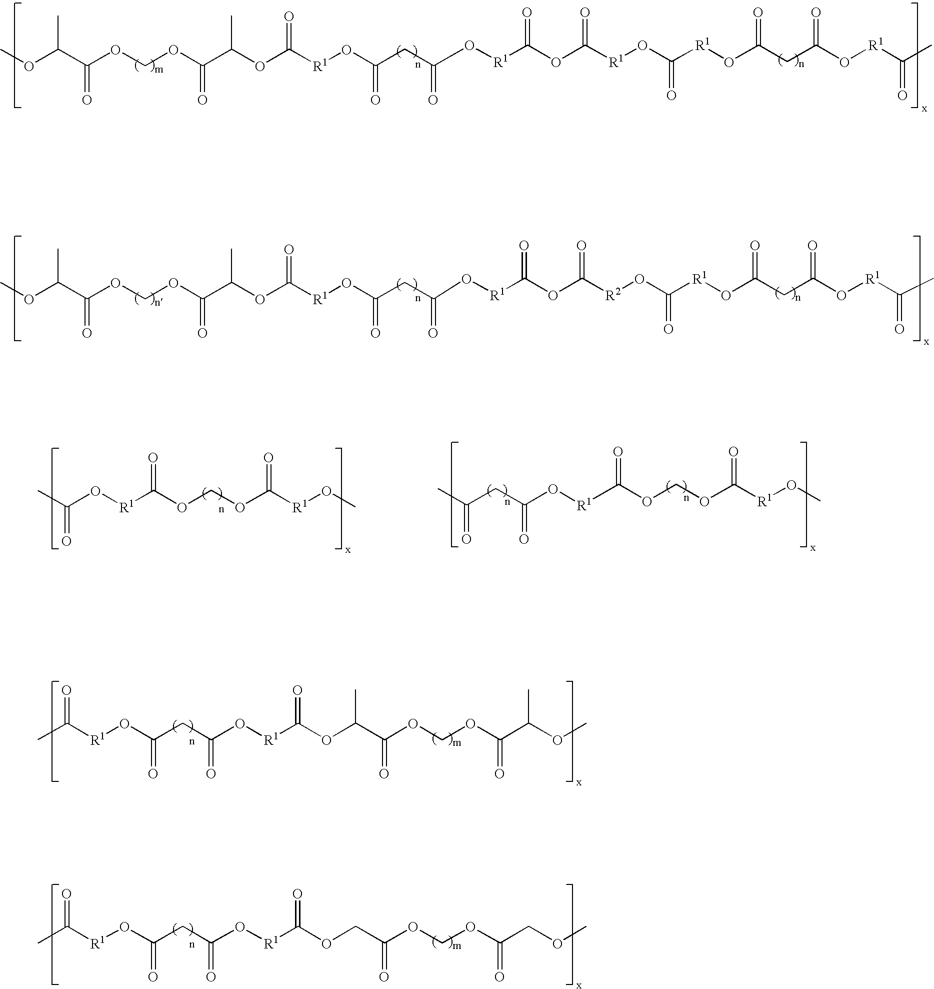 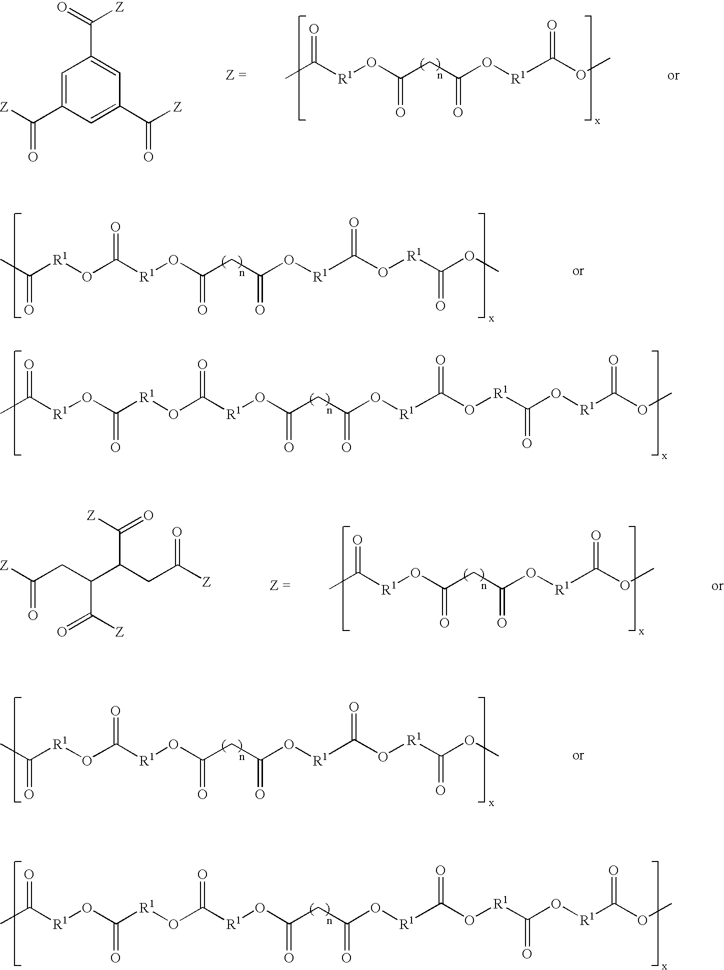 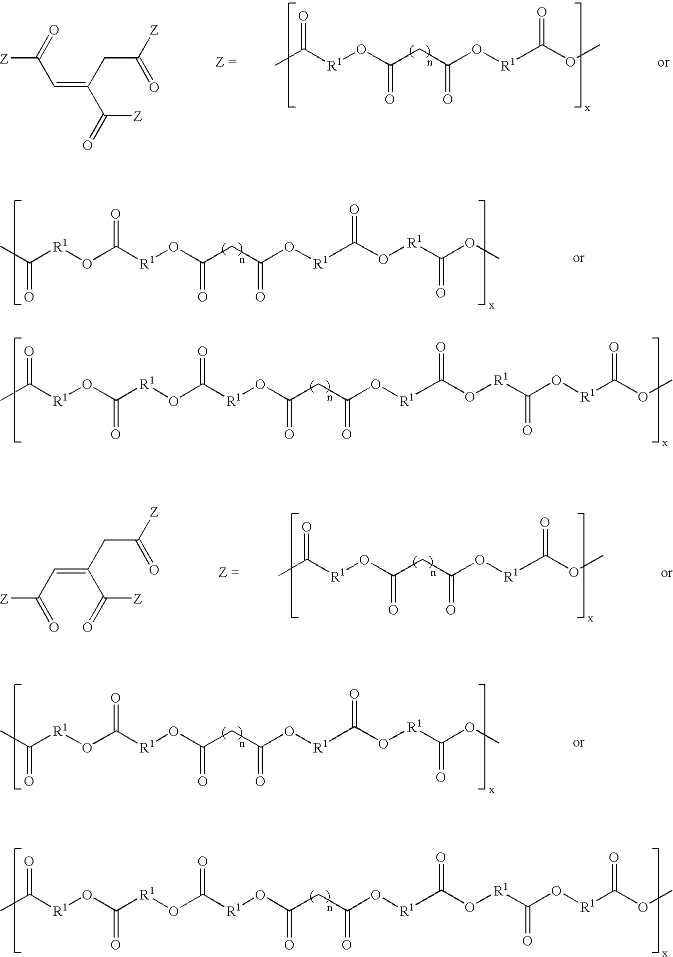 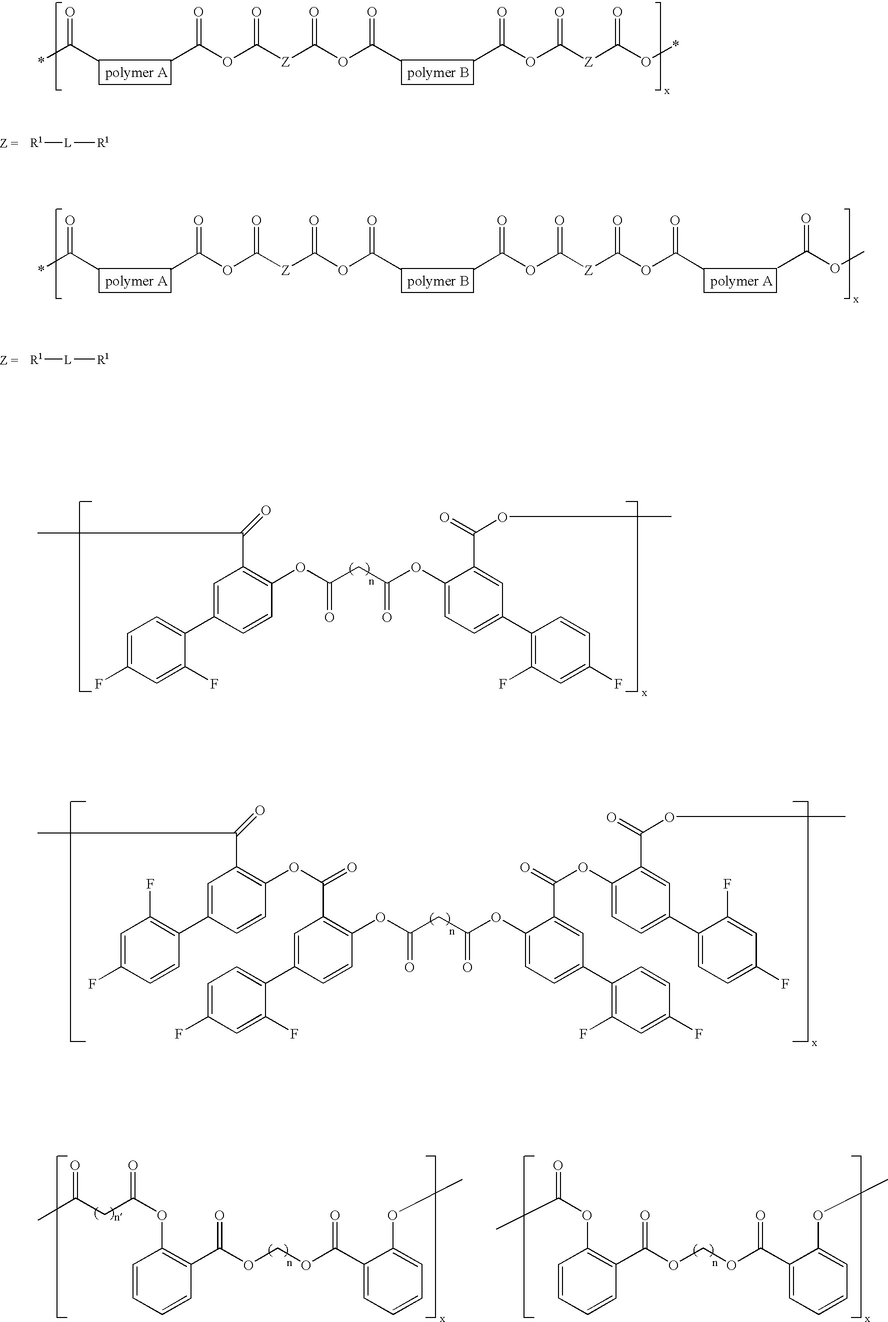
wherein all the variables are as defined above.
c. Linking Groups

The mechanical and degradation properties, e.g. hydrolytic properties, of monomers, oligomers and polymers comprising an agent(s) or compound(s) may be determined by incorporating and/or modifying a linking group into the monomer, or oligomer or polymer backbone. Among other properties, selecting molecular weight and chemical composition of a linking group will critically affect the polymer's glass transition temperature (Tg) and, accordingly, the mechanical properties of the monomer, oligomer or polymer(s) and coatings they form at various temperatures. In general, the higher the molecular weight, the greater the toughness of the material in terms of elasticity and tear strength. The oligo and polymers of the invention may comprise backbones wherein the agent(s) or compound(s) and a linking group(s), and optionally another agent(s), are bonded together through breakable linkages, such as ester, thioester, amide, carbonate, and many others known in the art and their combinations. These structures may have an anti-inflammatory agent(s) and other agents mixed, blended, or dispersed therein. The backbone linkages form biodegradable bonds with the drugs that are hydrolyzed, broken by proteolysis, or broken by other biological of biochemical processes when placed in contact with the appropriate medium, e.g. body tissues or fluids, to release an agent(s) or compound(s). In some embodiments, the linking group(s) may be selected in coordination with the actual agent(s) to impart desirable physical, chemical, and biological properties, such as adhesion to smooth and porous surfaces, e.g. metallic, polymeric, ceramic, or glass surfaces. Such surfaces may be located in diverse environments, including various types of surfaces, structures, including plastic and other polymeric artifacts, alloys, stainless steel, and other metals, or on implantable dental, medical and veterinary devices to allow formation of a coating that may withstand handling, coating, implantation, and exposure to body fluids and tissues, and the like. Other desirable characteristics that are critically influenced by the linker type are mechanical strength, flexibility, and ability to withstand application of mechanical stress without failure, low sticking to a surface so that adhesion to delivery vehicles and neighboring surfaces may be minimized, e.g. when implanted in an animal or human. Also important is resistance to sterilization conditions by different methods, e.g. gamma irradiation, electron beam (E beam), treatment with ethylene oxide, or other chemical or physical treatments providing sterilization. Suitable linking groups typically comprise a divalent organic residue of molecular weight about 25, 40, 75, 100, 130 Dalton to about 100, 170, 250, 330, 400, 520 Dalton. In one embodiment, L comprises a divalent, branched or unbranched, saturated or unsaturated (C₁-C₂₅) hydrocarbon chain, where one or more carbon atoms may be further substituted by —O—, —NR⁷—, an amino acid, a peptide, (C₁-C₆) alkoxy, (C₃-C₆) cycloalkyl, (C₁-C₆) alkanoyl, (C₁-C₆) alkanoyloxy, (C₁-C₆) alkoxycarbonyl, (C₁-C₆) alkylthio, azido, cyano, nitro, halo, hydroxy, oxo, carboxy, aryl, aryloxy, heteroaryl, or heteroaryloxy.

In one embodiment, the monomer, oligomer or polymer may be employed to coat the surface of an article or device in a manner that it will allow for its expansion, contraction or torsion during the application and useful life of the article. In such case a linking group(s) may be a (C₃-C₅₀) dicarboxylic acid hydrocarbon residue.

In one embodiment, the monomer, oligomer or polymer of the invention may comprise a linking group(s) that may be present in the monomer, oligomer or polymer backbone along with the agent(s) through bonds that release the agent(s) under certain environmental conditions. Examples of bonds are esters, thioesters, amides, thioamides, urethanes, carbamates, thiocarbamates, carbonates, thiocarbonates, and any others than fulfill a similar function. This includes combinations and mixtures thereof. The linking bonds may comprise other groups, and atoms, including P, C, O, N, S, halogens, metals, and other inorganic and organic atoms provided that they form labile bonds that may release under appropriate circumstances the agent(s) within the backbone, and the agent(s) mixed into the monomer, oligomer or polymer. The linking group(s) may be selected as well to impart to the monomer, oligomer or polymer desirable physical, chemical, and/or biological properties. Examples of these are adhesion to metallic, polymeric, ceramic or glassy surfaces on implantable medical and veterinary devices to allow formation of a coating that may withstand handling, implantation, and exposure to body tissues and/or fluids post-implantation; sufficient mechanical strength, flexibility, and ability to withstand without failure application of mechanical stress without failure; minimal stickiness on the surface of the resulting coating to minimize adhesion to vehicles used in the delivery or implantation of the medical or veterinary device in the body of a human or animal; and the ability to sterilize the coating and the associated medical or veterinary device by the application of gamma irradiation, electron beam (E beam), treatment with ethylene oxide, or other chemical or physical treatments providing sterilization. Suitable linking groups are widely known in the art, and need not be fully detailed here. Examples are described in U.S. Pat. Nos. 6,613,807; 6,328,988; 6,365,146; 6,468,519; 6,486,214; 6,497,895; 6,602,915; 6,613,807; U.S. Published Patent Applns. 2002/0071822 A1; 2002/0106345 A1; 2003/0035787 A1; 2003/0059469 A1; 2003/0104614 A1; 2003/0170202 A1; U.S. Ser. Nos. 09/508,217; 10/368,288; 10/622,072; 10/646,336; 10/647,701; and International Patent Applications WO 99/12990; WO 01/28492; WO 01/41753; WO 01/58502; WO 02/09767; WO 02/09768; WO 02/09769; WO 03/005959; WO 03/046034; WO 03/065928; and WO 03/072020. The nature of the linking group (L) in a monomer, oligomer or polymer of the invention may be employed to provide the monomer, oligomer or polymer of the invention with one or more desirable physical, chemical, and/or biological properties, such as mechanical and thermal properties; adhesiveness; wetability; hardness; drug generation, and release kinetics and solubility; and tissue compatibility and response for the selected therapeutic application. The linking group L comprises typically a divalent organic radical having a molecular weight (MW) about 25, or 40 daltons to about 200, or 400 daltons. The mechanical and degradative properties, e.g. hydrolytic properties, of the monomer, oligomer or polymer of the invention may be controlled by incorporating and/or modifying a specific linking group (L) into the monomer, oligomer or polymer structure. The mechanical and degradative properties, e.g. hydrolytic properties, of the monomer, oligomer or polymer of the invention may be controlled by incorporating and/or modifying a specific linking group (L) into the polymer structure. L may be any substituted or unsubstituted hydrocarbon unit, such as, for example, propane, butane, pentane, etc. A suitable number of carbon atoms includes any number of carbon atoms that will result in a functional oligomer, e.g. about 2 to about 20 carbon atoms, about 2 to about 18 carbon atoms, about 4 to about 16 carbon atoms, about 4 to about 14 carbon atoms, about 6 to about 16 carbon atoms, about 8 to about 12 carbon atoms, or about 6 to about 10 carbon atoms. Further, the nature of the linking group L in a monomer, oligomer or polymer of the invention may not be critical provided they possess acceptable mechanical properties and release kinetics for the selected therapeutic application. The linking group L comprises typically a divalent organic radical having a molecular weight of from about 5, about 10, about 15, about 20, about 25, or about 40 to about 100, about 200, about 300, or about 400 Dalton, and a length of from about 5, about 10, about 30, or about 40 to about 50, about 75, or about 100 Angstrom using standard bond lengths and angles. The linking group may be biologically inactive, or may itself possess biological or other activity. One preferred monomer, oligomer or polymer comprises L representing a residue of a linking group(s) that, independently from one another, comprises linear or branched (C₃-C₃₀) aliphatic, alicyclic or aromatic residue that may be further substituted. Although any agent(s) may be polymerized in this manner, particularly suited are aliphatic, alicyclic, aromatic small and large organic molecules that have at least two functional groups, and optionally additional groups such as OH, SH, COOH, COOR, phosphate, amine, amide, thioester, thiamide, S, P, N, halogen, ether, aldehyde, ketone, and many others; such molecules being known as suitable for regulation of properties such as hydrophilicity, solubility, and the like. In one embodiment the agent comprises salicylic acid, and the linker comprises a dicarboxylic acid hydrocarbon chain with an even number of carbon atoms. The nature and presence of the linking group L in the monomer, oligomer or polymer may not be critical as long as it does not negatively impact the monomer, oligomer or polymer's acceptable mechanical properties and release kinetics for the selected therapeutic application.

In one embodiment, the linking group L typically comprises a divalent organic residue of molecular weight about 25, about 40, about 60, about 100, about 130, or about 150 Daltons to about 80, about 110, about 125, about 140, about 170, about 250, about 370, or about 400 Daltons, and any combination thereof. In another embodiment the linking group(s) L typically comprises a length of about 5, about 10, about 15, about 20, or about 25 Angstrom to about 30, about 35, about 45, about 50, about 75, or about 100 Angstrom using standard bond lengths and angles. In one embodiment, the linking group may be biologically inactive, and in another it may possess biological activity. The linking group may also comprise other functional groups including hydroxy, mercapto, amine, halo, SH, —O—, —C═O, —N═, —P═, or carboxylic acid, as well as others that may be used to modify the properties of the monomer, oligomer or polymer. These may be employed for example for polymer branching, cross-linking, appending other molecules, e.g. another compound(s), to the polymer, changing the polymer solubility, or affecting the biodistribution of the polymer, among others. In one embodiment, the linking group may incorporate other biodegradable groups such as alpha-ester (lactate, glycolate), ε-caprolactone, ortho-ester, or enzymatically biodegradable groups such as amino acids. In another embodiment, the linking group may be a water-soluble, non-biodegradable segment such as a polyethylene glycol (PEG), polyvinyl alcohol (PVA) or polyvinyl pyrrolidone (PVP). In yet another embodiment, the linking group may be a water-insoluble, non-biodegradable segment such as polypropylene glycol (PPG), polyetherurethane (PEU), or poly(n-alkyl ether). In still another embodiment, the linker may be an amorphous or semicrystalline biodegradable polymer, such as poly(D,L-lactide), poly(trimethylene carbonate), poly(dioxanone), polyanhydridepoly(orthoester) poly(glycolide), poly(L-lactide) poly(ε-caprolactone) and co-polymers of α-caprolactone, glycolide, trimethylene carbonate, dioxanone, D,L-lactide, L-lactide and/or D-lactide. In another embodiment, the linking group may have surfactant properties, such as a Pluronic block copolymer with polyethylene glycol and polypropylene glycol blocks, and in another it may have polar or charged moieties, including carboxylic acid groups from poly(acrylic acid) and poly(alginates), sulfonic acid groups from poly(2-acrylamido-2-methyl-propanesulfonicacid) (AMPS), hydroxy groups from poly(vinyl alcohol), polysaccharides and poly(alginates), and amino groups from poly(L-lysine), poly(2,2-dimethylaminoethyl methacrylate) and poly(amino acids).

In addition, the linking group may be a segment that undergoes thermoreversible gellation, such as Pluronic F127 and poly(N-isopropyl acrylamide). It may incorporate structurally-reinforcing segments, such as polyetherurethane, polyesterurethane, etc. In yet another embodiment, the linking group may be a divalent, branched or unbranched, saturated or unsaturated, hydrocarbon chain, having from 1 to 25 carbon atoms, wherein one or more, e.g. 1, 2, 3, or 4, of the carbon atoms may be optionally replaced by (—O—), (—S—), (—P—), or (—NR⁷—), and wherein the chain may be optionally substituted with one or more, e.g. 1, 2, 3, or 4, substituents comprising (C₁-C₆) alkoxy, (C₃-C₆) cycloalkyl, (C₁-C₆) alkanoyl, (C₁-C₆) alkanoyloxy, (C₁-C₆) alkoxycarbonyl, (C₁-C₆) alkylthio, azido, cyano, nitro, halo, hydroxy, oxo, carboxy, aryl, aryloxy, heteroaryl, or heteroaryloxy, among others. The linking group may be a divalent (C₂-C₃₂) branched or unbranched, saturated or unsaturated hydrocarbon chain optionally further substituted with one or more, e.g. 1, 2, 3, or 4, substituents comprising (C₁-C₆) alkoxy, (C₃-C₆) cycloalkyl, (C₁-C₆) alkanoyl, (C₁-C₆) alkanoyloxy, (C₁-C₆) alkoxycarbonyl, (C₁-C₆) alkylthio, azido, cyano, nitro, halo, hydroxy, oxo, carboxy, aryl, aryloxy, heteroaryl, or heteroaryloxy, among many others. The linking group may be also a biological molecule such as a carbohydrate, saccharide, polysaccharide, fatty acid, lipid, nucleic acid, peptide, amino acid, or combinations thereof. One preferred linking group comprises a divalent, branched or unbranched, saturated or unsaturated (C₁-C₂₀) hydrocarbon, which may be optionally substituted with, e.g. 1, 2, 3, 4, or more, substituents comprising (C₁-C₆)alkoxy, (C₃ C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo, carboxy, aryl, aryloxy, heteroaryl, and heteroaryloxy. Other specific substituents comprise —(CHR⁷)₄—, where each R⁷ comprises hydrogen, —C(═O)—(CH₂)₁₀CH₃, or —O—P(═O)—O(CH₂)₁₀CH₃, among others. Other preferred linking groups comprise an amino acid(s), peptide(s), protein(s), divalent, branched or unbranched, saturated or unsaturated (C₁-C₁₀) hydrocarbon residue(s), wherein one or more carbon comprise(s) or is(are) substituted by —O—, or —NR⁷—. Still other preferred linking groups comprise divalent, branched or unbranched, saturated or unsaturated (C₃-C₂₀) hydrocarbon residue(s), wherein one or more, e.g. 1, 2, 3, 4, or more, carbon atoms is(are) optionally replaced by —O—, or —NR⁷—, and may be further substituted by (C₁-C₆) alkoxy, (C₃-C₆) cycloalkyl, (C₁-C₆) alkanoyl, (C₁-C₆) alkanoyloxy, (C₁-C₆) alkoxycarbonyl, (C₁-C₆) alkylthio, azido, cyano, nitro, halo, hydroxy, oxo, carboxy, aryl, aryloxy, heteroaryl, and/or heteroaryloxy, among others. Still other preferred linking groups comprise divalent, branched or unbranched, saturated or unsaturated (C₃-C₂₀) hydrocarbon, wherein one or more, e.g. 1, 2, 3, 4, or more, atoms is(are) substituted by —O—, —C(═O)O—, —C(═S)O—, —C(═O)S—, —C(═O)NR⁷—, —C(═S)NR⁷—, or —NR⁷—, wherein R⁷ comprises hydrogen, or (C₁-C₆) aliphatic residue. Another group of monomer, oligomer or polymers comprise a linking agent(s) that comprise(s) a divalent, branched or unbranched, saturated or unsaturated (C₃-C₂₀)hydrocarbon, more preferably a (C₄-C₁₅) hydrocarbon, and even more preferably n-butyl, n-hexyl, n-octyl, n-decyl, n-dodecyl or n-tetradecyl.

In another embodiment the polymer may be employed to coat a rigid article, e.g. an implantable orthopedic device, including a hip, knee, shoulder, disk, rib, foot and arm bones, or elbow replacement, a fixation device(s) for other orthopedic applications, and many others. In such case, the linking group(s) may be a (C₃-C₃₅) dicarboxylic acid hydrocarbon residue(s). The linking group contributes to the control of a monomer, oligomer or polymer's characteristics, mechanical properties and release kinetics for selected applications. The linking group(s) typically is(are) about 5, about 10, about 15, about 25, about 50, about 80, or about 120 Angstroms to about 75, about 100, about 140, about 180, about 230, or about 300 Angstroms employing standard bond lengths and angles. The linking group may be biologically inactive, or may itself possess biological activity, and may further comprise O, N, P, halogen, etc. Suitable functional groups that may be attached to the linking group(s) is(are) hydroxy, keto, aldehyde, lactam, mercapto, amide, acryl, vinyl, amine, carboxyl, halo, and many others that may be used to modify the properties of the monomer, oligomer or polymer for example by branching, cross linking, for appending other molecules, e.g. other biologically active of activatable compound(s), to the monomer, oligomer or polymer, for changing the solubility of the monomer, oligomer or polymer, or for affecting the biodistribution of the monomer, oligomer or polymer, etc. Thus, different embodiments may be prepared changing the chemical structure of the linker that will evidence a direct or reverse correlation with the Tg of the specific type of polymers. This invention provides an improved process for the preparation of the present monomer, oligomer or polymers which permits the synthetic design of monomer, oligomer or polymers with pre-designed properties and, moreover, of properties never before attained with prior synthetic processes. For example, in one embodiment the monomer, oligomer or polymer may be prepared from an agent(s) or compound(s) of chemical formula Z¹—R¹-Z² and a linker precursor of formula X¹-L-X², wherein Z¹, Z², X¹, and X², independently from one another, comprise functional groups that are able to form degradable bonds in situ. Examples of these functional groups are shown in Table 3 below.

TABLE 3
Functional Groups & Monomer, Oligomer and Polymer Bonds

| Agent | | |
|---|---|---|
| Group | Linker Group | |
| (Z¹ or Z²) | (X¹ or X²) | Bond (A) |
| —COOH | —OH | Ester |
| —COOH | —NHR | Amide |
| —COOH | —SH | Thioester |
| —OH | —COOH | Ester |
| —SH | —COOH | Thioester |
| —NHR | —COOH | Amide |

Z¹⁻R¹⁻Z² + X¹-L-X² → —[C(═O)—R¹-A-L-A-R¹—C(═O)—O]ₓ— (XIII) wherein x is a positive integer showing the degree of polymerization

An agent(s) or compound(s) and a linker precursor may be polymerized, for example, by condensation, to provide a oligomer or polymer such as, for example, that of chemical formula (XIII), wherein each A, independently from one another, comprises a bond that may be degradable in situ, e.g. in vivo when administered to a living organism. Examples of breakable bonds comprise an ester, thioester, thioamide, azo, carbonate, or amide. Depending on the reactive functional groups Z¹ and Z² present in the agent(s) or compound(s), a corresponding functional group X¹ or X² may be selected for the linking group or second functional group of the agent(s) or compound(s) to provide one or more of the breakable bonds described above in the formation of the backbone. The oligomers and polymers of the present invention may be prepared in at least two general manners or embodiments, which embodiments are expanded by the addition, and various permutations, of the optional steps that each of the illustrative methods shown in the Schemes shown below. In one embodiment, the polymerization step occurs in a non-aqueous dispersion medium. Once a pre-polymer(s) or a diacid monomer(s) is synthesized and activated as a mixed anhydride, it may be heated above its melting point in the presence of a solvent for the pre-polymer(s), e.g. an inert, high boiling point pre-polymer solvent, to allow polymerization to occur while the thus produced oligomer or polymer remains out of solution as it is generated. This process is capable of yielding polymers of high molecular weights, e.g. in excess of 40,000 Dalton. Vigorous mechanical mixing or stirring may be favorably employed with an optional addition of a minor amount of, or even without, a non-aqueous dispersing agent or surfactant that will foster the formation of a suitable emulsion of molten droplets of the polymerization phase.

d. Other Agents and Compounds

Any diagnostic agent(s) may be incorporated into the backbone of the monomer, oligomer or polymers of the invention, or be dispersed into, or carried by them. Examples are phosphorescent agents, fluorescent agents, radioactive agents, enzymatic agents, among others. Any additional therapeutic agent(s) is(are) suitable for use in the backbone, or dispersed into, or carried by the monomer, oligomer or polymer. Examples of therapeutic agent classes include antibacterial, antiviral, antiproliferative, anticancer, anti-inflammatory, analgesic, anesthetic, antipyretic, antiseptic, and antimicrobial compounds. Examples of compounds in those classes include salicylic acid, 4-aminosalicylic acid, 5-aminosalicylic acid, 4-(acetylamino) salicylic acid, 5-(acetylamino) salicylic acid, 5-chlorosalicylic acid, salicylsalicylic acid (salsalate), 4-thiosalicylic acid, 5-thiosalicylic acid, 5-(2,4-difluoro-phenyl) salicylic acid (diflunisal), 4-(trifluoromethyl) salicylic, sulfasalazine, diclofenac, penicillamine, balsalazide, olsalazine, mefenamic acid, carbidopa, levodopa, etodolac, cefaclor, captopril, and the like. Any traceable agent(s) or compound(s) may be suitable for use in this invention. A synthetic process described below enables the preparation of different embodiments by modifying the chemical structure of a linker taking into consideration that such change will evidence a direct or reverse correlation with the T_{g} of the specific polymers. This process enables the preparation of polyanhydrides that release a broad scope of families of agents and drugs, such as those disclosed in U.S. Pat. No. 6,486,214. Compounds suitable for incorporation into or dispersion in, blending or mixing with the monomer, oligomer or polymer of this invention preferably have relatively low molecular weights, e.g. up to 1000 dalton. The compounds generally contain within their molecular structure at least one functional group, and preferably two functional groups, more preferably one of the functional groups comprises carboxylic acid. The functional groups of the compound(s) are preferably hydroxy (—OH), thiol (—SH), amine (—NHR), amide (—C(═O)—NR⁷), azo, carbonate (—O—C(═O)O—), carboxy (—C(═O)—OH), and similarly breakable groups. These functional groups form breakable, e.g. biodegradable, bonds within the monomer, oligomer or polymer and are able to release the compound in its active form or as a precursor. The monomer, oligomer or polymer bonds may be broken by hydrolysis, such as proteolysis, or by other biological or biochemical processes when placed in contact with the target environment, e.g. body tissues or fluids. The compounds may also comprise other functional groups, including hydroxy, phenol, ketone, aldehyde, double and triple bond C—C substituents, amide, mercapto, amine, halide, carboxylic acid, and many others known in the art, all of which may be used to modify the properties of the monomer, oligomer or polymer, such as for branching, cross-linking, appending other molecules to the monomer, oligomer or polymer, changing their characteristics such as solubility, consistency, adhesiveness, or rigidity, among others, or for affecting their distribution in a specific system, e.g. biodistribution. One skilled in the art will be able to readily select from the listed compounds those that possess, or may be modified by methods known in the art to possess, the necessary functional groups for polymerization in accordance with the method of the invention. Suitable therapeutic and diagnostic compounds may be found, for example, in the Physician's Desk Reference, 55 Ed., Medical Economics Company, Inc., Montvale, New Jersey (2001); USPN Dictionary of USAN and International Drug Names, The United States Pharmacopeia Convention, Inc., Rockville, Md. (2000); The Merck Index, 12 Ed., Merck & Co., Inc., Whitehouse Station, New Jersey (1996). Any suitable agent may be employed in the monomer, oligomer or polymers of the invention. In one embodiment the agent(s) possess(es) at least two functional groups that may be incorporated into an ester, thioester, urethane, carbamate, carbonate, or amide linkage, among others, of a monomer, oligomer or polymer, such that, upon erosion, hydrolysis or enzymatic degradation of the monomer, oligomer or polymer, the agent(s) may be released. The functional groups may independently comprise hydroxy (—OH), mercapto (—SH), amine (—NHR), or carboxylic acid (—COOH), among others. These functionalities form biodegradable bonds with the agent(s) to be polymerized. The latter are hydrolyzed, or broken by a proteolytic process, or other biological or biochemical processes when placed in contact with body tissues or fluids. The agent(s) may also comprise other functional groups, (including hydroxy, mercapto, amine, and carboxylic acid, as well as others, that may be used to modify the properties of the monomer, oligomer or polymer, e.g. for branching, for cross linking, for appending other molecules, e.g. another active compound, to the monomer, oligomer or polymer, for changing their solubility, or for effecting their biodistribution. One skilled in the art may readily select agents that possess the necessary functional groups for incorporation into the monomer, oligomer or polymers of the invention from these lists. The agent may comprise a biological, diagnostic, therapeutic, or other type of agent such as suitably functionalized analgesics, anesthetics, anti-infectives including disinfectants, antiseptics, antibiotics, anti-fungal agents, anti-viral agents, anti-microbial agents, or bacteriostatic agents, among others, anti-diabetic agents, anti-dyskinetics, anti-fibrotic agents, anti-inflammatory agents, anti-neoplastic agents, anti-osteoporotic agents, bone resorption inhibitors, hormones, immunomodulating agents such as immunosuppressive and immunostimulating agents, muscle relaxants, anti-cancer agents such as antibodies and their fragments, radioactive materials, anti-angiogenic agents, carcinolytic agents, nucleoside analogs, anti-sense agents, anti-oxidant agents, metabolic and anti-metabolic agents, vasodilators, prostaglandins, and their inhibitors, ultraviolet, radioactive and phosphorescent screening agents, agents for the treatment of osteoporosis, sclerosis, aesophagal, respiratory, tracheal, buccal, laryngeal, nasal, and throat conditions and ailments, among many others. See, for example, Physicians' Desk Reference, 55 Ed., pp. 201-202 (2001). Examples of specific therapeutic, screening and diagnostic agents or compounds that may be incorporated into the monomer, oligomer or polymers of the invention are acriflavine; acyclovir; amoxicillin; albuterol; alendronate; amicarbilide; aminoquinuride; arsphenamine; atorvastatin; azithromycin; benazepril; bialamicol; budesonide; bupivacaine; buprenorphine; butorphanol; capecitabine; captopril; carboplatin; cefaclor; ceftazidime; ceftriaxone; chloroazodin; cilastatin; ciprofloxacin; clarithromycin; cladribine; cyclosporin; cytarabine; diclofenac; daunorubicin; diflunisal; docetaxel; dopamine; doxorubicin; enalapril; famotidine; floxuridine; fludarabine phosphate; fluvastatin; idarubicin; imipenem; indinavir; lamivudine; leuprolide; lisinopril; mepivacaine, 6-mercaptopurine; metformin; metoprolol; mitomycin C; mitoxantrone; morphine; nalbuphine, nizatidine; oxymorphone; paclitaxel; pentostatin; phenamidine; plicamycin; podophyllinic acid 2-ethylhydrazine; pravastatin; quinapril; ranitidine; salmeterol; streptozocin; thioguanine; xinafoate; zidovudine; podophyllotoxin; etoposide; gemcitabine; camptothecin; topotecan; irinotecan; vinorelbine; vincristine; vinblastine; teniposide; tamoxifen; melphalan; methotrexate; 2-p-sulfanilyanilinoethanol; 4,4′-sulfinyldianiline; 4-sulfanilamidosalicylic acid; acediasulfone; acetosulfone; amikacin; ampicillin; amphotericin B; ampicillin; apalcillin; apicycline; apramycin; arbekacin; aspoxicillin; azidamfenicol; aztreonam; bacitracin; bambermycin(s); biapenem; brodimoprim; butirosin; capreomycin; carbenicillin; carbomycin; carumonam; cefadroxil; cefamandole; cefatrizine; cefbuperazone; cefclidin; cefdinir; cefditoren; cefepime; cefetamet; cefixime; cefinenoxime; cefininox; cefodizime; cefonicid; cefoperazone; ceforamide; cefotaxime; cefotetan; cefotiam; cefozopran; cefpimizole; cefpiramide; cefpirome; cefprozil; cefroxadine; cefteram; ceftibuten; ceftizonam; cephalexin; cephaloglycin; cephalosporin C; cephradine; chloramphenicol; chlortetracycline; clinafloxacin; clindamycin; clomocycline; colistin; cyclacillin; dapsone; demeclocycline; diathymosulfone; dibekacin; dihydrostreptomycin; dirithromycin; doxycycline; enoxacin; enviomycin; epicillin; erythromycin; flomoxef; fortimicin(s); gentamicin(s); glucosulfone solasulfone; gramicidin S; gramicidin(s); grepafloxacin; guamecycline; hetacillin; isepamicin; josamycin; kanamycin(s); leucomycin(s); lincomycin; lomefloxacin; lucensomycin; lymecycline; meclocycline; meropenem; methacycline; methsalamine; micronomicin; midecamycin(s); minocycline; moxalactam; mupirocin; nadifloxacin; natamycin; neomycin; netilmicin; norfloxacin; oleandomycin; oxytetracycline; p-sulfanilylbenzylamine; panipenem; paromomycin; pazufloxacin; penicillin N; pipacycline; pipemidic acid; polymyxin; primycin; quinacillin; ribostamycin; rifamide; rifampin; rifamycin SV; rifapentine; rifaximin; ristocetin; ritipenem; rokitamycin; rolitetracycline; rosaramycin; roxithromycin; salazosulfadimidine; salicylic acid, sancycline; sisomicin; sparfloxacin; spectinomycin; spiramycin; streptomycin; succisulfone; sulfachrysoidine; sulfaloxic acid; sulfamidochrysoidine; sulfanilic acid; sulfoxone; teicoplanin; temafloxacin; temocillin; tetroxoprim; thiamphenicol; thiazolsulfone; thiostrepton; ticarcillin; tigemonam; tobramycin; tosufloxacin; trimethoprim; trospectomycin; trovafloxacin; tuberactinomycin; vancomycin; azaserine; candicidin(s); chlorphenesin; dermostatin(s); filipin; fumgichromin; mepartricin; nystatin; oligomycin(s); perimycin A; tubercidin; 6-azauridine; 6-diazo-5-oxo-L-norleucine; aclacinomycin(s); ancitabine; anthramycin; azacitadine; azaserine; bleomycin(s); carubicin; carzinophillin A; chlorozotocin; chromomycin(s); denopterin; doxifluridine; edatrexate; eflornithine; elliptinium; enocitabine; epirubicin; mannomustine; menogaril; mitobronitol; mitolactol; mopidamol; mycophenolic acid; nogalamycin; olivomycin(s); peplomycin; pirarubicin; piritrexim; prednimustine; procarbazine; pteropterin; puromycin; ranimustine; streptonigrin; thiamiprine; tamoxifen; Tomudex® (N-[[5-[[(1,4-Dihydro-2-methyl-4-oxo-6-quinazolinyl)methyl]methylamino]-2-thienyl]carbonyl]-L-glutamic acid), trimetrexate, tubercidin, ubenimex, vindesine, zorubicin; argatroban; coumetarol; dicoumarol; ethyl biscoumacetate; ethylidene dicoumarol; iloprost; lamifiban; taprostene; tioclomarol; tirofiban; amiprilose; bucillamine; gusperimus; mycophenolic acid; procodazole; romurtide; sirolimus (rapamycin); tacrolimus; butethamine; fenalcomine; hydroxytetracaine; naepaine; orthocaine; piridocaine; salicyl alcohol; 3-amino-4-hydroxybutyric acid; aceclofenac; alminoprofen; amfenac; bromfenac; bromosaligenin; bumadizon; carprofen; diclofenac; diflunisal; ditazol; enfenamic acid; etodolac; etofenamate; fendosal; fepradinol; flufenamic acid; gentisic acid; glucamethacin; glycol salicylate; meclofenamic acid; mefenamic acid; mesalamine; niflumic acid; olsalazine; oxaceprol; S-adenosylmethionine; salicylic acid; salsalate; sulfasalazine; and tolfenamic acid, among many other suitable. Any combination of these classes of agents may be made, including groupings in twos, threes, fours, . . . , ns, for different applications, all in accordance with this invention.

Other examples of suitable agents are argatroban; arsphenamine; azacitadine; azaserine; azithromycin; bleomycin(s); bromfenac; bromosaligenin; bucillamine; budesonide; bumadizon; buprenorphine; butethamine; butirosin; butorphanol; candicidin(s); captopril; carbenicillin; carboplatin; carprofen; carubicin; carumonam; carzinophillin A; ceftriaxone; chloroazodin; chloroazodin; chlorozotocin; chlorphenesin; chromomycin(s); cladribine; clarithromycin; coumetarol; cyclosporin; denopterin; dermostatin(s); diclofenac; dicoumarol; diflunisal; ditazol; docetaxel; dopamine; doxifluridine; edatrexate; eflornithine; elliptinium; enalapril; enfenamic acid; enocitabine; epirubicin; ethyl biscoumacetate; ethylidene; etodolac; etofenamate; etoposide; famotidine; fenalcomine; fendosal; fepradinol; filipin; floxuridine; fludarabine phosphate; flufenamic acid; fluvastatin; fortimicin(s); fungichromin; gemcitabine; gentisic acid; glucamethacin; glucosulfone; glycol salicylate; gramicidin S; gramicidin(s); grepafloxacin; guamecycline; gusperimus; hetacillin; hydroxytetracaine; idarubicin; iloprost; imipenem; indinavir; isepamicin; josamycin; kanamycin(s); lamifiban; lamivudine; leucomycin(s); leuprolide; lincomycin; lisinopril; lisinpril; lomefloxacin; lucensomycin; lymecycline; mannomustine; meclocycline; meclofenamic acid; mefenamic acid; melphalan; menogaril; mepartricin; meropenem; mesalamine; metformin; methacycline; methotrexate; methsalamine; metoprolol; micronomicin; midecamycin(s); minocycline; mitobronitol; mitolactol; mitomycin C; mitoxantrone; mopidamol; morphine; moxalactam; mupirocin; mycophenolic acid; nadifloxacin; naepaine; nalbuphine; natamycin; neomycin; netilmicin; niflumic acid; nizatidine; nogalamycin; norfloxacin; nystatin; oleandomycin; oligomycin(s); olivomycin(s); olsalazine; orthocaine; oxaceprol; oxymorphone; oxytetracycline; paclitaxel; panipenem; paromomycin; pazufloxacin; penicillin N; pentostatin; peplomycin; perimycin A; phenamidine; pipacycline; pipemidic acid; pirarubicin; piridocaine; piritrexim; plicamycin; podophyllinic acid 2-ethylhydrazine; polymyxin; pravastatin; prednimustine; primycin; procarbazine; procodazole; p-sulfanilylbenzylamine; pteropterin; puromycin; quinacillin; quinapril; ranimustine; ranitidine; ribostamycin; rifamide; rifampin; rifamycin SV; rifapentine; rifaximin; ristocetin; ritipenem; rokitamycin; rolitetracycline; romurtide; rosaramycin; roxithromycin; S-adenosylmethionine; salazosulfadimidine; salicyl alcohol; salicylic acid; salmeterol; salsalate; sancycline; sirolimus (rapamycin); sisomicin; solasulfone; sparfloxacin; spectinomycin; spiramycin; streptomycin; streptonigrin; streptozocin; succisulfone; sulfachrysoidine; sulfaloxic acid; sulfamidochrysoidine; sulfanilic acid; sulfasalazine; sulfoxone; tacrolimus; taprostene; teicoplanin; temafloxacin; temocillin; teniposide; tetracycline; tetroxoprim; thiamiprine; thiamphenicol; thiazolsulfone; thioguanine; thiostrepton; ticarcillin; tigemonam; tioclomarol; tirofiban; tobramycin; tolfenamic acid; topotecan; tosufloxacin; trimethoprim; trimetrexate; trospectomycin; trovafloxacin; tuberactinomycin; tubercidin; ubenimex; vancomycin; vinblastine; vincristine; vindesine; vinorelbine; xinafoate; zidovudine; zorubicin; and any enantiomers, derivatives, bases, salts or mixtures thereof.

In one embodiment, the agent comprises at least one non-steroidal anti-inflammatory drug(s) (NSAID(s)) such as those described in U.S. Pat. Nos. 6,486,214 and 6,613,807; the relevant texts of both of which are incorporated herein by reference. In another embodiment, the additional agent(s) include an anti-bacterial, for example, 2-p-sulfanilyanilinoethanol, 4,4′-sulfinyldianiline, 4-sulfanilamidosalicylic acid, acediasulfone, acetosulfone, amikacin, amoxicillin, amphotericin B, ampicillin, apalcillin, apicycline, apramycin, arbekacin, aspoxicillin, azidamfenicol, azithromycin, aztreonam, bacitracin, bambermycin(s), biapenem, brodimoprim, butirosin, capreomycin, carbenicillin, carbomycin, carumonam, cefadroxil, cefamandole, cefatrizine, cefbuperazone, cefclidin, cefdinir, cefditoren, cefepime, cefetamet, cefixime, cefinenoxime, cefminox, cefodizime, cefonicid, cefoperazone, ceforamide, cefotaxime, cefotetan, cefotiam, cefozopran, cefpimizole, cefpiramide, cefpirome, cefprozil, cefroxadine, ceftazidime, cefteram, ceftibuten, ceftriaxone, cefuzonam, cephalexin, cephaloglycin, cephalosporin C, cephradine, chloramphenicol, chlortetracycline, ciprofloxacin, clarithromycin, clinafloxacin, clindamycin, clomocycline, colistin, cyclacillin, dapsone, demeclocycline, diathymosulfone, dibekacin, dihydrostreptomycin, dirithromycin, doxycycline, enoxacin, enviomycin, epicillin, erythromycin, flomoxef, fortimicin(s), gentamicin(s), glucosulfone solasulfone, gramicidin S, gramicidin(s), grepafloxacin, guamecycline, hetacillin, imipenem, isepamicin, josamycin, kanamycin(s), leucomycin(s), lincomycin, lomefloxacin, lucensomycin, lymecycline, meclocycline, meropenem, methacycline, micronomicin, midecamycin(s), minocycline, moxalactam, mupirocin, nadifloxacin, natamycin, neomycin, netilmicin, norfloxacin, oleandomycin, oxytetracycline, p-sulfanilylbenzylamine, panipenem, paromomycin, pazufloxacin, penicillin N, pipacycline, pipemidic acid, polymyxin, primycin, quinacillin, ribostamycin, rifamide, rifampin, rifamycin SV, rifapentine, rifaximin, ristocetin, ritipenem, rokitamycin, rolitetracycline, rosaramycin, roxithromycin, salazosulfadimidine, sancycline, sisomicin, sparfloxacin, spectinomycin, spiramycin, streptomycin, succisulfone, sulfachrysoidine, sulfaloxic acid, sulfamidochrysoidine, sulfanilic acid, sulfoxone, teicoplanin, temafloxacin, temocillin, tetracycline, tetroxoprim, thiamphenicol, thiazolsulfone, thiostrepton, ticarcillin, tigemonam, tobramycin, tosufloxacin, trimethoprim, trospectomycin, trovafloxacin, tuberactinomycin, vancomycin and the like. In still another embodiment, the agent comprises an anti-fungal agent such as amphotericin B, azaserine, candicidin(s), chlorphenesin, dermostatin(s), filipin, fungichromin, lucensomycin, mepartricin, natamycin, nystatin, oligomycin(s), perimycin A, tubercidin, and the like. In another embodiment the agent comprises an anti-cancer, e.g., carcinomas, sarcomas, leukemias and cancers derived from cells of the nervous system, including anti-neoplastic, for example, 6-azauridine, 6-diazo-5-oxo-L-norleucine, 6-mercaptopurine, aclacinomycin(s), ancitabine, anthramycin, azacitadine, azaserine, bleomycin(s), capecitabine, carubicin, carzinophillin A, chlorozotocin, chromomycin(s), cladribine, cytarabine, daunorubicin, denopterin, docetaxel, doxifluridine, doxorubicin, edatrexate, eflornithine, elliptinium, enocitabine, epirubicin, etoposide, floxuridine, fludarabine, gemcitabine, idarubicin, mannomustine, melphalan, menogaril, methotrexate, mitobronitol, mitolactol, mitomycin C, mitoxantrone, mopidamol, mycophenolic acid, nogalamycin, olivomycin(s), paclitaxel, pentostatin, peplomycin, pirarubicin, piritrexim, plicamycin, podophyllinic acid 2-ethylhydrazine, prednimustine, procarbazine, pteropterin, puromycin, ranimustine, streptonigrin, streptozocin, teniposide, thiamiprine, thioguanine, Tomudex® (N-[[5-[[(1,4-Dihydro-2-methyl-4-oxo-6-quinazolinyl)methyl]methylamino]-2-thienyl]carbonyl]-L-glutamic acid), topotecan, trimetrexate, tubercidin, ubenimex, vinblastine, vindesine, vinorelbine, zorubicin and the like. In yet another embodiment, the agent comprises an anti-thrombotic, for example, argatroban, coumetarol, dicoumarol, ethyl biscoumacetate, ethylidene dicoumarol, iloprost, lamifiban, taprostene, tioclomarol, tirofiban and the like. The agent may also comprise an immunosuppressive, for example, 6-mercaptopurine, amiprilose, bucillamine, gusperimus, mycophenolic acid, procodazole, romurtide, sirolimus (rapamycin), tacrolimus, ubenimex and the like; a general or local anesthetic such as butethamine, fenalcomine, hydroxytetracaine, naepaine, orthocaine, piridocaine, salicyl alcohol and the like, and many others whose list is too extensive to incorporate into the text of this patent.

In still another embodiment the agent(s) and the additional agent(s) comprise a low molecular weight drug suitable for linkage into degradable co-oligomers and co-polymers via a polyanhydride. Such drug(s) typically has(have) relatively low molecular weight(s), e.g. about 1,000 daltons or less, and may comprise one or more of a carboxylic acid (—COOH), amine (—NH—, —NR⁷—), thiol (—SH, —SR—), alcohol (—OH), phenol (-Ph-OH), ester (—COO—), carbonate (OCOO—), or other labile bonds that are suitable as well. Suitable examples may be found in almost all classes of drugs. Any combination of an anti-inflammatory agent(s) with an additional agent(s), whether or not specifically named or described in this patent, is encompassed within the four corners of this invention. In yet another embodiment, each R¹, independently from one another, comprises at least one residue(s) of the chemical formula 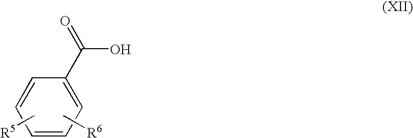
wherein R⁵ comprises amine, thiol, carbonate, amide, halo, or hydroxy; R⁶ comprises hydrogen, halo, NHR⁷, or aryl, which may be substituted with hydroxy, halo or halo (C₁-C₄)alkyl; and R⁷ comprises hydrogen, (C₁-C₆)alkyl, (C₃-C₆)cycloalkyl, (C₃-C₆)cycloalkyl (C₁-C₆)alkyl, aryl, heteroaryl, aryl (C₁-C₆)alkyl, heteroaryl (C₁-C₆)alkyl, or (C₁-C₄)alkylcarbonyl, all of which may be further substituted. In another embodiment R¹ comprises aryl comprising residue that will yield the agent(s) in an active or activatable form upon hydrolysis. In another embodiment each agent(s) and additional agent(s) comprise(s), independently from one another, at least one anti-inflammatory, analgesic, anesthetic, or anti-pyretic compound comprising carboxylic acid and at least one amine, thiol, amide, carbonate, or hydroxy. All specific and preferred values for residues, substituents, linking groups, and ranges in this patent are provided for illustration only, and should serve as mere guidance to an invention that is not limited by the specific information listed. More specifically, lower alkyl may be straight or branched (C₁-C₆)alkyl such as methyl, ethyl, propyl, isopropyl, butyl, iso-butyl, sec-butyl, pentyl, 3-pentyl, or hexyl, among others; (C₃-C₆)cycloalkyl such as cyclopropyl, cyclobutyl, cyclopentyl, or cyclohexyl; (C₃C₆)cycloalkyl (C₁-C₆)alkyl may be cyclopropylmethyl, cyclobutylmethyl, cyclopentylmethyl, cyclohexylmethyl, 2-cyclopropylethyl, 2-cyclobutylethyl, 2-cyclopentylethyl, or 2-cyclohexylethyl, among others; (C₁-C₆)alkoxy such as methoxy, ethoxy, propoxy, isopropoxy, butoxy, iso-butoxy, sec-butoxy, pentoxy, 3-pentoxy, or hexyloxy, among others; (C₁-C₆)alkanoyl such as acetyl, propanoyl or butanoyl, among others; (C₁-C₆)alkoxycarbonyl such as methoxycarbonyl, ethoxycarbonyl, propoxycarbonyl, isopropoxycarbonyl, butoxycarbonyl, pentoxycarbonyl, or hexyloxycarbonyl, among others; (C₁-C₆)alkylthio such as methylthio, ethylthio, propylthio, isopropylthio, butylthio, isobutylthio, pentylthio, or hexylthio, among others; (C₂-C₆)alkanoyloxy such as acetoxy, propanoyloxy, butanoyloxy, isobutanoyloxy, pentanoyloxy, or hexanoyloxy, among others; aryl such as phenyl, indenyl, or naphthyl, among others; and heteroaryl may be furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide) or quinolyl (or its N-oxide), among others.

Yet another group of monomer, oligomer or polymers includes an agent(s) or compound(s) where R⁵, independently from one another, comprise(s) HO(C₁-C₆)alkylene; HS(C₁-C₆)alkylene, R⁷HN(C₁-C₆)alkylene, —OH, —SH, —NH₂, —HNR⁷, wherein R⁷ comprises alkyl, alkenyl, alkynyl, alkoxy, carboxy, cycloaliphatic residue, aryl, among others, which may be further substituted with halogen, O, N, S, or P; R⁶, independently from one another, comprises halo, NHR, cycloaliphatic residue, or aryl, which may be substituted with hydroxy, halo or halo(C₁-C₄)alkyl, wherein R comprises hydrogen or (C₁-C₄) alkyl carbonyl, —NH₂, —NHAc, —Cl, 2,4-difluorophenyl, chloromethyl, difluoromethyl, —CF₃, with —Cl, and 2,4-difluoro-phenyl being highly preferred. Another preferred group of monomer, oligomer or polymers comprises a residue where R comprises H or (C₁-C₆) alkyl, more preferably methyl, ethyl or propyl. Another group of monomer, oligomer or polymers comprises a residue where R, independently from one another, comprises H, (C₁-C₆)alkyl, (C₃-C₆)cycloalkyl, (C₃-C₆)cycloalkyl (C₁-C₆)alkyl, aryl or aryl (C₁-C₆)alkyl. A group of compounds and monomer, oligomer or polymers is that where Y comprises O. Another group of monomer, oligomer or polymers releases an active or activatable agent(s) or compound(s) comprising a biologically active hydroxy-carboxylic acid(s), or that may be converted to such agent(s) upon release in situ. The hydroxy-carboxylic agents may be aliphatic or aromatic agents. Preferred among these are agents such as a (C₁-C₅₀) aliphatic carboxylic acid(s) comprising 1 to 5 hydroxy groups, including alpha-hydroxy carboxylics, beta-hydroxy carboxylic acids, and polyhydroxy carboxylic acids, of which one preferred group comprises C₈ and C₁₄ acids. Another preferred group of agents are hydroxy-aryl carboxylic acids, such as ortho-, meta- and para-hydroxy aryl carboxylic acids, among which preferred are those having anti-inflammatory activities. In another embodiment the agent(s) included in R¹ the monomer, oligomer or polymer, independently from one another, comprise(s) residues of different agents. This embodiment is particularly suitable for the administration of a combination of different or complementary agents, such as in adjunct therapy administered to a subject in medical and veterinary applications, among others. Such monomer, oligomer or polymers are also useful for applications other than screening, diagnosis and therapy where, for example, an additive such as an anti-infective(s) may be combined with a coating agent(s) in their backbone to seal an inanimate surface.

V. Monomer, Oligomer and Polymer Preparation

a. Introduction

The monomer, oligomer or polymers of the invention may be prepared by any suitable method known in the art. Examples are those described in WO 99/12990; U.S. Ser. Nos. 09/917,231; 09/917,194; 09/508,217; 09/422,294; 09/732,516; 60/220,707; 60/261,337; 60/058,328; and 60/220,998; and Conix, Macromol. Synth. 2: 95-99 (1966). When specific characteristics are desired, the monomer, oligomer or polymers may be prepared using processes described herein. The present oligomers and polymers may be produced by chemical connection of monomers (“-mers”). In one embodiment comprising repeating units, two drug molecules may be connected via labile bonds, e.g. ester bonds, to one linker molecule; and via anhydride bonds to one another to form a monomer, oligomer or polymer. In one of the polymerization schemes the monomers are dissolved in a solvent and stirred for several hours at relatively high temperatures. Polymers of molecular weights (MWs) of about 30,000 to about 90,000 Dalton and higher, and poly-dispersities, a measure of polymer homogeneity, of about 1.5 to about 3.0 may be produced by this method. This solution method permits the preparation of polymers of higher MWs, in higher yields, and of greater uniformity than prior art methods.

By definition, all biodegradable monomer, oligomer or polymers are designed to degrade and release its agent(s) over a period of time. Unlike other mono-, oligo- and poly-(anhydride-esters) reported in the literature, the present mono-, oligo- and polymers may be highly soluble in common industrial solvents, and are relatively stable (as measured by loss of molecular weight) both in bulk and in solution. Their desirable “bulk stability”, or molecular weight stability at room temperature is generally about 1 week, about 1 month, about 6 months to about 8 months, about 1 year, about 2 years, although longer periods of stability may be attained as well. The stability of the monomer, oligomer and polymers may be enhanced by storage under dry conditions and at low temperatures e.g. about −20° C. However, even under unprotected ambient conditions, polymers such as polyNSAIDs are stable for weeks. Storage-related changes in molecular weight, however, do not significantly affect monomer, oligomer or polymer performance for drug delivery.

b. Melt Polymerization

The aromatic polyanhydrides may be prepared by the method described by Conix in Macromol. Synth. 2: 95-99 (1966), in which dicarboxylic acids are acetylated in an excess of acetic anhydride followed by melt condensation of the resulting carboxylic acid anhydride at 180° C. for 2-3 hours. Other suitable methods for preparing these polymers are described below or in WO02/09768, WO02/09767, WO01/41753, WO99/12990, or are otherwise in the public domain.

c. Non-Aqueous Dispersion Process

The polymers for the present invention can be also prepared by non-aqueous dispersion polymerization process that attains high molecular weights, e.g. in excess of 40,000 Dalton, with negligible or no gel formation. It starts from a mixed anhydride of a dicarboxylic acid, and comprises heating a solution of the mixed anhydride above its melting point in the presence of a solvent, e.g. an inert high boiling point solvent that will not be a solvent for the monomer, oligomer or polymer, under conditions effective for removing a mixed anhydride evolved upon polymerization. The mild conditions of this process permit the extension of a polyanhydride to higher molecular weights than attainable by existing processes that form gelatinous or insoluble oligomer or polymer fractions that slow the polymerization reaction and impede the extension of the monomer, oligomer or polymer. The melt-polymerization of selected diacids formed as mixed anhydrides with lower molecular weight acids e.g. acetic or propionic acids permits the extension of the backbone by carrying out a non-aqueous dispersion (NAD) of molten droplets suspended in a stable high boiling heat-transfer fluid that is generally chemically unreactive with respect to the monomer, oligomer or polymer. The formation of a stable NAD may be carried out by any known method, such as by vigorous mechanical mixing or stirring, for example with a variety of agitator designs or proprietary mixing devices, or by incorporating a minor amount of a dispersing agent or surfactant, e.g. a non-aqueous agent or surfactant, to encourage the formation of a stable emulsion of molten droplets of the polymerization phase as a dispersion in the continuous phase of the inert fluid. In one embodiment, the dispersing agent should not react chemically with the polyanhydride, its chemical nature being free from any functional groups that would react with the anhydride moieties in the monomer, oligomer or polymer. In another embodiment, the reaction may be carried out in the absence of any surfactant. The particle size of the suspended droplets is preferably about 0.5, 1.0, 2.5, or 5.0 to about 7.5, 10, 25, 35, or 50 micron in diameter, and any combination thereof, although values for the droplet diameter outside of this range are also contemplated. A small particle size encourages rapid removal of volatile materials, for instance under vacuum, and provides uniform, constant heating to the system. Local overheating phenomena, or localized “hot spots” that are prone to occur in the monolithic melt procedures of the prior art led to undesirable side-reactions that may result, for example, in gel-formation and the like. Moreover, the viscous heating effects produced by stirring a high melt viscosity molten monomer, oligomer or polymer employed by the prior art also caused local overheating.

In this method the dispersion is almost always fluid to avoid the undesirable effects mentioned above. The heat transfer fluid itself (precursor solvent) is preferably not volatile, and a poor solvent or a non-solvent for the molten oligomer or polymer. The precursor solvent, in addition, should have a sufficiently high boiling point so that it will not distill extensively from the system under high vacuum during the course of polymerization. Examples of heat transfer liquids or precursor solvents comprise, although not being limited to, mineral oils, vegetable oils, silicone oils, napthalenes, biphenyls, decalines, and substituted benzenes, among others. The inventors have found that hydrocarbon oils such as “white mineral oils” are eminently suitable. Although generally conducted at ambient pressure, the polymerization reaction may be conducted at a pressure as low as about 0.00002 mmHg (absolute) with little loss of oil by distillation, and clearly at any pressure therebetween. Or it may be conducted at higher pressures, up to about 0.002 mmHg (absolute), and even higher. As the reaction progresses the reaction's volatile materials may be removed from the system, e.g. condensed separately in a trap cooled to −78° C. with a solid carbon dioxide/isopropanol mixture. Other methods for removal of volatile substances known in the art may also be employed. The polymerization is preferably conducted at a temperature of about 100, 120, 140, or 160° C. to about 160, 180, or 200° C., with a preferred temperature for certain polyanhydride esters being about 160±20° C. When polymerization is completed the reaction mixture may be allowed to cool with agitation, under e.g. constant and vigorous agitation, until the molten drops solidify and form a suspension of solid spherical particles in the matrix fluid. Upon cooling the particles may be separated from the reaction medium, e.g. by filtration, and washed with a substance that dissolves the mineral oil but not the particles. Although other substances may be employed, light petroleum fractions with an about 40° C. to about 60° C. boiling point were found particularly suitable for this purpose. The particles may be subjected to continuous extraction in a suitable apparatus, such as a Soxhlet apparatus, if desired. The temperature of the solvent during the extraction step should preferably not exceed the glass transition temperature (Tg) of the polymer to avoid causing sintering of the polymer particles. To this end, the use of a modified Soxhlet apparatus is preferred such that the extraction may be performed with cooling of the solvent.

d. Solution Polymerization Process

Monomers, oligomers and polymers of present invention can be prepared by a solution polymerization process which provides a very efficient control of their structure and molecular weight (MW) to attain monomer, oligomer or polymers of enhanced properties such as mechanical properties, stability, and hydrolytic stability, among others. The selection of a monomer structure and amount, feed ratio, and activation strategy permits to obtain polymers of molecular weight greater, and of enhanced performance. This method enables the choice and amount of monomer, solvent, and use of activation chemistry in a selection that impacts the performance characteristics of the resulting monomer, oligomer or polymer. The selection of these parameters may be irrelevant to the preparation of different types of monomer, oligomer or polymers of selected characteristics such as polyesters, polycarbonates, polyanhydrides, and polyamides, among others. This patent teaches how to produce oligomers and polymers of desired properties by choosing specific monomers, solvents, reaction conditions, and optional steps as described below. This process enables the selection of a plurality of monomers, and reaction conditions to produce a monomer, oligomer or polymer possessing a random array of conjoined monomer units imparting to the product desirable properties. One embodiment of this process employs an acylating or dehydrating agent, e.g. phosgene or phosgene analogue, equivalent or substitute e.g. triphosgene, preferably in stoichiometric combination with an aliphatic or aromatic diacid salt(s) in the presence of a solvent for the diacid salt(s) e.g. volatile organic solvent, comprising halogenated hydrocarbons e.g. chlorinated hydrocarbons, ethers, esters, amides, and sulfoxides having boiling points less than 200° C., among others. Preferred solvents include halogenated solvents e.g. chlorinated solvents with boiling points less than about 100° C., an example being dichloromethane. In a preferred embodiment, the aliphatic or aromatic diacid salt(s) may be monomeric, oligomeric or polymeric in nature. In another preferred embodiment, the monomeric, oligomeric, or polymeric diacid chloride may be replaced by phosgene and the corresponding diacid. In another embodiment, various diacid ammonium and alkali metal salts may be utilized as well. Still another embodiment of the solution polymerization process for preparation of the oligomers and polymers of this invention comprises employing the synthetic routes described below with or without different optional steps. Various permutations of the different steps shown in the overall schemes illustrated below provide the flexibility of designing monomer, oligomer or polymers of desired characteristics such as molecular weight, flexibility, hardness, adhesiveness, and the like by modulating different parameters associated with their manufacture, such as linker length, substituents, combining stretches of different polymers of different physical and chemical properties, end-capping, combining aromatic with aliphatic moieties in the linkers and co-polymer segments, and the like, as described below. Schemes 1(1) and 1(2) provided below show two embodiments of the process of this invention involving solution polymerization.

In the embodiment shown in Scheme 1(1), the hydroxyl group of each of two molecules of an agent(s) or compound(s) of interest is(are) reacted with a bi-functional linker(s) that has been activated by acylation to obtain the corresponding acid chloride(s) (2) in the presence of a solvent and allowed to form a diacid intermediate (4) comprising two end agent units, for example, two (2) drug molecules, with one linker between them. The diacid intermediate (4) may be then placed in the presence of an amine, e.g. a tertiary amine, such as triethyl amine, pyridine and/or di-isopropylethylamine to obtain a quaternary ammonium salt, which in the presence of an effective amount of triphosgene or similar agent dissolved in a solvent, e.g. an anhydrous solvent such as dichloromethane or chloroform, that may be preferably added slowly to the quaternary ammonium salt of the diacid mixture to form a desired polyanhydride (5). In this embodiment, the molecular weight may be determined by the amount of triphosgene as well as the period of time the reaction may be allowed to proceed. The growth of the molecular weight may be monitored as the monomer, oligomer or polymer may be extended, for example by GPC as is known in the art. The reaction may be conducted across a wide range of temperatures, e.g. about −20, −15, −10, −5, 0, or 5° C. to about 5, 7, 10, 15, or 20° C., or ambient temperature, provided that the temperature does not facilitate the occurrence of side reactions that might impede the linear growth of the oligomer or polymer, e.g. about <25° C. If practiced in the manner described, this process produces an oligomer or polymer comprising alternating units of the agent(s) or compound(s) and the linking group(s).

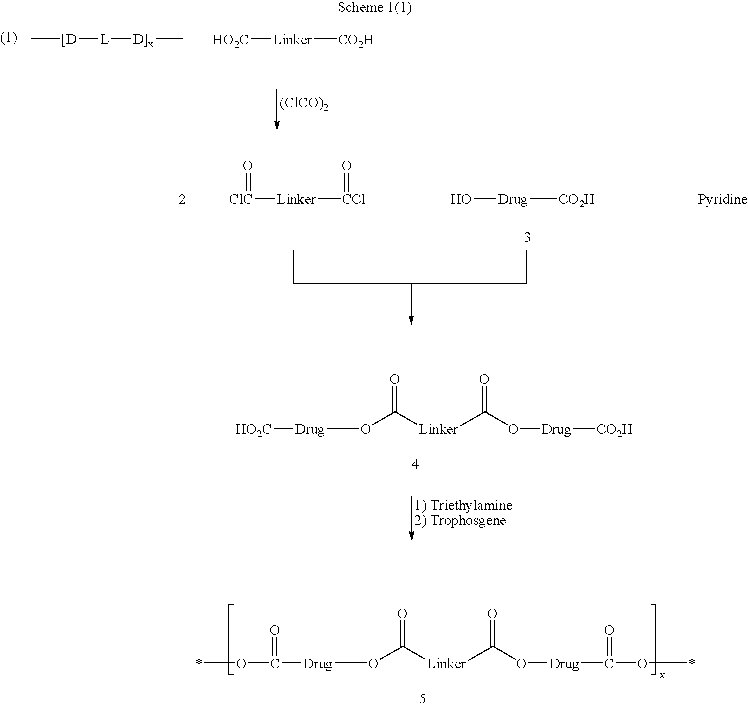

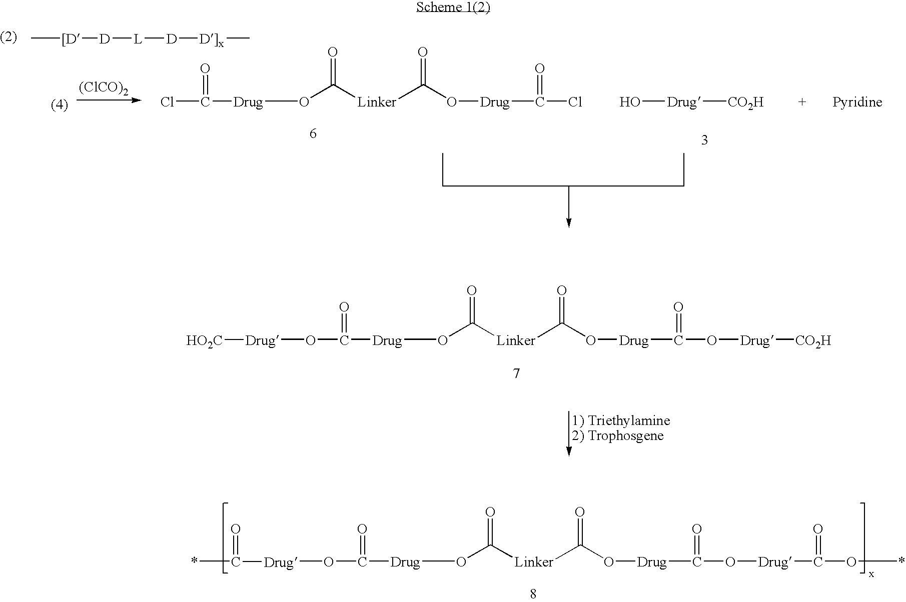

In the embodiment shown in Scheme 1(2) the diacid intermediate (4) may be activated by acylation to attain a diacid halide (6) comprising two molecules of agent(s) and one linker that may be reacted then with the hydroxyl of two molecules of agent(s) or compound(s) to form a diacid comprising four agent(s) units, and so on. This diacid may then be subjected to the remaining steps of the process described above to form its triethylammonium salt, and then placing the salt in the presence of triphosgene to form an oligomer or polymer in accordance with this invention comprising alternating units of one linker and four drug moieties. In the same manner the process may be adapted to design monomer, oligomer or polymers of varying numbers of agent(s) units bonded to one another and then linked through one linker, or by employing the same or other linkers and other agents to vary the chemical sequence of the resulting oligomers and polymer. Still another embodiment of the process of the invention is shown in Scheme 2. This embodiment comprises generating an oligomer with a relatively low molecular weight, e.g. about <40,000, by reacting two different diacids that may be activated as acid chlorides (6) and (9) with, e.g. a triethyl ammonium salt of an agent(s) or compound(s) in an anhydrous solvent. The thus formed pre-polymer may be isolated and linked together by addition of, for example triphosgene, to the quaternary ammonium salt of the pre-polymer to achieve a higher molecular weight, e.g. about >50,000, by end-linkage. Depending on the composition and the order of addition of the different components, the arrangement around the agent(s) or compound(s) units may be modified by using this procedure to attain sequences such as -L-D-L-, -L-D-D-L-, -L-D-D-D-L-, or -L-D-D-D-D-L-, wherein D comprises an agent(s), and L comprises a linking group(s), among many others. The thus produced bonds between linker and agent(s) or compound(s), agent(s)-agent(s), or linker(s)-linker(s) may comprise ester or anhydride or other degradable bonds depending on the combination of process. It will be appreciated by those skilled in the art that the compounds of the invention may comprise a chiral center(s) and, therefore, may exist in and be isolated in optically active and racemic forms. Some compounds may exhibit polymorphism. The present monomer, oligomer or polymers comprise any racemic, optically-active, polymorphic, or stereoisomeric form, and their mixtures, including those of an agent(s) or compound(s) possessing the useful properties described herein. An artisan will know how to prepare optically active forms, for example by resolution of the racemic form by recrystallization techniques, by synthesis from optically-active starting materials, by chiral synthesis, and by chromatographic separation using a chiral stationary phase, among others, and how to determine cADPR agonist or antagonist activity of the monomer, oligomer or polymers and agents or compounds using standard tests that are either described here or are well known in the pertinent art.

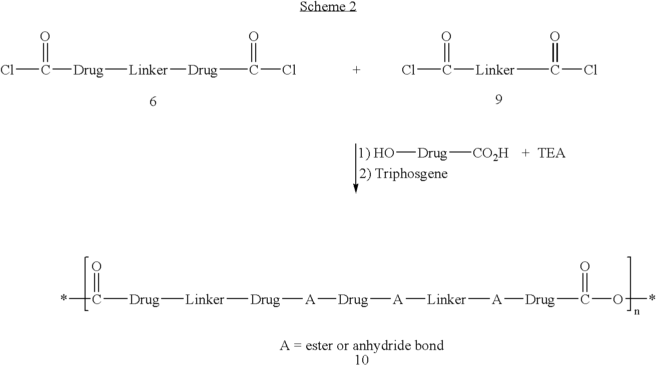

Intermediates useful for preparing compounds of formula (Ia) or (IIb) are also provided. In cases where compounds are sufficiently basic or acidic to form acid or base salts, use of the compounds as salts may be appropriate. Examples of acceptable salts are organic acid addition salts formed with acids that form a physiological acceptable anion, for example, tosylate, methanesulfonate, acetate, citrate, malonate, tartarate, succinate, benzoate, ascorbate, α-ketoglutarate, and α-glycerophosphate, among others. Suitable inorganic salts may also be formed, including hydrochloride, sulfate, nitrate, bicarbonate, and carbonate salts, among many others. Acceptable salts may be obtained using standard procedures well known in the art such as by reacting a sufficiently basic compound such as an amine with a suitable acid affording a physiologically acceptable anion. Alkali metal (for example, sodium, potassium or lithium) or alkaline earth metal (for example, calcium) salts of carboxylic acids may also be made. The ability of a compound of the invention to be polymerized may be determined using polymerization techniques that are well known to the art. The activity of the monomer, oligomer or polymers may be determined using assays that are well known to the art or described herein.

e. Solution Process—Structure & Molecular Weight Control

The solution polymerization process comprises novel and unobvious steps, and produces monomers, oligomers and polymers that exhibit marked improvements over prior art products in terms of various structural and performance properties. The process improves on the desirable characteristics with respect to prior art monomer, oligomer or polymers, particularly in attaining higher molecular weights e.g. up to about 100,000; 200,000; 350,000; 500,000; 750,000; 1,000,000 Dalton, and higher. This process results in monomers, oligomers and polymers that exhibit specific unexpected properties that are described below.

1) Enhanced structural control may be employed to achieve targeted monomer, oligomer or polymer assembly characteristics by polymerization of pre-designed co-monomers and/or linking chemistries. The present oligomers and polymers attain configurations representing a broad spectrum ranging from purely alternating, to random, to tapered block, to multiblock oligo- and polymeric structures. An illustrative, non-limiting example includes hybrid ester-anhydride monomer, oligomer or polymers based on salicylic acid derivatives. These compounds contain relatively labile phenolate esters that are readily amenable to concerted trans-esterification and anhydride exchange. This feature may be controlled by the method of the invention to attain a targeted, controlled structure during the solution polymerization process.

2) Enhanced yield and purity are achieved through inhibition or suppression of deleterious oxidative, cross-linking, and other side-reactions by employing mild polymerization temperatures, e.g. ambient and lower solution-polymerization temperatures that are milder by comparison with the prior art melt-condensation temperatures typically well in excess of 100° C.

3) Enhanced capability to control monomer, oligomer or polymer molecular weight, and in particular a demonstrated capability to achieve high polymer molecular weights, both in one-pot, and in post-end-linking syntheses. These molecular weights are higher that were ever reported by the prior art using melt-condensation, and solution polymerization.

4) Greatly enhanced storage stability (shelf life) and “pot-life” stability, particularly in terms of hydrolytic stability in organic solvents and in the solid state, achievable with a wide range of alternating, randomized and block oligo- and polymer structures. This is generally imparted during an acidic treatment, and isolation of the oligomer and polymer following solution-polymerization.

5) Ability to control the performance properties of a monomer, oligomer or polymer e.g. degradation rate and mechanical strength, for instance by selection of appropriate co-monomer and linking chemistry configurations. This may be implemented by the following means.
i) Inhibition of, or decreased, pitting that may be mostly due to crystallization of a less randomized polymeric structure (a more regular crystalline structure) as it degrades. Bulk and surface integrity and mechanical strength may be maintained by preventing unwanted crystallization. This facilitates sustained oligomer and polymer surface erosion, inhibits transition to bulk erosion, and enhances long-term adhesion to surfaces and predictable/controllable compound generation rates from polymer films and other forms and shapes, even when wet.
ii) Prevention of formation of long block structures of phenolate-ester-linked diflunisal units that typically arise during melt polycondensation and are very slow to degrade. This prolongs the time to achieve complete elution of diflunisal.
iii) Prevention of formation of long block units that arise during melt-polycondensation and increase instability in organic solvents, and in the solid state due to enhanced hydrolytic lability of non-aromatic anhydride linkages.

6) Ability to obtain high polymer molecular weights of up to about 600,000 Dalton, and even higher, by solution polymerization. These molecular weights are substantially higher than those attained by melt-polycondensation by the prior art. Such high molecular weights enhance the mechanical strength, flexibility, and toughness, among other properties, of the polymer. The unexpected ability to control a polymer's structure, performance and stability provided by the process of the invention relative to the prior art melt-polycondensation processes arises largely from the interplay of various factors described later. The use of a solution medium in the polymerization process of this invention eliminates the occurrence of “melt incompatibility” that is prevalent in melt-polycondensation methods of the prior art. The net effect may be seen most readily when co-monomer units highly incompatible in the melt, such as fluorinated aromatic-fatty aliphatic co-monomer units, e.g. diflunisal-C14 diacids, are polymerized by these two distinct methods. In the melt-polymerization process of the prior art, this melt incompatibility may drive the ultimate formation of segregated, tapered block co-monomer arrangements. In melt polycondensation, melt segregation may also contribute to the formation of insoluble domains, or chemically- or physically-cross-linked gels. This significantly lowers the yield of useful polymer, and requires the extraction of soluble polymer portions upon completion of the synthesis. In the specific case of the mentioned C14 diflunisal polymer, for example, the occurrence of block sequences of bis-C14 anhydride may compromise the polymer's hydrolytic stability in organic solution and in the solid state whereas block sequences of phenolate-ester-linked diflunisal units degrade very slowly and, thereby extend the time for complete polymer degradation and diflunisal release.

7) The solution process of the invention utilizes highly-reactive linking chemistries in combination with low temperatures that facilitate the design and attainment of desired end-structures and disfavors unwanted side reactions that are prevalent under the high temperatures required by melt-polycondensation. The nature of the polymer end-groups, e.g. aromatic and aliphatic carboxylic acids, produced by solution polymerization in combination with an acidic aqueous workup procedure facilitates the conversion of anionic salts to carboxylic acids, and produces a marked improvement in both storage and “pot-life” hydrolytic stability. In the specific case of drug polymers such as a C₁₄-diflunisal ester-anhydride polymer employed in the examples, the addition of an end group contributes to the improved control of the polymer structure, and to the formation of lesser sequences of bis-C₁₄ anhydrides, all of which contribute to improving hydrolytic stability in organic solvents and in the solid state.

f. Process Employing End-Capping/End-Linking

This embodiment of the solution polymerization process may be designed to attain high molecular weight polymers by controlled addition of end-groups through solution end-linking chemistry. In one embodiment, end-linking or end-capping involves the use of an acylating or dehydrating agent e.g. phosgene, preferably in stoichiometric combination, with an aliphatic or aromatic diacid ammonium salt(s), preferably alkylammonium or alkali metal salt(s), in the presence of a solvent e.g. an organic solvent. For polymeric extension by coupling or polymer end-capping preferred diacid salts comprise oligomeric or polymeric aliphatic or aromatic diacid salts. An oligomeric or polymeric diacid halide, e.g. diacid chloride, may be substituted for phosgene, and the corresponding diacids and/or diacid ammonium and alkali metal salts utilized. The choice of end-linking chemistry for polymer extension to increase the polymer's molecular weight vs. reactive propagation of co-monomer functional groups impacts the type of structural arrangement produced, in terms of both linking bonds and co-monomer arrangement, the resulting configurations ranging from purely alternating to random to tapered block to multi-block structures. The following are non-limiting examples intended to illustrate the numerous conceivable synthetic permutations encompassed by this process.

1) Polymerization of two different diacid alkylammonium salts with phosgene produces a randomized co-monomer.

2) Polymerization of two different diacids, one present as an acid halide, e.g. chloride, and the other present as an alkylammonium salt, yields a strictly alternating co-monomer.

3) Polymerization of two different diacids forming part of an acid halide, and alkylammonium salt produces a randomized co-monomer.

4) Utilization of a co-monomer with a phenol group, e.g., salicylate drugs, in the form of an alkylammonium salt imparts an enhanced capability for concerted transesterification and anhydride exchange during the synthesis of ester-anhydride polymers based on them. The frequency may be modulated by starving the pot of free phenol groups to varying degrees. This method achieves a wide range of polymer structures incorporating varying degrees of randomization and/or blocking of both co-monomer units and linking structures.

The choice of chemistry for end-capping may differ from that for chain extension. The end-capping step utilizes a mono-functional rather than di-functional entity used for chain extension. Non-limiting examples of possible compounds for end-linking include acetyl chloride with an alkylammonium carboxylate-terminated polymer to produce a mixed acetic anhydride end-group. Conversely, alkylammonium acetate with a carboxylic acid chloride-terminated monomer, oligomer or polymer may be employed to produce a mixed acetic anhydride end-group. Fatty acid halides, e.g. chlorides, or fatty alkylammonium or metal salts, such as palmitoyl halides, e.g. chloride, may be similarly employed to produce fatty acid end-groups. Clearly, other structural end-groups may be used if suitably pre-functionalized to allow end-capping with the monomer, oligomer or polymer of interest. The chemical reactions or steps of the process involved in end-linking may be implemented in-situ as the last step of oligomer and polymer synthesis. In another embodiment this effect may be attained by end-capping a pre-synthesized oligomer or polymer. In this embodiment employing a pre-synthesized oligomer or polymer it may be preferable to use cross-linked acid-acceptor beads instead of tri-ethylamine or other tertary amine to make an alkylammonium carboxylate salt. This preferred modification may greatly facilitate the post end-capping work. A choice of linking chemistry may be implemented by selection of propagating co-monomer functional groups. This selection will impact the type of structural arrangement produced, e.g. linking bonds and co-monomer arrangement, resulting in structure configurations ranging from purely alternating to random to tapered-block to multi-block structures. Non-limiting examples intended to illustrate the numerous conceivable synthetic permutations are described below. This process conducts the polymerization with all acylating agents at temperatures e.g. ambient to about 0° C., and even lower temperatures. Such temperature range will generally suffice for the facile acylating propagation reactions, and polymerization may typically be achieved in times ranging from as little as about 1/2 hour to about 6 hours. In one embodiment low polymerization temperatures are more amenable to temperature sensitive co-monomer units than the prior art melt polycondensation process that requires long intervals of sustained high temperatures e.g. in excess of 100° C., typically in excess of 140° C., for more than 12 to 24 hours. In yet another embodiment where phosgene or phosgene-generating substitutes like triphosgene are employed it may be preferred to utilize a temperature below phosgene's boiling point (8° C.) to prevent its loss during the reaction. This embodiment extends the range of molecular weight and end-capping capabilities achievable with the above described solution method for synthesis of polyanhydrides and poly(ester-anhydrides), among others.

g. Process with Controlled Sequence Domains

This embodiment may employ either melt-condensation or solution-polymerization to produce new oligomers and polymers comprising two or more different monomeric units covalently joined in defined molar ratios. This embodiment results in an oligomer or polymer of predictable domains that may be constructed by careful selection of the nature and quantity of the input monomer feeds, and by an appropriate choice of reaction conditions. Each of the oligomer and polymer domains results from the structural characteristics of the individual monomers and imparts overall useful chemical and physical properties such as hardness, adhesion, hydrophobicity, permeability, crystallinity, flexibility, hydrolytic stability, intrinsic thermogravimetric profile, among many other properties that may be also enhanced and are contemplated in this invention. These properties may be altered in a predictable pattern by controlling the input molar % of monomer to obtain oligomers and polymers of unexpectedly superior chemical and other characteristics, and a freely tunable rate of degradation and, thereby, agent(s) or compound(s) released in situ. The process of this invention provides a means of designing a desired oligomer and polymer by correlation of the nature and mole ratio of constituent monomers with specific performance characteristics. The individual monomer, oligomer or polymer characteristics may be qualitative or quantitative measured as may be their contribution to the overall co-polymer characteristics. This permits the selection of a defined ratio of two or more constituent monomers or to alter the mole ratio of reactant monomers to design specific co-oligomers and co-polymers of predictable performance parameters. The following is an example provided for illustrative purposes only, and it relates to the formation of a co-oligomer or co-polymer comprising A and B monomer units, where monomer A comprises Diflunisal-Diflunisal-C14Linker-Diflunisal-Diflunisal (DF-DF-C14-DF-DF), and monomer B comprises diflunisal-C14Linker-Diflunisal (DF-C14-DF). An increase in the content of monomer A from 0% to 50 mol % in a mixture of monomers A and B, with monomer B going from 100% to 50%, resulted in polyanhydrides with regularly increasing hydrolytic stability and Glass Transition Temperatures (Tgs). Thus, it is clear that the performance characteristics of a resultant monomer, oligomer or polymer may be controlled by modifying the structure and, in the case of oligomers and polymers by modifying the mole fraction of the participating monomers. Given the relationship between individual monomer mole fraction and particular oligomer and polymer parameters, the solution process provides the unexpected advantage of designing them with pre-determined performance characteristics in mind by choosing of mole % monomer ratios. In the above example a step-wise increase in the mole fraction of monomer A in a solution-based process leads to polymers with increasingly different performance parameters, e.g. Tg, flexibility, and hydrolytic stability, among others. Thus, the inventors found unexpectedly that they could manufacture a polymer that possesses a desired Tg and hydrolytic stability profile by choosing the appropriate mole % fraction of one monomer over the other, e.g. monomer A over monomer B. The monomer, oligomer or polymers of the present invention possess refined performance characteristics, and may be employed, for example, as coatings, films, laminates, adhesives, formed implantable structures, e.g. drug-containing nano- and micro-spheres, medical devices, orthopedic and dental implants, and pharmaceutical formulations, among others.

h. Branching Process at Well-Defined Branch Points

This embodiment incorporates well-defined branch points into polymeric materials to permit branching to modify their performance characteristics. The process relies on the structure, synthesis, and deployment of branching agents as a preferred embodiment of either melt dispersion or solution phase polymerization processes of the invention. Suitable branching agents may comprise tri-, tetra-, penta-, hexa-, or higher-order functional groups. The functional group for a branching agent may be selected to impart properties such as increased elasticity, increased melt elasticity, change in toughness and fatigue resistance, among many others. The performance of each specific branched oligomer and polymer will be determined by factors such as the amount of each branched segment and the molecular weight of the segments between branching points. In one embodiment a branching agent(s) may be incorporated into the process at the beginning of polymerization to produce star-like polymeric structures. In another embodiment the branching agent(s) may be incorporated late in the polymerization process to yield highly networked structures. Another embodiment of this process provides for combinations of these two extreme modes by varying the ratio and time of incorporation into the polymerization step of the process. The molecular weight of the oligomeric or polymeric segments present between branch points may range from one unit to any number of repeating units. In one embodiment of the process molecular weights above those necessary for chain entanglement are produced and are preferred. Any percentage of branching agents may be effective with a demonstrated and preferred embodiment of about 1%, 2%, 3%, 5%, or 10%, and higher, but less than the amount necessary to cause significant gelation during polymerization. The prior art required branching to be incorporated as a random adjunct to polymerization in polyanhydrides. In still another embodiment the specific chemistries employed by this process enables a significant control of the polymer structure where the molecular weight of segments between branching points, branch point distribution, and branch point type may be selected to yield controlled structures of pre-determined erosion kinetics. In yet another embodiment this process permits control of mechanical properties such as fatigue resistance, elasticity, and others, which had heretofore not been engineered to the extent provided by this invention.

i. Process of Preparation of Thermoplastic Elastomers

This embodiment provides a process for the synthesis of a biodegradable monomer, oligomer or polymer with increased elasticity at its application temperature. The thus designed monomer, oligomer or polymer may be formed by heat-based synthesis, and cast using known coating technologies. The ability to increase the elasticity of a polymer provides advantages in terms of, for example, better flexibility, malleability, resilience, and flow behavior, among many others. The present inventors discovered that their specific solution chemistry process would help create the block structures needed to synthesize these materials. Applications where flexibility may be necessary, e.g. in medical and other devices, require a polymer of rubber-like behavior for enhanced or maintained performance. Examples of this type of applications are coatings of Nitinol and other similar nickel-based alloy implants and devices, ophthalmological applications requiring flexible erodable polymers to assist in non-inflammatory support or substance delivery, and many others. In one embodiment the solution polymerization process of this invention permits the design of materials that will lead to phase separation. Block co-polymers may be created from a repeating structure based on a linker and incorporating an agent(s) of one solubility factor, as determined by any acceptable solubility calculation and linker and incorporating an agent(s) of different solubility. This will generally result in phase separation of the two blocks observed as two distinct glass transition temperatures, as measured by any acceptable technique. The co-polymer blocks may be selected such that the glass transition temperature (Tg) of the two phases bracket the application temperature of interest. That is, the Tg of one phase may be lower while the Tg for the other phase may be higher than the target temperature. Various monomer, oligomer or polymers, such as mono-, oligo- and polyester, polycarbonate, polyamide, polyurethane, and polyanhydride, among others, may be prepared in this manner by proper choice of condensation conditions. As the block phases separate they form an extended network that results in increased elasticity. The new polymer will more likely be more rubber-like at the designed application temperature. Yet, when the polymer is heated above the glass transition temperature of the higher Tg block, it may be processed into a variety of shapes by standard polymer processing techniques. This embodiment of the process may be carried out by means of a solution based coupling process known to those skilled in the art. A non-limiting example comprises coupling of two pre-polymers having different Tgs in a volatile solvent for the pre-polymer employing a condensing agent(s) such as phosgene, diphosgene, triphosgene, oxalyl chloride, thionyl chloride, alkanedioic dichlorides, phosphochloridates, and carbodiimides, among many others known in the art. Suitable volatile solvents include, but are not limited to, chlorinated hydrocarbons, ethers, esters, amides, and sulfoxides having boiling points less than about 200° C., among others known in the art. A group of preferred solvents includes chlorinated solvents with boiling points less than about 100° C. In another embodiment the thermoplastic elastomeric block co-polymer may be synthesized by other polymerization techniques such as a melt process.

j. Processes for the Synthesis of Oligomers and Polymers in the Examples

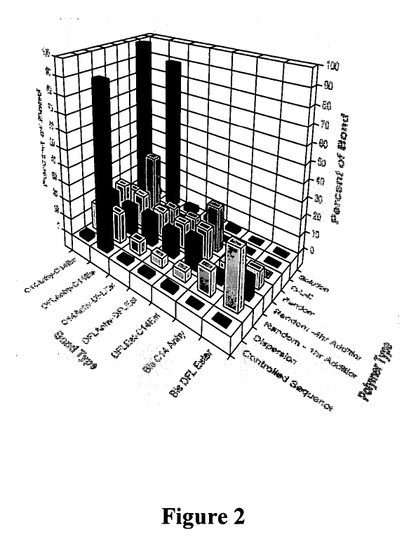

The following Schemes 3-21 along with FIG. 2 are illustrative of the synthetic process for the preparation of various inventive compounds described in the examples. The numbers assigned to each of the monomers, oligomers and polymers are referred to below in the examples with the description of the compound's synthesis and/or its use in the preparation of another compound. 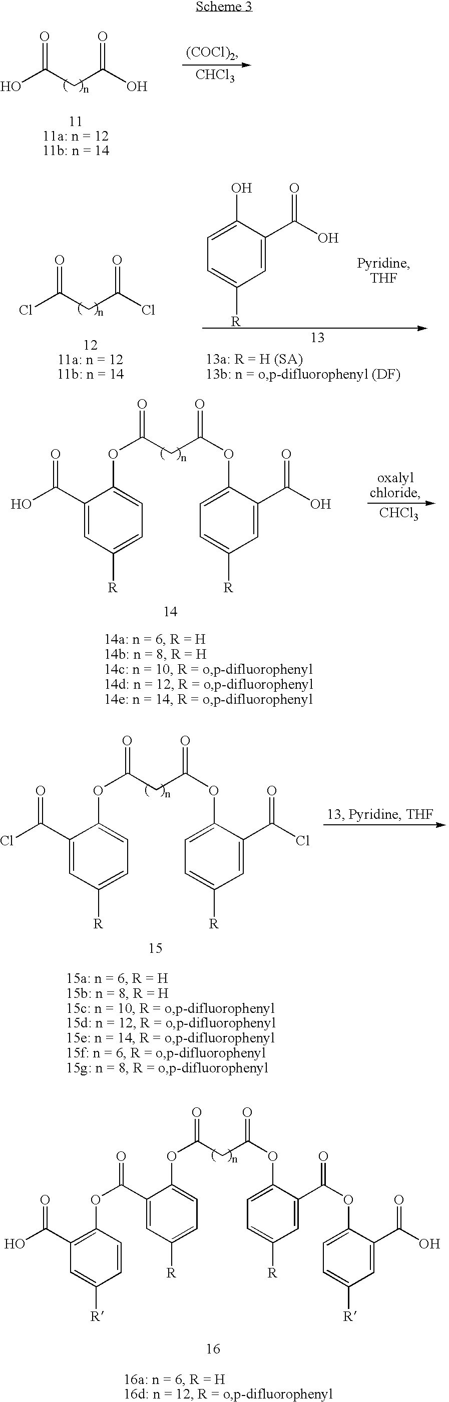 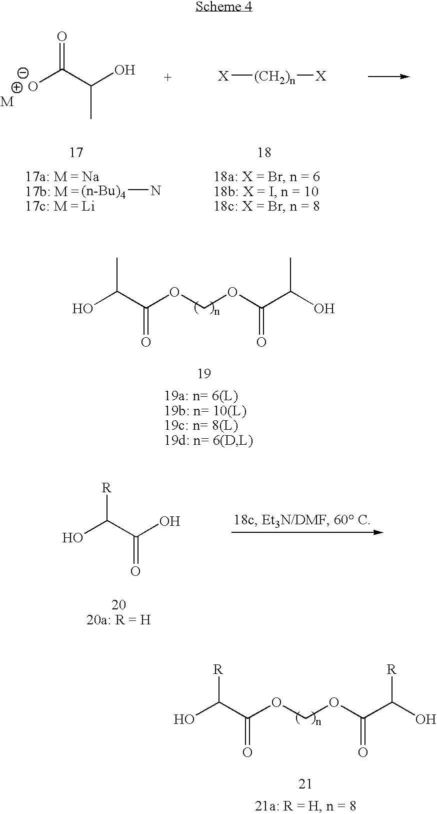 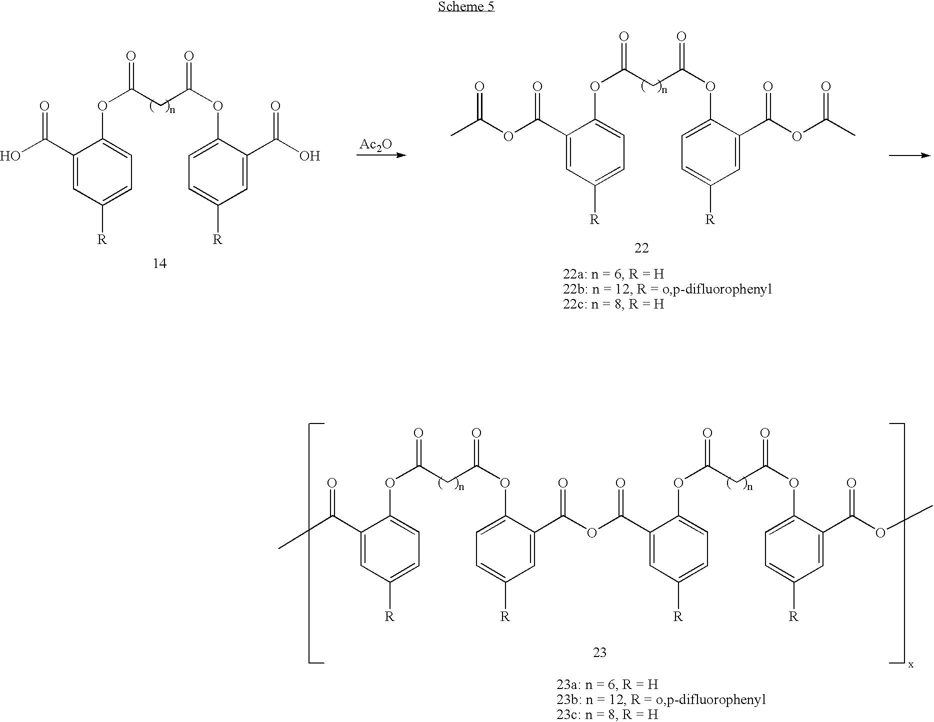 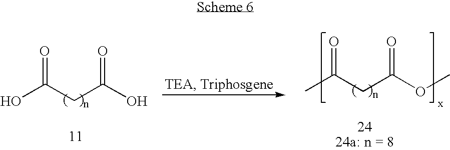 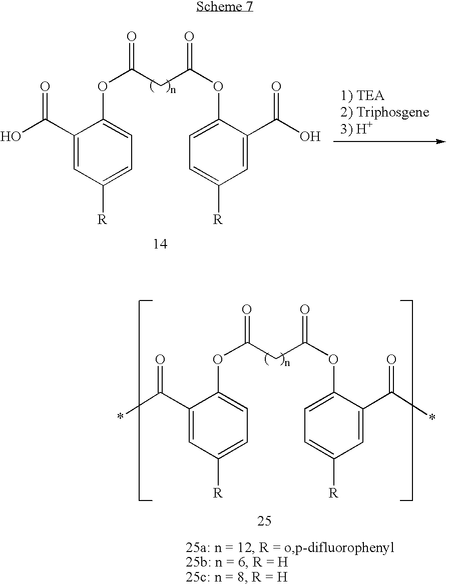 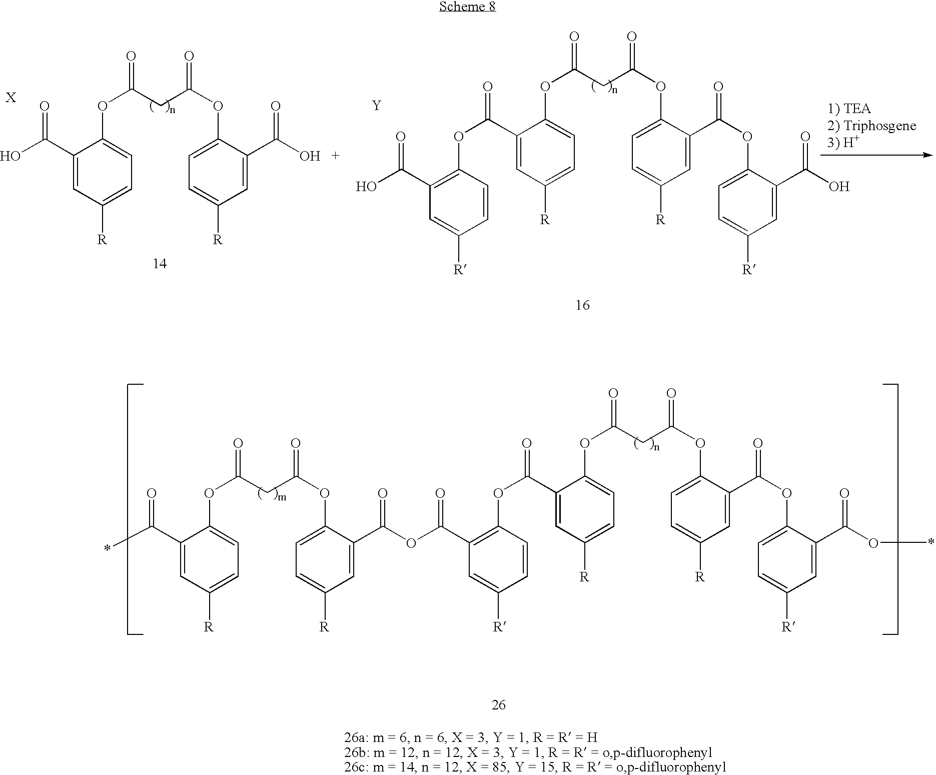 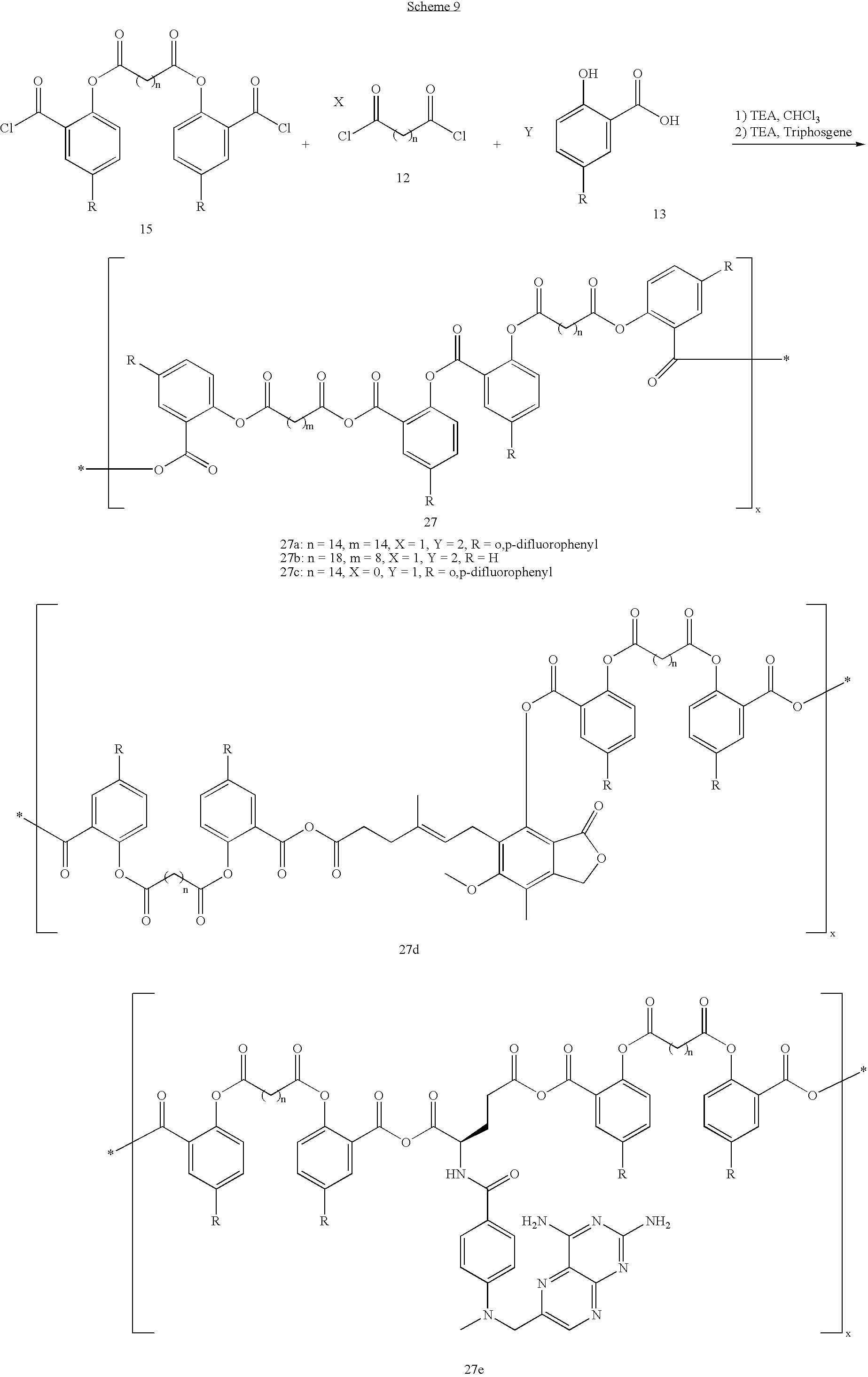 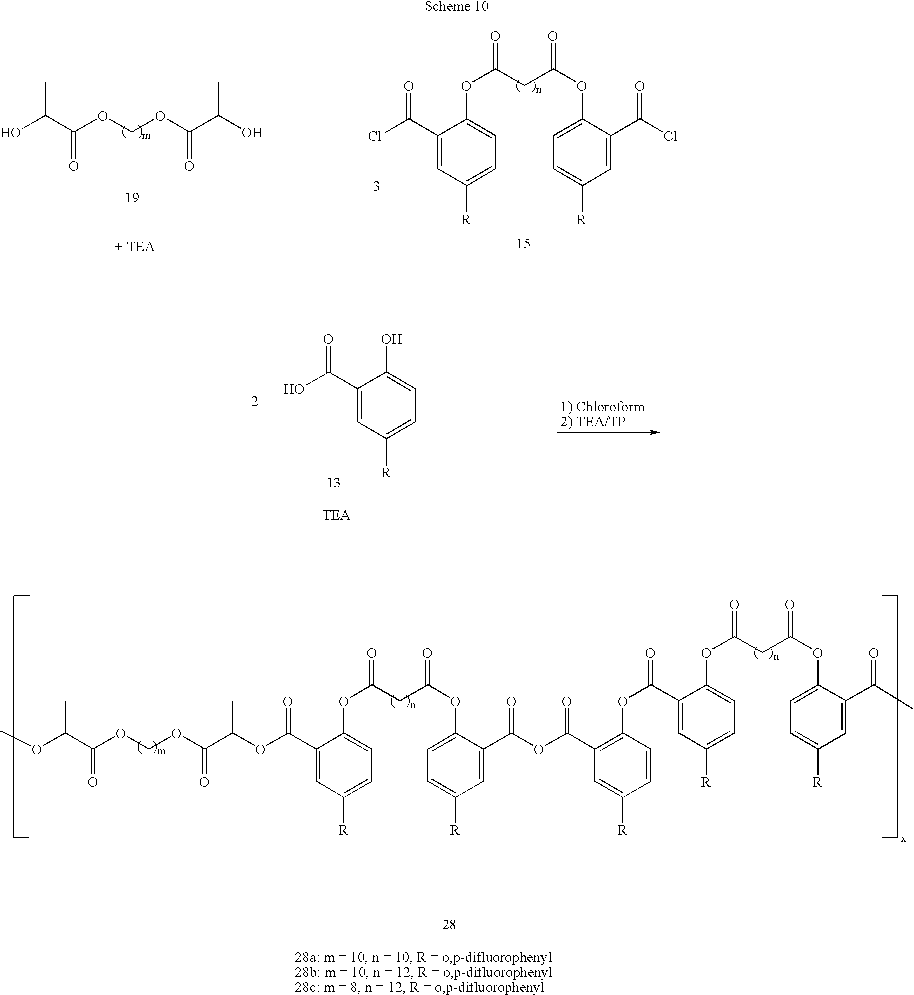 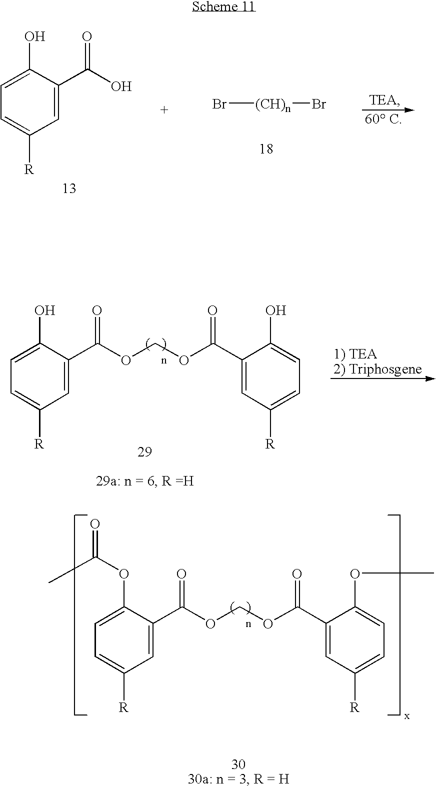 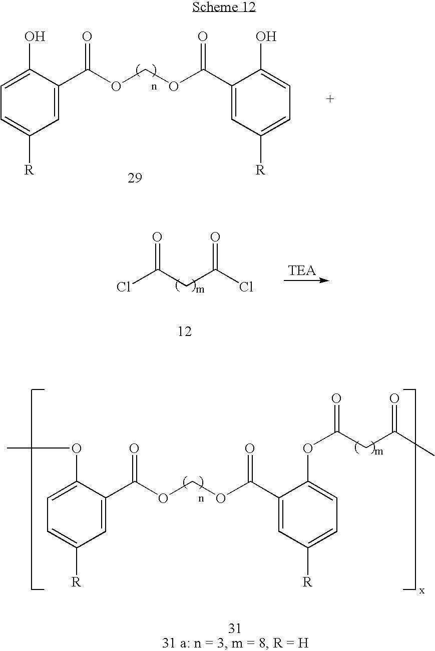 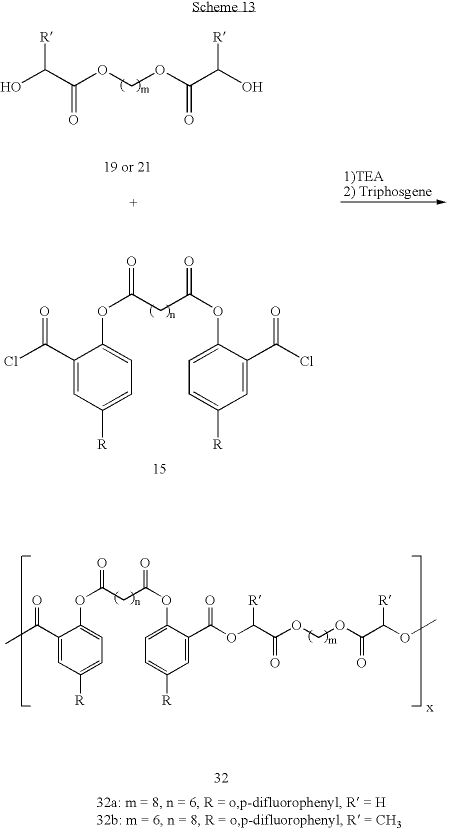 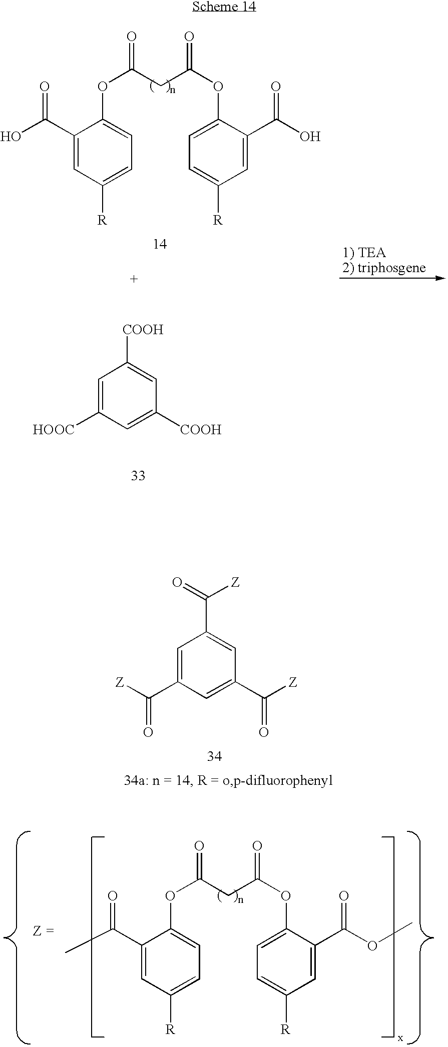 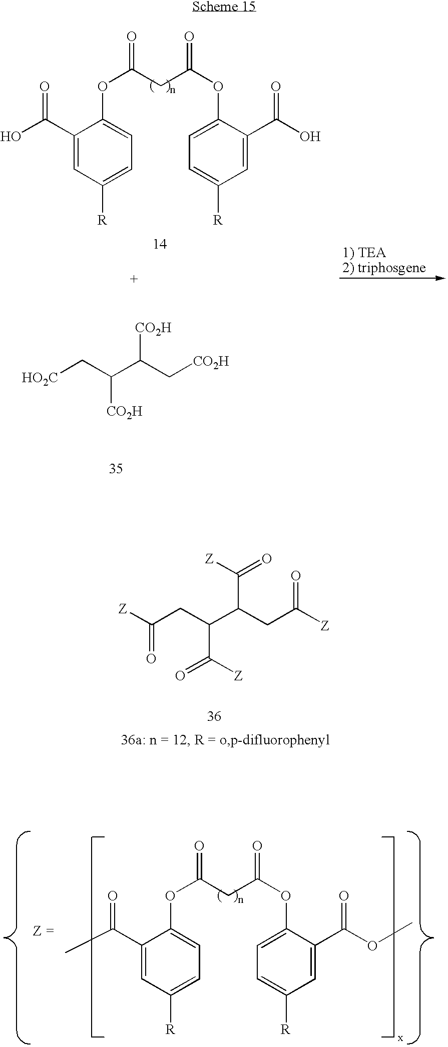 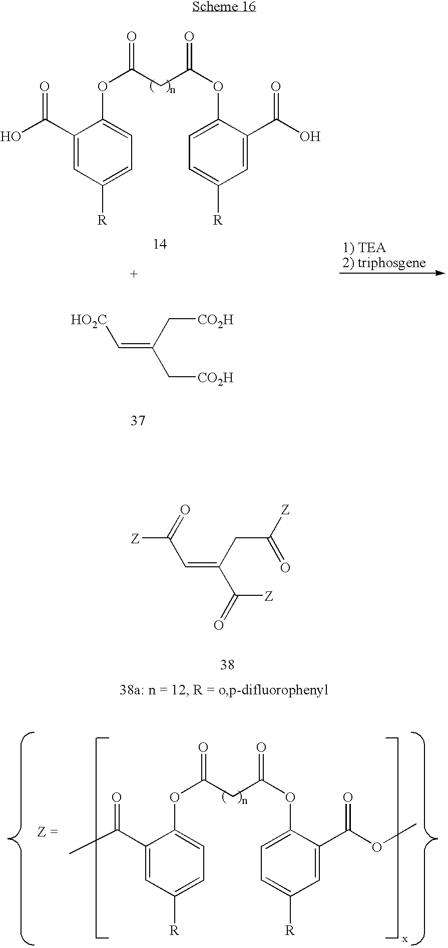 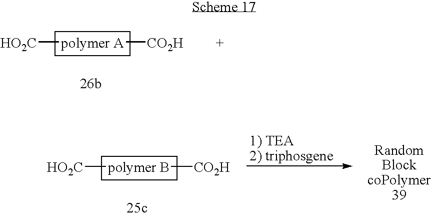 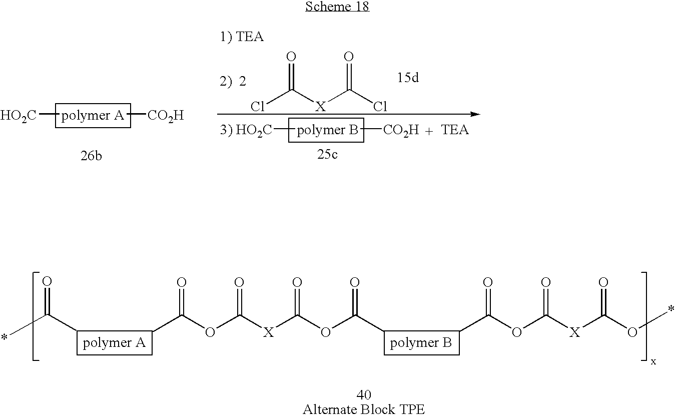 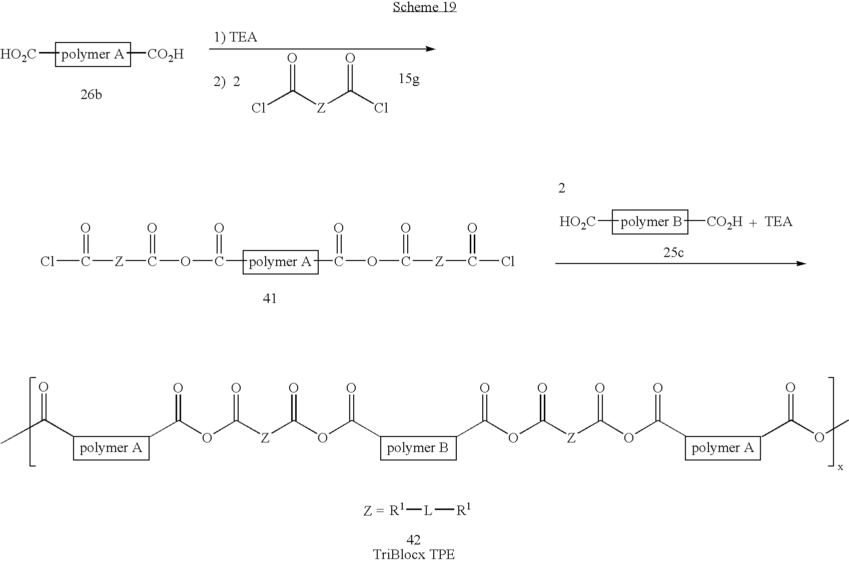 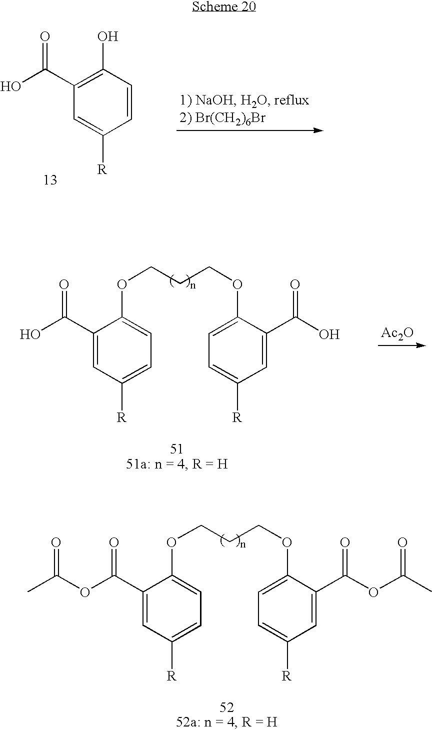 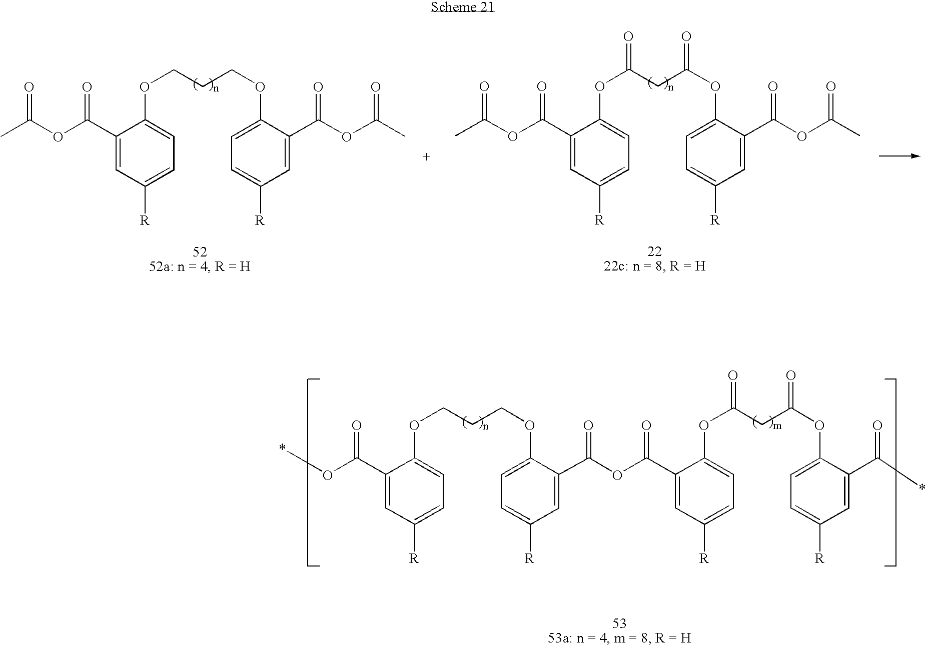
VI. Sterilization

All implantable and percutaneous medical devices require sterilization before utilization, e.g., before or after packaging. Commonly employed sterilization methods are gamma ray irradiation, electron beam (“E-beam”), and ethylene oxide treatment. Gamma ray irradiation penetrates objects deeply, and may be used for sterilizing foodstuffs and many medical device products. This method, however, requires relatively prolonged exposure times. E-beam sterilization requires shorter exposure times but has poor object penetration making the procedure useful mainly for surfaces. Ethylene oxide sterilization is generally more complex and more aggressive on organic materials than the other two methods, and may be being replaced by them wherever possible because it is an environmental hazardous agent. The relatively high temperatures and humidity conditions required by many ethylene oxide sterilization protocols make it not to be highly compatible with many (anhydride-ester) monomers, oligomers and polymers. The methods of choice for sterilization of the monomer, oligomer or polymers of this invention, therefore, comprise gamma radiation or E-beam sterilization. Experimental results show that E-beam (3.5 mRad) and gamma radiation (25-35 Kgys) sterilization have no effect on the pattern of diflunisal release from polydiflunisal (polyDF) coated stainless steel samples incubated in serum at 37° C. Notwithstanding its lack of effect on monomer, oligomer or polymer degradation, gamma ray and e-beam irradiation sterilization do produce some changes in the molecular weight and mechanical properties of polymers. The tensile modulus of melt-polymerized polySalicylic Acid (polySA), for example, was seen to decrease at room temperature by about a third after gamma sterilization (25-35 Kgys). There was no change in either variable, however, at 37° C. Gamma radiation had no effect on the molecular weight, flexibility, or adhesiveness of the polymers of the invention, such as polySA and poly DF, and only minor effects on their hardness.

VII. Layering Coatings of Polymers

The polymers described herein may be layered onto devices to form coatings with desirable properties. The polymers may be structured and/or layered as a coating with one or more additional coatings that may or may not be biodegradable, i.e. degradable by hydrolysis or enzymatic/proteolytic activity when placed in contact or exposed to body tissues or fluids. The additional coatings may contain the same or different polymerized agent(s), no polymerized agent(s), or one or more admixed agents. This structuring may be in the form of a layer of a coating on the exposed surface of the coating of the therapeutic polymer such that this coating lies between the polymerized agent(s), and the body tissues and/or fluids following implantation. Alternatively, a second polymer or smaller molecular-weight species may be physically blended with the therapeutic polymer, and a series of layered coatings of therapeutic polymer compositions that have different chemical compositions and/or physical, e.g. mechanical, properties. In some embodiments layering permits refinement of the rate or duration of generation, release, or elution of agents over time, including the possibility of having one or more outer coatings with higher or lower permeability to modulate the breakdown of one or more inner coatings and thereby result in a more constant release of agent(s) over particular periods of time. In embodiments in which one or more outer coatings are biodegradable, the breakdown and resulting increase in permeability of these outer coatings may compensate for a rate of generation (by breakdown of the polymer) or release of an agent(s) that varies with time by increasing the rate of permeation of the agent(s) from the inner coating through the outer coatings. Such embodiments may be used to create a rate of delivery of drug from the coatings on the device that vary less temporally (i.e., are more closely more zero-order) and that may be adjusted based on the preferred shape and, therefore, surface area of the device and changes in surface area that occur as the coatings erode. Multiple layers of polymers generating, eluting, or releasing inert and products upon breakdown may be designed for specific applications, including those applications where one class, or member of a class, of agents is to be generated, eluted, or released from the coating before a second class or a second member of the first class of agents is generated, eluted, or released from the coating. Possible structuring of layers of coatings, in which one or more of these layers contains a polymerized agent(s) or compound(s), e.g. drug, for implantable medical and veterinary devices are contemplated within this invention. Examples of these are a single layered coating, a multiple layered coating in which the layers may have different compositions and physical properties, including thickness, molecular weight, and others, and in which the top layer(s) comprise(s) or do(does) not comprise(s) the polymerized agent(s) or compound(s) and the bottom layer(s) comprise(s) or do(does) not comprise(s) a polymerized agent(s) or compound(s), a bilayered or multilayered coating in which the top and bottom layers comprise(s) a polymer of the invention of different composition(s). An example of such a layered coating releases an anti-inflammatory agent, e.g. an NSAID(s) substantially before an anti-proliferative agent is generated, eluted, or released from the coating. Such types of layered coatings enable tuning of the rate of generation, elution, or release of drugs from the coating over time, such that a near constant, gradually increasing, gradually decreasing, or a combination thereof amount of drug most appropriate for treatment of tissues in the vicinity of the device may be delivered to these tissues. In one embodiment of the invention, an inert polymer coating(s) may applied as a top coat(s) on one or more polymer coatings, even those that have drugs or other agents admixed therein. A top coating(s) may be applied to increase the hardness and/or lubricity of an outer coating(s) to facilitate use and insertion of a device. A top coating may be applied also to vary, e.g. increase or decrease, the rate of hydration or enzyme penetration to vary, e.g. increase or decrease, the rate of backbone or admixed drug release, or the release of other agent(s) from underlying coating(s). A top coating(s) may be applied as well to increase the shelf life of the final product by limiting water and/or oxygen contact with the underlying therapeutic polymer coating. In one preferred embodiment the top coatings comprises a biodegradable polymer. The polymers of this invention achieve degrees of hardness suitable for a variety of applications. Typically, the polymer of the invention may attain a hardness of about 24, 26, 28, 35, 45, 55 to about, 60, 70, 80, 95, 101, based on a Shore hardness range. Polymers of the invention have different degrees of hardness that are suitable for different applications, such as for use in the devices of the invention.

VIII. Admixing Component Materials

The formation of a composite of two or more materials results in a new material that may have physical properties and performance characteristics substantially different from any of the individual component materials comprising the new material. In the case of polymers, these altered physical properties may include an increase or decrease in glass transition temperature, tensile or shear moduli, effective viscosity, yield strength and elongation, elongation at failure, tackiness or adhesiveness, hardness, color, rate of thermal or biological breakdown, surface texture, or wettability by water or other fluid. For example, the mechanical properties of bone, a composite of inorganic calcium phosphates and organic collagen molecules, are distinct from the mechanical properties of either calcium phosphates or collagen alone. In one embodiment, a monomer, oligomer or polymer of the invention may be admixed with an anti-proliferative agent, such as sirolimus, everolimus, mycophenolic acid and/or paclitaxel, or other material or agent, such as specific RNA and DNA sequences and their chemical mimics or derivatives, calcium phosphate, hydroxyapatite, an antibiotic, an immunosuppressive agent, or another agent. These added compounds may alter the mechanical properties of the monomer, oligomer or polymer, e.g. by modifying the degradation rate, the tensile modulus, the yield strength, and/or the elongation at which failure of the material occurs. Coatings made from the therapeutic polymer will also exhibit the altered mechanical properties. The extent to which the admixture of one or more drugs or other therapeutic agents changes the physical properties and performance characteristics of the coating will depend on the amount or concentration of each of the drugs or agents, with a trend that increasing the amount or concentration of a drug or agent is expected to increase, if at any changes occurs at all, one or more of these properties or characteristics. In practice, coatings with about 0.1, 1, 3, 5, 10 wt % or more to about 15, 20, 30, 35, 40, 45 wt % admixed drug or agent may be achieved by blending the admixed compound into the polymer prior to coating or by first applying the polymer as a coating and then absorbing the compound to be admixed into the coating by exposing the coating to a solution with the compound. In an exemplary embodiment, a coating of a polymer with an admixed drug that may be applied on an expandable article comprises a dicarboxylic acid with more than six carbon atoms in the linear alkyl chain, or a co-polymer or physical blend of polymers or co-polymers that approximate the physical properties and performance characteristics of the polymer with a linker with more than six carbon atoms in the linear alkyl chain, such that these polymers approximate the physical properties and performance characteristics of a polymer with a linker of suberic acid (C8). In another exemplary embodiment, a coating of a polymer with an admixed drug, applied on an orthopedic implant, comprises a dicarboxylic acid with more than four carbon atoms in the linear alkyl chain, or a co-polymer or physical blend of polymers or co-polymers that approximate the physical properties and performance characteristics of the polymer with a linker with more than four carbon atoms in the linear alkyl chain, such these polymers approximate the physical properties and performance characteristics of a polymer with a linker of succinic (C4) or adipic (6C) acid. In some embodiments, compositions comprising polymers may have optimum physical and chemical properties derived by blending compounds into the polymer that decrease or increase the rate of penetration of water and/or enzymes into the polymer matrix and, thereby, decrease or increase the rate of breakdown of the polymer, thereby modulating the duration of generation of drug from the components of the polymer backbone and/or the release of admixed drug or agent. In addition, qualities such as shelf life, e.g. stability in the presence of elevated temperature, humidity, or electromagnetic radiation, rates of depolymerization, e.g. by hydrolysis or proteolytic activity, or oxidation, and rates of hydration may be varied by adding antioxidants or lipophilic molecules to reduce oxidation or hydration of the polymer blend, respectively. In some cases, the qualities of the admixed drug or agent may influence the physical or chemical properties, including shelf life, tolerance to sterilization methods, or degradation rate of the final product. For example, the admixed drug or agent may extend the shelf life, increase the types and/or dosages of sterilant that may be applied without changing other properties of the material, or decrease or increase the degradation rate of the final product.

IX. General Overview of Uses of the Inventive Polymers

The present invention provides compositions, articles comprising at least one agent(s) linked or appended to a monomer, oligomer or polymer or dispersed or blended within it, and methods of using them for delivering the agent(s) to a site of injury, surgery, bone replacement or mending, and the like, to prevent bone growth and other undesirable effects that occur before proper attention may be given to the wound. A route of delivery may be selected in accordance with the drug being administered and the condition being treated. In one embodiment, the monomer, oligomer or polymers decompose harmlessly while delivering a selected low molecular weight drug at the site of implantation within a known time period. Another embodiment provides a method for site-specific or systemic drug delivery by implanting in the body of a patient in need thereof an implantable drug delivery device containing a therapeutically effective amount of a biological, veterinary or pharmaceutical agent(s) in combination with the monomer, oligomer or polymer. In one embodiment, the monomer, oligomer or polymers of the invention may be particularly useful for the controlled delivery of an agent(s), or as a medium for the localized delivery of an agent(s) to a selected site. For example, the monomer, oligomer or polymers of the invention may be used for the localized delivery of a therapeutic agent to a selected site within the body of a human patient, i.e. within or near a tumor, where the monomer, oligomer or polymer degradation provides a localized, controlled release of the therapeutic agent(s).

In another embodiment a method for delivering an agent(s) to a patient comprises providing a medical device having at least one surface, comprising a first monomer, oligomer or polymer on all or a portion of the surface, wherein the monomer, oligomer or polymer is generally capable of breaking down, e.g. including but not limited to hydrolyzing, in the physiologic milieu to form a first agent(s), and administering the device to the patient so that the first agent(s) is(are) delivered to the patient's site. The device may comprise additional monomer, oligomer or polymers and/or additional agents such as a second agent, third agent, and so on, where the additional agents are, e.g. incorporated, blended, attached, appended or dispersed within the monomer, oligomer or polymer as described herein, or otherwise annexed to or associated with the monomer, oligomer or polymer such that the additional agent(s) dissociate from the monomer, oligomer or polymer upon hydrolysis and are delivered to the patient. The device may comprise an agent(s) that combine in vivo to form a new agent(s) that may be delivered to the patient. The agent(s) may be delivered to any suitable site(s) in a patient, such as the circulatory system e.g. a vein or artery, a tissue, an organ e.g. lung, liver, spleen, kidneys, brain, eye, heart, muscle, and the like, a bone, cartilage, connective tissue, epithelium, endothelium, nerves, a tumor, or other site suitable for delivery of an agent(s). Suitable sites will typically be sites that are or will be in need of treatment with an agent(s), such as, e.g., an injured site or a site that may become injured, for example, due to a disease, a medical condition, or during or after a medical or veterinary surgical procedure, e.g. implantation of an artificial limb or device. The method provides a medical or veterinary device having at least one surface, comprising a first monomer, oligomer or polymer on all or a portion of the surface, wherein the monomer, oligomer or polymer is capable of breaking down, e.g. hydrolyzing, in the physiologic milieu to form a first agent(s), and positioning the medical device at or near the interior surface of the vein or artery such that the first agent(s) dissociates upon hydrolysis and is delivered to the pre-selected site. The device may comprise additional monomer, oligomer or polymers and/or additional agents, e.g. an additional agent(s), where the additional agent(s) may be incorporated, attached, appended or dispersed within the monomer, oligomer or polymer, as described herein, or otherwise annexed to or associated with the monomer, oligomer or polymer such that the additional agents dissociate upon hydrolysis and are delivered to the interior surface of the vein or artery. The device may comprise agents that combine in vivo to form a new agent or agents that are delivered to the interior surface of the vein or artery. In one embodiment, the method prevents, reduces, and/or inhibits the development of restenosis in the blood vessel. Restenosis may be defined as, for example, the narrowing of the vessel to about 80%, about 70%, about 60%, about 50%, about 40%, about 30%, about 20%, about 10% or less, of the diameter of the vessel after removal of any blockages from the vessel and the placement of the device into the vessel.

The monomers, oligomers and polymers of this invention may be subject to methods commonly employed in synthetic polymer chemistry to produce a variety of useful articles with valuable physical and chemical properties. They may also be readily processed into microparticles, nanoparticles, pastes and gels, or solvent cast to yield films, membranes, coatings, chips and fibers with different geometric shapes for design of various medical devices and implants, and may also be processed by compression molding and extrusion as well as coated devices and implants, films, tamponades, and many other articles. One preferred embodiment of the invention incorporates the monomer(s), oligomer(s) and/or polymer(s) or their salt(s), mixture(s), dispersion(s), or blend(s) into films, membranes, pastes, gels, microspheres, nanoparticles, or fibers useful in orthopedic and ancillary applications. The present monomers, oligomers and polymers may be formed into shapes to be placed in contact with the ends of fractured bones, to coat a medical or orthopedic device such as an artificial joint or bone, or to coat or fill a cavity left behind by the removal of a device. In one particularly important embodiment the monomers, oligomers and/or polymers may be formed into articles, including portions of or full bone replacements, coatings, pastes, tablets, wafers and other forms that are intended for implantation at, or near, the site of injury. In one embodiment, these objects are intended for contact with exposed bone to provide a sustained delivery of an anti-inflammatory agent(s), and other drugs to the bone. The quantity of monomer, oligomer and polyanhydride that hydrolyzes to form a therapeutic amount of anti-inflammatory agent(s), or an amount effective to inhibit or reduce growth of bone, or to inhibit or reduce its resorption or breakdown, may be readily determined by those of ordinary skill in the art, for instance, by in vivo experimentation such as that described in Example 63 below. The delivery or application of these compositions, by preventing degradation of bone and growth of new bone at the site, preserves the site of injury until it may be repaired. The selection of the form of the polymer, composition or device for use in a specific application may be performed routinely by those of skill in the art based upon the type of injury and the site stabilization needed. Compositions and devices comprising the polyanhydrides of the invention may be used to coat devices, such as orthopedic devices for fixation of bone fractures, e.g. pins, cuffs or screws, to decrease local inflammation and bone resorption normally associated with placing of these devices, as well as to decrease or delay the growth of bone on these devices. Coatings comprising an anti-inflammatory agent(s) placed on an orthopedic device may also inhibit bone degradation or resorption that often arises from infection at the implantation site, e.g. deep bone infection. Another advantage of the incorporation of the present agent(s) into the compositions, devices and methods of the invention is that they lower the pH crating an environment unfavorable to bacterial growth that inhibits the development of infection. They may be readily processed into pastes, films, coatings, nanoparticles, microparticles, gels, powders, sprays, creams, ointments, tablets, capsules, emulsions, solutions, suspensions, granules, fillers, covers, linings, grids, meshes, gramps, and fibers for use in the design of articles, e.g. devices and implants, of different geometric shapes using techniques known in the art, such as solvent casting, solution or suspension spraying, compression molding or extrusion. Some applications will benefit from incorporating short half-life oligomers and polymers that will disappear after a pre-determined initial stage. Other applications are more suited for the use of oligomers and/or polymers, and their mixtures and blends, having longer half-lives that will preserve the activity of the agent for extended periods of time, e.g. months or years, or for the duration of the life of the article if the monomer, oligomer and/or polymer may be incorporated into the article itself or mixed therein with other monomers, oligomers and/or polymers and/or other agents or natural substances such as wood derivatives and the like.

The compositions, devices and methods of the present invention are useful for treating a wide array of diseases and conditions, including, for example, those set forth below and/or otherwise described herein. Compositions, devices and methods described in this patent may be used, for example, as nanospheres and/or microspheres, e.g. anti-inflammatory microspheres or nanospheres with e.g. amoxicillin for reconstructive surgery, bone restructuring by direct injection, in cases of bone inflammation by injection of microspheres; or in the form of dry powders. In bone and orthopedic applications, the compositions, devices and methods may be used, for example, in the manufacture of injections for use in orthopedic surgery; for bone implants; for the prevention of bone changes; for reconstructive plastic surgery involving bone structures, for wound healing by inhibition of osteoclasts and prevention of spurious bone growth; as bone putty; for spinal cage bone pins, e.g. an admixture of at least one anti-inflammatory agent(s), monomer, oligomer, polymer, or their blends or dispersions, with hydroxyapatite fillers and other fillers; as a coating for orthopedic implants to decrease pain, inflammation, bone erosion and infections; as combinations of poly-NSAIDs plus poly-antibiotics to treat osteomyelitis or other bone infections by direct injection into the marrow; for the treatment of bone cancer with antiproliferatives and/or anti-neoplastic drugs; for the treatment of trauma; as prosthetic devices and coatings therefore; or other devices used in bone and orthopedic applications as otherwise referenced herein. In addition, tablets, pellets and articles of various other forms comprising the agent(s), monomer(s), oligomer(s), polymer(s), blends, mixtures or dispersions may be implanted at or near a site of injury prior to a full operative procedure for interim relief of pain and inflammation and for the prevention of bone growth and resorption. In oncology, such compositions, devices and methods may be used for treating bone, medular cancer, for delivery to any surgical site where cancer tissue or bone may be removed and there exists a concern that not all cancer cells were removed; or for delivering compositions of poly-antiproliferatives sprinkled into the peritoneum, which slowly erode and circulate through the lymphatic system where the primary metastases congregate. In dentistry, such compositions, devices and methods may be used in implanting alveolar bridges, tooth implants, and for preventing bone and gum erosion. The compositions may be also be in the form of microparticles e.g. microspheres, microplatelets or other microstructures, as a powder or pellets to be applied locally e.g. by sprinkling or implantation without or before invasive surgery, to the affected area, and many others.

X. Compositions

a. Formulations

The monomers, oligomers and polymers of the invention may be formulated as pharmaceutical compositions and administered to a mammalian host in a variety of forms adapted to the chosen route of administration, whether topical or systemic, e.g. by oral, rectal, parenteral, intravenous, intramuscular, intraperitoneal, transcutaneous, intraspinal, intracranial, topical, ocular, in situ, pulmonary, or subcutaneous routes, among others. For some routes the monomer, oligomer or polymer may conveniently be formulated as micronized particles. Preferred are those routes that permit the substantially localized administration of the agent(s). Thus, the present compounds may be systemically administered in combination with a pharmaceutically acceptable vehicle such as an inert diluent or an assimilatable edible carrier. They may be enclosed in hard or soft shell gelatin capsules, may be compressed into tablets, or may be incorporated directly with the food of the patient's diet. For oral therapeutic administration, the compound may be combined with one or more excipients and used in the form of ingestible tablets, buccal tablets, troches, capsules, elixirs, suspensions, syrups, wafers, and the like. Such compositions and preparations preferably contain at least 0.1% of monomer, oligomer or polymer by weight. The percentage of agent or monomer, oligomer or polymer in the compositions and preparations may, of course, be varied and may conveniently be about 0.1, 1, 25, 10, 30, 45 to about 50, 60, 75, 80 wt %, and any ranges defined by their combination, and of a given unit dosage form. The amount of monomer, oligomer or polymer in such therapeutically useful compositions may be such that an effective dosage level will be obtained. The tablets, troches, pills, capsules, and the like may also comprise binders such as gum tragacanth, acacia, corn starch, gelatin or others; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form comprises a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the compound may be incorporated into sustained-release preparations and devices.

The monomer, oligomer or polymer may be administered subcutaneously, intramuscularly, intravenously, intraspinally, intracranially, intrauterally, rectally, intraperitoneally, and into and around any applicable body part close to the site of injury, bone damage, wound or surgical site, e.g. by infusion or injection. Solutions of the monomer, oligomer or polymer may be prepared with a suitable solvent such as an alcohol, optionally mixed with a nontoxic surfactant. Dispersions may also be prepared in glycerol, liquid polyethylene glycols, triacetin, and mixtures thereof and in oils. Under ordinary conditions of storage and use, these preparations contain a preservative to prevent the growth of microorganisms. Pharmaceutical dosage forms suitable for injection or infusion may include sterile solutions or dispersions or sterile powders comprising the monomer, oligomer or polymer comprising the ingredient. Such dosage forms may be adapted for the extemporaneous preparation of sterile injectable or infusible solutions or dispersions, optionally encapsulated in liposomes. In all cases the ultimate dosage form should be sterile, fluid and stable under the conditions of manufacture and storage. The liquid carrier or vehicle may be a solvent or liquid dispersion medium comprising, for example, ethanol, a polyol e.g. glycerol, propylene glycol, liquid polyethylene glycols, and the like, vegetable oils, nontoxic glyceryl esters, and suitable mixtures thereof. The proper fluidity may be maintained, for example, by the formation of liposomes, by the maintenance of the required particle size in the case of dispersions or by the use of surfactants. The prevention of the action of microorganisms may be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions may be brought about by the use in the compositions of agents delaying absorption, for example, aluminum monostearate and gelatin. Sterile injectable solutions are prepared by incorporating the monomer, oligomer or polymer in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the ingredient plus any additional desired ingredient present in the previously sterile-filtered solutions.

For topical administration to the site the agent(s) may be applied directly or by incorporation into a monomer(s), oligomer(s), polymers, their salts, compositions, blends, mixtures, dispersions, or article of manufacture. These products may be administered in conjunction with a biologically acceptable carrier, which may be solid or liquid, many of which are known to the art. See, for example U.S. Pat. Nos. 4,608,392; 4,992,478; 4,559,157; 4,820,508. Useful solid carriers include finely divided solids such as talc, clay, microcrystalline cellulose, silica, alumina and the like. Useful liquid carriers include alcohols or glycols or alcohol/glycol blends, in which the present compounds may be dissolved or dispersed at effective levels, optionally with the aid of non-toxic surfactants. Adjuvants such as fragrances and additional antimicrobial agents may be added to optimize the properties for a given use. The resultant liquid compositions may be applied from absorbent pads, used to impregnate bandages and other dressings, or sprayed onto the affected area using pump-type or aerosol sprayers. Thickeners such as synthetic polymers, fatty acids, fatty acid salts and esters, fatty alcohols, modified celluloses or modified mineral materials may also be employed with liquid carriers to form spreadable pastes, gels, ointments, soaps, and the like, for application directly to the skin of the user. The agent(s), monomer(s), oligomer(s), polymer(s), their blend(s), mixture(s) or dispersion(s) may be formulated so that it will be released over an extended period of time when administered in accordance with the invention, e.g. over at least about 2, 5, 7, 10, 20, 40, 60, 80, 100, 120, 140, 160, or 180 to about 200, 220, 240, 260, 280, 300, 320, 340, or 360 days, and even longer. For example, when applied for treatment of hard tissue the agent(s), monomer, oligomer, polymer compositions and artifacts may be formulated for release over a period of about 30 to about 90 days; for treatment of soft tissue about 1, 2, 5, or 10 to about 12, 15, 20, or 30 days, or over about 1 to 2 years. A monomer, oligomer or polymer composition or article of this invention may have for example properties compatible with dosage of drug delivered, pharmacokinetics, rate of generation, elution or release, duration of release, elution or generation of the drug, agent solubility and binding characteristics to other agents and substances in the environment, another agent interaction, e.g. drug interaction. The monomer, oligomer or polymer composition or article may have properties compatible with the physical, chemical, and/or biological requirements for matching the environment for which it is intended, e.g. coating with the surface and bulk of a medical or veterinary device, or implant, such as the coating's adherence to the surface of the implanted medical or veterinary device or bone replacement during processing/coating as well as during implantation, coating stability on the device, coating reproducibility and reliability, non-planar coating ability, porous, and textured geometries, the void filling ability for providing agent reservoirs, and the ability of the coating to withstand mechanical e.g. tensile, compressive, torsicional, and shear, and frictional forces generated during coating processing/application, implantation and subsequent use. One example is the behavior of a coating during subsequent tissue response of an implanted medical or veterinary device. The monomer, oligomer or polymers of the present invention may also be incorporated into systemic and topical formulations and, among these, preferred are formulations that are suitable for nasal, intracavitary, external to bone structures, topical, parenteral e.g. near afflicted tissue or bone, into and around the spine, disks, etc., and intraarticularly, and transdermal administration, among others. The compositions and articles of this invention may conveniently be presented in single or multiple unit dosage forms as well as in bulk, and may be prepared by any methods well known in the art of pharmacy and marketed in the form of a kit with the requisite articles of manufacture and instructions for its use. The kit may comprise already formulated compositions, or it may contain its elements and instructions for its formulation and administration regime. The kit may also contain other agents, such as those described in this patent and, for example, when for parenteral or topical administration, it may also include a carrier in a separate container, cartridge, pack or pouch, which may be sterile. The present compositions may also be provided in a sterile contained for addition of a liquid carrier prior to administration. See, e.g. U.S. Pat. No. 4,956,355; UK Patent 2,240,472; EPA 429,187; PCT 91/04030; Mortensen, S. A., et al., Int. J. Tiss. Reac. XII(3): 155-162 (1990); Greenberg, S., et al., J. Clin. Pharm. 30: 596-608 (1990); Folkers, K., et al., Proc. Nat'l. Acad. Sci. 87: 8931-8934 (1990), the relevant preparatory and compounding portions of all being incorporated herein by reference. Formulations suitable for in situ delivery, topical and parenteral administration are preferred. The formulation of the composition of the invention may include placing at least one agent(s) monomer(s), oligomer(s), polymer(s), or their blend(s), mixture(s) or Dispersion(s) in contact with a carrier and one or more accessory ingredients. In general, the formulations are prepared by uniformly and intimately bringing the agent, monomer, oligomer or polymer into contact or association with any agents that will be mixed, blended or dispersed therein, and optionally with a liquid or solid carrier and then, if necessary, shaping the product into desired formulations described elsewhere in this patent.

Compositions suitable for implantation may be presented in discrete units, such as capsules, cachets, lozenges, or tablets, each containing a predetermined amount of the compound. Examples are a powder or granules; a solution, or suspension in an aqueous or non-aqueous liquid, or mixtures thereof; or an oil-in-water or water-in-oil emulsion. Such compositions may be prepared by suitable pharmaceutical methods known in the art. For example, a tablet may be prepared by compressing or molding a power or granules containing the compound, optionally with one or more accessory ingredients. Compressed tablets may be prepared by compressing, in a suitable machine, the compound in a free-lowing form, such as a powder or granules optionally mixed with a binder, lubricant, inert diluent, and/or surface active/dispensing agent(s), among other formulation ingredients known in the art. Tablets may be made by molding in a suitable machine, the powdered monomer, oligomer or polymer moistened with an inert liquid binder. Compositions suitable for parenteral administration comprise sterile aqueous and non-aqueous injection solutions of the monomer, oligomer or polymer, and are preferably isotonic with the blood of the intended recipient, and may contain in addition to other agents antioxidants, buffers, bacteriostats and solutes which render the compositions isotonic with the blood of the intended recipient. Aqueous and non-aqueous sterile suspensions may include suspending agents and thickening agents. The compositions may be presented in unit-dose or multi-dose containers, for example sealed ampoules and vials, and may be stored in a freeze-dried (lyophilized) condition requiring only the addition of the sterile liquid carrier, for example, saline or water-for-injection immediately prior to use. Extemporaneous injection solutions and suspensions may be prepared from sterile powders, granules and tablets of the kind previously described.

Compositions suitable for topical application to the site of injury or bone implantation or replacement preferably take the form of an ointment, cream, lotion, paste, gel, spray, aerosol, powder, or oil, and the carriers that may be used include vaseline, lanoline, polyethylene glycols, alcohols, transdermal enhancers, and many others known in the art, as well as combinations of two or more of them. Compositions suitable for transdermal administration to sites close to the skin, e.g. arm, cranial, spinal and facial bones, may be presented as discrete patches adapted to remain in intimate contact with the epidermis of the recipient for a prolonged period of time, and may be delivered by iontophoresis and typically take the form of an optionally buffered aqueous solution of the compound. See, e.g. Pharmaceutical Research 3: 318 (1986), the relevant portion of which is incorporated herein by reference.

The agent may be loaded in the monomer, oligomer and/or polymer, or it may be dispersed, mixed or blended therewithin within broad amounts of the composition. For example, the agent(s) may be contained in the composition in amounts of about 0.001%, about 1%, about 2%, about 5% to about 5%, about 10%, about 20%, about 40%, about 90%, about 98%, about 99%, or about 99.999% of the composition. These amounts may be adjusted when and if additional agents with overlapping activities are included as discussed above. Dosage will vary depending on the agent(s), age, weight, and condition of the subject, and the treatment may be initiated with small dosages less than optimal doses of the monomer, oligomer or polymer of the invention, and increased until a desired or even an optimal effect under the circumstances, may be reached. In general, the dosage comprises about 1, 5, 10, or 20 mg monomer, oligomer or polymer/kg body weight to about 100, 200, 500 or 1000 mg monomer, oligomer or polymer/kg body weight. Higher or lower doses, however, are also contemplated depending on the actual loading of the agent(s) in the monomer, oligomer or polymer and are, therefor, within the confines of this patent. In general, the content of the agent in the amount of monomer, oligomer or polymer delivered is preferably such that when administered it will provide a concentration at the desired site that will afford effective results without causing unduly harmful or deleterious side effects, and may be administered either as a single unit dose, or if desired in convenient subunits administered at suitable times throughout the day. The additional agent(s) are administered in amounts that are known in the art to be effective for the intended application. In cases where the additional agent in the composition has overlapping activities with the principal agent, i.e. an additional NSAID and its salts, the dose of one, the other or both agents may be adjusted to attain a desirable effect without exceeding a dose range which avoids untoward side effects. Thus, when other analgesic and anti-inflammatory agents are added to the composition, they may be added in amounts known in the art for their intended application or in doses somewhat lower that when administered by themselves. In general, the present composition may be provided as various systemic and topical formulations, which includes, but is not limited to, oral, intrabuccal, intrapulmonary, rectal, intrauterine, intradermal, topical, dermal, parenteral, intratumor, intracranial, buccal, colonic, sublingual, nasal, injectable such as intramuscular, subcutaneous, intraglandular, intraorgan, intralymphatic, intraarticular, intravascular, intravenous, or intrathecal, inhalable, transdermal, intraarticular, intracavitary, implantable, transdermal, iontophoretic, intraocular, ophthalmic, vaginal, otical, implantable, slow release and enteric coating formulations and those suitable for in situ delivery of the agent(s). Preferred for the purposes of the present applications are topical, injectable, intraarticular, at or near a site of injury, next to a bone structure, etc. The actual preparation and compounding of these different formulations is known in the art and need not be detailed here. The monomer, oligomer or polymer of the invention may be administered once or several times per day, per week, per month, or per year, depending on its half life. The monomer, oligomer or polymer may be administered to the inhalation system, e.g. to the lungs or nasally by any suitable means, but are preferably administered by generation of an aerosol comprised of respirable particles that the subject inhales. Respirable particles may be liquid or solid, and are of respirable size; that is particles of a size sufficiently small to pass through the mouth and larynx upon inhalation and into the bronchi and alveoli of the lungs. In general, particles ranging from about 0.5, about 1, about 2, or about 5 micron to about 5, about 7, about 10, or about 20 micron in size are respirable, whereas those larger than respirable size tend to deposit in the throat and be swallowed. Thus, the quantity of non-respirable particles in the aerosol is preferably minimized. For nasal administration, a particle size in the range of about 10, about 15, about 20, about 30, or about 50 μm to about 20, about 75, about 100, about 200, about 350, or about 500 μm is preferred to ensure retention in the nasal cavity. Liquid pharmaceutical compositions of monomer, oligomer or polymer for producing an aerosol may be prepared by combining the monomer, oligomer or polymer alone or in admixture or dispersion with other polymers or agents with a stable vehicle, such as sterile pyrogen free water, or other known carriers. Solid particulate compositions containing dry respirable particles of micronized compound may be prepared by grinding dry monomer, oligomer or polymer(s) with/without dispersed agents with a mortar and pestle, and then passing the micronized composition through a 400 mesh screen to break up or separate out large agglomerates. A solid particulate composition comprised of the monomer, oligomer or polymer may optionally comprise a dispersant that facilitates aerosol formation. A suitable dispersant may be lactose, which may be blended with the compound in any suitable ratio, e.g. an about 1:1 wt:wt ratio. Other dispersants, however, are also suitable and their identities and formulation characteristics may be learned from their use in the art. Aerosols of liquid particles comprising the monomer, oligomer or polymer of the invention may be produced by any suitable means, such as with a Nebulizer. See, e.g. U.S. Pat. No. 4,501,729. Nebulizers are commercially available devices that transform solutions or suspensions of the ingredient into a therapeutic aerosol mist either by means of acceleration of a compressed gas, typically air or oxygen, through a narrow venturi orifice, or by ultrasonic agitation. Suitable compositions for use in a nebulizer consist of the monomer, oligomer or polymer in a liquid carrier, the monomer, oligomer or polymer comprising about 0.01, 1, 5, 10 w/w % to about 20, 30, 40 w/w % of the formulation, and some times even higher amounts. The carrier is typically water, or a dilute aqueous alcoholic solution, preferably made isotonic with body fluids by the addition of, for example sodium chloride. Optional additives include preservatives if the compositions are not prepared sterile, for example, methyl hydroxybenzoate, antioxidants, flavoring agents, volatile oils, buffering agents and surfactants. Likewise, aerosols of solid particles comprising the monomer, oligomer or polymer with/without other polymers and/or agents may be produced with any sold particulate aerosol generator. Suitable aerosol generators for administering solid particulate medicaments to a subject produce respirable particles, and generate a volume of aerosol containing a predetermined metered dose of a medicament at a rate suitable for human administration. Examples of such aerosol generators include metered dose inhalers and insufflators. The dispersed agent(s) may be administered concurrently with the monomer, oligomer or polymer(s), and may be an agent suitable for preventing and treating sleeplessness, mood disorders, anxiety, irritability, wasting, bulimia, anorexia nervosa, cancer, viral and microbial infections, heart conditions, ischemia, menopause, pain, inflammation, wounds and burns, muscle tension, low bone calcification, inflammatory diseases such as auto-immune diseases, COPD, and inflammatory bowel disease, and many more, and to treat and prevent steroid intake secondary effects and to improve body weight and increase muscle mass, preferably in the same composition, as described above.

The phrase “concurrently administering” as used herein refers to the monomer, oligomer or polymer(s) and the dispersed or appended agent(s) being administered either (a) simultaneously in time, and preferably by formulating the two together in a common pharmaceutical carrier, or (b) at different times during the course of a common treatment schedule. In the latter case, the two may be administered at times effective to complement their half lives and, thereby offset a reduction in peak level of one with an increasing level of the other and, thereby, counter balance any decrease in activity of one with an increase in activity of the other as a result of their alternate administration schedule. Thus, the monomer, oligomer or polymer may or may not be administered for a time sufficient to bring endogenous levels of an agent(s) back to prior levels in the subject. If the present composition or formulations are administered for a time sufficient to replenish endogenous levels of an agent(s) (if lowered with respect to prior levels in the same subject), then the agent(s) or its(their) precursor(s) present in the monomer, oligomer or polymer, or their dispersions or mixtures with other monomer, oligomer or polymers and/or agents are administered in amounts effective to increase levels to a desired level. Thereafter, the doses of the two or more monomer, oligomer or polymers and agents may be reduced so as to maintain desired levels, whether the dispersed, appended or admixed monomer, oligomer or polymer(s) or agent(s) has(have) overlapping activity(ies) with the agent(s) or compound(s) released or, if of different activity, the dose of the admixed, appended or dispersed monomer, oligomer or polymer(s) and/or agent(s) may be reduced along with that of the compound released in cases of reduced risk of relapse. If the monomer, oligomer or polymer(s) is(are) administered for a time sufficient to replenish endogenous levels, and this is attained, the continuation of treatment will depend on whether levels are maintain in the absence of treatment or not. Moreover, whether the admixed, appended or dispersed agent(s)' dose may be reduced or not will depend on whether or not it may be necessary to continue its administration or the subject remains stable in its absence. If the practitioner perceives a need to offset a future relapse, be it as a decrease in agent(s) levels or even its depletion and/or a need or benefit from a continued administration of the dispersed, appended or admixed monomer, oligomer or polymer(s) and/or agent(s), the treatment may be continued under close monitoring. The admixed, appended or dispersed monomer, oligomer or polymers and agents, examples of which are listed above, may be administered per se or in the form of their biologically, physiologically, pharmacologically, pharmaceutically or veterinarily acceptable salts. When used in medicine, the salts of these agents should be pharmacologically and pharmaceutically acceptable, but non-pharmaceutically acceptable salts may be used to prepare the free compound or pharmaceutically acceptable salts thereof and are appropriately included within the scope of this invention. Such pharmacologically and pharmaceutically acceptable salts include, but are not limited to, those prepared from the hydrochloric, hydrobromic, sulphuric, nitric, phosphoric, maleic, acetic, salicylic, p-toluenesulfonic, tartaric, citric, methanesulphonic, formic, malonic, succinic, naphthalene-2-sulphonic and benzenesulphonic acids, among others. Pharmaceutically acceptable salts also may be prepared as alkaline metal or alkaline earth salts, such as sodium, potassium or calcium salts of the carboxylic acid group. The present pharmaceutical formulations, whether for veterinary or human use, may comprise, in addition to the monomer, oligomer or polymer(s) and one or more appended, admixed, blended or dispersed monomer, oligomer or polymers and/or agents, one or more pharmaceutically acceptable carriers, and other markers, diagnostic, prophylactic and/or therapeutic ingredients suitable for specific applications. The carrier(s) should be biologically, physiologically, pharmacologically, pharmaceutically or veterinarily acceptable in the sense of being compatible with the other ingredients of the formulation and not unduly deleterious to the recipient thereof.

Formulations of the present invention suitable for oral administration may be presented in discrete units such as powders, granules, dragees, capsules, cachets, tablets or lozenges, each containing a pre-determined amount of the monomer, oligomer or polymer that will release a desired dose of the agent(s) in the form of a powder or granules; or a suspension in an aqueous liquor or non-aqueous liquid such as a syrup, elixir, emulsion or draught. Tablets may be made by compression or molding of the monomer, oligomer or polymer(s), optionally with one or more agents and accessory ingredients. Compressed tablets may be prepared by compressing in a suitable machine, with the compound being in a free-flowing form such as a powder or granules that may be mixed with a binder, disintegrate, lubricant, inert diluent, surface active agent or dispersing agent, among other ingredients. Molded tablets comprised of a mixture of the powdered compound with a suitable carrier may be made by a suitable molding machine. Formulations suitable for parenteral administration may be prepared as a sterile aqueous formulation of the monomer, oligomer or polymer(s) and agent(s), preferably isotonic with the blood of the recipient.

Coating or filler formulations for applications other than those mentioned above are suitably prepared by methods known in the art, by mixing the monomer, oligomer or polymer(s) of the invention and other desired ingredients in a manner suitable for the intended purpose. Other monomer, oligomer or polymers and agents may be appended to, mixed with, or dispersed within the monomer, oligomer or polymer(s) as desired. The addition of other admixed or dispersed monomer, oligomer or polymers, agents and accessory ingredients may be desirable. In addition to the aforementioned ingredients, the formulations of this invention may further include one or more accessory ingredient(s) such as diluents, buffers, flavoring agents, binders, disintegrant, surface active agents, thickeners, lubricants, preservatives (including antioxidants), colorants, perfumes, preservatives, and the like. Other ingredients may also be utilized as is known in the art.

Useful doses of the monomer, oligomer or polymers may be determined using techniques known in the art, such as, e.g., by comparing their in vitro activity with the in vivo activity of the therapeutic agent in animal models. Methods for the extrapolation of effective doses in mice, and other animals, to humans are known to the art; for example, see U.S. Pat. No. 4,938,949. Additionally, useful doses may be determined by measuring the rate of hydrolysis or enzymatic degradation for a given monomer, oligomer or polymer under various physiological conditions. The amount of a monomer, oligomer or polymer required for use in treatment will vary not only with the particular monomer, oligomer or polymer selected but also with the route of administration, the nature of the condition being treated and the age and condition of the patient and will be ultimately at the discretion of the attendant physician or clinician, and is easily determinable by one of ordinary skill in the art. The quantity of monomer, oligomer or polymer drug to be administered to a host that is effective for the selected use may be readily determined by those of ordinary skill in the art without undue experimentation. The quantity essentially corresponds stoichiometrically to the amount of drug that may be known to produce an effective treatment for the selected use. The desired dose may conveniently be presented in a single dose or as divided doses administered at appropriate intervals, for example, as two, three, four or more sub-doses per day. The sub-dose itself may be further divided, e.g., into a number of discrete loosely spaced administrations. The total amount of an agent(s) released will vary depending on the particular agent(s) and treatment protocol involved, as may be easily determined by one ordinarily skilled in the art. The amount of agent released will typically be from about 0.1 μg to about 10 g, preferably from about 1 μg to about 100 mg, more preferably from about 10 μg to about 10 mg, more preferably from about 50 μg to about 1 mg. Preferably, the monomer, oligomer or polymers are formulated to provide local release of an effective amount of an agent or agent over a period of at least about 2, about 5, about 10, about 20, or about 40 days. The compositions may also preferably be formulated to provide local release of an effective amount of the agent over a period of up to about 3 months, about 6 months, about 1 year, or about 2 years. The agent(s) may be released from the monomer, oligomer or polymer at any rate suitable for appropriate delivery of the agent to the patient. In one embodiment, the agent may be released at a rate from about 0.01 μg per day to about 100 mg per day, from about 1 μg per day to about 10 mg per day, or from about 10 μg per day to about 1 mg per day. It will be appreciated that the greater the potency of the coating, the better with regard to minimizing the space required for the administered product, the potential cost of the product, the ease of manufacturing the product, and the potential impact on other desired properties of the medical implant. The monomer, oligomer or polymers of the present invention may be characterized by techniques known in the art. Degradation and drug release profiles of the drug delivery systems of the present invention may also be determined routinely. The range of therapeutically effective dosages, that is, the dosage levels necessary to achieve the desired result, of a microparticle of the invention will be influenced by the route of administration, the therapeutic objectives, and the condition of the patient. As such, an agent(s) or a monomer, oligomer or polymer of the invention may be administered as a single daily dose, several times daily, every other day, weekly, etc. depending on the dosage requirements. Individual determinations may need to be made to identify the optimal dosage required as is known in the art. An agent(s), monomer(s), oligomer(s) or polymer(s)' dosage(s) may be determined by comparing their in vitro activity, and in vivo activity of an agent(s), compound(s) or monomer, oligomer or polymer(s) in an animal model. Methods for the extrapolation of effective dosages in mice, and higher animals, to humans are known to the art as well. See, for example, U.S. Pat. No. 4,938,949. Useful dosages may be determined also by measuring the rate of hydrolysis or enzymatic degradation for a given monomer, oligomer or polymer under various physiological conditions. The amount of a monomer, oligomer or polymer required for use in treatment will vary not only with the particular monomer, oligomer or polymer selected but also with the route of administration, the nature of the condition being treated and the age and condition of the patient and will be ultimately at the discretion of the attendant physician or clinician. The desired dose may conveniently be presented as a single daily dose, or as divided doses administered at appropriate intervals, for example, as multiple daily sub-doses. Each sub-dose itself may be further divided, e.g., into a number of discrete loosely spaced administrations. The monomer, oligomer or polymers of the invention are also useful for the application, administration and release of a combination of agents typically by 1) dispersing a second agent(s) or compound(s) within a monomer, oligomer or polymer matrix of the invention comprising a first agent(s) or compound(s); both the first and second agents will be released upon degradation; 2) appending a second therapeutic agent to a monomer, oligomer or polymer of the invention, i.e. not incorporated into the monomer, oligomer or polymer, through hydrolyzable bonds; 3) incorporating into the monomer, oligomer or polymer backbone more than one single agent, e.g. a monomer, oligomer or polymer comprising different agent units; and/or 4) administering more than a single monomer, oligomer or polymer, each comprising a different therapeutic agent(s) either as a blend, a mixture or as separate entities administered together or within a short period of time.

b. Co-Polymers and Polymer Blends

The invention thus provides a composition comprising a polymer of the invention incorporating a first agent(s) in its backbone, and optionally a second agent(s) that may be either part of the polymer backbone or blended or admixed with, or dispersed within the polymer matrix. Another embodiment provides a pharmaceutical composition comprising a polymer of the anti-inflammatory agent(s), and the further agent(s) appended to the polymer, e.g. through hydrolyzable bonds that will release the second agent(s) under appropriate conditions. The polymers of the invention may be employed, applied, or administered to a target site by themselves, or in combination with other agents that are effective to prevent, contain, or treat a given condition, such as may be the case in combination therapy. In the veterinary and medical fields, the method may take the form of the prevention, containment, or treatment of a bone or tissue condition comprising the application, delivery, or administration of an effective amount of at least one agent(s), monomer(s), oligomer(s), polymer(s), blend(s0, mixture(s), or dispersion(s) by itself(themselves) or along another prophylactic, containment, therapeutic and/or traceable agent(s) and/or formulation ingredients. The therapeutic polymers and compositions thereof used in some applications, such as for coating implantable medical and veterinary devices, including orthopedic implants, may require greater elasticity or flexibility while retaining sufficient hardness and adhesiveness to remain intact on the device as the device is handled or otherwise manipulated by the clinician or surgeon or within the body of the patient, such as, e.g. when the device interacts mechanically and chemically, with the surrounding bone, tissue, fluid or luminal wall, or, in the case of a. To provide desired physical properties, including mechanical strength, modulus and elongation without failure, it may be possible to create coatings comprised of a co-polymer of two or more monomers used to create the more than one polymer having physical properties and other performance characteristics that differ from, e.g. bracket the ones desired. In one embodiment, co-polymers of similarly sized or “sequential” linkers, e.g. adipic acid (C₆) and suberic acid (C₈) are made in order to “fine tune” the physical properties of the polymer to a state between the two available linkers. However, “non-sequential” co-polymers are also contemplated, for example a co-polymer containing adipic acid (C₆) and sebacic acid (C₁₀) linkers. Additionally, co-polymers comprising three or more linker group moieties are also contemplated. In one embodiment, the co-polymer may comprise monomers of salicylic acid and adipic acid, and salicylic acid and suberic acid, at about 50% or more mole % co-polymer is the monomer salicylic acid and adipic acid, respectively. However, proportions of any of the agent monomers, and oligomers may be employed, for example, about 5, about 10, about 20, about 30, about 40, or about 50 to about 60, about 70, about 80, about 90, about 95, or about 99 wt %. Alternatively or in combination with one or more of the co-polymers described above, it is possible to create a physical blend of two or more polymers or co-polymers in which the individual polymers or co-polymers blended each have a set of physical properties and performance characteristics that meet or exceed requirements for a coating for the specified implantable medical or veterinary device. Each individual polymer may have one or more physical properties and/or performance characteristics that are insufficient for that device and its application. The combination of properties and characteristics provided by the blend, however, may be made to meet or exceed the required properties and characteristics needed for the device and application.

These blends may be of monomer, oligomer or polymers that are miscible or immiscible in each other. For example, it is possible to make a co-polymer or blend of monomer, oligomer or polymers or co-polymers in which one monomer in the co-polymer or one monomer, oligomer or polymer or co-polymer in the blend has a hardness that exceeds the coating requirements for a device and its application but insufficient flexibility while another monomer in the co-polymer or another monomer, oligomer or polymer or co-polymer has sufficient flexibility but insufficient hardness. The physical properties and performance characteristics of the copolymer may be fine tuned further by selecting the percentage of each monomer in the copolymer or the percentage of each monomer, oligomer or polymer or co-polymer in the blend towards the combination of monomers, oligomers and/or polymers or co-polymers that produce a coating that has desired physical properties and performance characteristics. In an exemplary embodiment, a polymer comprising salicylic acid or a derivative of salicylic acid, such as diflunisal, and linkers of dicarboxylic acids in which the pair of carboxylic acids within the diacid are separated by a linear alkyl chain may be coated on an article or device. A coating comprising a polymer in which the alkyl chain comprises six atoms of carbon (adipic acid) may crack or craze upon change in dimensions, e.g. during expansion, whereas a coating comprising a polymer in which the alkyl chain comprises eight atoms of carbon (suberic acid) may be excessively tacky or otherwise adhere to the materials used in handling and implantation. For such applications, in the absence of an admixed drug or other additive that alters the physical properties and performance characteristics in a predictable and repeatable manner, a suitable coating may comprise, for example, a polymer of salicylic acid and suberic acid or a copolymer of monomers of salicylic acid and dicarboxylic acid or a physical blend of polymers or co-polymers of salicylic acid and dicarboxylic acid that approximate the tradeoffs in physical properties and performance characteristics, including hardness, tackiness, and flexibility, of polymers created with a linker of suberic acid. In another exemplary embodiment, a polymer comprising salicylic acid or a derivative of salicylic acid, such as diflunisal, and linkers of dicarboxylic acids with linear alkyl chains, and may be coated on an orthopedic implant for use as a hip, knee, shoulder, elbow replacement, a fixation device, or another orthopedic application. In the absence of an admixed drug or other additive that alters the physical properties and performance characteristics in a predictable and repeatable manner, a suitable coating may comprise, e.g. a polymer of salicylic acid and a dicarboxylic acid linker with four, six, eight or ten carbon atoms in the linear alkyl chain (known as succinic and adipic acids, respectively) or a copolymer of monomers of salicylic acid and dicarboxylic acid or a physical blend of polymers or co-polymers of salicylic acid and dicarboxylic acid that approximate the tradeoffs in physical properties and performance characteristics, including hardness, tackiness, and flexibility, of polymers created with a linker of succinic or adipic acids.

c. Combination Therapy

The polymers of the invention are also useful for administering a combination of therapeutic agents. Such a combination therapy may be carried out in the following ways: 1) A second therapeutic agent may be dispersed within the monomer, oligomer or polymer matrix, and may be released upon degradation; 2) A second therapeutic agent may be appended to a monomer, oligomer or polymer of the invention, i.e. not in the backbone with bonds that hydrolyze to release the second therapeutic agent under physiological conditions; 3) The monomer, oligomer or polymer of the invention may incorporate two therapeutic agents into the backbone; or 4) two monomer, oligomer or polymers of the invention, each with a different therapeutic agent may be administered together, or within a short period of time. More than one therapeutic agent may be used in each of the above cases. Thus, the invention also provides a medical device comprising a monomer, oligomer or polymer that hydrolyzes to form a first agent and a second agent that may be dispersed within a matrix of a polymer of the invention. The invention also provides a medical device comprising a monomer, oligomer or polymer that hydrolyzes to form a first agent having a second agent appended to the monomer, oligomer or polymer, e.g. with bonds that will hydrolyze to release the second therapeutic agent under physiological conditions. The monomer, oligomer or polymers of the invention may also be administered in combination with other agents that are effective to treat a given condition to provide a combination therapy, or with an agent(s) that provides an ancillary activity in addition to that of the main agent(s). Thus, the invention also provides a method for treating a disease in a mammal comprising administering an effective amount of a combination of a monomer, oligomer or polymer of the invention and another therapeutic agent. The invention also provides a pharmaceutical composition comprising at least one anti-inflammatory agent(s), monomer, oligomer or polymer of the invention, another therapeutic agent, and a pharmaceutically acceptable carrier. Suitable drug combinations for incorporation into the polymers or the compositions of the invention include for example, a first agent that may be classified as a non-steroidal anti-inflammatory drug (NSAID), such as, e.g., salicylic acid or diflunisal, combined with a second agent classified as an anti-cancer and/or anti-neoplastic agent, e.g. paclitaxel or methotrexate, or as an immunosuppressive, e.g. rapamycin. Preferred drug combinations for incorporation into the polymers or the compositions of the invention include amoxicillin/clavulanic acid; and imipenem cilastatin, among others.

d. Slow Release (SR) Injectable Formulations

Although rheumatoid arthritis (RA) will be discussed as an example of a group of immune diseases, this section is intended to cover immune, and particularly, auto-immune diseases, among many others, that afflict joints, bones and their surrounding tissues. By far the most troubling symptoms of RA are severe pain and swelling of the joints of the wrists, hands, ankles and feet, which occur when the body's immune system mistakenly attacks the synovium (the cells lining the joints), causing intense inflammation. The therapeutic mainstay of RA comprises oral NSAIDs, including non-selective COX inhibitors like aspirin and diflunisal, as well as the newer COX 2-specific NSAIDs, rofecoxib and celecoxib. As disease severity progresses, disease-modifying anti-rheumatic drugs (DMARDs) such as methotrexate, azothioprine, gold salts and immunosuppressive agents are used, despite their serious side effects. More recently, injectable biological response modifiers that block the action of tumor necrosis factor (etanercept and infliximab) have shown great promise, despite their high cost and associated risk of tuberculosis and cancer. Another injectable protein (anakinra) blocks the effects of IL-1, an inflammatory protein over-expressed in RA patients. Notwithstanding the effectiveness of these newer treatments, RA remains a chronic disease, the severity of which fluctuates over time. When pain and swelling flare, a standard treatment is to inject steroids directly into the affected joint, sometimes in combination with a local anesthetic. Such intra-articular injections provide rapid and long-lasting relief of pain and swelling, but only a few steroid injections may be administered safely at any one time, and repeated injections into the same joint may destroy cartilage. These drawbacks have spurred the development of “steroid-sparing” treatments for flared joints. A PLGA microsphere-based intra-articular product is being currently tested to provide slow-release of betamethasone, with the goal of minimizing tissue damage whereas intra-articular hyaluronic acid products are used mostly for osteoarthritis.

In one embodiment, the present invention comprises an injectable composition of the anti-inflammatory agent, monomer, oligomer, polymer, e.g. a polyNSAID, blend, mixture, co-oligomer, co-polymer, or articles made thereof. In one preferred embodiment the product comprises microparticles designed to provide sustained relief of swollen and painful joints after intra-articular injection, surgery, bone and fragment replacement, and the like. On preferred embodiment comprises a micro formulation of mono-, oligo- and/or polyDF, or their blends or mixtures. In another embodiment other drugs, including analgesics such as morphine, may be added during preparation of the microparticle formulation, as a coating or core of the formulation, or in the material employed for shaping articles. Long considered to produce analgesia by the activation of receptors located only within the central nervous system, new evidence demonstrates that narcotic analgesics have a potent local analgesic effect when injected into chronically-inflamed tissue. Clinical studies demonstrated profound pain relief from 1 mg morphine injected into chronically-inflamed (but not acutely-inflamed) gum tissue, and pain relief similar to that of 4 mg dexamethasone by the intra-articular injection of 3 mg morphine in RA patients. The addition of strong analgesics, such as narcotic analgesics, e.g. morphine, to the monomer, oligomer or polymer of the invention presents little or no abuse potential because only low concentrations of morphine are required, generally less than about 5 to 10 wt % as is known in the art, and morphine release from polyDF will be retarded generally by an about 15- to about 18-hr induction period before the onset of biodegradation. For more extended effects, e.g. analgesia, antibiotic and antiseptic action, and the like, drugs such as narcotic analgesics, antibiotics, and others may be incorporated into the backbone.

e. Nanoparticle and Microparticle Formulations

All of the foregoing pharmaceutical applications may employ nano- and/or micro-particular formulations. Nanospheres and microspheres have been made form polydiflunisal having a mean diameter of about 10 to about 100 nm and about 10 to about 100 μm, with an average of 45-50 nm, and 45-50 μm, respectively, the latter being slightly smaller than the size commonly used for drug delivery. A process for preparing microencapsulated monomer, oligomer or polymers of this invention, e.g. of chemical formula IIa or IIb, or for preparing intermediates useful for preparing compounds of formula II are provided in Table 4 below, and also as further embodiments of the invention.

TABLE 4
Microencapsulation Process

| | Microencapsulated |
|---|---|
| Advantages | Agent |
| U.S. Pat. No. 5,407,609 | Proteins |
| Fast Encapsulation Time (msec.) | Peptides |
| Minimal Exposure to Polymer Solvent | Small Molecules |
| High Encapsulation Efficiency | Water-Soluble Drugs |
| Good Yield | Hydrophobic Drugs |
| Yields Small Microparticles | Drugs Encapsulated in |
| (<100 μ; <10 μ) | Lactide/Glycolide Polymers |

Processes for making nanoparticle formulations are also known in the art, and need not be fully described in this patent. The surface eroding property of polymers such as polydiflunisal makes for solid, non-porous particles, e.g. nano- and micro-spheres, useful for sustained drug delivery, and their release duration may be controlled by varying particle diameter, e.g. larger microparticles biodegrade more slowly than smaller ones. Nano- and microparticles for pharmaceutical formulations may be designed to deliver an agent(s) or compound(s) incorporated into the monomer, oligomer or polymer backbone and optionally an agent(s) blended, appended to, or dispersed in the polymer. When rats were administered a single subcutaneous injection of 250 mg polydiflunisal microspheres containing about 192 mg diflunisal formulated in a standard aqueous vehicle a peak plasma diflunisal of 35 μg/ml was achieved within two days, and thereafter the drug level declined slowly over about two weeks whereas a single oral dose of diflunisal produced a drug level that declined rapidly. Microparticle formulations of about 1, 2, 5, 7.5, 10, 25, 50 to about 10, 15, 30, 50, 75, 100, 250 μm are suitable for use in a pharmaceutical, veterinary or other type of formulations. Similarly, nanoparticle formulations may be administered for various applications, having a particle size about 1, 2, 5, 10 to about 15, 20, 30, 50, 100, 250, 500 nm, or various ranges between any two of these values. These and other polymers lacking a drug(s) in their backbone may also be employed as carriers for other agent(s) as has been demonstrated with polymers of the invention carrying paclitaxel and sirolimus. The anti-inflammatory properties of polyNSAIDs when employed as delivery vehicles for an admixed pharmaceutical agent(s) and biological agent(s) will significantly diminish the foreign body response associated with polymers commonly used for injectable depot products, such as PLGA. While the injection of a drug or biological agent carried by a monomer, oligomer or polymer of the invention, e.g. a polyNSAID, may be expected to generate significant drug, e.g. NSAID, concentrations in tissues near the injection site, their systemic levels in most cases will remain less than about 0.1μ, which are far below therapeutic levels. The microparticles of the invention may be formed into various shapes and geometries e.g. spheres, and regular or irregular spheroid shapes. They may also be incorporated into various formulations or compositions, e.g. gelatin capsule, liquid formulation, spray dry formulations, formulations for use with dry powder or aerosol inhalers, compressed tablet, topical gels, topical ointments, topical powder. As would be understood by one of skill in the art, the desired size of a microparticle of the invention will depend on the desired application and mode of delivery. Modes of administration or delivery of a microparticle and nanoparticle formulations of the invention include those set forth herein, including orally, by inhalation, by injection, and topically.

The present invention contemplates the administration of microparticle and nanoparticle formulations that upon degradation or bioerosion may be delivered as is, or yield a smaller particle and/or agent for the effective treatment of a targeted organ or tissue. The present invention also contemplates administration of one or more of the same or different microparticle or nanoparticle formulations of the invention having either all the same size or a mixture of two or more different sizes. By varying the size of the microparticle, the rate of bioerosion and/or the rate of generation of agent(s) and/or the location of agent(s)' generation may be controlled. As a result, timed, e.g. delayed and/or sustained, generation of agent(s) may be achieved. For example, treatment of the inflamed wall of the colon, e.g. inflammatory bowel disease (I.B.D.), infections, and the like, may be achieved by oral administration of microparticles of the invention comprising an anti-inflammatory drug as its active ingredient. Such a microparticles of about 1 to about 10 μm in size may be administered such that upon reaching the ileum region of the small intestine, the microparticle may be about 0.1-1.0 μm in size, and about 0.01 to about 0.1 μm in size upon reaching the colon. See, for example Lamprecht et al., Abstracts/Journal of Controlled Release 72: 235-237 (2001). Once in the intestine, the microparticle may be physically entrapped by the villi and/or microvilli of the intestinal wall and/or by the mucous lining of the intestinal wall, thereby retarding expulsion, and prolonging gastrointestinal residence time and enabling timed sustained generation of the agent(s) in the proximity of the intestinal wall upon bioerosion. The microparticles of the invention may be of about 0.1, 1, 10, 20, 50 to about 60, 70, 80, 90-100 μm, preferably about 0.1 to about 10 μm, and any ranges therewithin. The microparticle of the invention may be administered orally such that blood levels of the microparticle enable perfusion of the agent(s) into the surrounding tissue upon bioerosion. In yet another example, oral administration of microparticles of about 0.1 μm, about 0.3 μm, or about 0.6 μm, or any sizes therebetween and outside this range, may be used to deliver an active drug through the intestine and eventually to the liver via the lymph system. See, for example Jani et al., Pharm Pharmacol. 42: 821-826 (1990); Desai et al., Pharmaceutical Research 13 (12): 1838-1845 (1996).

Microparticles of the invention of about 1 to about 50 μm may be applied preferably at the site of implant, injury, or surgery, topically, or ocularly. One preferred group of microparticles are preferably about 5, about 7, about 10 to about 12, about 15, about 18, about 20 μm. Another preferred group of microparticles suitable for subcutaneous or intramuscular injection are preferably about 1, about 5, about 10, about 15, about 20 to about 25, about 30, about 40, about 50, about 60, about 70 μm, although sizes outside this range may also be employed. In one preferred embodiment microparticles about 10 to about 70 μm may be employed for subcutaneous or intramuscular injection. In another preferred embodiment, microparticles less than about 10 μm may be used to create a smooth product for application to human skin. In another preferred embodiment microparticles about 1 to about 3 μm may be used for skin penetration. However, many other microparticle sizes may be used as well, as exemplified by Smart Particle™ and others (PowderJect Pharmaceuticals, U.K.); U.S. Pat. Nos. 6,328,714, 6,053,889 and 6,013,050, in tissue e.g. skin, mucosa penetration applications that appear to rely more on shape and strength of the microparticle rather than size. The microparticles of the invention may also be used in an inhaled delivery, e.g. direct inhalation at a certain velocity, or by aerosol spray, to the lungs, including deep lungs, or pulmonary region. For example, a microparticle of the invention of about 0.5 to about 10 μm, preferably about 1-5 μm, more preferably about 1-3 μm, even more preferably about 1-2 μm may be formulated into an aerosol. For direct inhalation, about 0.5-6 μm, more preferably about 1-3 μM, microparticle may be used. See, for example AERx® System (Aradigm Corporation, Hayward, Calif.) as well as those described in U.S. Pat. Nos. 6,263,872, 6,131,570, 6,012,450, 5,957,124, 5,934,272, 5,910,301, 5,735,263, 5,694,919, 5,522,385, 5,509,404, and 5,507,277, and MicroDose DPI Inhaler (MicroDose Technologies Inc., Monmouth Junction, N.J.) as well as those described in U.S. Pat. Nos. 6,152,130, 6,142,146, 6,026,809, and 5,960,609. Microparticles of the invention of about <10 μm may be used for intraarticular injections in the treatment of, for example, arthritis. A microparticle of the invention of about 0.1 to about 100 μm, preferably about 0.1 to about 10 μm, more preferably about 0.1-1 μm, may be admixed with a suppository, e.g. glycerin suppository. Nanoparticle formulations of this invention have diameters (average or range of size) about 2, 5, 10, 20, 50, 100 nm to about 150, 250, 350, 500, 700, 850 nm may be applied to therapeutic and prophylactic applications, such as healing of wounds and the like.

The monomer, oligomer or polymer, agent(s) and/or composition(s) of the invention may also be formed into pellets, “biobullets”, i.e. bullet shaped, or seeds, e.g. bullet-shaped seeds, for inclusion in an implantable and/or injectable bioerodable, hollow carrier e.g. barrel, bullet, capsule, syringe or needle that are known in the art. Both animal and human applications are contemplated. Hollow needle-type carriers are also contemplated for use in the invention. In one embodiment, a hollow carrier may have a diameter ranging from about 0.5 to about 10 mm, although other gauges are also suitable. Pellets, “biobullets”, and/or seeds of the invention may be placed inside the hollow cavity or chamber of a bioerodable needle-type carrier. According to the invention, one or more of the same or different pellet(s), “biobullet(s)” or seed(s) of the invention may be placed inside a hollow carrier or delivery device. The pellet, “biobullet” or seed may be any size that will enable placement inside the hollow carrier. The oral, injectable, implantable and topical formulations of the invention are suitable for uses in sub-cutaneous, intra-muscular, intradermal, and many other types of injections, site-specific injection by themselves or at site of other implant placement e.g. by other medical devices, in conjunction with other implanted materials such as bone cement and other adhesives, xenographs, collagen and other fillers, resorbable biomaterials, biodegradable and non-degradable biomaterials, in conjunction with excipients for oral and tablet formulation, in creams, ointments and topical formulations and solutions, suspensions and emulsions intended for application on external and internal surfaces of the body. Particularly preferred particle diameters include nanoparticle and microparticle ranges of about 10⁻⁹, 10⁻⁸, 10⁻⁷ to about 10⁻⁶, 10⁻⁵ m, among others. Useful formulations of the present monomer, oligomer or polymers comprise particles similar to those described for other uses as well as for topical applications, e.g. creams, ointments, suspension, and the like, including encapsulation of particles (coated particles) and particles coated with the polymers of this invention. The pellets, “biobullets”, and seeds of the invention, all of which are forms known in the art, release upon bioerosion one or more agents. These products may be stored in hollow carriers that may itself comprise a monomer, oligomer or polymer, compound and/or composition of the invention such that, when the hollow carrier is eroded it releases the pellets, “biobullets” and/or seeds of the invention. In one preferred embodiment, the pellets, “biobullets”, and seeds comprise or are made from a monomer, oligomer or polymer of the invention containing salicylic acid admixed with follicle stimulating hormone (FSH) and/or leuteinizing hormone (LH) which are then placed in the hollow cavity or chamber of a bioerodable hollow carrier or as part of a depot formulation, e.g. Lupron Depot®, for a timed release delivery of the hormones up to about 96 hours in order to stimulate ovulation. According to the invention, a pellet, “biobullet” or seed of the invention and/or one or more hollow carriers containing a pellet, “biobullet,” or seed of the invention may be placed in a delivery device, e.g. injector, gas-driven applicator. In one embodiment, the delivery device may be further equipped with an axially slideable sleeve e.g. plunger, protrusions to prevent movement of the delivery device upon application e.g. chamfered protrusions, and handgrips. Examples of suitable carriers and/or delivery devices include, but are not limited to, those described in U.S. Pat. Nos. 6,001,385, 5,989,214, 5,549,560; WO 96/13300, WO 96/09070, WO 93/23110, and EPA 068053, each of which is herein incorporated by reference in its entirety. U.S. Pat. No. 5,989,214 and WO 96/13300, for example, describe an apparatus for injecting the body of humans or animals with a pharmaceutical preparation, wherein the preparation may be arranged in a rigid carrier, wherein the apparatus includes: a chamber into which the carrier may be transported; and a channel connecting onto the chamber for transporting the carrier into the body including fixation means for fixing the end of the channel relative to the skin of the body for injecting in order to prevent a movement of the channel in the direction perpendicularly of the axis of the barrel and where according to one embodiment the fixation means are formed by chamfered protrusions formed on the part adapted for contact with the skin of the body and extending substantially in the direction of the axis of the channel. U.S. Pat. Nos. 5,549,560, WO 93/23110, and EPA 068053 describe a device for injecting humans and animals with a pharmaceutical preparation, wherein the preparation may be held in a rigid carrier and the carrier may be carried through the skin into the body by means of gas pressure, and wherein during carrying of a rigid carrier into the body by means of gas pressure the device with which the carrier is carried into the body may be held against the body. See, e.g. U.S. Pat. No. 5,549,560; WO 93/23110; EPA 068053. These patents describe a device for injecting animals or humans with a pharmaceutical preparation, wherein a chamber comprising a carrier with the pharmaceutical preparation may be placed, a barrel connecting onto this chamber and means for carrying the carrier by means of gas pressure through the barrel into the body for injecting, wherein means are present for blocking the use of the device when it is not pressed against a body. U.S. Pat. No. 6,001,385 and WO 96/09070, for example, describe “bullets” that are at least partly manufactured from substantially fully destructurized starch, particularly implants, suitable as vehicles for introducing agents into the human or animal body in a transdermal manner.

f. Microparticles and Nanoparticles for Pharmaceutical Products

In one embodiment microspheres may be made from a diflunisal agent, monomer, oligomer, polymer, blends, mixtures and dispersions thereof having various diameters, e.g. mean diameter, for example, about 45 μm, or slightly smaller than the size commonly used for drug delivery. Polymers having surface eroding properties, e.g. polyDF, are extremely suitable for making solid, non-porous microparticles, e.g. microspheres and nanospheres, useful for sustained drug delivery, particularly suitable for injectable formulations of particle size smaller than red blood cells (RBCs). The duration release for any agent(s) or compound(s) may be controlled by varying the particle diameter, e.g. larger particles biodegrade more slowly than smaller ones. Microparticles for pharmaceutical products may be designed to deliver a drug(s) incorporated into the monomer, oligomer or polymer backbone as well as an agent(s) admixed or dispersed into the polymer. When rats were subcutaneous injected 250 mg polydiflunisal (polyDF) microspheres containing about 192 mg diflunisal formulated in a standard aqueous vehicle (FIG. 20) a peak plasma diflunisal of about 35 μg/ml was achieved within 2 days, thereafter the drug level declined slowly for about 2 weeks. In contrast a single oral dose of diflunisal produced a level of the drug that declined rapidly. Similarly, nanoparticle formulations of similar composition may be administered for various applications, having a particle size about 0.5, 1, 2, 5, 10, 20, 35, 50, 75 to about 15, 20, 30, 50, 100, 250, 500 nm, or various ranges between any two of these values. One very preferred embodiment comprises a nanoparticular formulation comprising a particle size range smaller than red blood cells in a form suitable for intravenous (I.V.) injection. These polymers may also be employed as carriers for other drugs, as has been demonstrated with paclitaxel and sirolimus. The anti-inflammatory property of PolyNSAIDs as a delivery vehicle for admixed drugs and biologicals is expected to additionally diminish the foreign body response associated with polymers commonly used for injectable depot products, such as PLGA. The injection of an agent(s) or compound(s) or a biological agent(s) carried in a polymer of this invention, e.g. a polyNSAID, will generate a significant agent(s) concentration, e.g. NSAID(s) concentrations, in tissues near the injection site. The systemic level of the agent(s), however, in most cases will remain less than about 0.1μ/ml; that is far below therapeutic levels.

g. Microparticle and Nanoparticle Formulations for Injectable Biological Products

In the pharmaceutical arena, the major marketed products in this area, LUPRON DEPOT® (leuprolide for prostate cancer and endometriosis), NUTROPIN DEPOT® (human growth hormone), TRELSTAR DEPOT® (triptorelin for prostate cancer), and SANDOSTATIN LAR® (octreotide for acromegaly), account for a market that is increasing very rapidly. Key drivers for growth are branded drug and biological products requiring product line extension, and new drug and biological products requiring delivery systems that improve patient compliance. Several leading products are summarized in Table 5 below.

TABLE 5
Injectable Drug and Biological Depot Products

| Company | Product | Drug | Delivery System |
|---|---|---|---|
| SkyePharma | DEPOCYTE ® | Cytarabine | Depofoam ™ |
| | | | liposome |
| SkyePharma | DepoMorphine ™ | Morphine | Depofoam ™ |
| | | | liposome* |
| TAP Pharma | LUPRON DEPOT ® | Leuprolide | Medisorb ™ |
| | | | (PLGA) |
| Genentech | NUTROPIN DEPOT ® | HGH | Medisorb ™ |
| | | | (PLGA) |
| Pharmacia | TRELSTAR DEPOT ® | Triptorelin | Medisorb ™ |
| | | | (PLGA) |
| Novartis | SANDOSTATIN | Octreotide | PLGA |
| | LAR ® | | |
| J & J | Risperdal | Resperidone | Medisorb ™ |
| | Consta ™ | | (PLGA)* |

*Currently in Development

Many new products contain proteins formulated with aqueous suspensions of PGLA microspheres. While generally considered to have acceptable biodegradation kinetics, safety and biocompatibility, PLGA elicits localized inflammation and foreign body response, which may be severe depending on the tissues involved. This is evidenced by clinical studies involving 138 pediatric patients who received subcutaneous injections of NUTROPIN DEPOT®, a recombinant human growth hormone formulated with PLGA microspheres. Almost every patient reported two or three “injection site reactions” per injection, most of which represent hallmark foreign-body reactions, as shown in Table 6 below whereas patients receiving aqueous formulations of NUTROPIN reported infrequent foreign-body reactions. The monomer, oligomer or polymers of the invention, such as e.g., polyNSAID microparticles, provide safe injectable depot formulations for proteins, monoclonal antibodies, polysaccharide, and nucleic acid prophylactic and therapeutic products with improved tolerability, enhanced bioavailability, and lower production costs compared to PLGA-based products.

TABLE 6
Reported Injection Site Reaction with NUTROPIN Products

| Product | Reaction | Incidence |
|---|---|---|
| NUTROPIN DEPOT ® | Granuloma (nodules) | 61% |
| | erythema (redness) | 53% |
| | pain after injection | 47% |
| | pain during injection | 43% |
| | bruising | 20% |
| | itching | 13% |
| | swelling/puffiness | 8% |
| NUTROPIN AQ ® | injection site discomfort | “reported” |
| NUTROPIN ® | injection site plan | “reported |
| | | infrequently” |

XI. Devices
a. Formation and Types

Articles of manufacture such as grafts, medical implant and devices include the use of the agent(s), monomer(s), oligomer(s), polymer(s), blends, mixture(s) and/or the agent(s) and/or additional agent(s) appended or diffused thereof to form shaped articles such as grafts and implants such as grafts, plates, e.g., bone plates and teeth; cuffs; pins; sutures; stitches; implantable sensors and drug delivery devices, and other articles that erode or decompose to release a desired agent(s) and non-toxic, non-inflammatory components within a period of time. The present products may be used also to form coatings and layers for similar articles that are made of other materials, including grafts, meshes, bone plates, sutures, implantable sensors, implantable drug delivery devices, articles for tissue and bone regeneration, and other that may require the release of a compound(s). In one embodiment, the polymers described herein may be used to form, coat or otherwise treat articles such as implants and other medical devices. The polymers of the invention may be employed for forming or coating shaped articles such as grafts, plates, e.g., bone, dental, and orthodontic plates; sutures; wound fillers; surgical meshes; dental and bone implants; implantable sensors; cuffs; pins; sutures; implantable drug delivery and sensory or diagnostic devices; and other articles suitable for implantation into a patient. Suitable medical devices include, for example, free standing films of about 0.08, 0.1, 0.2, 0.4 or 0.6 nm to about 0.5, 0.75, 0.9, 1, 1.5, or 2 mm, and in some cases even thicker, suitable for surgical coverings to prevent surgical adhesion and other uses; solutions, suspensions, emulsions, powders, gels, sprays, coats, creams, gels, in situ solidifying formulations, and semi-liquid and liquid formulations for “painting” surgically treated areas; anatomical replacement tubes; grafts; and orthopedic implants including, e.g., hip, knee and shoulder implants, internal and external fixation devices and spinal cages and dental tooth implants; dry sockets; biosensor implants, e.g., for preventing fibrosis, ophthalmic cavity implants and replacements; prolene mesh or thread; anti-infective coating on articles such as bone replacements and bandages of the sort shown in U.S. Pat. No. 5,814,031; for employing products such as mepivacaine, e.g. AP Pharma, injectable formulations (e.g. Injectile Technologies), all of the relevant information relating to these products from publicly available sources being incorporated herein by reference.

In one embodiment, the present devices comprise a monomer, oligomer, polymer, blend, or mixture thereof(s) that will break down to release an agent(s), either active or that may be activated in situ, for example, at physiological conditions. In one embodiment, the medical device comprises a monomer, oligomer, polymer, blend, or mixture thereof comprising at least one agent(s) or a pro-agent(s) that is (are) incorporated into the backbone. In another embodiment, the monomer, oligomer, polymer, blend, or mixture thereof further comprises at least one agent(s) that is not incorporated into the backbone. The agent(s) present in the backbone, appended to it, or otherwise admixed may be the same or different. The medical devices of the invention may comprise at least one monomer, oligomer, polymer, blend, or mixture thereof(s) on all or a part of their surface, and may be used, for example, to deliver the agent to a pre-determined site for effecting a specified action, such as to reduce or eliminate an adverse condition associated with the use of the device. In another embodiment the medical device may be entirely formed of a polymer(s) that break(s) down in situ, e.g. by hydrolysis or enzymatic activity. The medical devices may be formed in their entirety of the polymer, or comprise layers thereof, or be coated by a polymer(s), or many other possible configurations that will permit, for example, the release of an agent or different agents at different rates or times. One or more polymers may be arranged in accordance with this invention in alternating layers or coatings either in the formation of the device or formulation, or by subsequent coating of a device or formulation. The present device may be in the form of a mesh, pin, cuff, reconstructive dental structure, tooth, orthopedic structure, drug delivery device, sensor, implant, and the like. These devices may be formed of one or more polymers, and in addition may comprise an agent(s), monomer(s), oligomer(s), mixtures and/or blends mixed therein.

The devices of the invention may be employed for delivering an agent(s) to a specific site, such as may be the case with a bone implant, where the delivery may be to the bone environment or site of injury or surgery. The polymers, medical devices, pharmaceutical compositions and methods of treatment provided herein may be designed to reflect advantages such as, e.g., the ability to deliver a high potency or concentration of drug by weight if desired; a near “zero-order” drug release over short or long periods if desired; ease of fabrication into coatings, fibers, microspheres, pellets, etc.; little or no evidence of a “burst effect” or initial spike of drug; predictable breakdown products; multiple routes of administration; and localized delivery for improved efficacy and reduced side-effects. Furthermore, the polymers, medical devices, pharmaceutical compositions and methods of treatment provided herein may be designed such that they do not induce an inflammatory response when administered to or implanted within a host. In one embodiment, the present invention comprises the control of the onset and progression of adverse physiological conditions at a targeted site by means of a medical device or method of treatment in accordance with this invention. A directed application of pharmaceutical treatment circumvents the need for a general or systemic, i.e. “whole-body”, or oral administration of the necessary therapeutic agent(s). Accordingly, such directed application of therapeutics provides faster, more targeted relief of the adverse conditions while minimizing side effects of the administration of the therapeutics. Medical devices employed, for example, as implants, typically elicit foreign body responses characterized by thrombosis, inflammation, and infection, among others. Polymers of this invention, such as polyNSAIDs and others having anti-inflammatory and antiseptic properties, are extremely well suited for these applications. Other types of polymers described herein are well suited to impart properties such as biological, pharmaceutical, therapeutic or diagnostic properties. The polymers of this patent have a broad range of fracture toughness, as measured in ksi (or 1000 psi), or times the square root of an inch. Generally, the fracture toughness values for the polymers of the invention fall in the range of about 0.2, about 0.4, about 0.5 ksi to about 0.6, about 0.8, about 0.9, about 1.0, about 1.2 ksi. Higher and lower ksi values, are also attainable. The polymers of the invention are suitable for releasing the contained agent(s) for a broad period of time, including, but not limited to, about 1 hour, about 2 hours, about 12 hours, about 24 hours, about 2 days, about 8 days, about 2 weeks, about 4 weeks, about 3 months, about 6 months to about 8 months, about 12 months, about 15 months, about 18 months, about 2 years, and even longer periods of time, and combinations of any and all values listed herein, in applications that are specifically tailored for such a purpose.

Such device may comprise a coating(s) of a thickness of about 100 nm, 1 μm to about 30 μm, 100 μm, and values therebetween and outside of this range as needed. Typically, devices for use in medical or veterinary applications as described herein may be applied coatings or layers of coatings preferably have a thickness less than about 100 μm. One preferred rate of drug delivery may be achieved by using multiple layers of polymer with the same or different concentrations of the same or different drug in the backbone, appended, blended and/or admixed in each layer, or different co-polymers having different rates of drug generation and/or polymers with different breakdown rates for release of backbone and/or admixed drugs or agents may be used in each layer to achieve a predictable and repeatable timing of delivery of the agent(s)s. Such layering effects may be enhanced by a combination of layers of inert polymer and/or layers with inert polymer with an admixed agent(s)s and/or a layer(s) comprising a therapeutic polymer(s) and an admixed agent(s) and/or a layer(s) comprising only a therapeutic polymer(s). In one embodiment an outer coating providing an initially high dose(s) of anti-inflammatory agent(s) may be followed by the release or generation of an anti-proliferative agent(s) from an underlying layer(s). In another embodiment a medical device may be coated with more than one polymer layer, where at least one layer comprises at least one therapeutic polymer(s) of the invention. The polymers include but are not limited to “inert” polymers that do not breakdown, as well as polymers that breakdown into non-therapeutic agents. One or more coatings or layers of an inert or therapeutic polymers may be used to advantage with the therapeutic polymers of the invention to regulate the release of agents released from or generated by therapeutic polymer underlying the coating or layer of polymer. In more preferred embodiments, the agent(s) may be predictably and repeated released over time. For example, the agent(s) may be released from the set of coatings at a steadily increasing or decreasing rate, or at a nearly constant rate over time. In other more preferred embodiments, the outer layer(s) of polymer slow or prevent the penetration of water and/or enzymes to the inner layer(s) of therapeutic polymer. These embodiments are useful to lengthen the shelf-life of the medical or veterinary device, and/or to regulate the release or generation of the agent(s) in underlying layers. In a most preferred embodiment the layer(s) of therapeutic polymer on the medical or veterinary device are further coated with a layer of polymer that may be a polyorganic acid, e.g. polylactic acid, a polymerized form of amino acids, a polymerized form of fatty acid metabolites, and derivatives and/or combinations of any of these. Table 7 below provides various examples.

TABLE 7
Straight-Chain Dicarboxylic Acid Linkers

| Linker | Chemical Formula | Comments |
|---|---|---|
| Succinic Acid | HO₂C(CH₂)₂CO₂H | Rat Oral LD50 = |
| | | 8,530 mg/kg |
| Adipic Acid | HO₂C(CH₂)₄CO₂H | Rat Oral LD50 = |
| | | 5,050 mg/kg |
| Suberic Acid | HO₂C(CH₂)₆CO₂H | |
| Sebacic Acid | HO₂C(CH₂)₈CO₂H | Rat Oral LD50 = |
| | | 14,470 mg/kg |
| Dodecanoic Acid | HO₂C(CH₂)₁₀CO₂H | Marketed as |
| | | dietary supplement |
| Tetradecanoic Acid | HO₂C(CH₂)₁₂CO₂H | In Foods (e.g., |
| | | butter) |
| Hexanedecanoic Acid | HO₂C(CH₂)₁₄CO₂H | — |

All of these molecules may be produced enzymatically by fatty acid synthase and are routinely present in the body (and in foods) in varying amounts. The data indicate that they are highly non-toxic upon oral administration. In fact, one form is currently being marketed in the U.S. as a dietary supplement. While many effects of these molecules administered directly to tissues are not fully known, they are likely to be innocuous. As noted above one of these molecules, sebacic acid, was approved by the FDA as a linker in a wafer for insertion into brain tissue (GLIADEL®, Guilford Pharmaceuticals).

In another embodiment the medical device may comprise an orthopedic implant such as a hip, knee, or shoulder implant, or an internal or external fixation device or spinal implant. These orthopedic devices may be made of many kinds of materials well known in the art such as electropolished stainless steel, other metallic alloys, inorganic ceramics such as calcium phosphate and/or hydroxyapatite, human and animal cadaveric bone, naturally-occurring and synthetic bone analogs, degradable and non-degradable polymers such as glycolic acid, lactic acid and/or caprolactone polymers and their co-polymers with other agents and/or their blends. The orthopedic implants may be coated with a polymer(s) of the invention comprising preferably about 1 μm to about 1 mm thickness. Some entirely porous implants may benefit from a longer lasting effect that is enabled by a coating that fills the device's interstices with a thin coating on areas proximal to a target bone or tissue. In some cases it may be preferable to employ a nano- and/or micro-sphere formulation of a diameter typically less than about 10 μm for in situ administration or application, or for application to the surface of a device before placement. A sterile liquid may be used to coat the device to foster adherence of the nano- or microspheres for minutes to weeks to enable uncoated devices to act as coated devices do. These are described in more detail below

b. Metal and Non-Metal Devices and Surface Adhesion

The metallic components of many implantable orthopedic devices maybe made of various alloys, such as nickel-titanium and cobalt-chromium. The adhesion load displacement profile of polymers in accordance to this invention, e.g., polyDF, on these metals at ambient temperature, were measured by testing polymers that were melt-coated directly onto clean, dry 1.25 metal butt-joints. On one type of satin-finish titanium alloy, polyDF exhibited a load failure of 2,030 PSI. Testing of the polymer on a cobalt-chromium alloy was interrupted at 1,630 PSI when the metal grip pins used to hold the meal test cylinder broke. These results demonstrate that polyDF adheres to these metals as tightly as commonly used epoxies and glues. ASTM test methods were used to demonstrate the strong adhesion of polymers of the invention such as polySA and polyDF to a metal such as electro-polished 316L stainless steel. This property is in sharp contrast to other polymers, many of which adhere to metals only after special treatment of the metal surfaces. In general, the polymers of the invention exhibit excellent adhesion to non-metallic surfaces, including polymers such as biopolymers, polyanhydrides and other biocompatible and non-biocompatible polymers, nickel alloys, PMMA based materials, and the like. The polymers of this patent may be employed in conjunction and for covering and adhering to any material suitable for use in the applications mentioned here. The polymers of this invention achieve a broad range of cohesive failure values as measured by a 1.1″ Butt Weld test. Generally, cohesive values of about 100, about 200, about 300, about 400, about 600, about 700, about 1000 to about 1500, about 2000, about 2500, about 3000 psi are easily attained. The lower value represents minimal adhesion whereas the higher value represents cohesive failure of the polymer. Much broader range values are consistently achieved on surfaces such as titanium alloys, stainless steel, cobalt alloys, and chromium alloys.

c. Biodegradation

Different polymers degrade differently, i.e. at different rates over different periods of time. Degradation of polymers of the invention, such as polySA and polyDF, was tested with polymers coated onto samples of electro-polished 316L stainless steel. The polymers were dissolved in anhydrous chloroform and spread into thin films onto dry metal surfaces that had been cleaned with acetone, after which the solvent was removed overnight in a 40° C. vacuum oven. A 5 μm layer of polySA incubated in pH 7.4 PBS at 37° C. generated salicylic acid for about one week as shown in Table 8 below. While not apparent from the data shown in the table, it should be noted that polySA did not begin to degrade until 8-10 hours after exposure to buffer or serum. This “induction period” is characteristic of poly(anhydride-ester) polymers; in general, the higher the molecular weight, the longer the induction time. In contrast, a similar 5 μm layer of polyDF generated diflunisal for a period of time of over 2 months.

TABLE 8
Coated⁺ Polymer Erosion

Cumulative NSAID Release (%)*

| Time (Days) | Salicylic Acid - 261PL | Diflunisal - 657PL |
|---|---|---|
| 0 | — | 0.0 |
| 1 | 0.0 | 0.0 |
| 2 | 16.0 | 0.8 |
| 3 | 40.0 | 3.2 |
| 5 | 69.4 | 11.7 |
| 8 | 73.3 | 25.4 |
| 11 | 77.9 | 39.2 |
| 14 | 78.7 | 47.9 |
| 17 | ND | 54.7 |
| 20 | ND | 58.5 |
| 25 | ND | 71.4 |
| 32 | ND | 71.9 |
| 62 | ND | 97.8 |
| 74 | ND | 103.1 |

*NSAID released into PBS pH 7.4 at 37° C. + 5 μm-thick Polymer Coating on 316 SS Plates.
ND Not Determined.

Kinetic analysis of the results shown in Table 8 above evidence that the generation of salicylic acid from polySA proceeded in a sharply bi-phasic, non-linear rate, while the generation of diflunisal from polyDF was mono-phasic and linear. These different kinetic profiles may be partly explained by the different degradation mechanisms of polySA versus polyDF. So-called “bulk eroding” polymers degrade throughout their structure, like a lump of sugar in water. Because essentially the whole polymer mass may be available for degradation, the greater the amount of a bulk eroding polymer, the more breakdown product generated over time. This is the case with polySA; when solid disks of this polymer were incubated in 37° C. PBS, the thicker disks generated more salicylic acid, as shown in Table 9 below.

TABLE 9
Effect of Coating Thickness on Drug Release

| | Salicylic Acid Generated (Cumulative μg/cm²) | | |
|---|---|---|---|
| Time lapsed | Polymer Coating Thickness (mm) | | |
| (days) | 0.1 | 0.2 | 0.8 |
| 0 | 0.00 | 0.00 | 0.00 |
| 1 | 644.83 | 0.00 | 1563.63 |
| 4 | 2644.67 | 3415.78 | 14349.41 |
| 7 | 3338.06 | 5989.35 | 18160.02 |
| 11 | 3721.91 | 8698.98 | 20373.20 |
| 15 | 3646.73 | 9222.90 | 20968.46 |
| 20 | 3655.84 | 9041.59 | 19183.40 |
| 22 | 3632.68 | 8892.61 | 19024.90 |

* 261PL Melt Polymer Coated on 6.7 mm Diameter Wafer placed in PBS at 37° C.

“Surface-eroding” polymers, on the other hand, degrade only from their surface, like a bar of soap. Since only the polymer surface may be available for degradation, the generation or breakdown products over time generally does not vary with polymer mass. This may be the case with polyDF; when disks of this polymer were incubated in 37° C. PBS, the same amount of diflunisal was generated regardless of disk thickness. The surface eroding property of polyDF makes it ideal for use as coatings in settings where a constant, controlled rate of drug delivery may be desired. This property of polyDF enabled an evaluation of the effect of polymer molecular weight on the generation of diflunisal.

Two preparations of polyDF (molecular weights 33K and 100K) produced by the melt-condensation method were solvent coated onto electro-polished stainless steel samples and incubated in 37° C. serum, which contains esterase enzymes that might be expected to contribute to polymer degradation in the body. As shown in Table 10, the 33,000 Dalton polymer started to degrade much more rapidly than the 100,000 Dalton polymer, which, in PBS, generated diflunisal for about two months.

TABLE 10
Effect of Molecular Weight on Polymer Erosion

Diflunisal Generated (Cumulative μg/cm²)
PolyDiflunisal (657PL)*

| Time | 33K - 9 μm | 100K - 22 μm |
|---|---|---|
| (days) | thick coating | thick coating |
| 0.17 | 0.00 | 0.00 |
| 0.33 | 3.90 | 0.00 |
| 1 | 41.73 | 7.14 |
| 3 | 471.55 | 101.55 |
| 5 | 751.92 | 267.76 |
| 7 | 753.74 | 346.20 |
| 13 | 959.21 | 658.26 |
| 19 | 814.59 | 756.35 |
| 25 | 731.07 | 796.82 |
| 34 | 835.77 | 945.26 |

*Diflunisal Released into Serum at 37° C. Polymer Coating of SS Plates

The molecular profile of the products of polymer degradation that may be generated over a period of time may be another important characteristic of biodegradable polymers. Polymers that biodegrade consistently into a small number of breakdown products generally have good biocompatibility, and will encounter fewer regulatory hurdles. In the case of polySA, the HPLC chromatograms showed only breakdown products that contained salicylic acid with the linker itself not being observed. After two days, the main breakdown product in serum was salicylic acid, which exhibited a 2-minute elution time. Also observed were minor amounts of the monomer and several oligomers. By day three, the elution profile indicated increasing amounts of salicylic acid, with smaller mounts of monomer and oligomers. After seven days, only salicylic acid and one other compound were apparent, and by day 13, only salicylic acid was observed. The pattern of soluble breakdown products generated during the degradation of polyDF in 37° C. serum was less complex, comprising diflunisal itself with a 7-minute elution time, with no other breakdown products observed in serum up to two days, and at every point thereafter.

Blend and/or Mixture Containing Admixed Drugs

For many medical device applications it may be desirable to use monomer, oligomer, polymer, blend, or mixture in accordance with this invention, e.g. polyNSAIDs, in combination with other drugs added to the monomer, polymer, blend, or mixture to produce additional therapeutic effects. Such “solid solution” preparations may be created by simply mixing a monomer, oligomer, polymer, blend, or mixture dissolved in a solvent with a solution of another drug dissolved in the same solvent, or by any other method known in the art. Evaporation of the solvent results in a homogeneous solid solution of drug in the monomer, oligomer, polymer, blend, or mixture. The usefulness of the invention's monomer, oligomer, polymer, blend, or mixtures in medical devices, such as drug-eluting bone implants and others, led the inventors to prepare and evaluate solid solutions of, for example polyDF containing 20 wt % paclitaxel or sirolimus, i.e., 1 mg of polymer/drug admixture contained 0.8 mg polymer and 0.2 mg drug. Table 11 below shows the concurrent release of paclitaxel from a polyDF/paclitaxel admixture coated onto electropolished stainless steel samples and incubated in 37° C. serum. Paclitaxel was released at the same rate at which the polymer biodegraded to generate diflunisal. The relatively small percentage of paclitaxel released reflects the inability of serum to hold this very poorly water-soluble drug. The incorporation of paclitaxel into the polymer did not affect the generation of diflunisal, which proceeded at the same rate as from polyDF without paclitaxel. Similar results were obtained with a polyDF/sirolimus admixture.

TABLE 11
Paclitaxel Release from Polymer
Alone & Admixed with Paclitaxel

| | Cumulative Drug Released (%) | |
|---|---|---|
| Time Elapsed | 0% PAC in Coating | 20% PAC in Coating |

| (Days) | DF* | DF* | PAC+ |
|---|---|---|---|
| 0 | 0.00 | 0.00 | 0.00 |
| 3 | 40.22 | 17.28 | 0.72 |
| 5 | 45.00 | 48.72 | 2.48 |
| 7 | 49.41 | 58.35 | 3.87 |
| 10 | 61.88 | 58.76 | 4.09 |
| 12 | 79.51 | 78.94 | 4.31 |
| 14 | 68.88 | 68.87 | 5.42 |
| 20 | 76.13 | 74.82 | 6.24 |
| 26 | 69.04 | 67.11 | 5.69 |

*Diflunisal Release from DF Polymer into 37° C. Serum from 5 μm Thick Coatings on 316 SS Plates.
+Paclitaxel Release from Admixture with DF Polymer into 37° C. Serum from 5 μm Thick Coatings on 316 SS Plates.

XII. Effect of Sterilization

All implantable and percutaneous articles and medical devices must be sterilized before or after packaging. Sterilization methods commonly employed are gamma irradiation, electron beam (“E-beam”), and ethylene oxide. Sterilization by gamma radiation penetrates objects deeply, and may be used for food and many medical and veterinary products, but the method requires relatively prolonged exposure times. E-beam sterilization allows shorter exposure times, but the electrons penetrate objects poorly, making the procedure useful mainly for surfaces. Ethylene oxide sterilization is more complex and more aggressive on organic materials than the other methods and is being replaced where possible due to environmental hazards. The relatively high temperatures and humidity employed in many ethylene oxide sterilization protocols is not very compatible with poly(anhydride-ester) polymers. Accordingly, gamma radiation and E-beam sterilization methods are preferred for use with such compositions. The sterilization with E-beam (3.5 mRad) and gamma radiation (25-35 Kgys) had no effect on the pattern of diflunisal generated from polyDF coated stainless steel samples incubated in 37° C. serum. See, Table 12 below.

TABLE 12
Cumulative Diflunisal Released by
Untreated & Sterilized Polymer

| | Cumulative Diflunisal* | | |
|---|---|---|---|
| | Released from 657PL Polymer (%) | | |
| Time Elapsed | Not | Gamma | E-Beam |
| (days) | Irradiated | Irradiated | Irradiated |
| 1 | 0.39 | 0.57 | 1.12 |
| 2 | — | 4.02 | 4.81 |
| 3 | 5.47 | — | — |
| 5 | 14.47 | — | — |
| 7 | 18.50 | 28.25 | 27.87 |
| 9 | — | 30.05 | 28.02 |
| 13 | 34.87 | 34.74 | 32.84 |
| 17 | — | 29.23 | 36.58 |
| 19 | 39.85 | — | — |
| 20 | — | 44.06 | 41.84 |
| 25 | 42.00 | — | — |
| 26 | — | 50.38 | 44.07 |
| 34 | 49.89 | — | — |

*Released by 5 μm Diflunisal Polymer Coated on 316LSS Plates & Placed in Serum at 37 C..

Comparative studies were conducted on the characteristics of salicylic acid and diflunisal polymers before and after sterilization by various methods commonly employed in the art. A comparison of the results obtained is provided in Tables 13a and 13b shown below. Notwithstanding the lack of effect on polymer degradation, sterilization does produce some changes in molecular weight and mechanical properties. For example, the tensile modulus of melt-polymerized polySA at room temperature decreased by about a third after gamma sterilization (25-35 Kgys that had no effect on the molecular weight, flexibility, or adhesiveness, and only minor effects on hardness.

TABLE 13a
Changes Produced by γ-Irradiation

| Property | 261PL | 657PL |
|---|---|---|
| Molecular Weight | about 20,000 | about 100,000 |
| (Non Irradiated) | | |
| Molecular Weight | N.C. | about 50,000 |
| (Irradiated) | | |
| Hardness | −2 Units | −3 Units |
| Flexibility | N.C. | — |
| Adhesion | N.C. | — |

N.C. No Change.

TABLE 13b
Changes Produced by E-Beam-Irradiation*

| | Polymer | |
|---|---|---|
| Property | 261PL | 657PL |

| | | | |
|---|---|---|---|
| Molecular Weight | about 20,000 | about 33,000 | about 80,000 |
| (Non Irradiated) | | | |
| Molecular Weight | −26% | +5% | −30% |
| (Irradiated) | | | |
| Hardness | −1 Unit | +2 Units | N.C. |
| Flexibility | N.C. | — | N.C. |
| Adhesion | −1 Unit | — | — |

*3-4.5 MRad E-Beam Radiation.
N.C. No Change.

XIII. Coatings for Implanted Orthopedic Joint-Replacement/Aid Devices

Joint-replacement implants and bone aid devices are widely used to restore quality of life for million of patients with irreparably damaged shoulders, knees, and hips as well as for repairing broken and splintered bones. These devices are generally made of titanium/nickel or cobalt/chromium alloys, with metal stems that are inserted into the hollow portion of the arm or leg bones. Some of these stems have smooth surfaces that require the use of bone cement to ensure strong connection, while others have highly engineered, honeycomb-textured surfaces that become partially filled with bone and marrow cells during insertion, thereby seeding the stem for in growth of new bone and reducing the need for cement. Orthopedic surgeons are eager to incorporate agents into these surfaces that may accelerate bond growth. A number of recombinant bone morphogenic proteins (BMPs) and other “osteogenic” proteins are in development for this purpose, notwithstanding their high manufacturing costs and product development challenges. The dynamics of bone formation, resorption, and repair are complex, and appear to vary for different types of bone. Dental studies showed that the inhibition of prostaglandin production by NSAIDs decreases bone resorption in the trabecular bone of the palate and alveolar bone of the jaw, causing a net increase in bond mass and density. This phenomenon was demonstrated in the mouse for “PolyAspirin” implants. In addition, polySA prevented bone erosion in a rat femur transaction model. Other animal studies suggest that the repair of long-bone fractures may be inhibited by long-term exposure to high levels of NSAIDs. Different forms of the present polymers may be prepared that are suitable for these and other applications in the orthopedics and dental fields, among others. Polymers of this invention, such as polyNSAIDs and others, may be employed as coatings to reduce pain and inflammation associated with device implantation and adjustment of dental and orthopedic aids, to reduce the incidence of infection, that may be a major problem associated with joint replacement devices, and to prevent and treat other conditions by delivering appropriate agents to the site. While infection at the implant/bone interface reportedly occurs in less than 1% of cases, the limited blood supply to the region makes these infections particularly hard to treat with systemic antibiotics. The antiseptic properties of a polymer of the invention, such as a polyNSAID, a polyantibiotic, a combination or mixture thereof, in a coating prevents or greatly reduces infection without the potential for bacterial resistance. Together with the properties of polymers such as polyNSAIDs summarized in Table 14 below, this characteristic makes PolyNSAIDs attractive for use on orthopedic, dental, ocular, and many other implanted medical devices.

TABLE 14
PolyNSAIDs vs. Current Polymer Coatings

| PolyNSAID Coatings | Current Polymer Coatings* |
|---|---|
| Biodegradable | Non-biodegradable |
| Pharmacologically active | Pharmacologically inactive |
| Little/no inflammation | Significant inflammation |
| Additional drug OK | Additional drug not OK |
| Easily applied to metals | Application complex |

*Commercially Available Coated Stents

Medical devices useful with coverings of the present invention include, but are not limited to, a fixation device, catheters, drain tubes, intravenous tubes, tamponades, ventilator tubes, arthroscopic articles, drug-based implants and patches of all sorts for drug delivery near a pre-selected site, e.g. devices, and surgical articles such as sponges and implants. The monomer, oligomer, polymer, blend, or mixtures compositions of the invention may be formed into a medical implant such as a medical, dental, orthopedic and surgical implant, or applied or coated onto such implant. In addition to the implants described above, other examples are implants for vascular, cardiovascular, coronary, peripheral vascular, orthopedic, dental, oro-maxillary, gastrointestinal, urogenital, ophthalmic, gynecological, pulmonary, surgical, physiological, metabolic, neurological, diagnostic and therapeutic uses, may be formed from or applied or coated with the above identified polymers, compounds and/or compositions. Such implants include, but are not limited to, bones and their fragments, guide wires and other fixing articles, grafts, sutures, meshes, joint and other prostheses, fracture management devices, drug dosing devices, dental and oro-maxillary implants, and many more known in the art.

Suitable medical implants also include, but are not limited to the ones described here.

a. Ethicon (a Johnson & Johnson Company, Piscataway, N.J.) products: Vicryl™ (resorbable braided coated), Pronova™, and Panacryl™.

b. USS/DG Sutures (U.S. Surgical, a division of Tyco Healthcare Group LP, Norwalk, Conn.) products: Decon II™ (coated, braided synthetic, absorbable), PolySorb™ (coated, braided synthetic, absorbable), Dexon S™ (Uncoated, braided synthetic, absorbable), Gut sutures (absorbable), Biosyn™ (synthetic monofilament, absorbable), Maxon™ (synthetic monofilament, absorbable), Surgilon™ (braided nylon, non-absorbable), Ti-Cron™ (coated, braided polyester, non-absorbable), Surgidac™ (coated, braided polyester, non-absorbable), SofSilk™ (coated, braided silk, non-absorbable), Dermalon™ (nylon monofilament, non-absorbable), Monosof™ (nylon monofilament, non-absorbable), Novafil™ (polybutester monofilament, non-absorbable), Vascufil™ (coated polybutester monofilament, non-absorbable), Surgilene™ (polypropylene monofilament, non-absorbable), Surgipro™ (polypropylene monofilament, non-absorbable), Flexon™ (stainless steel monofilament, non-absorbable), SURGALLOY™ needle, and SURGALLOY™ OptiVis™ needle.

c. Surgical Dynamics (Surgical Dynamics, Inc., North Haven, Conn.,) products: S*D*Sorb™ (suture anchor, Anchor Sew™ (suture anchor), S*D*Sorb E-Z Tac™ (bio-resorbable implant w/o sutures), S*D*Sorb Meniscal Stapler™ (delivers bio-absorbable repair implant), Ray Threaded Fusion Cage™ (spine), Aline™ (cervical plating system), SecureStrand™ (spinal reconstruction cable), and Spiral Radius 90D™(spinal rod system).

d. Zimmer (Zimmer, Warsaw, Indiana) products: VerSys™ cemented stem hip system, VerSys Heritage™ Hip cemented stem hip system, VerSys™ LD/Fx cemented stem hip system, CPT™ Hip cemented stem hip system, VerSys™ Cemented Revision/Calcar cemented stem hip system, Mayo™ Hip porous stem hip system, VerSys™ Beaded MidCoat porous stem hip system, VerSys™ Beaded FullCoat Plus porous stem hip system, VerSys™ Fiber Metal MidCoat porous stem hip system, and VerSys™ Fiber Metal Taper porous stem hip system, VerSys™ LD/Fx press-fit hip system, VerSys™ Cemented Revision/Calcar revision stem hip system, ZMR™ hip revision stem hip system, Trilogy™ Cup acetabular cup hip system, ZCA™ cup acetabular cup hip system, Longevity™ polyethylene hip system, Calcicoat™ coating hip system, NexGen™ Implant knee system, NexGen™ Instruments knee system, NexGen™ Revision Instruments knee system, IM™ Instruments knee system, MICRO-MILL™ 5-in-1 Instruments knee system, Multi-Reference™ 4-in-1 knee system, V-STAT™ Instruments knee system, Coonrad/Morrey™ elbow, Bigliani/Flatow™ shoulder, Cable Ready™ Cable Grip System, Collagraft™ Bone Graft Matrix, Herbert™ Bone Screw, M/DN™ Intramedullary Fixation, Mini Magna-Fx™ Screw Fixation, Magna-Fx™ Screw Fixation, Periarticular™ Plating System, Versa-Fx™Femoral Fixation system, Versa-Fix II™ Femoral Fixation System, and Trabecular™ Metal.

e. Alza technologies (ALZA Corporation, Mountain View, Calif.) products: DUROS® Implant, OROS™ osmotic, D-TRANS™ transdermal, STEALTH™ liposomal, E-TRANS™ electrotransport, Macroflux™, and ALZAMER depot. 13) described in Stuart, M., “Technology Strategies, Stent and Deliver,” Start-Up, Windhover's Review of Emerging Medical Ventures, pp. 34-38, June 2000); van der Giessen, Willem J., et al. “Marked Inflammatory Sequelae to Implantation of Biodegradable and Nonbiodegradable Polymers in Porcine Coronary Arteries,” Circulation 94: 7, pp. 1690-1697 (Oct. 1, 1996); Gunn, J. et al., “Stent coatings and local drug delivery,” European Heart Journal 20: 1693-1700 (1999); EP Applications 01301671, 00127666, 99302918, 95308988, 95306529, 95302858, 94115691, 99933575, 94922724, 97933150,95308988, 91309923, 91906591, and 112119841; WO 00/187372, WO 00/170295, WO 00/145862, WO 00/143743, WO 00/044357, WO 00/009672, WO 99/03517, WO 99/00071, WO 98/58680, WO 98/34669, WO 98/23244, and WO 97/49434; U.S. Ser. Nos. 061568, 346263, 346975, 325198, 797743, 815104, 538301, 430028, 306785, and 429459; and U.S. Pat. Nos. 6,325,825, 6,325,790, 6,322,534, 6,315,708, 6,293,959, 6,289,568, 6,273,913, 6,270,525, 6,270,521, 6,267,783, 6,267,777, 6,264,687, 6,258,116, 6,254,612, 6,245,100, 6,241,746, 6,238,409, 6,214,036, 6,210,407, 6,210,406, 6,210,362, 6,203,507, 6,198,974, 6,190,403, 6,190,393, 6,171,277, 6,171,275, 6,165,164, 6,162,243, 6,140,127, 6,134,463, 6,126,650, 6,123,699, 6,120,476, 6,120,457, 6,102,891, 6,096,012, 6,090,104, 6,068,644, 6,066,125, 6,064,905, 6,063,111, 6,063,080, 6,039,721, 6,039,699, 6,036,670, 6,033,393, 6,033,380, 6,027,473, 6,019,778, 6,017,363, 6,001,078, 5,997,570, 5,980,553, 5,971,955, 5,968,070, 5,964,757, 5,948,489, 5,948,191, 5,944,735, 5,944,691, 5,938,682, 5,938,603, 5,928,186, 5,925,301, 5,916,158, 5,911,732, 5,908,403, 5,902,282, 5,897,536, 5,897,529, 5,897,497, 5,895,406, 5,893,885, 5,891,108, 5,891,082, 5,882,347, 5,882,335, 5,879,282, RE36,104, 5,863,285, 5,853,393, 5,853,389, 5,851,464, 5,846,246, 5,846,199, 5,843,356, 5,843,076, 5,836,952, 5,836,875, 5,833,659, 5,830,189, 5,827,278, 5,824,173, 5,823,996, 5,820,613, 5,820,594, 5,811,814, 5,810,874, 5,810,785, 5,807,391, 5,807,350, 5,807,331, 5,803,083, 5,800,399, 5,797,948, 5,797,868, 5,795,322, 5,792,415, 5,792,300, 5,785,678, 5,783,227, 5,782,817, 5,782,239, 5,779,731, 5,779,730, 5,776,140, 5,772,590, 5,769,829, 5,759,179, 5,759,172, 5,746,764, 5,741,326, 5,741,324, 5,738,667, 5,736,094, 5,736,085, 5,735,831, 5,733,400, 5,733,299, 5,728,104, 5,728,079, 5,728,068, 5,720,775, 5,716,572, 5,713,876, 5,713,851, 5,713,849, 5,711,909, 5,709,653, 5,702,410, 5,700,242, 5,693,021, 5,690,645, 5,688,249, 5,683,368, 5,681,343, 5,674,198, 5,674,197, 5,669,880, 5,662,622, 5,658,263, 5,658,262, 5,653,736, 5,645,562, 5,643,279, 5,634,902, 5,632,763, 5,632,760, 5,628,313, 5,626,604, 5,626,136, 5,624,450, 5,620,649, 5,613,979, 5,613,948, 5,611,812, 5,607,422, 5,607,406, 5,601,539, 5,599,319, 5,599,310, 5,598,844, 5,593,412, 5,591,142, 5,588,961, 5,571,073, 5,569,220, 5,569,202, 5,569,199, 5,562,632, 5,562,631, 5,549,580, 5,549,119, 5,542,938, 5,538,510, 5,538,505, 5,533,969, 5,531,690, 5,520,655, 5,514,236, 5,514,108, 5,507,731, 5,507,726, 5,505,700, 5,501,341, 5,497,785, 5,497,601, 5,490,838, 5,489,270, 5,487,729, 5,480,392, 6,325,800, 6,312,404, 6,264,624, 6,238,402, 6,174,328, 6,165,127, 6,152,910, 6,146,389, 6,136,006, 6,120,454, 6,110,192, 6,096,009, 6,083,222, 6,071,308, 6,048,356, 6,042,577, 6,033,381, 6,032,061, 6,013,055, 6,010,480, 6,007,522, 5,968,092, 5,967,984, 5,957,941, 5,957,863, 5,954,740, 5,954,693, 5,938,645, 5,931,812, 5,928,247, 5,928,208, 5,921,971, 5,921,952, 5,919,164, 5,919,145, 5,868,719, 5,865,800, 5,860,974, 5,857,998, 5,843,089, 5,842,994, 5,836,951, 5,833,688, 5,827,313, 5,827,229, 5,800,391, 5,792,105, 5,766,237, 5,766,201, 5,759,175, 5,755,722, 5,755,685, 5,746,745, 5,715,832, 5,715,825, 5,704,913, 5,702,418, 5,697,906, 5,693,086, 5,693,014, 5,685,847, 5,683,448, 5,681,274, 5,665,115, 5,656,030, 5,637,086, 5,607,394, 5,599,324, 5,599,298, 5,597,377, 5,578,018, 5,562,619, 5,545,135, 5,544,660, 5,514,112, 5,512,051, 5,501,668, 5,489,271, 6,319,287, 6,287,278, 6,221,064, 6,113,613, 5,984,903, 5,910,132, 5,800,515, 5,797,878, 5,769,786, 5,630,802, 5,492,532, 5,322,518, 5,279,563, 5,213,115, 5,156,597, 5,135,525, 5,007,902, 4,994,036, 4,981,475, 4,951,686, 4,929,243, 4,917,668, 4,871,356, 6,322,582, 6,319,445, 6,309,202, 6,293,961, 6,254,616, 6,206,677, 6,205,748, 6,178,622, 6,156,056, 6,128,816, 6,120,527, 6,105,339, 6,081,981, 6,076,659, 6,058,821, 6,045,573, 6,035,916, 6,035,751, 6,029,805, 6,024,757, 6,022,360, 6,019,768, 6,015,042, 6,001,121, 5,987,855, 5,975,876, 5,970,686, 5,956,927, 5,951,587, RE36,289, 5,924,561, 5,906,273, 5,894,921, 5,891,166, 5,887,706, 5,871,502, 5,871,490, 5,855,156, 5,853,423, 5,843,574, 5,843,087, 5,833,055, 5,814,069, 5,813,303, 5,792,181, 5,788,063, 5,788,062, 5,776,150, 5,749,898, 5,732,816, 5,728,135, 5,709,067, 5,704,469, 5,695,138, 5,692,602, 5,683,416, 5,681,351, 5,675,961, 5,669,935, 5,667,155, 5,655,652, 5,628,395, 5,623,810, 5,601,185, 5,571,469, 5,555,976, 5,545,180, 5,529,175, 5,500,991, 5,495,420, 5,491,955, 5,491,954, 5,487,216, 5,487,212, 5,486,197, 5,485,668, 5,477,609, 5,473,810, 5,409,499, 5,364,410, 5,358,624, 5,344,005, 5,341,922, 5,306,280, 5,284,240, 5,271,495, 5,254,126, 5,242,458, 5,236,083, 5,234,449, 5,230,424, 5,226,535, 5,224,948, 5,213,210, 5,199,561, 5,188,636, 5,179,818, 5,178,629, 5,171,251, 5,165,217, 5,160,339, 5,147,383, 5,102,420, 5,100,433, 5,099,994, 5,089,013, 5,089,012, 5,080,667, 5,056,658, 5,052,551, 5,007,922, 4,994,074, 4,967,902, 4,961,498, 4,896,767, 4,572,363, 4,555,016, 4,549,649, 4,533,041, 4,491,218, 4,483,437, 4,424,898, 4,412,614, D260,955, 4,253,563, 4,249,656, 4,127,133, D245,069, 3,972,418, 3,963,031, 3,951,261, 3,949,756, 3,943,933, 3,942,532, 3,939,969, 6,270,518, 6,213,940, 6,203,564, 6,191,236, 6,138,440, 6,135,385, 6,074,409, 6,053,086, 6,016,905, 6,015,427, 6,011,121, 5,988,367, 5,961,538, 5,954,748, 5,948,001, 5,948,000, 5,944,739, 5,944,724, 5,939,191, 5,925,065, 5,910,148, 5,906,624, 5,904,704, 5,904,692, 5,903,966, 5,891,247, 5,891,167, 5,889,075, 5,865,836, 5,860,517, 5,851,219, 5,814,051, 5,810,852, 5,800,447, 5,782,864, 5,755,729, 5,746,311, 5,741,278, 5,725,557, 5,722,991, 5,709,694, 5,709,692, 5,707,391, 5,701,664, 5,695,879, 5,683,418, 5,669,490, 5,667,528, 5,662,682, 5,662,663, 5,649,962, 5,645,553, 5,643,628, 5,639,506, 5,615,766, 5,608,962, 5,584,860, 5,584,857, 5,573,542, 5,569,302, 5,568,746, 5,566,822, 5,566,821, 5,562,685, 5,560,477, 5,554,171, 5,549,907, 5,540,717, 5,531,763, 5,527,323, 5,520,702, 5,520,084, 5,514,159, 5,507,798, 5,507,777, 5,503,266, 5,494,620, 5,480,411, 5,480,403, 5,462,558, 5,462,543, 5,460,263, 5,456,697, 5,456,696, 5,442,896, 5,435,438, 5,425,746, 5,425,445, 5,423,859, 5,417,036, 5,411,523, 5,405,358, 5,403,345, 5,403,331, 5,394,971, 5,391,176, 5,386,908, 5,383,905, 5,383,902, 5,383,387, 5,376,101, D353,672, 5,368,599, D353,002, 5,359,831, 5,358,511, 5,354,298, 5,353,922, 5,350,373, 5,349,044, 5,335,783, 5,335,775, 5,330,442, 5,325,975, 5,318,577, 5,318,575, 5,314,433, 5,312,437, 5,310,348, 5,306,290, 5,306,289, 5,306,288, 5,294,389, 5,282,832, 5,282,533, 5,280,674, 5,279,783, 5,275,618, 5,269,807, 5,261,886, 5,261,210, 5,259,846, 5,259,845, 5,249,672, 5,246,104, 5,226,912, 5,225,485, 5,217,772, 5,217,486, 5,217,485, 5,207,679, D334,860, 5,197,597, 5,192,303, D333,401, D333,400, 5,181,923, 5,178,277, 5,174,087, 5,168,619, 5,163,946, 5,156,615, 5,154,283, 5,139,514, 5,133,738, 5,133,723, 5,131,534, 5,131,131, 5,129,511, 5,123,911, 5,121,836, 5,116,358, 5,102,418, 5,099,676, 5,092,455, 5,089,011, 5,089,010, 5,087,263, 5,084,063, 5,084,058, 5,078,730, 5,067,959, 5,059,213, 5,059,212, 5,051,107, 5,046,513, 5,046,350, 5,037,429, 5,024,322, 5,019,093, 5,002,550, 4,984,941, 4,968,315, 4,946,468, 4,932,963, 4,899,743, and 4,898,156; among many others available in the public domain, the relevant portions of all of the above listed being hereby incorporated by reference in their entireties.

Polymeric drug delivery systems comprising the polymers of the invention may be readily processed into pastes or solvent cast to yield films, coatings, nanoparticles e.g. nanospheres, microparticles e.g. microspheres and fibers with different geometric shapes for design of various medical devices, and may also be processed by compression molding and extrusion. In one embodiment, a polymer or polymers may be coated onto or applied onto a medical device, such as, e.g., by forming the polymer or polymers into a covering. In another embodiment, the polymer or polymers may be formed into a medical device, such as, e.g., an implant. In one embodiment of the present invention, a polymer comprising a functional group or agent may used to form a covering, such as, e.g., a coating or a sheath, that partially or completely covers and/or surrounds a medical device. Such a covering may cover a portion of the medical device or it may completely cover a medical device. The covering may be divided into separate portions or several smaller coverings may be present on the medical device. In another embodiment of the invention, a polymer may surround the medical device, or a portion thereof, and may have the form of a coating, a layer, a film, and combinations thereof. The polymer may be in the form of a solid or a semi-solid, such as a gel, sheath, a wrap, a tube or a cuff covering all or a portion of the medical device. The polymer may be rigid, semi-rigid, or non-rigid. The coating of polymer may comprise about 100 nm, 1 μm to about 1 mm, 1 cm thick, although some porous implants may benefit from longer lasting effects enabled by a coating that completely fills the interstices of the device with, in some cases, a thin coating on those surfaces proximal to bone or other tissue upon placement in the body. In one embodiment, the polymer coating may be comprised of microparticles, such as microspheres that may typically but not necessarily be less than 10 microns in diameter. These microparticles may be applied to the surface of a medical device before placement in the body. A sterile liquid may be used to coat the device to adhere such microspheres for minutes to weeks to enable uncoated medical devices to benefit from the same or similar therapeutic benefits as coated devices.

A polymer, compound and/or composition of the invention may be applied or coated onto a medical implant by any means known in the art including, but not limited to, solvent methods such as, for example, dipping and spray-drying, and non-solvent methods such as chemical vapor deposition, extrusion coating, covalently grafting or dipping in molten polymer, compound and/or composition of the invention. The method of preparation may vary depending on the polymer, compound and composition and/or the medical implant. The medical implant may be formed from or coated with one or more layers of the same or different polymer, compound and/or composition of the invention. In another example, a polymer, compound and/or composition of the invention may be coated onto a medical implant in the shape of a membrane or tube for use in the treatment of injury or damage to the peripheral nervous system or a block of solid or foamed composition containing pathways drilled or otherwise formed to encouraged nerve growth or bone growth. In the above instances, bioerosion of the disc, membrane, tube or block would yield or generate an agent included within the polymer or composition. The polymer may be formed into a device by any means known in the art including, but not limited to, molding e.g. compression or blow molding, and extrusion. The medical device may be formed from one or more of the same or different polymer, compound and/or composition of the invention. A polymer, compound and/or composition of the invention may be formed, that is, physically configured, into various shapes, geometries, structures and configurations including, but not limited to, a film, fiber, rod, coil, corkscrew, hook, cone, pellet, tablet, tube e.g. smooth or fluted, disc, membrane, microparticle, nanoparticle, “biobullet” i.e. bullet shaped, seed i.e. bullet shaped or targeted seeds, as well as those described in the above identified products, patents and articles, including in some cases forming medical implants that have the same, similar or completely different functional characteristics compared to those functional characteristics of the medical devices described in the above identified products, patents and articles. The above-mentioned shapes, geometries, structures and configurations may contain additional features that will further enhance the desired application or use. For example, a polymer, compound and/or composition of the invention in the form of a rod, coil, or cone may have barbs that spring out upon insertion from a needle or cannula or when warmed to body temperature to reduce movement and/or expulsion. The shape, geometry, structure or configuration of a device, such as a medical implant, will vary depending upon the use of the device. For example, for treatment of a spinal cord injury or concussion to the brain, a polymer, compound and/or composition of the invention may be formed into a medical implant in the shape of a disc for placement under the dura or dura mater, or a solution, suspension, emulsion, cream, gel, ointment, or other adhesive formulation form for covering the spine, dura or other surgically exposed areas, film, sprayed or coated formulation. In another example, a polymer, compound and/or composition of the invention may be formed into a medical implant in the shape of a membrane or tube for use in the treatment of injury or damage to the peripheral nervous system or a block of solid or foamed composition containing pathways drilled or otherwise formed to encourage nerve growth or bone growth. In another example, in the treatment of cancer, a polymer, compound and/or composition of the invention may be formed into a medical implant in the shape of a pellet, microparticle e.g. microsphere, nanoparticle e.g. nanosphere, rod, membrane, pin, cuff, disc, bullet, hook, rod or cone, with or without barbs, for insertion in a bone, joint, tumor excision site or other structures, or for insertion within the same and other structures. In the above instances, bioerosion of the medical implant would yield or generate an agent.

The invention also contemplates that the shape, geometry, structure or configuration of a medical implant of the invention may change depending on the mode of delivery or administration and may enhance the therapeutic effect of the medical implant. For example, a medical device of the invention may be in the form of a linear rod when inserted in needles and stored but may become coil-like or form a multiplicity of coils or corkscrew shapes as the medical implant may be pushed out of the needle by a trochar. As a result of the change of the shape, geometry, structure or configuration of the medical implant, expulsion from the tumor or tumor excision site by hydraulic pressures or body movements may be prevented and as much mass of ingredient may be delivered to a small region with as small a diameter needle as possible. The polymers of the present invention may take the form of a shape memory polymer that may be a stimulus-responsive material that may change its shape in response to outside stimuli. Usually this is a temperature-related effect. It depends on the morphology of the material in combination with various processing parameters. Thus, many materials of widely different polymeric chemistry may behave as shape memory. See, e.g. Lendlein and Kelch, on Shape Memory Polymers, Encyclopedia of Polymer Science and Technology, Ed III, Publ. J Wiley & Sons, New York (2003). The material may be programmed initially by deforming the sample, usually at an elevated transition temperature, and then cooled in a distorted form so that it remains in this temporary state. It will remain there a long time but on re-heating to above the programming transition temperature it will revert to its natural undeformed state. Shape memory materials are all elastomers. They have a molecular structure consisting of network linked at certain net points either by physical or chemical cross-linking processes. The elastomer contains two types of polymer blocks whose phases are immiscible and have differing Tₘ or T_{g} values. Shape memory effects are usually recognized by tensile tests in a hot chamber over a range of transitions and seeing how the dimensions alter. The upper limit may be the melting point of the highest Tₘ block. A cyclical regimen will show how well the polymer recovers its original shape. Examples of shape memory polymers are polyester-urethanes with hard and soft segments. A typical hard switching one may be made from butane-1,4-diol and MDI with low Tg but crystalline polycaprolactone blocks. The Tₘ of the hard 4G-MDI block may be the upper temperature limit. Another segmented polyether-urethane may be the one from polyTHF and butane diol with MDI. Here, the molecular weight of the soft poly (THF) segment is important—if it is too high the recovery may suffer. Biodegradable shape memory polymers are possible based upon polycaprolactone diols capped with methacrylate groups and co-polymerized with a low T_{g} amorphous vinyl component such as polybutyl acrylate. Other compositions may include block co-polyester-ethers with hard segments such as polylactide, glycolide and soft segments such as polyTHF diol or caprolactone-diol. Polyanhydride linkers could be incorporated and, if a phosgene route were used to make the polyanhydride, it could also generate carbamoyl chlorides and urethane links at the same time form suitable amine precursors. The polymers of this invention achieve a broad range of tensile modulus anywhere between about 500, about 1000, about 5000, about 10000, about 50000, about 100000, or about 300000 psi to about 500000, about 600000, about 850000, about 1000000, about 1200000, or about 1500000 psi, among others, as well as any combination of ranges therebetween.

The mode of delivery, application, or administration of a device or implant of the invention may vary depending upon the use and may include those known in the art as well as those set forth herein. The thickness or diameter of the monomer, oligomer, polymer, compound and/or composition as either the formulation or medical implant itself or as applied or coated onto a medical implant will vary depending upon one or more factors such as the physical and/or chemical characteristics of the polymer, compound and/or composition, the medical implant and/or the application or use. For example, some articles may be formed from, or applied or coated with a polymer or composition of the invention to a thickness of about 1, about 2, about 5, about 10 to about 30, about 40, about 50 μm while others may be applied or coated with a polymer, compound and/or composition of the invention to a thickness of about 1, about 5, about 10, about 30, about 50 μm to about 70, about 85, about 100 μm and others such drug delivery devices may be applied or coated with a polymer, compound and/or composition of the invention to a thickness of about 0.2, about 0.5, about 0.1, about 0.5, about 1μ to about 2, about, 3, about 4, about 5 mm. Other diameters and thicknesses outside of the listed ranges are also contemplated in this invention. In another embodiment round films/membranes and other articles may have diameters of about 0.2, about 1, about 5 up to about 5, about 8, about 10 mm (1 cm) and a thickness of about 0.1, about 0.3, about 0.5μ to about 0.8, about 1, about 2 mm. In the present invention, a covering may be affixed to a medical device in several ways. In another embodiment the covering may be placed on the outside of the medical device, and through the natural properties of the polymer, i.e. stickiness or adhesiveness, adhere to the device. In still another embodiment the covering may fit snugly, form-fittingly, or loosely around the medical device, such that no adhesive may be required to affix the covering to the medical device. In yet another embodiment a covering of the invention may be affixed to the medical device by means of a biocompatible adhesive, the characteristics of which will be readily understood by one skilled in the art. In one another embodiment a covering may be affixed to a medical device by means of a device external to both the covering and the medical device. For example, the covering may be affixed to the medical device by means of an external clamp, retaining pin, or other such device commonly known in the art. External retaining devices used to affix a covering to a medical device may also be used to retain the shape of the covering. External retaining devices may retain the covering adjacent to the medical device by existing on the outside of the covering, on the inside of the covering, i.e. in between the covering and the medical device, or as a combination both outside and inside of the covering. In yet another embodiment, the covering may be affixed to the medical device by means of a fastener. Non-limiting examples of materials that may be used to make an external fixing device for a covering of the present invention include surgical steel, nylon, polyethylene, and combinations thereof.

As a non-limiting example of the present invention, a medical device may be covered by a first covering in the form of a polymeric sheath that may be, in turn, covered by an external retaining device in the form of a semi-rigid or rigid sleeve. Such an external retaining device may be made of metal, plastic, a polymeric substance, or a combination thereof. Such an external retaining device may also be formed of, covered by, or impregnated with a polymer according to the present invention as described herein, or may be covered by or impregnated with the same or different agent(s) present in the first therapeutic device. An external retaining device may also contain a polymer that comprises a functional group as described above. In another embodiment of the invention an external retaining device that may be formed from a polymer according to the present invention may comprise at least one functional group and/or agent(s) in any of the forms as described above for a first covering. In one embodiment a cuff or sleeve comprises a polymer(s) that generates an agent(s), such as an anti-inflammatory, anti-infective, antiseptic and/or anti-proliferative agent(s). Such a cuff may be made entirely of the polymer(s), or made of an inert substance that may be coated with the polymer(s). The cuff may adjoin or penetrate tissue layers to ensure delivery to the most likely sites of infection. The simplest version of the embodiment would be to coat the surfaces of a suitable device with the polymer and thereby enable a slow release of agent(s) along its length within the moist and enzyme rich milieu of body tissue. In preferred embodiment, the medical device may be coated with a polymer composition comprising an agent(s) including, but not limited to, an anti-inflammatory, anti-infective, antiseptic, and anti-proliferative agent(s). Polymers and compositions thereof with specific physical properties may be developed by one of skill in the art using the guidance given herein. In some preferred embodiments, a device or implant maybe further coated with a polymer that has lubricating qualities.

A polymer, compound and/or composition of the invention may be combined or admixed with other ingredients prior to or while being formed into or coated onto a medical device or into a particular coating for a medical device. Examples of suitable additives include, but are not limited to, stabilizers, mechanical stabilizers, plasticizers, hardeners, emulsifiers, other polymers including other biocompatible and biodegradable polymers, e.g. biocompatible and biodegradable polyanhydrides as set forth in U.S. Ser. No. 09/917,231 and PCT US/01/23740, biocompatible and biodegradable polyazo compounds as set forth in U.S. Ser. No. 09/917,595 and PCT US/01/23748, biocompatible and biodegradable polyesters, polythioesters, and polyamides as set forth in U.S. Ser. No. 09/917,194 and PCT US/01/23747, the relevant portions of which are incorporated herein by reference in their entireties, radioopaque and/or radioisotopic materials, e.g., boron, iodine, etc., suppositories, and other diagnostic or therapeutic agents or drugs. An added ingredient may enhance stability of the polymer, compound and/or composition itself, the medical implant itself and/or may enhance the diagnostic or therapeutic effect and/or may enhance or enable diagnostic activity. For example, if the added ingredient is a diagnostic or therapeutic agent or drug, bioerosion would not only release the agent(s) but also the diagnostic or therapeutic agent(s). In another example, by adding a radioopaque material, visualization of both the targeted area e.g. tumor site, tumor, and the medical implant e.g. catheter would be enabled during and/or after, e.g. angioplasty, dental applications, joint injections, etc., insertion of the medical implant. In another example, the radioopaque material may also be used to control and/or enhance bioerosion of the medical implant and thereby control and/or enhance generation of the agent(s) by the generation of heat resulting from neutron capture. An added ingredient may also enhance the overall mechanical stability of the medical implant, e.g. carbon fibers. The type of additive used would vary and depend upon the desired property and application. In one embodiment a medical device may be coated with a therapeutic co-polymer of two or more monomers or more monomers that each independently have different linker groups. In other preferred embodiments, the medical device may be coated with a therapeutic polymer composition that may be comprised of at least two therapeutic polymers that are mixed after polymerization.

The first and second agents may be the same or different, and in one embodiment, the first and second agents may both be incorporated into the polymer backbone or attached directly to it, for example, through a linker or spacer, or by direct or indirect chemical linkage to a chemical group attached to the polymer backbone; or the second agent(s) may be dispersed within the polymer matrix or appended to the polymer, while the first agent(s) may be incorporated into the backbone of the polymer or attached directly to the backbone, for example, through a linker or spacer, or by direct or indirect chemical linkage to a chemical group attached to the polymer backbone; or the first and second agents may be dispersed within the polymer matrix of the polymer or appended to the polymer. The polymer may also comprise additional agents, such as a third agent(s), fourth agent(s), fifth agent(s), and so on, where the additional agents are released by degradation of the polymer. For example, the additional agent(s) may be incorporated into the backbone of the polymer or attached directly to it, for example through a linker or spacer, or attached to the backbone by direct or indirect chemical linkage to the polymer backbone; or dispersed within the polymer matrix of the polymer or appended to the polymer as described herein, or otherwise annexed to or associated with the polymer such that the additional agents dissociate from the polymer upon hydrolysis.

Another preferred embodiment comprises a device having at least one surface, the device comprising more than one polymer on all or a part of the surface, such as having first and second polymers that may be the same or different. For example, in one embodiment the polymer may be coated onto a device that experiences expansion, contraction or torsion during application or use. This polymer coating might be used to reduce the incidence of inflammation and resulting hyperproliferation of cells in the surrounding area. In one embodiment the linking group may be a dicarboxlyic acid hydrocarbon chain with about 2 to about 50 carbon atoms. In another embodiment the medical device comprises a polymer comprising at least one agent(s) that may be incorporated into the polymer backbone. The article may comprise additional polymers and/or additional agents, such as a second agent(s), third agent(s), and so on, where the additional agents may be incorporated, attached, appended, blended, dispersed or otherwise associated with the polymer, or otherwise annexed to or associated with the polymer such that the additional active agents dissociate from or are released by the polymer upon erosion or hydrolysis. The article may comprise at least one agent(s) that combine(s) in vivo to form an agent(s). In one embodiment an implantable article may be coated with at least one therapeutic polymer(s) of the invention. The implantable article may be made of any material known to those in the art including novel materials that may be developed later on, including but not limited to, electropolished stainless steel and other metallic alloys and polymeric materials. For such a device a preferred coating(s) have preferably a thickness from about 10 nm to about 100 μm, and most preferably has a thickness of about 1, about 2, about 3.5, about 5, about 7.5, about 10 μm to about 12.5, about 15, about 20, about 24, about 26, about 28.5, about 30 μm. For some articles used in medical or veterinary applications coatings or sets of coatings preferably have a thickness less than about 100 μm. As described above the therapeutic polymer may be applied as a coating(s) to an implantable orthopedic device and dental implant to maintain bone strength, to induce bone penetration of the device and to stabilize it and/or to reduce pain and inflammation and/or to reduce infections.

Compositions comprising a polymer also may be used to coat orthopedic devices for fixation of bone fractures such as pins or screws, thereby decreasing the local inflammation and bone resorption associated with these devices.

Films comprising an aromatic polyanhydride are also useful as orthopedic devices to enhance the healing process of bone fractures. A polymer may be coated or applied onto or formed into sutures, wound closures, stitches, staples and other related devices. In the case of sutures, staples and other devices such a coating could be used to reduce infections, pain and/or inflammation in the vicinity of the suture or staple. Fibers made of the present polymer(s) are useful as suture materials, and may be used in oral surgery to suture cleft palates. Use of a polymer that degrades to an agent, such as a therapeutic salicylate, would enhance the regeneration of the tissue via the sutures while decreasing the pain and inflammation associated with the surgery via the degradation products. Films, membranes, pastes, gels, chips and microspheres comprising the polymer may also be used to decrease dental pain and promote healing within a tooth, in the pulp chamber and root canal. Films or membranes comprising a polymer may also be used in guided bone or tissue regeneration.

In one embodiment, the polymers, compounds and/or compositions of the invention may be formed into micronized particles or microparticles, or nanoparticles e.g. microspheres, nanospheres, nanocapsules and/or microcapsules. Microparticles of a polymer, compound and/or composition of the invention may be prepared by any means known in the art and may include one or more of the same or different polymer, compound and/or composition of the invention. For example, the microparticles may be prepared using an oil-in-water emulsion method whereby a polymer of the invention may be dissolved in an organic solvent. The polymer solution may be then added to a stirring solution of water and polyvinyl alcohol (PVA) as a stabilizer to obtain the precipitation of the desired microparticles. Optionally, a homogenizer may be used. The solution may be then allowed to settle, the solvent decanted off the solution, and the microparticles dried. The microparticles, such as, e.g., microspheres may be applied to the surface of a medical device before placement in the body. A sterile liquid may be used to coat the device to adhere such microspheres for minutes to weeks to enable uncoated medical devices to benefit from the same or similar therapeutic benefits as coated devices. In one embodiment, the nanoparticles or microparticles are typically but not necessarily less than about 10 nm or microns in diameter. In another oil-in-water emulsion method, the polymer solution may be added to a solution of water and a surfactant such as PVA, which may be stirred rapidly at high shear rates with, for example, a homogenizer or dispersator. After the addition of the polymer solution, the solvent may be allowed to evaporate while stirring may be continued. The resulting microparticles are recovered by decantation, filtration or centrifugation and dried. Microparticles of the invention may also be prepared by known microencapsulation processes, e.g. the process described by U.S. Pat. No. 5,407,609, the relevant text of which may be incorporated herein by reference. The patent describes a continuous microencapsulation process whereby a polymer, protein, peptide, small molecule, water-soluble, hydrophobic drug, and drugs within a polymer may be added to a mechanically agitated water/surfactant mixture to form a microdroplet emulsion. Water may be then employed to extract or remove the solvent, and form hardened microcapsules or microspheres that are collected by centrifugation, filtration or the like. In accordance with this continuous microencapsulation process molecules such as nucleic acids, saccharides, lipids, proteins, peptides, small molecules, water-soluble drugs, hydrophobic drugs, and drugs may be encapsulated in lactide/glycolide polymers to sizes of about 1, 2, 5, 10, 15 to up to about 10, 50, 75, 100, 150, 200, 250 μm, with minimal exposure to polymer solvent and with high encapsulation efficiency and good yields.

Having now generally described this invention, the same will be better understood by reference to certain specific examples, which are included herein for purposes of illustration only and are not intended to be limiting of the invention or any embodiment thereof, unless so specified.

### EXAMPLES

The following abbreviations are employed throughout the examples: BPC (bupivacaine), D (drug), L (linker), DCM (dichloromethane), DF (diflunisal), MPA (mycophenolic acid), MTX (methotrexate), PAC (paclitaxel), SA (salicylic acid), TEA (triethylamine), TFA (trifluoroacetic acid), THF (tetrahydrofuran), TP (triphosgene). All solvents and reagents employed in the following examples were purchased and used as received. Proton nuclear magnetic resonance (¹H NMR) spectra were recorded on a Varian 300 MHz Mercury VX-300 spectrometer using an appropriate deuterated solvent. Chemical shifts (δ) are reported in parts per million (ppm) downfield from tetramethylsilane (TMS) and coupling constants (J values) are given in hertz (Hz). Molecular weights (M_{w}) and polydispersity indices (PDI) were determined by gel permeation chromatography (GPC) on a Viscotek TDA 301 system consisting of a refractive index detector and a Viscotek VE 1122 pump using Omnisec software for data collection and processing. Molecular weights were calibrated relative to a narrow molecular weight polystyrene standard (Viscotek, Houston, Tex.). The HPLC impurity profile may be performed on an Agilent Rapid Phase C18 column 4.6×70 mm column with a flow rate of 1.8 ml/min and a gradient of 6%/min of mobile phase B (0.1% (v/v) TFA in acetonitrile) in mobile phase A (0.1% (v/v) TFA in water). The gradient runs on an ambient column with a VWD at 225 nm.

### Example 1

### General Procedure for Preparation of Linker-Diacid Chloride (Compound 12)

0.48 mol oxalyl chloride was added to a mixture of 0.16 mol diacid (Compound 11) in 320 ml anhydrous chloroform, and the mixture stirred overnight at room temperature, gently refluxed for 1 hour, and cooled to room temperature. The solvent was then removed in vacuo, and the residue dried in vacuo at 45° C. to obtain the product.

### Example 2

### Preparation of C14 Diacid Chloride (Compound 12a)

1,12-Dodecanedicarboxylic acid (Compound 11a) was subjected to the conditions described in Example 1. Yield C14 Diacid Chloride: 99%. The structure of the product was confirmed by ¹H NMR.

### Example 3

### Preparation of C16 Diacid Chloride (Compound 12b)

1,16-Hexadecanedioic acid (Compound 11b) was subjected to the conditions shown in Example 1. Yield C16 Diacid Chloride: 99%. The structure of the product was confirmed by ¹H NMR.

### Example 4

### General Procedure for Preparation of D-L-D Aromatic Diacids (Compound 14)

mol pyridine was added to a solution of 0.325 mol of compound 13 in 800 ml anhydrous THF, and then 125 ml solution of 0.16 mol linker diacid chloride in anhydrous THF was added drop-wise. The reaction mixture was stirred for 45 minutes, and poured into an 80 ml solution of HCl (conc.) maintained in 2.4 liters of ice-cold water. The mixture was stirred for 1 hr, and the solid produced was isolated by decanting then supernate, and washing the solid with 1 liter of cold water. The crude solid product was washed with cold water, filtered, and dried in a vacuum oven at 45° C. overnight, and the dried solid was purified twice from 3:1 (v:v) hexane-ethyl acetate.

### Example 5

### Preparation of Salicylic Acid-C8-Salicylic Acid (SA-C8-SA; Compound 14a)

The diacid was prepared from SA and suberoyl chloride using the general procedure given in Example 4. The structure of the product was confirmed by ¹H NMR.

### Example 6

### Preparation of Salicylic Acid-C10-Salicylic Acid (SA-C10-SA; Compound 14b)

The diacid was prepared from SA and sebacoyl chloride employing the procedure described in example 4 above. Yield SA-C10-SA: 97%. The structure of the product was confirmed by ¹H NMR.

### Example 7

### Preparation of Diflunisal-C12-Diflunisal (DF-C12-DF; Compound 14c)

The diacid was prepared from DF and 1,10-decane dicarboxylic acid chloride using the general procedure provided in Example 4 above. The structure of the product was confirmed by ¹H NMR.

### Example 8

### Preparation of Diflunisal-C14-Diflunisal Diacid (DF-C14-DF; Compound 14d)

The diacid was prepared from diflunisal (DF) and 1,12-dodecane dicarboxylic acid dichloride (Compound 12a) at 99% yield using the general procedure given in Example 4. Yield DF-C14-DF: 99%. The structure of the product was confirmed by ¹H NMR.

### Example 9

### Preparation of Diflunisal-C16-Diflunisal Diacid (DF-C16-DF; Compound 14e)

The diacid was prepared from DF and 1,16-hexadecanedioic acid dichloride (Compound 12b) using the general procedure given in Example 4. The structure of the product was confirmed by ¹H NMR.

### Example 10

### General Procedure for Preparation of D-L-D Diacid Chloride (Compound 15)

106.6 mol oxalyl chloride were added to a solution of 34.59 mol D-L-D diacid in 200 ml anhydrous chloroform, and the reaction mixture was refluxed gently for three hours. The clear reaction solution was concentrated in vacuo, and the residue recrystallized in 1:1 (v:v) anhydrous DCM-heptane to obtain a white solid. The solid was filtered and washed with heptane to obtain the product.

### Example 11

### Preparation of Salicylic Acid-C8-Salicylic Acid Diacid Chloride (SA-C8-SA Diacid Chloride; Compound 15a)

The diacid chloride was prepared from SA-C8-SA diacid using the general procedure shown in Example 10 above. The structure of the product was confirmed by ¹H NMR.

### Example 12

### Preparation of Salicylic Acid-C10-Salicylic Acid Diacid Chloride(SA-C10-SA Diacid Chloride; Compound 15b)

The diacid chloride was prepared from SA-C10-SA diacid using the general procedure given in Example 10 above. The structure of the product was confirmed by ¹H NMR.

### Example 13

### Preparation of Diflunisal-C12-Diflunisal Diacid Chloride (DF-C12-DF Diacid Chloride; Compound 15c)

The diacid chloride was prepared from DF-C12-DF diacid employing the procedure provided in Example 10 above. The structure of the product was confirmed by ¹H NMR.

### Example 14

### Preparation of Diflunisal-C14-Diflunisal Diacid Chloride (DF-C14-DF Diacid Chloride; Compound 15d)

The diacid chloride was prepared from DF-C14-DF diacid in 99% yield using the general procedure given in Example 10. The structure of the product was confirmed by ¹H NMR.

### Example 15

### General Procedure for Preparation of D-D-L-D-D Diacid (Compound 16)

0.189 mol anhydrous pyridine was added to a solution of 0.077 mol Compound 13 in 150 ml anhydrous THF, the mixture was stirred for 5 minutes, and a solution of 0.035 mol D-L-D diacid chloride in 150 ml anhydrous THF was added drop-wise. The reaction mixture was stirred for 30 minutes at room temperature, and was poured into a mixture of 180 ml cold water and 20 ml HCl (conc.). The mixture was extracted three times with 150 ml ethyl acetate, and the combined organic layer was washed twice with 100 ml water and 100 ml brine, and was dried over anhydrous sodium sulfate. The solvent was removed in vacuo, and the residue was purified twice from 1:1 (v:v) ethyl ether-pentane to obtain the product.

### Example 16

### Preparation of Salicylic Acid-Salicylic Acid-C8-Salicylic Acid-Salicylic Acid Diacid (SA-SA-C8-SA-SA Diacid; Compound 16a)

The diacid was prepared from SA and SA-C8-SA diacid chloride (Compound 15a) employing the general procedure given in Example 15. Yield: 85%. The structure of the product was confirmed by ¹H NMR.

### Example 17

### Preparation of Diflunisal-Diflunisal-C14-Diflunisal-Diflunisal Diacid (DF-DF-C14-DF-DF Diacid; Compound 16d)

The diacid was prepared from DF and DF-C14-DF diacid chloride (Compound 15d) employing the procedure described in Example 15 above. Yield: 95%. The structure of the product was confirmed by ¹H NMR.

### Example 18

### Preparation of C6 bis-L-Lactate Diol (Compound 19a)

33.60 g 1,6-dibromo hexane (Compound 18a; 0.15 mol) was added to a solution of 33.62 g sodium L-lactate (Compound 17a; 0.3 mol) in 60 ml anhydrous DMF, and the mixture was heated at 60° C. for 3 days. The reaction mixture was cooled to room temperature and poured into 500 ml cold water, acidified to about pH 4 with 1N HCl, and extracted 4 times with 75 ml ethyl acetate. The organic layers were combined and washed with water, dried over anhydrous sodium sulfate, and the solvent removed in vacuo to obtain a slightly brownish oily product. The product was filtered over silica gel with 1:1 (v:v) ethyl acetate-hexane. Thirty-two g of pure product were obtained. The structure of the product was confirmed by ¹H NMR.

### Example 19

### Preparation of C10-bis-L-Lactate Diol (Compound 19b)

25.0 g 1,10-diiododecane (Compound 18b) was dissolved in 7 ml DCM, and the solution added to 120 g tetrabutylammonium-L-lactate (Compound 17b). The reaction mixture was placed in a 40° C. rotary evaporator bath, and rotated at top speed for 20 hours. The solution was then diluted with 100 ml dichloromethane, and washed with 100 ml water. 750 ml diethyl ether were placed into a 2-liter Erlenmeyer flask and stirred magnetically. The lower organic phase from the separatory funnel was dripped into the diethyl ether with stirring until a precipitate appeared. The precipitated salt (tetrabutylammonium iodide) was vacuum-filtered through a medium porosity frit, and the filtrate was collected in a 1-liter round-bottom flask and washed once with 400 ml 1.25% sodium thiosulfate in water, and twice with 400 ml water. The ether layer was dried over anhydrous magnesium sulfate and the solvent was removed in vacuo to produce 15.5 g of the product.

The structure of the product was confirmed by ¹H NMR.

### Example 20

### Preparation of C8 bis-L-Lactate Diol (Compound 19c)

The diol was prepared from 1,8-dibromoooctane (Compound 18c) and Compound 17a, employing the same conditions given in Example 18. The structure of the product was confirmed by ¹H NMR.

### Example 21

### Preparation of C6 bis-D,L-Lactate Diol (Compound 19d)

The diol was prepared from Compound 18a and lithium D,L-lactate (Compound 17c) employing the same conditions given in Example 18. The structure of the product was confirmed by ¹H NMR.

### Example 22

### Preparation of C8-bis-Glycolate Diol (Compound 21a)

4.2 ml triethyl amine (30 mmol) were added to a solution of 2.28 g glycolic acid (Compound 20a; 30 mmol) in 10 ml anhydrous DMF. The mixture was stirred for 5 minutes at 60° C., 4.08 g 1,8-dibromooctane (Compound 18b; 15 mmol) was added, and the reaction mixture was stirred at 60° C. for 24 hours and then cooled to room temperature, poured into 75 ml cold water, and acidified to about pH 4 with 1N HCl. A white precipitate that appeared was filtered and dried to obtain 2.8 g of the product. The structure of the product was confirmed by ¹H NMR.

### Example 23

### Preparation of C8 Salicylic Acid Polymer (Compound 23a) by Non-aqueous Dispersion Method with Dispersing Agent

A 50 ml reaction vessel fitted with a 3-neck flanged lid, carrying a sealed Teflon paddle stirrer, a rubber septum over one side neck, and a short Vigreux distillation column and receiver flask, was cooled in a dry ice bath. Fifty ml light white mineral oil, 8.60 g suberoyl bis-salicylic acid-acetic acid mixed anhydride (Compound 22), and 0.26 g polyvinylpyrrolidone/eicosane co-polymer (ISP Corp., Antaron 220) as dispersing agent were added to the reaction vessel, and the mixture was briskly mixed. A slow stream of Argon gas was passed through the stirred mixture as a sparge, and the mixture was heated to 120° C. in an oil bath and maintained in these conditions under Argon for 30 minutes. The vessel was then slowly placed under vacuum at 120-140° C. with constant vigorous stirring to a final vacuum of 2.0 mTorr, and the oil was refluxed halfway in a Vigreux column. The reaction was allowed to proceed for 6 hours, then allowed to cool to 70° C. under vacuum with stirring while the volatile products, e.g., acetic anhydride, were collected in a chilled receiver flask. The vacuum was then released with Argon, and the vessel cooled to room temperature. The reaction mixture was diluted with anhydrous petroleum ether, and centrifuged for 30 minutes to collect the product. The supernate was removed, and the residual solid was washed 3 times with dry petroleum ether, and dried at 40° C. in a vacuum oven for several hours to obtain 4.38 g of the product. Yield: 64%; MW=51,000 Dalton, as determined by GPC as compared with a limiting 14,000 Dalton MW obtained by standard bulk-melt polymerization. Polymer particles were amorphous, clear, and formed perfectly spherical 5 to 50μ diameter particles as determined in a low power optical microscope.

### Example 24

### Preparation of C14 Diflunisal Polyanhydrides (Compound 23b) by Non-Aqueous Dispersion Method Without Dispersing Agent

A 50 ml reaction vessel fitted with a 3-neck flanged lid, in turn carrying a sealed Teflon paddle stirrer, a rubber septum over one side neck and a short Vigreux distillation column and receiver flask, was cooled in a dry ice bath, and was charged with 50 mL of light white mineral oil, 8.54 g of bis(2-carboxy-4-(2,4-difluorophenyl)tetradecane dicarboxylate-acetic acid mixed anhydride (22b). The reactor was evacuated to 40 mTorr, and heated to 110° C. for 1 hour with constant vigorous stirring. The temperature was increased to 160° C. and held for the duration of the reaction. A final vacuum of 30 mTorr was achieved, and the oil was refluxed part of the way up a Vigreux column. The volatile reaction products, e.g. acetic anhydride, were collected in a chilled receiver flask. The reaction was allowed to proceed overnight and was cooled to room temperature under vacuum with stirring. The solution consisted of a polymer mass on the bottom of the reactor, and oil above the solids. The oil was decanted off and the residue was washed with petroleum ether twice. The residue was dissolved in anhydrous DCM, and a white precipitate was obtained by precipitation into anhydrous ethyl ether. The white precipitate was dried at 40° C. under vacuum to give the product as a solid (5.6 g). Yield: 75%; Mw=405,000; PDI=1.75

### Example 25

### Preparation of High Molecular Weight Poly(Sebacic Anhydride) (Compound 24a)

27.8 ml anhydrous TEA were added to a solution of 20.226 g sebacic acid in 100 ml anhydrous chloroform, and the mixture was cooled to 0° C. in an ice bath. A solution of 9.892 g triphosgene in 25 ml anhydrous chloroform was added very slowly to the reaction mixture at 0° C. with vigorous stirring. The reaction mixture was then warmed up to room temperature and mildly refluxed for 3 hours. To reduce the viscosity of the thin layer of undissolved polymer that remained at the bottom of the flask, 250 ml anhydrous chloroform were added, and the system was heated until the flask's contents completely dissolved. A sample of the polymer solution was removed from the flask, and a conventional calibration was performed with GPC using narrow polydispersity polystyrene standards. MW=626,000; PDI=1.79

### Example 26

### Preparation of Poly Diflunisal-C14-Diflunisal Ester Anhydride (DF-C14-DF Ester Anhydride; Compound 25a)

7.70 ml anhydrous TEA were added to a solution of 20.0 g DF-C14-DF diacid (Compound 14d) in 80 ml anhydrous chloroform at 0° C., the solution was stirred for 30 minutes, and a solution of 2.750 g triphosgene in 20 ml anhydrous chloroform was added drop-wise. The reaction mixture was stirred at 0° C. for 30 minutes, diluted with 40 ml chloroform, and washed once with 100 ml 1N HCl, and once with 100 ml distilled water. The organic layer was dried over anhydrous magnesium sulfate, and the solvent was removed in vacuo. The residue was dissolved in DCM and poured into diethyl ether in a Teflon beaker with stirring to precipitate the product. The supernate was decanted, and the residue washed with additional ether before drying in a vacuum oven at 45° C. overnight to obtain 10.7 g of the product. Mw=176,000; PDI=1.85

### Example 27

### Preparation of Poly Salicylic Acid-C8-Salicylic Acid Ester Anhydride (SA-C8-SA Ester Anhydride (Compound 25b)

This polymer was prepared from SA-C8-SA diacid (14a) using the same conditions given in Example 24 above. MW=121,000; PDI=1.73

### Example 28

### Preparation of Poly Salicylic Acid-C8-Salicylic Acid Ester Anhydride (SA-C8-SA Ester Anhydride; Compound 25c)

The polymer was prepared from SA-CLO-SA diacid (14b) using the same conditions given in Example 24 above. MW=110,000; PDI=1.61 The structure of the product was confirmed by ¹H NMR.

### Example 29

### Preparation of Mixed Random Poly Anhydride of SA-C8-SA and SA-SA-C8-SA-SA (Compound 26a)

8.7 ml anhydrous TEA (61.6 mmol) were added to a solution of 8.7 g SA-C8-SA diacid (Compound 14a; 21 mmol) and 4.58 g SA-SA-C8-SA-SA diacid (Compound 16a) (7 mmol) in 70 ml anhydrous DCM at 0° C., and the solution was stirred for 30 minutes. A solution of 2.8 g triphosgene (9.34 mmol) in 20 ml anhydrous DCM was added drop-wise to the mixture at 0° C., stirred for 1 hour at 0° C., diluted with 25 ml DCM, washed once with 25 ml 1N HCl and twice with 100 ml distilled water, and dried over anhydrous magnesium sulfate. The solution was concentrated in vacuo to about 75 ml, and the was product precipitated by pouring the solution into anhydrous diethyl ether in a Teflon cylinder while stirring. The thus obtained solid was washed with diethyl ether and dried in a vacuum oven at 40° C. overnight to obtain 10.0 g of the product. MW=110,000; PDI=1.24

### Example 30

### Preparation of Mixed Random Poly Anhydride of DF-C14-DF and DF-DF-C14-DF-DF (Compound 26b)

21 mmol DF-C14-DF diacid (Compound 14d) and 7 mmol DF-DF-C14-DF-DF diacid (Compound 16d) were employed as described in Example 27 to obtain 25.2 g of the product. MW=163,000; PDI=1.32. The structure of the product was confirmed by ¹H NMR.

### Example 31

### Preparation of Mixed Random Poly Anhydride of DF-C16-DF and DF-DF-C14-DF-DF (Compound 26c)

42.5 mmol DF-C16-DF diacid (Compound 14e) and 7.5 mmol DF-DF-C14-DF-DF (DF-DF-C14-DF-DF diacid; compound 16d) were subjected to the conditions described in Example 27 above to obtain 30 g of product. MW=168,000; PDI=3.1

### Example 32

### Preparation of Random Poly Diflunisal-C14-Diflunisal-co DF Anhydride (DF-C14-DF-coDF Anhydride; Compound 27a)

A solution of 6.579 g DF and 7.35 ml TEA in 20.0 ml anhydrous chloroform was slowly added to a solution of 10.0 g compound 15d and 3.895 g compound 12b in 80 ml anhydrous chloroform at 0° C.±4° C. The reaction mixture was stirred for 1 hour at 0±4° C., and washed with 100 ml 1N HCl, and 100 ml distilled water. The organic layer was dried over anhydrous magnesium sulfate, the solvent was removed in vacuo, and then the solid was dried in a vacuum oven at 40° C. overnight. Fifteen g of the dried polymer were redissolved in 70 ml anhydrous chloroform and 0.625 ml TEA was added to the solution at 0° C. The reaction solution was stirred for 1 hour, and a solution of 87.2 mg triphosgene in 2.0 ml anhydrous chloroform at 0° C. was slowly added. The reaction mixture was stirred for 1 hour at 0±4° C., and was washed with 100 ml 1N HCl and 100 ml distilled water. The organic layer was dried over anhydrous sodium sulfate, and the solvent was removed in vacuo at 40° C. The crude polymer was dissolved in 120 ml DCM, and then slowly added to 1.2 l anhydrous diethyl ether that was placed in a Teflon cylinder while stirring vigorously. The supernate was decanted, and the residue was washed with anhydrous ethyl ether. The thus obtained gummy polymer was transferred into a Teflon dish and dried in a vacuum oven at 40° C. for 24 hours to obtain 11.4 g of product. MW=149,000; PDI=2.36

### Example 33

### Preparation of Random Poly Salicylic Acid-C8-Salicylic Acid-coSalicylic Acid Anhydride (SA-C8-SA-co-SA Anhydride; Compound 27b)

The polymer was prepared from SA-C8-SA diacid (Compound 14a), suberoyl chloride, and SA using the same conditions shown in Example 30 above. MW=79,000; PDI=2.66

### Example 34

### Preparation of Random Poly (DF-C14-DF-coDF) Anhydride (Compound 27c)

A mixture of 3.971 g DF and 4.64 ml TEA in 18 ml anhydrous DCM was added drop-wise to a solution of 10.0 g of DF-C14-DF diacid chloride (Compound 15d) in 30 ml anhydrous DCM at 5° C., and the reaction mixture was stirred for 30 minutes at 5° C. The mixture was then diluted with 40 ml DCM, washed with 100 ml 1N HCl and 100 ml distilled water, and dried over anhydrous magnesium sulfate. The solution was concentrated to about 50 ml in vacuo, and was poured into anhydrous diethyl ether in a Teflon cylinder with stirring to precipitate the product. The supernate was decanted, and the solid was washed with ethyl ether, and dried in a vacuum oven at 40° C. overnight to obtain 9.3 g of the product. MW=106,000; PDI=1.88

### Example 35

### Preparation of Random Poly (DF-C14-DF co-Mycophenolic Acid) Anhydride (Compound 27d)

Compound 27c was prepared from DF-C14-DF diacid (Compound 15d) and MPA using the conditions shown in Example 32 above.

### Example 36

### Preparation of Random Poly (DF-C14-DF co-Methotrexate) Anhydride (Compound 27e)

Compound 27e was prepared from DF-C14-DF diacid and MTX using the same conditions given in Example 32.

### Example 37

### Preparation of Random Poly (DF-C12-DF co-Diflunisal) Anhydride (Compound 28a)

A solution of 2.755 g C10-bis-L-lactate diol (Compound 19b) and 3.623 ml anhydrous TEA in 25 ml anhydrous THF was added to 19.00 g of a solution of DF-C12-DF diacid chloride (Compound 15c) in 125 ml anhydrous THF. The reaction mixture was stirred for 12 hours at 30° C., concentrated in vacuo, co-evaporated twice with 200 ml additional chloroform, and dried in a vacuum oven at 30° C. overnight. The dried intermediate (pre-polymer) was re-dissolved in 100 ml anhydrous chloroform, and was cooled to 0° C. in an ice bath. A mixture of 3.466 g DF and 4.058 ml anhydrous TEA was made in 100 ml anhydrous chloroform, and was slowly added to the pre-polymer solution at 0° C. The reaction mixture was stirred for 1 hour at 0° C., washed with 200 ml 1N HCl and 200 ml distilled water, dried over anhydrous magnesium sulfate, concentrated in vacuo, and dried in a vacuum oven at 40° C. overnight. 0.317 ml anhydrous TEA were added to a solution of 21.6 g intermediate prepolymer in 140 ml DCM, and a solution of 111 mg triphosgene in 5.0 ml anhydrous chloroform was added drop-wise at 0° C. The reaction mixture was stirred at 0° C. for 1 hour, diluted with 40 ml chloroform, washed with 100 ml 1N HCl and twice with 500 ml distilled water, and dried over anhydrous MgSO₄. The solution was concentrated in vacuo to about 50 ml, and poured into anhydrous diethyl ether in a Teflon cylinder with stirring to precipitate the product. The supernate was decanted, and the solid was washed with ethyl ether, and dried in a vacuum oven at 40° C. overnight to yield 13.3 g of product. MW=120,000; PDI=1.35 Example 38: Preparation of Random Poly(Tetradecanedioic Acid-bis-Diflunisal Phenolate DF-C14-DF-co-C10-bis-Lactate-co-DF) Anhydride (Compound 28b)

A random tetradecanedioic acid-bis-diflunisal phenolate ester-co-decanediol-bis-L-lactate-co-diflunisal anhydride polymer (Compound 28b) was prepared from (DF), DF-C14-DF diacid chloride (Compound 15d), and C10-bis-lactate diol (Compound 19b) employing the conditions given in Example 35 above. MW=89,000; PDI=1.29

### Example 39

### Preparation of Random Poly (DF-C14-DF-co-C8-bis-Lactate-co-DF) Anhydride (Compound 28c)

A random tetradecanedioic acid-bis-diflunisal phenolate ester-co-octanediol-bis-L-lactate-co-diflunisal anhydride polymer (Compound 28c) was prepared from DF-DF-C14-DF diacid chloride (Compound 15d), and C8-dilactate diol (Compound 19c) using the conditions shown in Example 35 above. MW=63,000; PDI=1.46

### Example 40

### Preparation of 1,3-Propanediyl Bissalicylate (Compound 29a)

13.94 ml TEA (100 mmol) were added to a solution of 13.81 g salicylic acid (SA; 100 mmol) in 40 ml DMF at 60° C., the reaction mixture was stirred for 20 minutes at 60° C., and 12.2 g 1,3-dibromo propane (Compound 18d; 50 mmol) were added. The reaction mixture was stirred at 60° C. for 24 hours, cooled to room temperature, poured into 250 ml cold water, and acidified to about pH 4 with 1N HCl. A white precipitate separated. The precipitate was filtered, washed with water, and dried in a vacuum oven at 40° C. overnight. The thus obtained crude product was recrystallized from n-heptane to obtain a pure product. Yield: 85%

### Example 41

### Preparation of a Poly (SA-C6-SA) Carbonate (Compound 30a)

2.09 ml TEA (15 mmol) and 0.18 g DMAP were added to a solution of 1.89 g Compound 29a (6 mmol) in 30 ml anhydrous DCM at 0° C. The reaction mixture was stirred for 10 minutes and 20 wt % toluene solution containing 3.18 ml phosgene (6 mmol) in 5 ml anhydrous DCM was added drop-wise. The mixture was warmed to room temperature, stirred for 3 hours, and diluted with 30 ml DCM. The solution was washed with 20 ml 1N HCl, washed three times with 25 ml water, and was dried over anhydrous sodium sulfate, and concentrated in vacuo. The polymer residue was redissolved in 10 ml anhydrous DCM, and was added to 150 ml anhydrous ether with stirring until an insoluble polycarbonate separated. The polymer was washed with ethyl ether and dried in a vacuum oven at 40° C. to obtain 1.3 g of the product. MW=73,643; PDI=1.85

### Example 42

### Preparation of a Poly (SA-C6-SA-co-diacid) Ester (Compound 31a)

0.42 ml TEA (3 mmol) and 10 mg DMAP were added to a solution of 0.316 g Compound 29a (1 mmol) in 5 ml anhydrous DCM at 0° C. The reaction mixture was stirred for 10 minutes, and 0.239 g sebacoyl chloride (1 mmol) in 2 ml anhydrous DCM was added thereto. The mixture was then warmed to room temperature, stirred for 3 hours, and diluted with 20 ml DCM. The solution was then washed with 20 ml 1N HCl, three times with 25 ml water, dried over anhydrous sodium sulfate, and concentrated in vacuo to produce 0.4 g of the product. MW=25,293; PDI=1.6

### Example 43

### Preparation of a Poly (DF-C8-DF co-C8-bis-Glycolate) Ester (Compound 32a)

0.7 ml anhydrous TEA (5 mmol) were added to a solution of 0.53 g bis-glycolate diol (Compound 21a; 2 mmol) in 10 ml anhydrous DCM at 0° C. A solution of 2.70 g DF-C8-DF diacid chloride (Compound 15f; 4 mmol) in 15 ml anhydrous DCM was prepared, and added drop-wise to the reaction mixture. The mixture was allowed to warm to room temperature, and maintained at this temperature with stirring for 4 hours. The reaction solution was diluted with 25 ml DCM, washed once with 20 ml 1N HCl, twice with 20 ml distilled water, and was then dried over anhydrous magnesium sulfate, filtered, and concentrated in vacuo to about 5 ml. The polymer solution was poured into 40 ml anhydrous diethyl ether in a 1 liter Teflon cylinder while stirring with a magnetic stir bar to precipitate the product. The supernate was decanted and the remaining was solid rinsed with anhydrous diethyl ether and dried in a vacuum oven at 40° C. overnight to yield 1.8 g of the product. MW=8,349; PDI=1.33

### Example 44

### Preparation of a Poly (DF-C10-DF co-C8-bis-D,L-Lactate Ester) (Compound 32b)

Compound 32b was prepared from Compound 19c and Compound 15e employing the procedures described in Example 43. MW=42,785; PDI=1.68. The structure of the product was confirmed by ¹H NMR.

### Example 45

### Preparation of a Branched Poly (DF-C14-DF) Anhydride with 1,3,5-Benzene Tricarboxylic Acid (Compound 34a)

3.2 ml anhydrous TEA were added slowly to a mixture of 6.86 g (9.5 mmol) DF-C14-DF diacid (14d) and 0.106 g (0.5 mmol) 1,3,5-benzenetricarboxylic acid (Compound 33) in 40 ml anhydrous DCM at 0° C. A solution of 0.99 g triphosgene in 25.0 ml anhydrous DCM was then added into the reaction flask in a slow drop-wise manner at 0° C., and the reaction was stirred for 1.5 hours at 0±4° C. under Argon. The reaction mixture was diluted with anhydrous 50 ml DCM, and washed once with 50 ml 1N HCl and twice with 50 ml distilled water, dried over anhydrous magnesium sulfate, filtered, and concentrated in vacuo to about 20 ml. The polymer solution was then poured into anhydrous 500 ml diethyl ether contained in a 1 Teflon cylinder while stirring with a magnetic stir bar to precipitate the product. The supernate was decanted, and the solid was rinsed with anhydrous diethyl ether, and dried in a vacuum oven at 40° C. overnight to obtain 4.0 g of the product as a solid. MW=223,000; PDI=4.2

### Example 46

### Preparation of a Branched Poly (DF-C14-DF) Anhydride with 1,2,3,4-Butane Tetracarboxylic Acid (Compound 36a)

Compound 36a was prepared from DF-C14-DF diacid (Compound 14d) and 1,2,3,4-butanetetracarboxylic acid (Compound 35) using the conditions described in Example 42 above.

### Example 47

### Preparation of a Branched Polymer Using Trans-Aconitic Acid (Compound 38a)

Compound 38a was prepared from DF-C14-DF diacid (Compound 14d), and trans-aconitic acid (Compound 37) employing the conditions shown in Example 42 above.

### Example 48

### Preparation of a Random Block Polyanhydride (Compound 39)

0.256 ml anhydrous TEA was added drop-wise to a solution of 10.83 g Compound 26b (Mn=51,000; 0.21 mmol)) and 5.00 g of Compound 25c (Mₙ=22,000; 0.23 mmol) in 70 ml anhydrous DCM at 0° C. The reaction mixture was stirred for 30 minutes, and a solution of 76.4 mg triphosgene (0.26 mmol) in 10 ml DCM was added drop-wise in an ice water bath. The resulting reaction mixture was stirred for 30 minutes at 0° C., and diluted with 70 ml DCM. The solution was washed once with 150 ml 1N HCl, and twice with 50 ml of water, and dried over anhydrous MgSO₄. The solution was concentrated in vacuo to about 50 ml, and poured into anhydrous ethyl ether placed in a Teflon cylinder to precipitate the product. The solid was washed with anhydrous ethyl ether and dried in a vacuum oven at 40° C. overnight to obtain 12.2 g of the product. MW=112,000; PDI=1.53

### Example 49

### Preparation of Alternating Block Thermoplastic Elastomeric Polyanhydride (Compound 40)

A solution of 0.921 g of Compound 25c (Mₙ=96,000) and 3.2 μl TEA in 19 ml anhydrous chloroform was slowly added to a solution of 7.29 mg Compound 15d in 20 ml anhydrous chloroform. The mixture was stirred for 30 minutes, and then was slowly added to a solution of 1.2 g of Compound 26b and 6.4 μl anhydrous TEA in 20 ml anhydrous chloroform. The reaction mixture was stirred at room temperature for 17 hours, diluted with 20 ml chloroform, washed with 25 ml 1N HCl and then with 25 ml distilled water, and was dried over anhydrous magnesium sulfate. The dried solution was concentrated in vacuo to about 10 ml, and poured into anhydrous ethyl ether in Teflon cylinder to precipitate the polymer. The solid was washed with ethyl ether and dried in the vacuum oven at 40° C. overnight to obtain 1.8 g of the product. MW=167,000; PDI=1.27

### Example 50

### Preparation of a Triblock Thermoplastic Elastomeric Polyanhydride (Compound 42)

A solution of 2.849 g Compound 25c (Mn=30,000; 0.095 mmol) and 0.0477 ml anhydrous TEA (0.34 mmol) in 30 ml anhydrous chloroform was added drop-wise to a solution of 219.3 mg Compound 15 g (0.31 mmol) in 15 ml anhydrous chloroform. The reaction solution was stirred at 18° C. overnight, and then concentrated in vacuo. The residue was dissolved in 1 ml DCM, and anhydrous ethyl ether was added to precipitate a crude diacid chloride (Compound 41). The supernatant was decanted, the dissolution/precipitation process was repeated three times, and the solid was finally washed with ethyl ether, and dried in a vacuum oven at 40° C. for 5 hours to obtain Compound 41. A solution of the dried solid (Compound 41) in 25 ml anhydrous chloroform was addedd drop-wise to a solution of 12.008 g Compound 26b (Mₙ=67,000; 0.18 mmol) in 45 ml anhydrous chloroform at 18° C., and the solution was stirred overnight at room temperature. The reaction mixture was then washed once with 75 ml 1N HCl and twice with 50 ml water twice, and was dried over anhydrous sodium sulfate. The solution was concentrated to 30 ml, and dripped into 880 ml anhydrous ethyl ether placed in a Teflon beaker to precipitate the crude product. The crude product was washed with ether and dried in a vacuum oven at 40° C. overnight to produce 12.5 g of the product. Yield: 84%; MW=129,000; PDI=1.693. The Structure was confirmed by NMR.

### Example 51

### Preparation of Polymer Microspheres

One gram of polymer was dissolved in 5 ml DCM, and 0-500 mg of a drug was added to the solution. The mixture was mixed thoroughly and poured into 1.0-2.5% aqueous solution of PVA while agitating at 3,000-5,000 rmp. The mixture was agitated for 1 hour, magnetically stirred for 2 hours, centrifuged, washed with water several times, and lyophilized to obtain microspheres.

### Example 52

### Content Uniformity Determination of Drug (Methotrexate) Admixed with Polymer in Microspheres

The weight per weight percent (wt/wt %) loading of methotrexate with various polymer polyanhydrides was determined by a liquid-liquid extraction procedure. 5-10 mg polymer were weighed and dissolved with 3 ml ethyl acetate. The methotrexate was then extracted from the ethyl acetate layer into 5 ml of an aqueous phosphate buffer saline (PBS) layer. A 0.3 ml aliquot was removed and was filtered with a 0.45 μm filter into an HPLC vial with a 300 μl insert. The methotrexate response and extraction efficiency were tested by extracting methotrexate-free microspheres and adding between 50 μg and 200 μg of methotrexate into the polymer extract and filtering as above. The HPLC procedure used a Rapid Resolution RP-1, 0.1 v/v % TFA in aqueous as mobile phase A and 0.1 v/v % TFA in acetonitrile as mobile phase B at a 1.0 ml/minute flow rate. The compositions of the microspheres prepared are presented in Table 15 below.

TABLE 15
Drug Compositions in Microspheres

| | | | Drug Content |
|---|---|---|---|
| COMPOUND | POLYMER NO. | Drug | (% wt/wt) |
| 43 | 26b | N/A | N/A |
| 44 | 26b | BPC | 26.5^{α} |
| 45 | 26b | BPC | 14.8^{α} |
| 46 | 26b | MTX | 10^{β} |
| 47 | 26b | MTX | 16^{β} |
| 48 | 26a | MTX | 13^{β} |
| 49 | 32a | MTX | 10^{β} |
| 50 | 25a | MTX | 16^{β} |

^{α}Measured by ¹H NMR
^{β}Measured by the method of Example 49

### Example 53

### Determination of Elution Profile of Methotrexate-Loaded Microspheres

The in vitro release of drug (methotrexate) present in microspheres was determined by kinetic elution. The calculated level of w/w % loading described in Example 51 above was used to calculate the expected % release for about 10 mg methotrexate-loaded microspheres. Aliquots of approximately 10 mg microspheres prepared as in Example 51 above were weighed and placed in 50 ml conical tubes provided with screw cap closures. A 40 ml aliquot of release media, either PBS or serum, was added to each tube with a pipet, and the tubes were capped and placed in a 37° C. incubator chamber for periodic sampling. The test tubes were removed for sampling, centrifuged for 5 minutes and 1 ml samples of either PBS or serum were withdrawn for analysis. The samples were initially withdrawn at 1 hour intervals, then daily until changes were noted in either the presence of polymer microspheres or color of the medium. The samples were removed from the PBS, filtered, and typically injected directly or diluted 10 times (100 μl to 900 μl) with PBS. Any samples removed from serum were extracted with a common solid phase extraction (SPE) procedure, and analyzed by high pressure liquid chromatography (HPLC). The HPLC method was also used for determining content uniformity. The results obtained are shown in Table 16 below.

TABLE 16
MTX-DF Microspheres' Elution Profile

| Elapsed Time | Cumulative % MTX | | |
|---|---|---|---|
| (Days) | Compound 48 | Compound 49 | Compound 50 |
| 0 | 0.00 | 0.00 | 0 |
| 1.00 | 18.80 | 34.40 | 13.09 |
| 2.0 | 48.26 | 52.45 | 37.83 |
| 3.0 | 52.14 | 85.88 | 40.59 |
| 7.0 | 52.68 | 85.81 | 46.77 |
| 10.0 | ND | ND | 45.89 |
| 14.0 | ND | ND | 55.50 |

ND not determined

### Example 54

### Biodegradation of Polymer-Coated Coupons

Metal coupons were labeled, cleaned, and air-dried for about 15 minutes. 100 mg polymer were prepared in 400 mg anhydrous DCM and vortexed, and the coupons were coated with 150 μm gap width from an air pressured spray nozzle, air dried for 2 hours, and placed in a vacuum oven at 50° C. for 4 hours. The thickness and mass of the coatings were measured, and the following results were obtained. Typical results obtained are shown in Table 17 below.

TABLE 17
Polymer Coated Coupons

| Coupon | Total Mass(mg) | Coating Mass (mg) | Thickness (μm) |
|---|---|---|---|
| A | 4851.0 | 36.8 | 24.8 ± 17.2 |
| B | 4932.1 | 23.6 | 18.8 ± 4.2 |

All coupons were immersed in a phosphate buffered saline medium (pH=7.4) and incubated at 37° C. The release of the drug (diflunisal) was evaluated by periodic sampling of the medium and quantitation by high pressure liquid chromatography (HPLC) as described above. The data are shown in Table 18 below.

TABLE 18
Polymer Elution* Profiles

| Time Elapsed | Cumulative % SA | Cumulative % DF |
|---|---|---|
| (Days) | (Compound 26a) | (Compound 26c) |
| 0 | 0.00 | 0.00 |
| 1.0 | 0.00 | 0.00 |
| 2.0 | 0.94 | 2.02 |
| 3.0 | 2.96 | 5.00 |
| 5.0 | 33.40 | 11.68 |
| 8.0 | 56.74 | 44.07 |
| 13.0 | 99.31 | 68.60 |
| 15.0 | 99.73 | 73.50 |
| 21.0 | 100.15 | 74.30 |
| 27.0 | 101.69 | 76.94 |
| 31.0 | 101.69 | 79.31 |
| 36.0 | 100.51 | 81.75 |

*Eluted from Unsterilized Coupons of Polymers 26a and 26c in PBS at 37° C..

### Example 55

### Effect of Sterilization Method and Measurement of Polymer Degradation

The degradation of polymers from coupons, with and without E-beam sterilization, with a 3 μm thick coating was measured in PBS (pH=7.4) at 37° C. The results are shown in Table 19, below.

TABLE 19
Polymer Elution Profile with/without E-Beam Sterilization

| Time | Compound 26a (1.7 mg) | | Compound 26c (3.4 mg) | | Compound 28a (3.6 mg) | |
|---|---|---|---|---|---|---|
| Elapsed | Cum. % SA | Cum. % SA | Cum. % DF | Cum. % DF | Cum. % DF | Cum. % DF |
| (days) | No E-Beam | E-Beam | No E-Beam | E-Beam | No E-Beam | E-Beam |
| 0 | 0.00 | 0.00 | 0.00 | 0.00 | 0.00 | 0.00 |
| 1.0 | 0.00 | 0.00 | 0.00 | 0.00 | 0.21 | 0.00 |
| 2.0 | 0.85 | 3.88 | 2.19 | 2.94 | 0.21 | 0.00 |
| 3.0 | 4.61 | 12.60 | 13.59 | 15.46 | 0.31 | 0.45 |
| 4.0 | 17.10 | 23.74 | 21.85 | 25.19 | 1.08 | 2.93 |
| 5.0 | 27.39 | 32.27 | 32.29 | 35.83 | 3.41 | 8.10 |
| 6.0 | 45.73 | 46.10 | 54.72 | 53.92 | 9.39 | 18.28 |
| 9.0 | 65.51 | 64.90 | 79.20 | 81.37 | 67.26 | 87.98 |
| 18.0 | 69.88 | 65.59 | 88.99 | 92.72 | 97.49 | 87.98 |
| 21.0 | 74.38 | 72.09 | 92.84 | 98.20 | 98.16 | 76.60 |
| 30.0 | ND | ND | 97.13 | 98.20 | 99.95 | 77.27 |
| 44.0 | ND | ND | 98.49 | 98.41 | 102.33 | 75.76 |

ND not determined

As shown in Table 18 above, E-Beam sterilization (3.5 mRad) had substantially no effect on the pattern of diflunisal released from the polymer-(polyDF- or poly SA) coated stainless steel samples incubated in serum at 37° C. Notwithstanding the lack of effect on polymer degradation, sterilization may produce some changes in the molecular weight and mechanical properties of a polymer. For example, the tensile modulus of a melt-polymerized salicylic acid polymer (polySA) decreased by about ⅓ after gamma sterilization (25-35 Kgys) at room temperature although no change occurred when irradiated at 37° C. Gamma radiation had no effect on the molecular weight, flexibility, or adhesiveness of polySA, and only a very minor effect on its hardness. The effects of gamma radiation and E-beam sterilization on polyDF were similar to those observed with polySA.

### Example 56

### Biodegradation of Polymers Containing Admixed Drug (Paclitaxel)

Paclitaxel (PAC) was admixed in a solution of polymer at a concentration of 0 to about 40 wt %, i.e., 1 mg of polymer-drug admixture contained 0.8 mg polymer and 0.2 mg drug. Paclitaxel was released at the same rate at which the polymer biodegraded to generate diflunisal (the relatively small amount of paclitaxel released reflects the inability of serum to hold this relatively insoluble drug). Tables 20a, 20b, 20c and 20d below show the concurrent release of paclitaxel from a polydiflunisal (polyDF)-paclitaxel admixture coated onto electro-polished stainless steel samples and incubated in serum or PBS at 37° C.

TABLE 20a
Elution of PAC admixed into Diflunisal (DF) Polymer

| | 0% PAC | 5% PAC | 40% PAC |
|---|---|---|---|
| Time Elapsed | Cumulative % DF | Cumulative % DF | Cumulative % DF |
| (days) | Generated | Generated | Generated |
| 0 | 0.0 | 0.0 | 0.0 |
| 1.0 | 13.1 | 8.7 | 11.6 |
| 2.0 | 38.3 | 42.1 | 38.3 |
| 3.0 | 40.5 | 50.1 | 47.3 |
| 4.0 | 44.4 | 53.6 | 57.7 |
| 5.0 | 48.4 | 58.0 | 61.7 |
| 6.0 | 50.4 | 61.0 | 69.0 |
| 7.0 | 52.8 | 64.0 | 74.0 |
| 10.0 | 57.4 | 69.4 | 80.8 |
| 12.0 | 60.6 | 78.8 | 95.1 |
| 14.0 | 67.8 | 82.2 | 99.6 |
| 17.0 | 76.6 | 93.4 | 110.1 |
| 19.0 | 79.5 | 96.9 | 112.2 |
| 21.0 | 82.0 | 100.6 | 115.0 |
| 28.0 | 88.9 | 110.0 | 122.0 |

*Elution of <5 μm Coating of Compound 27a on 1 cm² Coupons in Serum with 0%, 5% and 40% PAC Loading.

TABLE 20b
Elution* of PAC admixed into Polymer

PAC Released (Cumulative μg)

| Time Elapsed | Compound 26b | Compound 27a |
|---|---|---|
| (days) | With 10% PAC | with 5% PAC |
| 0 | 0.0 | 0.0 |
| 1.0 | 13.8 | 6.3 |
| 2.0 | 9.7 | 8.2 |
| 3.0 | 13.6 | 8.2 |
| 4.0 | 11.2 | 9.1 |
| 5.0 | 12.8 | 9.7 |
| 6.0 | 14.2 | 13.1 |
| 7.0 | 20.5 | 17.3 |
| 10.0 | 21.2 | 21.7 |
| 12.0 | 31.5 | 21.9 |
| 14.0 | 33.4 | 25.2 |
| 17.0 | 41.7 | 27.4 |
| 19.0 | 41.6 | 27.4 |
| 21.0 | 50.9 | 34.4 |
| 28.0 | 42.0 | 40.4 |

*Elution of Paclitaxel from <5 μm Coating of Compound 26b and Compound 27a on 1 cm² Coupons in Serum

TABLE 20c
Elution of Paclitaxel

PAC Released* (Cumulative %)

| Time Elapsed | Compound 27a | Compound 27a |
|---|---|---|
| (days) | with 0% PAC | with 40% PAC |
| 0 | 0.00 | 0.00 |
| 1.0 | 5.79 | 12.16 |
| 2.0 | 7.56 | 11.22 |
| 3.0 | 7.56 | 10.56 |
| 4.0 | 8.38 | 9.67 |
| 5.0 | 8.96 | 17.02 |
| 6.0 | 12.11 | 17.24 |
| 7.0 | 16.05 | 19.19 |
| 10.0 | 20.05 | 19.69 |
| 12.0 | 20.23 | 22.59 |
| 14.0 | 23.34 | 23.05 |
| 17.0 | 25.37 | 27.84 |
| 19.0 | 25.37 | 28.19 |

*Elution of Paclitaxel from <5 μm Compound 27a Coating on 1 cm² Coupons in Serum

TABLE 20d
Elution of PAC Admixed into Polymer*

PAC Released (Cumulative μg)

| Time Elapsed | Compound 26b | Compound 26b |
|---|---|---|
| (days) | With 0% PAC | with 10% PAC |
| 0 | 0 | 0 |
| 1.0 | 59.75 | 38.5 |
| 2.0 | 140.39 | 76.42 |
| 3.0 | 199.18 | 136.22 |
| 4.0 | 307.39 | 174.94 |
| 5.0 | 268.49 | 282.67 |
| 6.0 | 410.89 | 333.27 |
| 7.0 | 485.14 | 407.52 |
| 10.0 | 617.14 | 539.52 |
| 12.0 | 661.33 | 752.02 |
| 14.0 | 671.89 | 779.52 |
| 17.0 | 785.64 | 955.77 |
| 19.0 | 807.97 | 965.04 |
| 21.0 | 842.47 | 1001.29 |
| 28.0 | 837.73 | 985.2 |

*Elution from <5 μm Coating of Compound 26b with 0% and 10% PAC Loadings on 1 cm² Coupons in PBS

### Example 57

### Determination of Polymer Glass Transition Temperature (T_{g}) in a Differential Scanning Calorimeter

Approximately 10 mg polymer were accurately weighed, and the mass was recorded in a pre-tared aluminum pan (no-hermetic seal). The pan was crimped to complete a seal and to facilitate good heat transfer. The pan was placed in the calorimeter opposite an empty reference pan of mass similar to the sample pan. The calorimeter was closed and sealed in a nitrogen sweep gas atmosphere. The sample temperature was controlled at a program rate of 10° C./min from room temperature to −20° C., followed by heating to 110° C. The sample was then cooled to −20° C., and was heated at the same rate a second time to 110° C. The T_{g} was observed as the mid-point in the heat capacity inflection. The measurements were made using Thermal Analytical Instruments Q-100 with a circulation bath chiller. The data obtained from each of ten polymers are shown below in Table 21.

TABLE 21
Glass Transition Temperatures

| | Compound | | | | | | | | | | |
|---|---|---|---|---|---|---|---|---|---|---|---|
| | 23b | 23b | 23b | 23b | 23b | 23b | 23b | 23b | 23b | 23b | 23b |
| T_{g} (° C.) | 40 | 40 | 40 | 40 | 40 | 40 | 40 | 40 | 40 | 40 | 40 |

### Example 58

### NMR Analyses of Different Bond Types

The polymerization of a Diflunisal-Linker-Diflunisal was conducted to demonstrate the ability of the process of the invention to control bond types and bond type distribution. The bond type was determined by NMR and the results are shown in FIG. 2 accompanying this patent. Melt polymerization produced a distribution labeled as “Dispersion” in the polymer type axes. This polymer released 70% of the contained Diflunisal in approximately 28 days with a more gradual release for another 14+days. By applying the synthetic methods presented here, a polymer was created with the same % of ingredients but with only one bond type (“Controlled Sequence”). By creating a strictly alternating repeat structure, only one bond type predominates. This polymer will release more rapidly, and will form crystals in the later stage of elution. By altering the rate of addition of phosgene or by changing the pre-polymer, other precise distributions and sequences maybe created which result in changes in crystallinity, release kinetics (hours to months from a 5 micron thick coating) and other physical attributes such as compatibility & solubility. “Random” represents a polymer in which the polymerization reaction is allowed to take place in once step. By slowing down the addition rate of phosgene, additional distributions of bond types were achieved (“Random—1 hour Addition” and “Random—6 hour Addition”). As the % of weaker bonds was altered, the breakdown rate, stability, sterilization breakdown, etc. was altered as well but in a controlled manner. Thus, the design capabilities of these polymers are far beyond those of typical melt polymerization or solution polymerization prior to this art.

### Example 59

### Effect of Linker Chain Length on Glass Transition Temperature and Mechanical Properties

A polymer's glass transition temperature (T_{g}) is a key parameter that significantly influences its mechanical, physical chemical and handling properties. The molecular weight and chemical composition of the linking group may affect the polymer's glass transition temperature (T_{g}), and accordingly, the mechanical properties of the therapeutic polymers and coatings of the therapeutic polymers at body temperatures. The higher the molecular weight, the greater the toughness of the material in terms of elasticity and tear strength. A polymer's tensile modulus may be taken as an index of the polymer's rigidity. The glass transition temperatures and tensile moduli for several polymers are listed in Table 22 and Table 23 below.

TABLE 22
Aliphatic Linker: Chain Length and Tensile Modulus Effect*

| | | Carbon Atom Number (Linker) | | | |
|---|---|---|---|---|---|
| | | 6 | 6:8 | 8 | 10 |
| Glass Transition Temp (Tg, ° C.) | | 44 | 38 | 29 | 16 |
| Tensile Modulus (kPa) | 25° C. | 3300 | 2100 | 140 | 7 |
| | 37° C. | 480 | 45 | 4 | NO |

*Polymer prepared by Solution Process.
NO Not observed

TABLE 23
Tg Versus Linker of polySA and polyDF

| | |
|---|---|
| Carbon Atom Number | Tg (° C.) |

| (Linker) | PolyAspirin I | PolyAspirin II |
|---|---|---|
| 6 | 46 | 76 |
| 8 | 30 | — |
| 10 | 19 | 54 |
| 12 | 6 | 48 |
| 14 | — | 38 |
| 16 | — | 12 |

Table 22 shows a salicylic acid polymer with a C₆ linker molecule as having a T_{g}=44° C., and that the polymer is relatively hard at room temperature. Increasing the carbon-chain length will generally lower the glass transition temperature (T_{g}) of the resulting polymer in a somewhat linear manner, so that a polymer of salicylic and (polySA) produced with a C₁₂ linker molecule has a T_{g}=8° C. and is a rubbery, elastic material at room temperature. A similar profile is seen with polymers of diflunisal (polyDF), a potent derivative of salicylic acid. For a specific linker chain length, a polydiflunisal will generally exhibit a much higher T_{g} when compared to the same linker in the corresponding polysalicylic acid (Table 23). Thus, the data provided in Table 22 show that in one embodiment of the invention, the tensile modulus (polymer rigidity) and glass transition temperature are inversely proportional to linker chain length. In addition, for each specific linker, the polymer's rigidity decreased with increased temperature from 25° C. to body temperature (37° C.). Table 24 below shows data for another embodiment of the invention. In this embodiment, the polymer's T_{g} increases with increasing chain carbon number, e.g., glutaric acid vs. adipic acid. A linker of a very short carbon chain, for instance, less than about C₅, provides a lesser chance for cross-linking by generally known synthetic methods, e.g., melt polymerization variations, probably due to steric hindrance, and the polymer products may have a lower T_{g}, e.g., about 58° C., than with a longer chain linker that may favor more extensive cross-linking and, therefore, higher T_{g}, e.g. about 76° C. This potential cross-linking reactivity generally decreases as the molecular weight of the polymer increases and as the linker chain length increases sufficiently. Dodecanedioic acid, for example, has a T_{g} of about 53° C.

TABLE 24
Aliphatic Linker in PolySalicylic Acid (Anhydride Ester)

| Polymers*/Linkers | MW | PDI | T_{g} (° C.) | Tₘ (° C.) | T_{d} (° C.) |
|---|---|---|---|---|---|
| Glutaric acid | 3,206 | 1.1 | 58 | 175 | 424 |
| Adipic acid | 2,221 | 1.7 | 76 | N.C. | 391 |
| Dodecanedioic acid | 18,427 | 1.8 | 53 | 178 | 434 |
| Diglycolic acid | 3,051 | 1.0 | 68 | N.C. | 408 |

N.C. Not observed.
*Synthesized at 180° C. for 2.5 hr under vacuum

TABLE 25
Aromatic Linker in Salicylic Acid (Anhydride Ester) Polymer*

| Polymers/Linkers | MW | PDI | T_{g} (° C.) | Tₘ (° C.) | T_{d} (° C.) |
|---|---|---|---|---|---|
| Terephthalic | 2,101 | 1.3 | 111 | N.O. | 436 |
| 1-4′-Phenyldiacetic | 1,584 | 1.0 | 89 | N.O. | 386 |
| 4-4′-Biphelyldicarboxylic | 5,531 | 1.1 | 150 | N.O. | 463 |
| 4-4′-Oxybisphenyldicarboxylic | 9,064 | 1.1 | 103 | N.O. | 387 |
| 4-4′-(Hexafluoroisoporpylidene) dicarboxylic | 9,436 | 1.2 | 149 | 315 | 464 |

*Synthesized at 180° C. for 2.5 hr under Vacuum
N.O. Not observed

In another embodiment of this invention, the linkers are aromatic molecules that have different structural rigidity (Table 25). Table 25 provides information that corresponds to a salicylic acid polymer having an aromatic linker, where the introduction in the polymer chain of aromatic linkers of different characteristics, such as structural rigidity, results in different T_{g} values. The data provided in the previous tables show that the transition temperature T_{g} may vary with the number of carbons of a straight aliphatic chain linker as well as with other parameters of the linker molecule such as, but not limited to, hydrophobicity, structural rigidity, presence of heteroatoms, etc. The polymers of the invention evidence an extraordinary range of properties that may be varied as required by any one specific application, as exemplified in Tables 23, 24 and 25. These data also show that a great variety of polymers, e.g. poly-NSAIDs as well as polymers of other types of molecules, may be created from combinations of a monomer(s) and different linkers that may have varied chain lengths and chemical structures, to attain a polymer of pre-determined physical properties, e.g. in-between those of the respective homologous polymers. In addition, the process of the invention also allows the formation of polymers of desired characteristics by a combination of molecules with certain linkers in pre-selected proportions to obtain desired values for the polymer characteristics. For example, a co-polymer made from equal amounts of a monomer attached to C₆ and C₈ linkers should have an intermediate T_{g} and tensile modulus with respect to those of the C₆ and C₈ polymers. In addition, different molecules may be introduced into the polymer to obtain a compound of combined activities. For example, NSAIDs of the type of salicylic acid, diflunisal, salsalate (a di-salicylic acid), analgesics, hemostatics, antibiotics, etc., and in general any polymerizable molecule may be employed, examples of which are given herein. This flexibility in the design of a polymer extends to the synthesis of all polymers of the invention, e.g. polymers of salicylic acid, diflunisal, salsalate, etc., and thereby allows the control of polymer properties by varying monomer ratio, e.g. 20:80, 50:50, 80:20, linker combinations, linker structure, molecules in the form of monomers, dimers, trimers, tetramers, etc. combinations of molecules, and others. Varying the linker chain length may have an inverse influence on the polymer's shore hardness. When measured by ASTM methods, the relative hardness of the polymer of the invention, e.g., with polyNSAIDs such as poly-salicylic acid and poly-diflunisal, was seen to decrease with increasing linker chain length. That is, shorter linkers produced harder polymers compared to longer linkers. As the carbon number in the linker chain was increased, the polymers also became slightly softer when hydrated. When the data are normalized to the intended use temperature (T-T_{g}) a roughly linear relationship for all polymers is observed, thereby providing a powerful tool for designing polymers of pre-selected characteristics.

### Example 61

### Preparation of Poly 1,8-bis(o-Dicarboxyphenyl)Octanoate Homopolymer by Melt Polymerization (Compound 23c)

The diacid (compound 14b) was activated into monomer using previously described methods. See, Campo et al., Polym. Bull.: 42-61 (1999); Anastasiou and Ulhrich, Macromolecules 33: 6217 (2000). The diacid was added to an excess of acetic anhydride (100 ml), and then stirred at reflux temperature until the appearance of a homogenous solution (approximately 120 min). The monomer (compound 22c) was isolated by removing excess acetic anhydride under vacuum, and was then washed with diethyl ether (50 ml). Monomer (500 mg) was placed in an appropriate reaction vessel, and heated to 180° C. using a temperature controller (Cole Parmer) in a silicone oil bath under high vacuum (<2 mmHg) for 1 to 3 hours. During this time, the melt was actively stirred at about 100 rpm by the overhead stirrer (T-line Laboratory Stirrer, Talboys Engineering). Polymerization was complete when the viscosity of the melt remained constant and/or solidified. The polymer (compound 23c) was cooled to room temperature, dissolved in a minimal volume of methylene chloride (15 ml), and precipitated into a 20-fold excess of diethyl ether (300 ml).

### Example 62

### Preparation of 1,6-Bis(o-Carboxyphenoxy) Hexane Dicarboxylic Acid (Compound 51a)

To a mixture of salicylic acid (77.12 g, 0.5580 mole) and distilled water (84 mL), sodium hydroxide (44.71 g, 1.120 mole) was added, the reaction was brought to reflux temperature, and then 1,6-dibromohexane (45.21 g, 0.2790 mole) was added drop-wise. Reflux was continued for 23 hours, and then additional sodium hydroxide (11.17 g, 0.2790 mole) was added, and the mixture refluxed for an additional 16 hours, cooled, filtered, and washed with methanol. Yield=48.8%.

### Example 63

### Preparation of 1,6-Bis(o-Carboxyphenoxy) Hexane Monomer (o-CPH) (Compound 52a)

The dicarboxylic acid of Example 2 was acetylated in an excess of acidic anhydride at reflux temperature. The resulting monomer was precipitated with methylene chloride into an excess of diethyl ether. Yield=66.8%.

### Example 64

### Preparation of Poly[(1,8-bis(o-Dicarboxyphenyl) Octanoate)(1,6-bis(p-Carboxyphenoxy) Hexane]Co-polymers (Compound 53a)

1,8-bis(o-dicarboxyphenyl)octane was copolymerized with 1,6-bis(p-carboxyphenoxy)hexane as described in Example 3 of WO01/41753. Briefly, polymerization was in a melt condensation performed at 180° C. for 3 hours under vacuum in a reaction vessel with a side arm. The polymerization vessel was flushed with nitrogen gas at frequent intervals, and the polymer was isolated by precipitation into diethyl ether from methylene chloride. Yield was quantitative.

### Example 65

### Bone Formation and Resorption Inhibition by Salicylic Acid-derived Poly(Anhydride Esters) in Long Bone Critical Size Defect Model

This study evaluates the effect of polymers that release salicylic acid on bone growth when implanted into the appendicular skeleton using two compositions of the polymer (compound 53a and Compound 23c). Nineteen (19) Sprague Dawley retired breeder male rats were employed in this experiment. In each animal, a 5 mm mid-shaft defect was created using an oscillating saw. The limbs were then stabilized using a custom 4-hole polymer plate and screws. In two groups of 6 rats each, the defects were filled with either the homopolymer or the copolymer. The remaining 7 rats had their defects filled with a collagen sponge to serve as controls. An equal number of animals from each group was sacrificed at 4 and 8 weeks post surgery. The only exception was that four control rats were used in the 8-week group. The defect sites were radiographed using a Hewlett-Packard Faxitron model 43804 and high resolution mammography film every 2 weeks until sacrifice. At term, the limbs were collected and prepared for non-decalcified histology. Mid-sagittal ground sections were prepared and stained using Sanderson's Rapid Bone Stain and Stevenel's Blue. New bone formed between inner-most fixation screws was measured on 2 slides per animal and averaged. Bone resorption, or loss, was also measured by approximating the original contours of the cortices, and measuring the difference in area relative to the remaining bone.

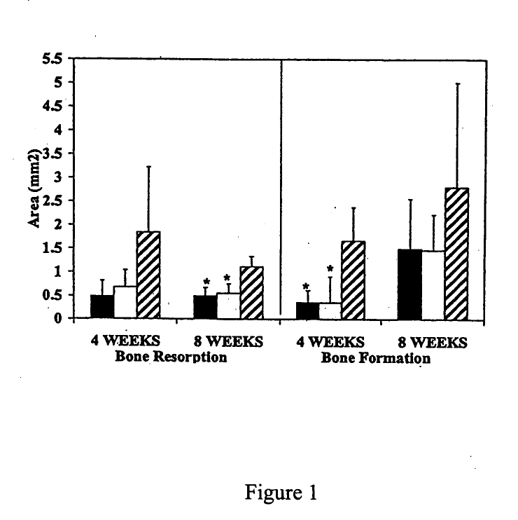

No radiographic evidence of healing the defect was observed in any of the animals studied. In fact, there appeared to be less new bone formed in X-rays of the polymer-implanted animals. This was confirmed by the histological results. There was less new bone formed, as well as less bone resorption, in the polymer groups. The histo-morphometric data showed there was less new bone formed in both of the polymer groups than in the collagen group (FIG. 1). The difference in bone formation between both of the two polymer groups and the collagen group was found to be statistically significant at 4 weeks, but did not reach the level of statistical significance at 8 weeks. The data also showed there was less bone resorption in both the homo-polymer and co-polymer groups than in the collagen group at both 4 weeks and 8 weeks. Although the magnitude of the difference had decreased from 4 weeks to 8 weeks, the difference at 8 weeks was statistically significant while that at 4 weeks was not. The gross appearance of the implant sites at harvest supported the histological data. Even at 8 weeks after surgery the bone ends in the polymer groups looked as though they were freshly cut, the cut edges of the bones were very sharp, and no appreciable bone formation was seen in the immediate vicinity of the defect. This result was in marked contrast to what was observed in the animals that received the collagen implant, in which the bone ends appeared rounded and irregular with varying degrees of callus formation. In all, the polyanhydrides of this invention caused the bone tissue at the defect to maintain the status quo following initial surgery. These results demonstrate that the salicylate-releasing polymers significantly affect bone cell activity in long bones. This effect is likely a result of the released salicylic acid inhibiting the synthesis of prostaglandins (PGs). It is known that increased PG synthesis during inflammation leads to increased bone resorption. The amount of salicylate delivered in this experiment was very different from that delivered in the study of Example 9 of WO 01/41753 where the salicylate promoted new bone formation. In WO 01/41753, thin films were placed on the palate bone and had nominal dimensions of 0.5 mm×0.3 mm×0.3 mm, yielding an approximate volume of 0.045 mm³. In this experiment polymer microspheres were packed together to form a semi-solid cylindrical pellet that was roughly 4 mm in diameter by 5 mm in length, yielding an approximate volume of 63 mm³, over 1400 times greater. This experiment was performed on long bone, which is formed by endochondral bone formation. Similar results are expected with other types of bones, such as those originating by intra-membranous bone formation, such as the palate, skull, and jaw.

### Example 66

### Salicylic Acid Polymer Inhibition of Bone Loss in Dogs

A polymer wafer was tested in dog for their effect on inhibition of bone growth. A wafer of polyNSAID was made with a polyanhydride (749PL, M_{w}=20,000, PDI=2.4) in 4 mm diameter and 0.4 mm thickness. Each wafer contained 5 mg of salicylic acid (by equivalent). A wafer of placebo polymer was made with polyanhydride of ortho-carboxyphenoxyhexane (o-CPH)ₙ (325PL, M_{w}=20,000, PDI=2.6). Twelve 6-7 year old beagles with moderate to severe chronic periodontitis were studies for 3 months. The dogs randomly received a 0.4 mm salicylic acid polymer wafer on one side at the teeth (test side), and a 0.4 mm placebo polymer wafer on the opposite side (placebo side). Silk ligatures were tied onto the teeth being studied for the first 6 weeks to exacerbate periodontal destruction. Clinical data on probing depth, attachment levels, gingival and plaque indices were collected at baseline, at 6 weeks (1½ months) and at 12 weeks (3 months). Standardized intra-oral radiographs were taken utilizing custom stents at baseline and at 12 weeks. An ANOVA t-test was used for comparing the test and placebo sides.

The results observed were as follows. The primary outcome was alveolar bone loss. The change in alveolar bone height from baseline to the final time point was calculated by using the initial bone level as a covariate and taking into account the differences between test (Salicylic Acid Polymer) and placebo sides at baseline. The results indicated a statistically significant difference (p=0.02) between the test side wafer (−0.42±0.09 mm) and placebo wafer (−0.89±0.22 mm). The salicylic acid polymer had no effect on the clinical parameters. Taking the difference between initial (baseline) and final (12 weeks) bone height as a measure of differential bone size at the two time points it was observed that while the bone in untreated side had a reduction of height of 0.89±0.09 mm, the test side (Polysalicylic acid) only had a reduction in bone height of 0.42±0.09 mm. Thus, the salicylic acid polymer reduced the bone loss progression by greater than one half. And it did so in a statistically significant manner. This shows that the local sustained delivery of salicylic acid is beneficial in the management of bone destruction associated with periodontitis. It is possible that salicylic acid may exert its retardatory effect on bone loss by inhibition of the local production of arachidonic acid metabolites and/or microbial infection. A sustained release of salicylic acid (or other non-steroidal anti-inflammatories) within a periodontal pocket may suffice to alter the progression of bone loss observed in its absence. And this drug appears to do this without the risk of side effects observed with the use of long term non-steroidal anti-inflammatory drugs. The in situ administration of a salicylic acid polymer clearly had an inhibitory effect of alveolar bone loss in naturally occurring periodontitis.

All patents, publications and patent applications listed herein are incorporated by reference in their entirety, as though individually incorporated by reference. The invention has been described with reference to various embodiments and techniques. However, it should be understood that many variations and modifications may be made while remaining within the spirit and scope of the invention.

### REFERENCES

Erdmann, L., and Uhrich, K. E., Biomaterials 21: 1941-1946 (2000).
“Polymer Painkiller,” Science 278: 32-33 (1999).
Erdmann, L., Macedo, B. and Uhrich, K. E., Biomaterials 21: 2507-2512 (2000).
Morrow, J. D. and Roberts, L. J. “Lipid-Derived Autacoids: Eicosanoids and Platelet-Activating Factor” in Goodman and Gilman's The Pharmacological Basis of Therapeutics, 10th Edition, J. G. Hardman, L. E. Limbird and A. G. Goodman, eds., McGraw-Hill, New York, N.Y., pp 669-686 (2001).
Herman, J. H., Sowder, W. G., and Hess, E. V., J. Rheumatol. 21: 338-343 (1994).
Soekanto, A., Ohya, K., and Ogura, H., Calcified Tissue Internat. 54(4): 290-295 (1994).
Soekanto, A., Jap. J. Pharmacol. 65(1): 27-324 (1994).
Roberts, L. J., and Morrow, J. D., “Analgesic-Antipyretic and Antiinflammatory Agents and Drugs Employed in the Treatment of Gout” in Goodman and Gilman's The Pharmacological Basis of Therapeutics, 10th Edition, J. G. Hardman, L. E. Limbird, and A. G. Goodman, eds., McGraw-Hill, New York, N.Y., pp 687-732 (2001).
The Merck Index, 10th Edition, M. Windholz, ed., Merck & Co., Inc., Rahway, N.J., 1983, pp 123, 456, and 1200.
The Merck Index, 10th Edition, M. Windholtz, ed., Merck & Co., Inc., Rahway N.J., p 1200 (1983).
The United States Pharmacopeia (USP XXI)/The National Formulary (NF XVI), United States Pharmacopeial Convention, Inc., Rockville Md., p 1195 (1985).
Remington's Pharmaceutical Sciences, 17th Edition, A. Gennaro, Ed., Mack Publishing Co., Easton, Pa., p 785 (1985).
Stedman's Medical Dictionary, 27th Edition, M. B. Pugh, Ed., Lippincott Williams & Wilkins, PA, p 103 (2000).
Chambers, H. F. “Antimicrobial Agents: General Considerations” in Goodman and Gillman's The Pharmacological Basis of Therapeutics, 10th Edition, J. G. Hardman, L. E. Limbird, A. G. Goodman, Eds., McGraw-Hill, New York, N.Y., pp 1143-1170, 638-681 (2001).
The United States Pharmacopeia (USP XXV)/The National Formulary (NF XX), United States Pharmacopeial Convention, Inc., Rockville Md., Procedure GM201PMC.01 (2003).
Van de Belt, H., Neut, D., Schenk, W. et al., Acta Orethop. Scand. 72(6): 557-571 (2001).
Category IV Monograph: Antiseptic Skin Cleansers. Drugs Directorate, Health Canada, 11 September (1995).
Domb, A. J., and Langer, R., Solid-state and solution stability of poly(anhydrides) and poly(esters). Macromolecules, 22, 2117-2122 (1989).
Schierholz, J. M., and Beuth, J., Med. Dev. Tech. 11(2): 12-17 (2000).
D'Emanuele, A., Hill, J., Tamada, J., et al., Pharm. Res. 9: 1279-1283 (1992).
Dang, W., Daviau, T., Ying, P. et al., J. Controlled Release 42: 83-92 (1996).
Figure from Edelmann, E. R., and Rogers, C., A. J. Cardiol. 81(7A): 4E-6E (1998).
Van der Giessen, W. J., Lincoff, A. M., Schwartz, R. S. et al., Circulation 94: 1690-1697 (1996).
U.S. Pat. No. 6,153,252, “Process for Coating Stents” issued to Ethicon, Inc.
U.S. Pat. No. 6,358,556 B1, “Drug Release Stent Coating” issued to Boston Scientific Corp.
Holick, M. F., and Krane, S. M., “Introduction to Bone and Mineral Metabolism: Bone Structure and Metabolism,” in Harrison's Principles of Internal Medicine, 15th Ed., Braunwald, E., Fauci, A. S., Kasper, D. L. et al, Eds., McGraw-Hill Medical Publishing Division, New York, N.Y., 2001, pp 2192-2205.
Keila, S., Kelner, A., and Weinreb, M., J. Endocrinol., 168(1), 131-139, 2001.
Simon, A. M., Manigrasso, M. B., and O'Connor, J. P. J. Bone Min. Res. 17: 963-975 (2002).
Dziak, R., J. Periodont. 64: 407415 (1993).
Alexander, M., and Damoulis, P., The role of cytokines in the pathogenesis of periodontal disease, Current Opinions in Periodont. 1: 39-53 (1994).
Weibe, S., Hafezi, M., Sandhu, H., et al., Oral Disease 2: 167-180 (1996).
Harten, RD; Svach, DJ; Schmeltzer, R; and Uhrich, KE “Salicylic Acid-derived Poly(anhydride-esters) Inhibit Bone Formation In Vivo”, J. Biomed. Mater. Res.: Part A, accepted. (2003)
Harten, RD; Svach, DJ; Schmeltzer, R. and Uhrich, KE “Salicylic Acid-Derived Poly(Anhydride-Esters) Inhibit Bone Formation and Resorption in a Long Bone Critical Size Defect Model” Trans. Soc. Biomater. (2003).
Einhorn, T. A., Arthritis Res. Ther. 5: 5-7 (2003).
Persson, U., Persson, M., and Malchau, H., The economics of preventing revisions in total hip replacement, Acta Orthop. Scand. 70: 163-169 (1999).
Lipsky, P. E. “Rheumatoid Arthritis,” in Harrison's Principles of Internal Medicine, 15′ Ed., Baunwald, E., Fauci, A. S., Kasper, D. L. et al., Eds. McGraw-Hill Medical Publishing Division, New York N.Y., pp 1928-1937 (2001).
Goronzy, J. J., and Weyand, C. M., in Primer on the Rheumatic Diseases, 12^{th} Ed., Klippel, J. H., Crofford, L. J., Stone, J. H. and Weyand, C. M., Eds., Arthritis Foundation, Atlanta Ga., pp 209-217 (2001).
Lewis, C. Arthritis: “Timely Treatments for an Ageless Disease,” FDA Consumer Magazine, U.S. Food and Drug Administration (May-June 2000).
Matteson, E. L. “Rheumatoid Arthritis C. Treatment,” in Primer on the Rheumatic Diseases, 12^{th} Ed., Klippel, J. H., Crofford, L. J., Stone, J. H. and Weyand, C. M., Eds., Arthritis Foundation, Atlanta Ga., pp 225-232 (2001).
Ishikawa, K., Ohira, T., and Sakata, H., Effects of intraarticular injection of halopredone diacetate on the articular cartilage of rabbit knees: a comparison with methylprednisolone acetate, Toxicol. Appl. Pharmacol. 75: 423-436 (1894).
Stefanich, R. J. Intraarticular corticosteroids in treatment of osteoarthritis. Orthop. Rev. 32: 65-71 (1986).
Kongtawelert, P., Brooks, P., and Ghosh, P., J. Rheumatol. 16: 1454-1459 (1989).
Hochberg, M. C., Altman, R. D., Bradt, K. D., et al., Arthritis Rheum. 38: 1541-1546 (1995).
Horisawa, E., Hirota, T., Kawashima, Y. et al., Pharm. Res. 19: 403-410 (2002).
“Joint Injection/Aspiration”, Amer. College of Rheumatol. Fact Sheet @ www.rheumatology.org.
“Joint Injections” @ (www.mayoclinic.com.
“Arthritis,” American Academy of Orthopedic Surgeons @ www.orthoinfo.aaos.org
Gutstein, H. B. and Akil, H., “Opioid Analgesics,” in Goodman and Gilman's The Pharmacological Basis of Therapeutics, 10′ Edition, J. G. Hardman, L. E. Limbird, and A. G. Goodman, eds., McGraw-Hill, New York, N.Y., 2001, pp 569-620.
Stein, C. and Yassouridis A., Pain 71: 119-121 (1999).
Dionne, R. A., Lepinski, A. M., Gordon, S. M. et al., Clin. Pharmacol. Ther. 70: 66-73 (2001).
Likar, R., Koppert, W., Blatnig, H. et al., J. Pain Symptom Manage 21: 330-337 (2001).
Stein, A., Yassouridis, A., Szopko, C. et al., Pain 83: 525-532 (1999).
Chaubal, M., Drug Delivery Technology 2: 34-36 (2002).
NUTROPIN®, NUTROPIN AQ®, NUTROPIN DEPOT® Product Labeling, Genetech, Inc. Physician's Desk Ref. 57^{th} Ed.

The relevant portions of all publications, patents and patent publications cited in this patent are incorporated herein by reference. The invention now being fully described with reference to some preferred embodiments and examples, it will be apparent to one of ordinary skill in the art that many changes and modifications may made thereto without departing from the spirit or scope of the invention as set forth herein.

1-75. (canceled)

76. A method of inhibiting bone growth or bone resorption comprising administering to a site where inhibition of bone growth or resorption is desired an effective amount of at least one anti-inflammatory agent having at least two reactive functional groups, which agent is incorporated into a biodegradable monomer or into the backbone of a biodegradable oligomer or polymer.

77. The method of claim 76, wherein the site of administration comprises a bone injury, osteophytes, osteoclasts, alveolar bone destruction, endochondral bone formation, or intra-membranous ossification and the bone growth or bone resorption is inhibited by at least 25% as compared to the bone growth or bone resorption in the absence of the anti-inflammatory agent.

78. The method of claim 76, wherein the biodegradable oligomer or polymer is selected from the group consisting of a repeating unit of Formula IV and Formula VI
—R¹-A-L-A- (IV)
—R²-A-L-A-R³-A-L-A- (VI)
wherein each R¹, R², and R³, independently from one another, comprises a residue that releases at least one anti-inflammatory agent upon biodegradation of an oligomer or polymer; each A, independently from one another, comprises an anhydride, ester, thioester, amide, thioamide, urethane, carbamate or carbonate, each L comprises a linking group whose reactive functional group forms a part of a and wherein a reactive functional group of R¹, R² or R³ forms a part of A.

79. The method of claim 78, wherein the anti-inflammatory agent is a polymer, the site of administration comprises a bone injury, osteophytes, osteoclasts, alveolar bone destruction, endochondral bone formation, or intra-membranous ossification and the bone growth or bone resorption is inhibited by at least 25% as compared to the bone growth or bone resorption in the absence of the anti-inflammatory agent.

80. The method of claim 76, wherein the anti-inflammatory agent is incorporated into the backbone of a polymer having repeating units selected from the group consisting of
—Y—C(═Y)—R¹-A-R¹—C(═Y)—Y—and
—Y—C(═Y)—R²-A-L-A-R³—C(═Y)—Y—
wherein each
each R¹, independently from one another, is at least one residue of a substituted or unsubstituted aromatic group;
each Y, independently from one another, comprises one or more O, S, NR⁷, wherein R⁷ comprises H, linear, branched or cyclic (C₁-C₄₀) alkyl, alkenyl, alkynyl or aryl, optionally substituted with an aliphatic residue, all of which may be substituted with O, N, S, P or halogen; and
A, independently from one another, is ester, amide, thioester, carbonate or thioamide and A is substituted on R¹ ortho to the Y-containing moiety and L comprises an organic linker selected from straight, branched or cyclic (C₁-C₅₀) alkyl, alkenyl, or alkynyl, or —C₂—C₅₀—(—CH₂—CH₂—O—)ₘ, —(CH₂—CH₂—CH₂—O—)ₘ or —CH₂—CHCH₃—O—)ₘ wherein m is about 2-50.

81. The method of claim 79, wherein the anti-inflammatory agent is enfenamic acid, aceclofenac, glucametacin, alminoprofen, carprofen, ximoprofen, salsalate, 3-amino-4-hydroxybutyric acid, ditazol, fepradinol, oxaceprol, zileuton, flufenamic acid, meclofenamic acid, mefenamic acid, niflumic acid, tolfenamic acid, amfenac, bromfenac, diclofenac sodium, etodolac, bromosaligenin, diflunisal, fendosal, gentisic acid, glycol salicylate, mesalamine, olsalazine, salicylamide o-acetic acid, salicylic acid, sulfasalazine, 5-chlorosalicylic acid or 5-trifluoromethylsalicylic acid.

82. The method of claim 80, wherein the anti-inflammatory agent is incorporated into the backbone of a polymer, R¹ is a phenyl or substituted phenyl group, Y is oxygen, A is an ester, L is (C₂-C₂₀)alkyl and the polymer has a molecular weight of at least 20,000 Daltons.

83. The method of claim 82, wherein the anti-inflammatory agent incorporated into the polymer backbone is glycol salicylate, salicylic acid, diflunisal, gentisic acid, salsalate, 5-chlorosalicylic acid or 5-trifluoromethylsalicylic acid.

84. The method of 79, wherein the bone growth or bone resorption is inhibited by at least 50% as compared to the bone growth or bone resorption in the absence of the anti-inflammatory agent.

85. The method of claim 76, wherein the monomer, oligomer or polymer is part of a device, implant or dressing.

86. The method of claim 76, wherein the anti-inflammatory agent is administered as a controlled release form.

87. The method of claim 82, wherein the anti-inflammatory agent incorporated into the backbone of a polymer is salicylic acid, salsalate or diflunisal.

88. The method of claim 87, wherein the polymer is part of a film, paste, gel, fiber, chip, microparticular or nanoparticular formulation, wherein the amount of the anti-inflammatory agent is sufficient to inhibit the bone growth or bone resorption by at least 25 percent as compared to the bone growth or bone resorption in the absence of the anti-inflammatory agent.

89. The method of claim 88, wherein the microparticular formulation comprises particles of about 0.5 micron to about 100 micron diameter or size, or the nanoparticular formulation comprises particles of about 0.5 nm to about 100 nm diameter or size.

90. The method of claim 87, wherein the site of administration comprises a bone injury resulting from bone breakage, implant, implant removal or infectious disease.

91. The method of claim 76, wherein at least 0.1 mg of the anti-inflammatory agent is released per day at the site.

92. The method of claim 76, wherein the administering causes a free concentration of the anti-inflammatory agent in tissue or extracellular fluid at the site of at least 0.1 mg/cm³.

93. The method of claim 76, wherein the concentration of the anti-inflammatory agent in tissue or extracellular fluid at the site is at least 0.1 mg/cm³ for at least 12 hours.

94. The method of claim 76, wherein the anti-inflammatory agent is incorporated into the backbone of a polymer and wherein the polymer also has an anti-inflammatory agent entrapped in its matrix.

95. The method of claim 76, wherein the anti-inflammatory agent is incorporated into the backbone of a polymer and wherein the polymer also has an anti-inflammatory agent appended to the backbone.
